(19) United States
(12) Patent Application Publication
(10) Pub. No.: US 2015/0218220 A1
(43) Pub. Date: Aug. 6, 2015

(54) AMATOXIN DERIVATIVES AND CELL-PERMEABLE CONJUGATES THEREOF AS INHIBITORS OF RNA POLYMERASE

(71) Applicants: Brian Alan MENDELSOHN, Newcastle, WA (US); Sung Ju MOON, (US); Agensys, Inc.., Santa Monica, CA (US)

(72) Inventors: Brian Alan Mendelsohn, Newcastle, WA (US); Sung Ju Moon, Santa Monica, CA (US)

(21) Appl. No.: 14/427,297

(22) PCT Filed: Sep. 12, 2013
(86) PCT No.: PCT/US2013/059536

§ 371 Date: Mar. 10, 2015

Related U.S. Application Data
(60) Provisional application No. 61/700,281, filed on Sep. 12, 2012.
(60) Provisional application No. 61/860,837, filed on Jul. 31, 2013.

(51) Int. Cl. C07K 7/64; C07K 19/00

(52) U.S. Cl. CPC C07K 7/64; C07K 19/00; A61K 38/00

(57) ABSTRACT

The present invention relates to analogs of alpha-amanitin, methods of inhibiting RNA polymerase with such compounds, conjugates comprising such compounds, compositions comprising such compounds and conjugates, and methods of treatment using such conjugates.

### CROSS-REFERENCE TO RELATED APPLICATIONS

This application claims priority to U.S. Provisional Application Nos. 61/700,281, filed Sep. 12, 2012, and 61/860,837, filed Jul. 31, 2013, the contents of each of which are incorporated by reference herein in their entireties.

### FIELD OF THE INVENTION

The present invention relates to chemical derivatives of compounds in the Amatoxin family, such as alpha-amanitin, to compositions comprising such derivatives, and to methods for using the same to modulate RNA polymerase activity. The present invention also contemplates use cell-permeable conjugates of the compounds, pharmaceutical compositions comprising the same, and methods of treating cancer, autoimmune diseases, infectious diseases, or other pathological conditions, with said conjugates.

### BACKGROUND OF THE INVENTION

Cancer is the second leading cause of human death next to coronary disease. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise.

Worldwide, several cancers stand out as the leading killers, including carcinomas of the lung, prostate, breast, colon, pancreas, ovary, and bladder. These and virtually all other carcinomas share a common lethal feature, the potential for metastasis. With few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure and/or physical debilitations following treatment. Furthermore, many cancer patients do experience a recurrence.

The amatoxins are rigid bicyclic peptides, comprised of eight amino acid units. These compounds are isolated from a variety of mushroom species (e.g., Amanita phalloides (also known as green death cap mushroom), Galerina marginata, Lepiota brunneo-incarnata) or are prepared synthetically. Different mushroom species contain varying amounts of different Amatoxin family members. A member of this family, alpha-amanitin, is known to be an extremely potent inhibitor of eukaryotic RNA polymerase II (EC2.7.7.6) and to a lesser degree, RNA polymerase III, thereby inhibiting transcription and protein biosynthesis. Wieland (1983) Int. J. Pept. Protein Res. 22(3):257-276. Alpha-amanitin binds non-covalently to RNA polymerase II and dissociates slowly, making enzyme recovery unlikely. Prolonged inhibition of transcription is thought to induce cellular apoptosis.

The use of antibody-drug conjugates (ADCs) for the local delivery of cytotoxic or cytostatic agents, including drugs that kill or inhibit tumor cells, allows targeted delivery of the drug moiety to tumors, and intracellular accumulation therein. Syrigos and Epenetos (1999) Anticancer Res. 19:605-614; Niculescu-Duvaz and Springer (1997) Adv. Drug Delivery Rev. 26:151-172; U.S. Pat. No. 4,975,278; Baldwin et al. (1986) Lancet (Mar. 15, 1986):603-05; Thorpe (1985) “Antibody Carriers of Cytotoxic Agents in Cancer Therapy: A Review,” in Monoclonal Antibodies '84: Biological and Clinical Applications, A. Pinchera et al. (eds.), pp. 475-506. This type of delivery mechanism helps to minimize toxicity to normal cells that may occur from systemic administration of unconjugated drug agents. The toxins may cause their cytotoxic and cytostatic effects through a variety of mechanisms including tubulin binding, DNA binding, or topoisomerase inhibition. Both polyclonal antibodies and monoclonal antibodies have been reported as useful in these strategies. Rowland et al. (1986) Cancer Immunol. Immunother. 21:183-87. Toxins used in antibody-toxin conjugates include radioisotopes, bacterial toxins such as diphtheria toxin, plant toxins such as ricin, fungal toxins such as amatoxins (WO2010/115629, WO2012/041504 or WO2012/119787), and small molecule toxins such as geldanamycin (Mandler et al. (2000) J. Natl. Cancer Inst. 92(19):1573-1581; Mandler et al. (2000) Bioorg. Med. Chem. Lett. 10:1025-1028; Mandler et al. (2002) Bioconjugate Chem. 13:786-791), maytansinoids (EP 1391213; Liu et al. (1996) Proc. Natl. Acad. Sci. USA 93:8618-8623), calicheamicin (Lode et al. (1998) Cancer Res. 58:2928; Hinman et al. (1993) Cancer Res. 53:3336-3342), daunomycin, doxorubicin, methotrexate, and vindesine (Rowland et al. (1986), supra).

Several antibody-drug conjugates have shown promising results against cancer in clinical trials, including: 1) ZEVALIN® (ibritumomab tiuxetan, Biogen/Idec), an antibody-radioisotope conjugate composed of a murine IgG1 kappa monoclonal antibody (directed against the CD20 antigen found on the surface of normal and malignant B lymphocytes) connected with an ¹¹¹In or ⁹⁰Y radioisotope via a thiourea linker-chelator; 2) MYLOTARG® (gemtuzumab ozogamicin, Pfizer), an antibody drug conjugate composed of a hu CD33 antibody linked to calicheamicin that was approved in 2000 for the treatment of acute myeloid leukemia by injection, but discontinued in 2010; 3) cantuzumab mertansine (Immunogen, Inc.), an antibody drug conjugate composed of the huC242 antibody linked via a disulfide linker, N-succinimidyl 4-(2-pyridyldithio)pentanoate (SPP), to the maytansinoid drug moiety, DM1, that is advancing in human trials for the treatment of cancers that express CanAg, such as colon, pancreatic, gastric, and others; and MLN2704 (Millennium Pharm., BZL Biologics, Immunogen Inc.), an antibody drug conjugate composed of the prostate specific membrane antigen (PSMA) monoclonal antibody linked to the maytansinoid drug moiety, DM1, that is under development for the potential treatment of prostate tumors.

There remains a need for potent RNA polymerase inhibitors and for cell-permeable conjugates of such inhibitors with desirable pharmaceutical properties. Conjugates of certain amanitin derivatives have been found in the context of this invention to have RNA polymerase modulating activity.

### SUMMARY OF THE INVENTION

In one aspect, the present invention provides for a compound of Formula (I):

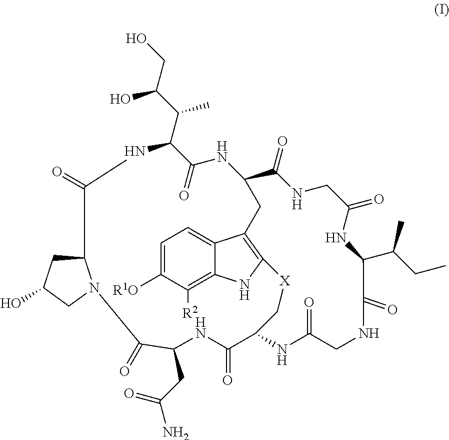

wherein:

### X is S, SO, or SO₂;

(a) R¹ is H and R² is a chemical moiety of Formula (A):

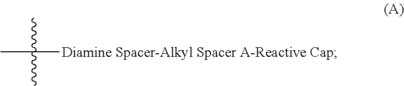

wherein
the diamine spacer is —NR^{x}—(C₂₋₂₀alkylene or C₂₋₂₀alkenylene)-NR^{y}—,
   wherein the nitrogen of the —NR^{y}— group is attached to the alkyl spacer;
   one carbon unit within the C₂₋₂₀alkylene or C₂₋₂₀alkenylene is optionally replaced with an NR^{z};
   R^{x} is H or C₁₋₄alkyl, or
   R^{x} taken together with a carbon or R^{z} within the alkylene or alkenylene forms a 3-8-membered heterocycloalkyl ring,
   R^{y} is H or C₁₋₄alkyl,
   or R^{x} and R^{y} taken together form a C₂₋₄alkylene; and
   R^{x} is H or C₁₋₄alkyl;
the alkyl spacer A is absent, or is —C(O)C₁₋₂₀alkylene- or —C(O)C₂₋₂₀alkenylene-, wherein the carbonyl is attached to the nitrogen of the NR^{y} group in the diamine spacer and the alkylene or alkenylene is attached to the reactive cap, and wherein one or more carbon units within the alkylene or alkenylene is optionally replaced with C₃₋₇cycloalkylene, —C(O)NH—, —NHC(O)—, —C(O)O—, —OC(O)—, —C(O)—, —NH—, or —O—; and
the reactive cap is —N₃, —C≡CH, —CO₂H, —ONH₂,

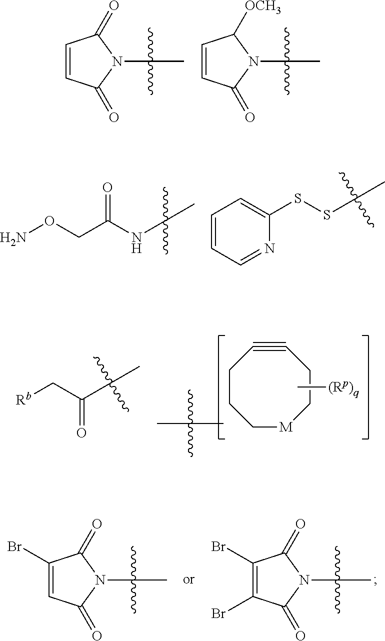

wherein R^{b} is a leaving group;
M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and
   R^{m} is phenyl or a bond;
      or
      (b) R² is H and R¹ is a chemical moiety of Formula (B):

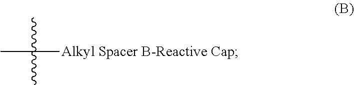

wherein the reactive cap is defined as above; and
alkyl spacer B is absent, or is —C₁₋₂₀alkylene- or —C₂₋₂₀alkenylene-, wherein one or more carbon units within the alkylene or alkenylene is replaced with C₃₋₇cycloalkylene, —C(O)NH—, —NHC(O)—, —C(O)O—, —OC(O)—, —C(O)—, —NH—, or —O—;
   or a salt thereof.

In another aspect, the present invention contemplates cell-permeable conjugates of the amanitin derivatives described herein with a cellular transport facilitator. In this context, the invention relates to conjugates of Formula (IA):

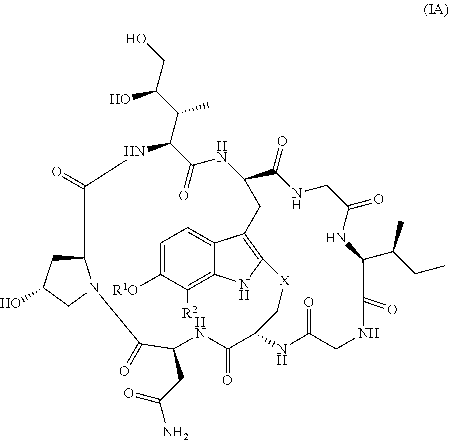

wherein:

### X is S, SO, or SO₂;

(a) R¹ is H and R² is a chemical moiety of Formula (A-1):

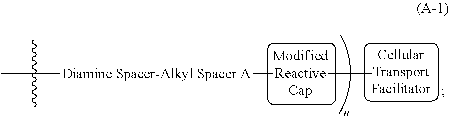

wherein
the diamine spacer and alkyl spacer A are defined as for Formula (I);
the modified reactive cap is —C(O)NH—,

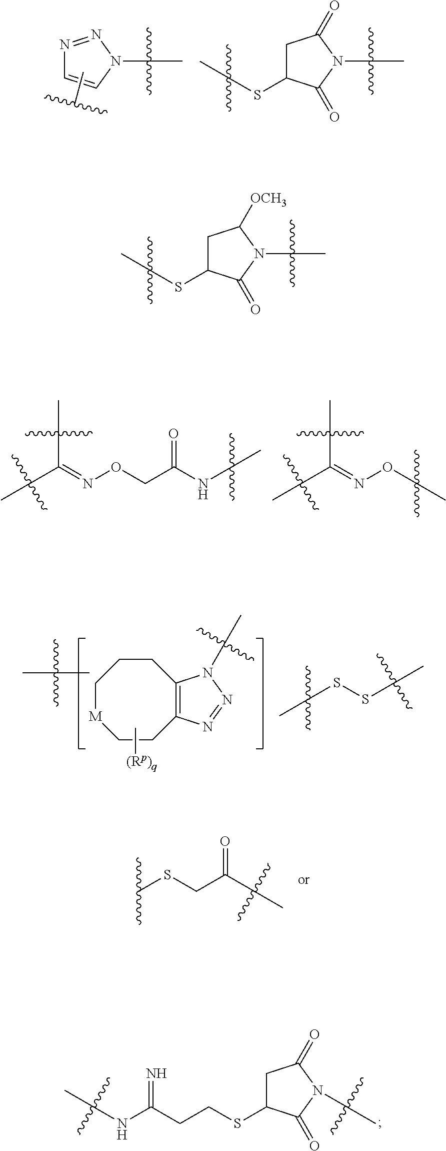

wherein M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond;
the cellular transport facilitator is an antibody, a peptide, a cationic polymer, or a liposome;
and
n is an integer from 1 to 20;
   or
   (b) R₂ is H and R₁ is a chemical moiety of formula (B-1):

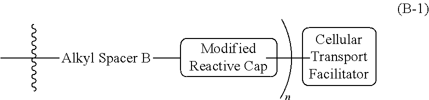

wherein alkyl spacer B is defined as for Formula (I); and
the modified reactive cap, cellular transport facilitator, and n are as defined for Formula (A-1).

In another aspect, the present invention provides for a compound of Formula (II):

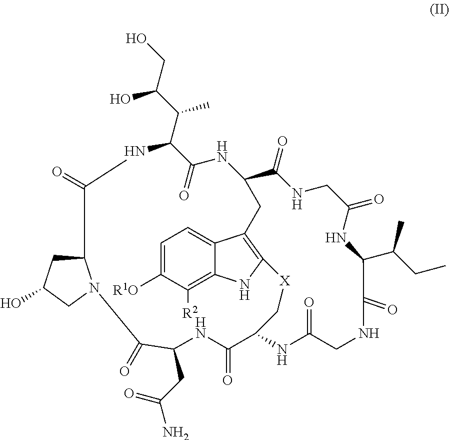

wherein:

### X is S, SO, or SO₂;

### (a) R¹ is H and R² is

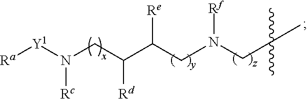 [0042] wherein x is 0, 1, or 2;
y is 0 or 1;
z is 0 or 1;
R^{c} is H or methyl;
R^{d} is H;
R^{e} is H;
R^{f} is H or methyl;
or R^{d} and R^{f} taken together form a bond, —CH₂—, or —CH₂CH₂⁻;
or R^{e} and R^{f} taken together form a bond;
or R^{c} and R^{f} taken together form —CH₂CH₂—;
Y¹ is absent, or is —C(O)C₁₋₁₆alkylene or —C(O)C₂₋₁₆alkenylene in which one or more carbon units are optionally replaced with C₃₋₇cycloalkylene, —C(O)NH—, —NHC(O)—, —C(O)O—, —OC(O)—, —C(O)—, NH, or O;
R^{a} is —N₃, —C≡CH, —CO₂H, —ONH₂,

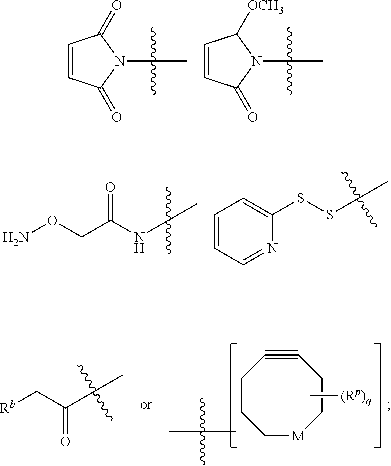

wherein R^{b} is a leaving group;
M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond;
   or

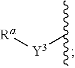

(b) R² is H and R¹ is wherein Y³ is absent or is C₁₋₁₆alkylene or C₂₋₁₆alkenylene in which one or more carbon units are replaced with C₃₋₇cycloalkylene, —C(O)NH—, —NHC(O)—, —C(O)O—, —OC(O)—, —C(O)—, NH, or O; and
R^{a} is defined as above within the definition of R²;
   or a pharmaceutically acceptable salt thereof.

In another aspect, the present invention contemplates cell-permeable conjugates of the amanitin derivatives of Formula (II) with a cellular transport facilitator. In this context, the invention relates to conjugates of Formula (IIA):

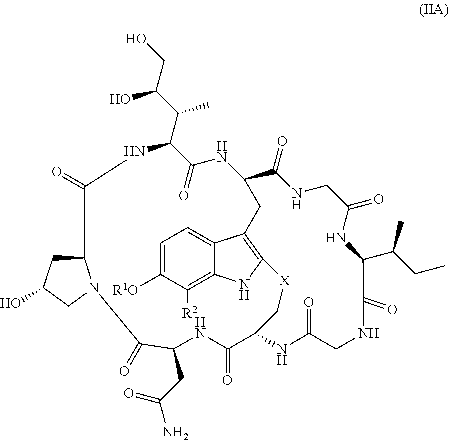

wherein:

### X is S, SO, or SO₂;

### (a) R¹ is H and R² is

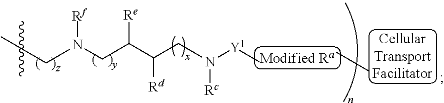 [0061] wherein x, y, z, R^{c}, R^{d}, R^{e}, R^{f}, and Y¹ are defined as for Formula (II); and
Modified R^{a} is —C(O)NH—,

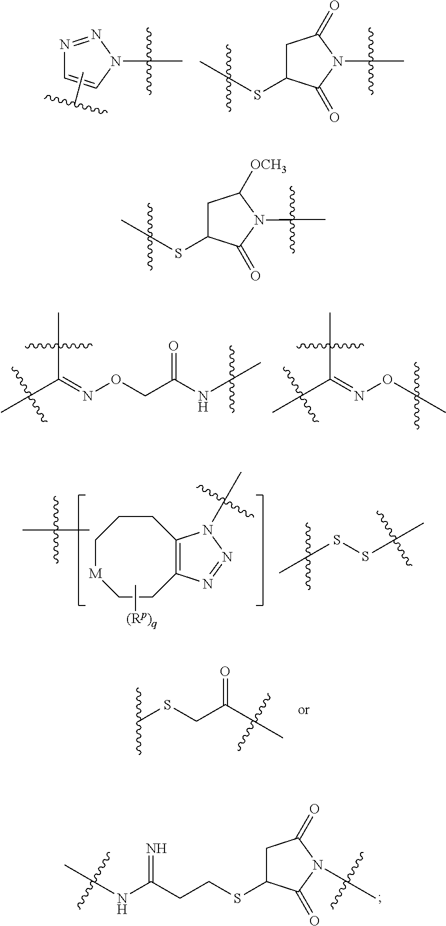

wherein M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond;
n is an integer from 1 to 20; and
the cellular transport facilitator is an antibody, a peptide, a cationic polymer, or a liposome;
   or

### (b) R² is H and R¹ is

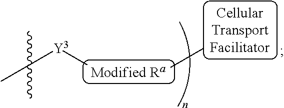 [0069] wherein Y³ is defined as for Formula (II); and
modified R^{a}, n, and cellular transport facilitator are defined as above within the definition of R².

In a further aspect, the invention relates to a composition comprising an effective amount of at least one compound of Formula (I), Formula (IA), Formula (II), or Formula (IIA), or a pharmaceutically acceptable salt thereof. In a further aspect, the invention relates to a pharmaceutical composition comprising an effective amount of at least one compound of Formula (I), Formula (IA), Formula (II), or Formula (IIA), or a pharmaceutically acceptable salt thereof. Such pharmaceutical compositions may further comprise a pharmaceutically acceptable carrier.

In yet another aspect, the invention relates to a method of modulating RNA polymerase, comprising contacting RNA polymerase with an effective amount of at least one compound of Formula (I), Formula (IA), Formula (II), or Formula (IIA), or a pharmaceutically acceptable salt thereof.

In yet another aspect, the invention relates to a method of preparing a conjugate of a compound of Formula (I) or Formula (II) with a cellular transport facilitator, such as a peptide, an antibody, a cationic polymer, or a liposome, and methods of treatment using such conjugates (compounds of Formula (IA) and (IIA)) to treat cancer, autoimmune diseases, infectious diseases, or other pathological conditions.

### BRIEF DESCRIPTION OF THE FIGURES

FIG. 1 shows the in vitro cytotoxicity data for ADCs of Examples 1 and 28 with Herceptin and IgG1 in HCC-1954 cells.

FIG. 2 shows the in vitro cytotoxicity data for ADCs of Examples 1 and 28 with Herceptin and IgG1 in MDA-MB-468 cells.

FIG. 3 shows the in vitro cytotoxicity data for ADCs of Examples 1, 3, and 4 with Herceptin in PC3 cells.

FIG. 4 shows the in vitro cytotoxicity data for ADCs of Examples 3 and 4 with Herceptin in HCC-1954 cells.

FIG. 5 shows the in vitro cytotoxicity data for ADCs of Examples 2, 27, and 76 with Herceptin in HCC-1954 cells.

FIG. 6 shows the in vitro cytotoxicity data for ADCs of Examples 2, 27, and 76 with Herceptin in MDA-MB-468 cells, as compared to paclitaxel.

FIG. 7 shows the in vitro cytotoxicity data for ADCs of Examples 5, 6, and 7 with Herceptin in HCC-1954 cells.

FIG. 8 shows the in vitro cytotoxicity data for ADCs of Examples 2, 5, 6, and 7 with Herceptin in PC3 cells.

FIG. 9 shows the in vitro cytotoxicity data for ADCs of Examples 8 and 9 with Herceptin in HCC-1954 cells, as compared to paclitaxel.

FIG. 10 shows the in vitro cytotoxicity data for ADCs of Examples 8 and 9 with Herceptin in PC3 cells, as compared to paclitaxel.

FIG. 11 shows the in vitro cytotoxicity data for ADCs of Examples 26, 31, and 32 with Herceptin in HCC-1954 cells.

FIG. 12 shows the in vitro cytotoxicity data for ADCs of Examples 26, 27, 31, and 32 with Herceptin in PC3 cells.

FIG. 13 shows the in vitro cytotoxicity data for ADCs of Examples 33 and 34 with Herceptin in HCC-1954 cells.

FIG. 14 shows the in vitro cytotoxicity data for ADCs of Examples 28, 33, and 34 with Herceptin in PC3 cells.

FIG. 15 shows the in vitro cytotoxicity data for ADCs of Examples 35, 36, and 37 with Herceptin in HCC-1954 cells.

FIG. 16 shows the in vitro cytotoxicity data for ADCs of Examples 35, 36, and 37 with Herceptin in PC3 cells.

FIG. 17 shows the in vitro cytotoxicity data for ADCs of Example 29 with Herceptin and IgG1, and Example 30 with Herceptin in HCC-1954 cells.

FIG. 18 shows the in vitro cytotoxicity data for ADCs of Example 29 with Herceptin and IgG1, and Example 30 with Herceptin in MDA-MB-468 cells.

FIG. 19 shows the in vitro cytotoxicity data for ADCs of Examples 39, 72, and 77 with Herceptin in HCC-1954 cells.

FIG. 20 shows the in vitro cytotoxicity data for ADCs of Examples 39, 72, and 77 with Herceptin in PC3 cells.

FIG. 21 shows the in vitro cytotoxicity data for ADCs of Examples 38, 40a, and 71 with Herceptin in HCC-1954 cells.

FIG. 22 shows the in vitro cytotoxicity data for ADCs of Examples 38, 40a, and 71 with Herceptin in PC3 cells.

FIG. 23 shows the effect of in vivo dosing with ADCs of Examples 1 (5 mg/kg), 2 (5 mg/kg), 27 (5 mg/kg), 28 (1 and 2.5 mg/kg), and 76 (1, 2.5, and 5 mg/kg) on mean tumor volume over time.

FIG. 24 shows the effect of in vivo dosing with ADCs of Examples 1, 2, and 76 at 5 mg/kg on mean tumor volume over time.

FIG. 25 shows the in vitro cytotoxicity data for an ADC of Example 40b with Herceptin in HCC-1954 cells.

FIG. 26 shows the in vitro cytotoxicity of an ADC of Example 40b with Herceptin compared to the IgG1 control isotype antibody on PC3 cells.

FIG. 27 shows the in vitro cytotoxicity of ADCs of Examples 81, 85 and 86 with Herceptin on HCC-1954 cells.

FIG. 28 shows the in vitro cytotoxicity of ADCs of Examples 81, 85, and 86 with Herceptin on PC3 cells.

FIG. 29 shows the effect of in vivo dosing of ADCs of Examples 27, 29, 30, 38, 39, 40a, 40b, 71, 72, 76, and 77, and a mixture of Examples 1 and 76 at 5 mg/kg on mean tumor volume over time.

FIG. 30 shows the effect of in vivo dosing of ADCs of Examples 1, 2, 27, 76, 39, and 40b at 5 mg/kg on mean tumor volume over time.

FIG. 31 shows the effect of in vivo dosing of ADCs of Examples 1, 3-9, 26, 28, and 31-37 at 5 mg/kg on mean tumor volume over time.

FIG. 32 shows the effect of in vivo dosing of an ADC of Example 76 at twice weekly doses of 0.25, 0.5, 1, and 2 mg/kg on mean tumor volume over time.

FIG. 33 shows the effect of in vivo dosing of Herceptin conjugates of Examples 1, 2, 34, and 76 at a dose of 5 mg/kg as compared to control Herceptin and IgG1 conjugates.

FIG. 34 shows the effect of in vivo dosing of a conjugate of Example 76 with anti-ENPP3 at 5 mg/kg on mean tumor volume over time.

FIG. 35 shows the effect of in vivo dosing of anti-ENPP3 conjugates of Examples 1, 2, 27, and 76 at 3 and 5 mg/kg on mean tumor volume over time.

FIG. 36 shows the effect of in vivo dosing of anti-ENPP3 conjugates of Examples 1, 2, 27, and 76 at 3 and 5 mg/kg on mean tumor volume over time.

FIG. 37 shows the effect of in vivo dosing of Herceptin conjugates of Examples 26 and 76 at 5 and 1, 5, 10, 20, and 30 mg/kg, respectively, on mean tumor volume over time.

FIG. 38 shows the effect of in vivo dosing of Herceptin conjugates of Examples 2, 81, 85, and 86 at 5 mg/kg on mean tumor volume over time.

FIG. 39 shows the in vitro cytotoxicity of ADCs of Example 76 with Herceptin on HCC-1954 cells.

FIG. 40 shows the in vitro cytotoxicity of ADCs of Examples 76 with Herceptin on PC3 cells.

FIG. 41 shows the in vitro cytotoxicity of ADCs of Example 76 with anti-CD33 and anti-CD71 on Hel92.1.7 cells.

FIG. 42 shows the in vitro cytotoxicity of ADCs of Example 76 with anti-CD33 and anti-CD71 on MOLM-13 cells.

FIG. 43 shows the in vitro cytotoxicity of ADCs of Example 76 with anti-CD33 and anti-CD71 on RS4-11 cells.

FIG. 44 shows the in vitro cytotoxicity of ADCs of Examples 1, 2, 27, and 76 with anti-FLT3 on MOLM-13 cells.

FIG. 45 shows the in vitro cytotoxicity of ADCs of Example 76 with anti-FLT3 on MOLM-13 cells.

FIG. 46 shows the in vitro cytotoxicity of ADCs of Examples 1, 2, 27, and 76 with anti-FLT3 on EOL-1 cells.

FIG. 47 shows the in vitro cytotoxicity of ADCs of Example 76 with anti-FLT3 on EOL-1 cells.

FIG. 48 shows the in vitro cytotoxicity of ADCs of Examples 1, 2, 27, and 76 with anti-FLT3 on Hel92.1.7 cells.

FIG. 49 shows the in vitro cytotoxicity of ADCs of Example 76 with anti-FLT3 on Hel92.1.7 cells.

FIG. 50 shows the in vitro cytotoxicity of ADCs of Example 76 with anti-CD33 on MOLM-13 cells.

FIG. 51 shows the in vitro cytotoxicity of ADCs of Example 76 with anti-CD33 on Pfeiffer cells.

FIG. 52 shows the effect of in vivo dosing of Anti-CD71 conjugates of Examples 1, 2, 27, and 76 at 2 mg/kg on mean tumor volume over time.

FIG. 53 shows the effect of in vivo dosing of Anti-CD33 conjugates of Examples 1, 2, 27, and 76 at 1 mg/kg on mean tumor volume over time.

FIG. 54 shows the effect of in vivo dosing of Anti-FLT3 conjugates of Examples 1, 2, 27, and 76 at 2 mg/kg on mean tumor volume over time.

FIG. 55 shows the effect of in vivo dosing of Anti-FLT3 conjugates of Examples 1, 2, 27, 76, and Prior Art ADC 2 at 2 mg/kg on mean tumor volume over time.

FIG. 56 shows an in vitro stability assay of ADC Herceptin-Prior Art ADC.

FIG. 57 shows an in vitro stability assay of ADC anti-PSCA-Example 1.

FIG. 58 shows an in vitro stability assay of ADC Herceptin-Example 30.

FIG. 59 shows an in vitro stability assay of ADC Herceptin-Example 71.

FIG. 60 shows an in vitro stability assay of ADC anti-PSCA-Example 76.

FIG. 61 shows an in vitro stability assay of ADC Herceptin-Example 27.

### DETAILED DESCRIPTION OF THE INVENTION

For the sake of brevity, the disclosures of the publications cited in this specification, including patents, are herein incorporated by reference.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The term “alkyl” refers to a straight- or branched-chain alkyl group having from 1 to 20 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkenyl” refers to a straight- or branched-chain alkenyl group having from 2 to 20 carbon atoms in the chain. Examples of alkenyl groups include vinyl (ethenyl), propenyl, isopropenyl, butenyl, tert-butylenyl, hexenyl, and groups that in light of the ordinary skill in the art and the teachings provided herein would be considered equivalent to any one of the foregoing examples.

The term “alkylene” refers to a straight- or branched-chain divalent alkyl group, where alkyl is defined above. The divalent positions may be on the same or different carbons within the alkyl chain. Examples of alkylene include methylene, ethylene, propylene, and isopropylene and groups that in light of the ordinary skill in the art and the teachings provided herein would be considered equivalent to any one of the foregoing examples.

The term “alkenylene” refers to a straight- or branched-chain divalent alkenyl group, where alkenyl is defined above. The divalent positions may be on the same or different carbons within the alkenyl chain. Examples of alkenylene include ethenylene, propenylene, isopropenylene, butenylene, and groups that in light of the ordinary skill in the art and the teachings provided herein would be considered equivalent to any one of the foregoing examples.

A “carbon unit” of an alkylene or alkenylene refers to one carbon within the chain along with one or more of its attached hydrogen atoms. Replacement of a carbon unit with another moiety may not include replacement of all of that carbon's attached hydrogen atoms if doing so would generate a valence-disallowed structure.

The term “amino acid” refers any naturally occurring or synthetic amino acid. Exemplary amino acids include: arginine, histidine, lysine, aspartic acid, glutamic acid, serine, threonine, asparagine, glutamine, cysteine, selenocysteine, glycine, sarcosine, proline, alanine, isoleucine, leucine, norleucine, methionine, phenylalanine, tryptophan, tyrosine, valine, para-aminobenzoic acid, meta-aminobenzoic acid, and ortho-aminobenzoic acid.

The term “cellular transport facilitator” refers to any one of a variety of molecules (including macromolecules) that facilitates uptake of a covalently linked molecule across cell membranes. Among the solutions proposed to facilitate cellular uptake have been the use of transporter moieties such as cationic (i.e., positively charged) polymers, peptides and antibody sequences, including polylysine, polyarginine, Antennapedia-derived peptides, HIV Tat-derived peptides, and the like. (See, for example, U.S. Pat. Nos. and Publications Nos. 4,847,240, 5,652,122, 5,670,617, 5,674,980, 5,747,641, 5,804,604, 5,888,762, 6,316,003, 6,593,292, US2003/0104622, US2003/0199677 and US2003/0206900, all of which are hereby incorporated by reference in their entirety.) A conjugate between a compound of Formula (I) or Formula (II) and a suitable cellular transport facilitator is generally selected based on certain stability, tolerability, and bioavailability characteristics. Such conjugates may be formulated as pharmaceutical compositions and administered to subjects in need of treatment in an effective amount.

The term “cycloalkyl” refers to a monocyclic, or fused, bridged, or spiro polycyclic ring structure that is saturated and has from 3 to 12 carbon ring atoms. Illustrative entities include cyclopropyl, cyclobutyl, cyclopentyl, cyclohexyl, cycloheptyl, cyclooctyl, and bicyclo[3.1.0]hexane, and groups that in light of the ordinary skill in the art and the teachings provided herein would be considered equivalent to any one of the foregoing examples.

The term “cycloalkylene” refers to a divalent cycloalkyl group, where cycloalkyl is defined above. The divalent positions may be on the same or different carbons within the ring structure. Examples of cycloalkylene include cyclopropylene, cyclobutylene, cyclopentylene, and cyclohexylene, and groups that in light of the ordinary skill in the art and the teachings provided herein would be considered equivalent to any one of the foregoing examples.

The term “heterocycloalkyl” refers to a monocyclic, or fused, bridged, or spiro polycyclic ring structure that is saturated and has from 3 to 12 ring atoms per ring structure selected from carbon atoms and up to three heteroatoms selected from nitrogen oxygen, and sulfur. The ring structure may optionally contain up to two oxo groups on carbon, nitrogen, or sulfur ring members. Illustrative entities, in the form of properly bonded moieties, include:

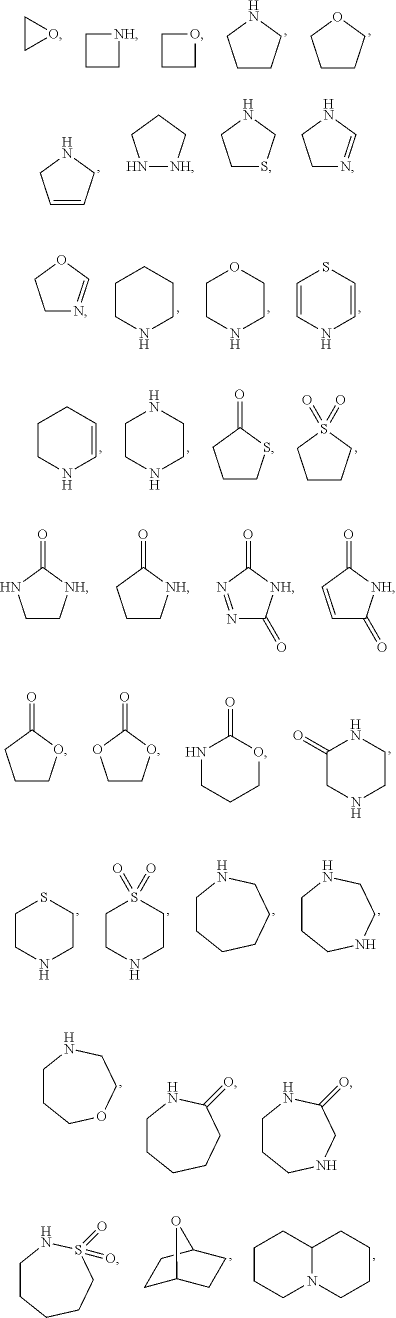

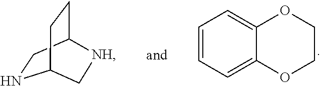 [SMILES: C1CC2CNC1CN2 ; "and" ; c1ccc2c(c1)OCCO2 ; "."]

The term “halogen” represents chlorine, fluorine, bromine, or iodine. The term “halo” represents chloro, fluoro, bromo, or iodo.

The term “leaving group” refers to a molecular fragment that is removed from a chemical compound with a pair of electrons during a nucleophilic bond cleavage reaction. Exemplary leaving groups are listed in Smith, March. Advanced Organic Chemistry 6^{th} ed. (501-502), including dinitrogen, dialkyl ethers, perfluroakylsulfonates (e.g., triflate), tosylates, mesylates, iodide, bromide, water, alcohols, chloride, nitrate, phosphate, other inorganic esters, thiolates, amines, ammonia, fluoride, carboxylates, phenoxides, hydroxide, alkoxides, and amides. Particular exemplary leaving groups are iodo, chloro, bromo, fluoro, methanesulfonate (mesylate), p-tolylsulfonate (tosylate), tetraalkylammonium, or phosphate.

The term “modified reactive cap” refers to the structure that remains of the reactive cap once the reactive cap reacts with a cellular transport facilitator to form a covalent bond with the facilitator, or with a linker moiety to form a covalent bond with the linker moiety.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valence-allowed position on the system.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of a general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃). For example, the term Cₙ₋ₘalkyl refers to an alkyl chain, as defined herein, with a total number N of carbon members in the chain that satisfies n≦N≦m, with m>n. Analogously, the term Cₙ₋ₘcycloalkylene refers to a divalent cycloalkyl ring with n to m carbon ring members.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In certain embodiments of Formulas (I) and (IA), X is S. In other embodiments, X is SO. In still other embodiments, X is SO₂.

In certain embodiments of Formulas (I) and (IA), R¹ is H and R² is a chemical moiety of Formula (A) and Formula (A-1).

In certain embodiments, the diamine spacer in Formula (A) and Formula (A-1) is —NR^{x}—(C₂₋₂₀alkylene)-NR^{y}—, wherein one carbon unit within the C₂₋₂₀alkylene is optionally replaced with an NR^{z}. In other embodiments, the diamine spacer is —NR^{x}—(C₂₋₁₀alkylene)-NR^{y}—, wherein one carbon unit within the C₂₋₁₀alkylene is optionally replaced with an NR^{z}. In still other embodiments, the diamine spacer is —NR^{x}—(C₂₋₅alkylene)-NR^{y}—, wherein one carbon unit within the C₂₋₅alkylene is optionally replaced with an NR^{z}. In still other embodiments, the diamine spacer is methyl(2-(methylamino)ethyl)amino, methyl(2-(methylamino)propyl)amino, methyl(2-(methylamino)butyl)amino, or piperazinyl, or is an aziridinyl, azetidinyl, pyrrolidinyl, piperidinyl, piperazinyl, or azepinyl, substituted with —(C₀₋₃alkylene)NH—. In still other embodiments, the diamine spacer is methyl(2-(methylamino)ethyl)amino, methyl(2-(methylamino)butyl)amino, methyl(4-(methylamino)butyl)amino, 2-(2-aminoethyl)-aziridin-1-yl, 3-aminomethyl-azetidin-1-yl, 3-aminomethyl-pyrrolidin-1-yl, 3-(2-aminoethyl)-pyrrolidin-1-yl, 4-amino-piperidin-1-yl, 4-(2-aminoethyl)-piperidin-1-yl, piperazin-1-yl, or 4-(2-aminoethyl)-piperazin-1-yl.

In certain embodiments, R^{x} is H or methyl. In other embodiments, R^{x} is H. In still other embodiments, R^{x} is taken together with R^{z} or with a carbon within the alkylene or alkenylene to form a 3-6-membered heterocycloalkyl ring. In still other embodiments, R^{x} is taken together with a carbon within the alkylene or alkenylene to form an aziridine, azetidine, pyrrolidine, or piperidine ring. In still other embodiments, R^{x} is taken together with R^{z} to form a piperazine ring.

In certain embodiments, R^{y} is H or methyl. In other embodiments, R^{x} and R^{y} are taken together to form ethylene (—CH₂CH₂—).

In some embodiments, R^{z} is H or methyl.

In some embodiments of Formulas (I) and (IA), alkyl spacer A in Formula (A) and Formula (A-1) is absent. In other embodiments, alkyl spacer A is —C(O)C₁₋₂₀alkylene-, wherein one or more carbon units within the alkylene is optionally replaced with C₃₋₇cycloalkylene, —C(O)NH—, —NHC(O)—, —C(O)O—, —OC(O)—, —C(O)—, NH, or O. In other embodiments, alkyl spacer A is —C(O)C₁₋₁₃alkylene-, wherein one or more carbon units within the alkylene is optionally replaced with C₃₋₇cycloalkylene, —C(O)NH—, —NHC(O)—, —OC(O)—, or O. In other embodiments, alkyl spacer A is absent or is —C(O)methylene-, —C(O)ethylene-, —C(O)propylene-, —C(O)pentylene-, —C(O)pentyl-NHC(O)-pentyl-, —C(O)cyclohexyl-methyl-, —C(O)pentyl-OC(O)-cyclohexylmethyl-, —C(O)pentyl-NHC(O)-cyclohexyl-methyl-, or —C(O)CH₂—(OCH₂CH₂)₄—.

In certain embodiments of Formula (I), the reactive cap in Formulas (A) or (B) is —N₃, —C≡CH, —CO₂H, —ONH₂,

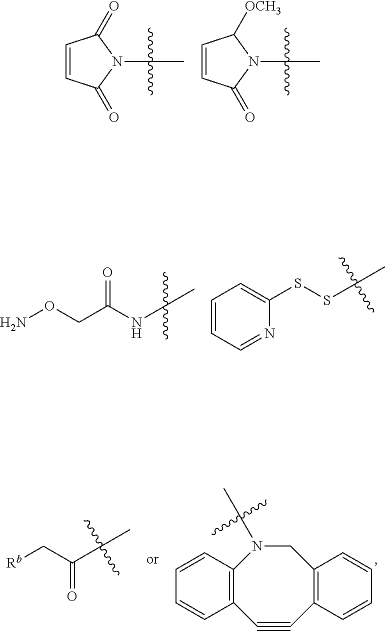

wherein R^{b} is a leaving group. In other embodiments, the reactive cap in Formulas (A) or (B) is —CO₂H, or is

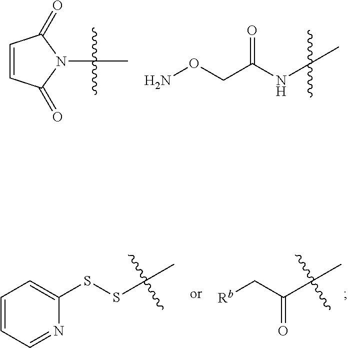

wherein R^{b} is a leaving group. In certain embodiments, R^{b} is iodo, chloro, bromo, or para-toluenesulfonate. In certain embodiments, R^{b} is chloro, bromo, or para-toluenesulfonate. In other embodiments, R^{b} is iodo or bromo. In other embodiments, R^{b} is chloro or bromo. In other embodiments, the reactive cap is

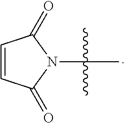 [SMILES: *N1C(=O)C=CC1=O]

In still other embodiments, the reactive cap is —N₃ or —C≡CH. In other embodiments, the reactive cap is —ONH₂. In still other embodiments, the reactive cap is:

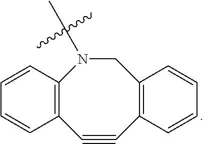 [SMILES: *N1Cc2ccccc2C#Cc2ccccc21]

In still other embodiments, the reactive cap is:

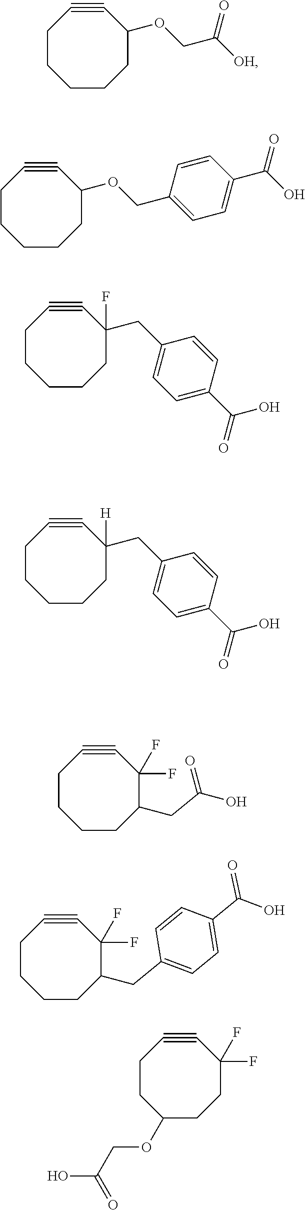

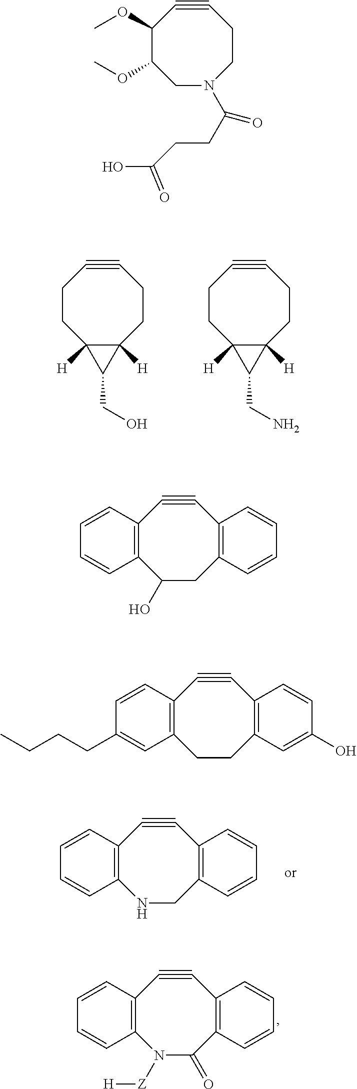

wherein Z is a bond, —C₁₋₄alkylene-O—, or —C₁₋₄alkylene-NH—.

In certain embodiments of Formula (IA), the modified reactive cap is —C(O)NH— or is:

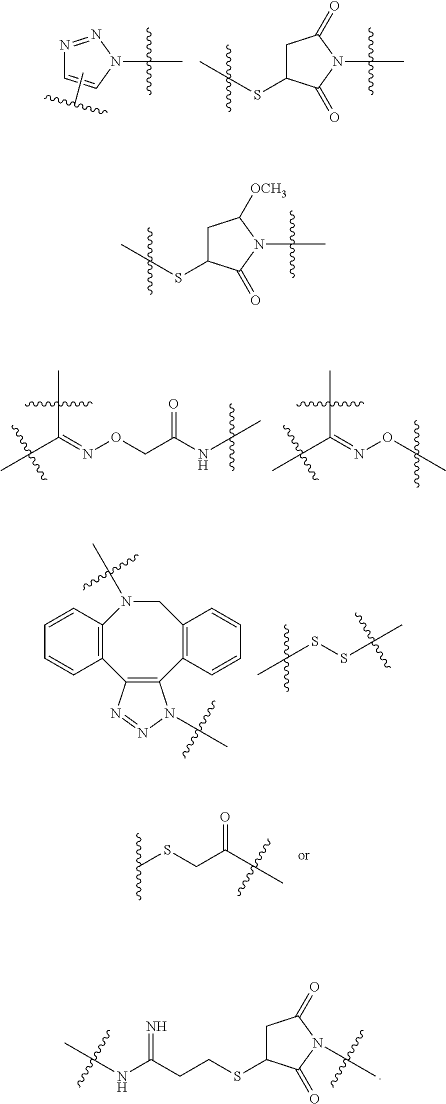

In other embodiments, the modified reactive cap is —C(O)NH—, or is:

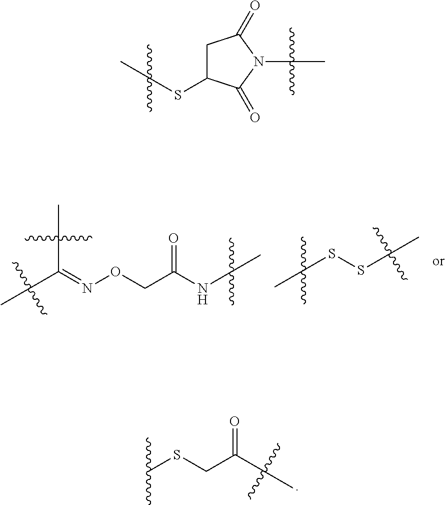

In other embodiments, the modified reactive cap is

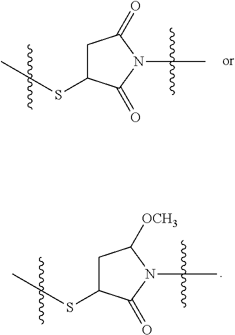 [SMILES: *SC1CC(=O)N(*)C1=O ; *SC1CC(OC)N(*)C1=O]

In still other embodiments, the modified reactive cap is

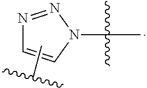

In still other embodiments, the modified reactive cap is

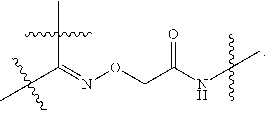 [SMILES: *NC(=O)CON=C(*)*]

In still other embodiments, the modified reactive cap is

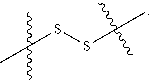 [SMILES: *SS*]

In still other embodiments, the modified reactive cap is

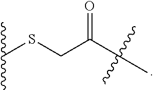 [SMILES: *SCC(*)=O]

In still other embodiments, the modified reactive cap is

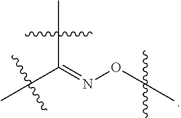 [SMILES: *ON=C(*)*]

In still other embodiments, the modified reactive cap is

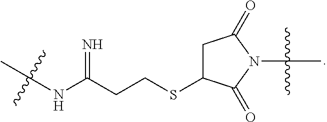 [SMILES: *NC(=N)CCSC1CC(=O)N(*)C1=O]

In still other embodiments, the reactive cap is

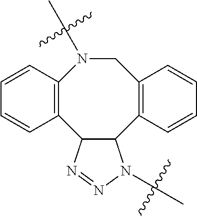 [SMILES: *N1Cc2ccccc2C2C(N=NN2*)c2ccccc21]

or a triazole regioisomer thereof. In still other embodiments, the reactive cap is:

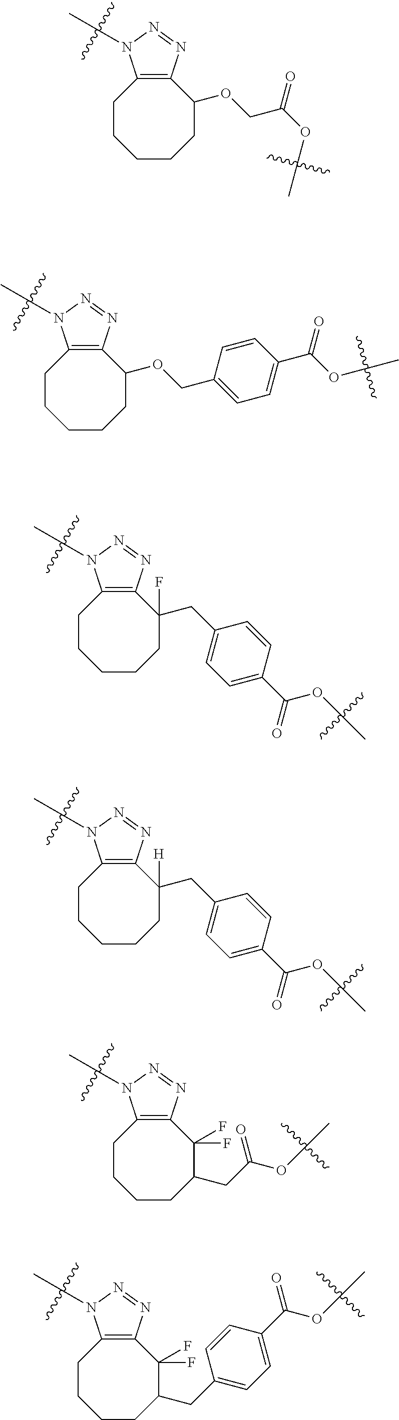

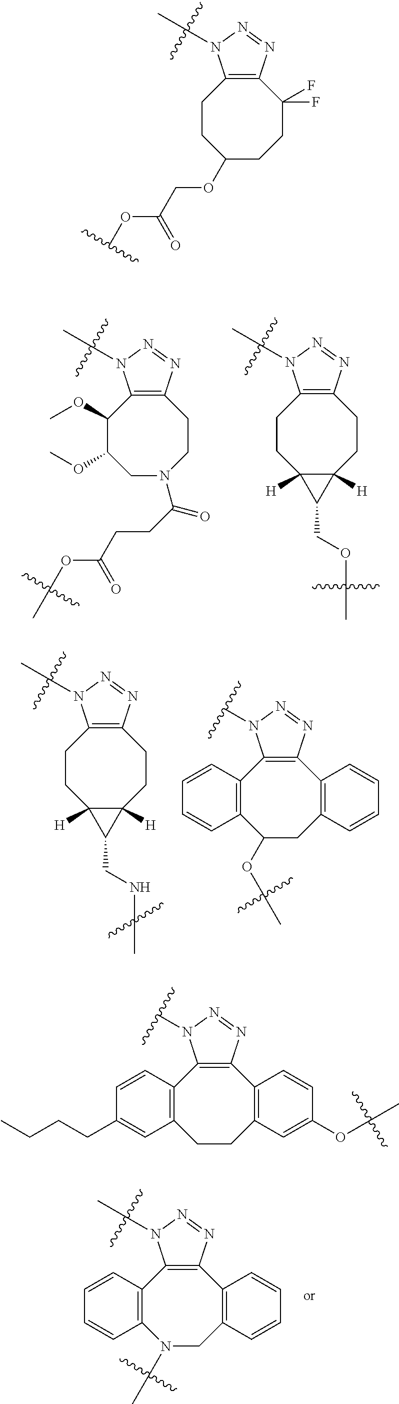

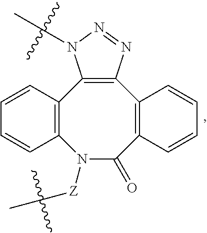

where Z is as defined above. For modified reactive caps containing a triazole ring, one of ordinary skill will recognize that the product may comprise a linkage to the cellular transport facilitator at the N1 or N3 triazole position, or a mixture thereof. For example, modified reactive caps may be a triazole regioisomer:

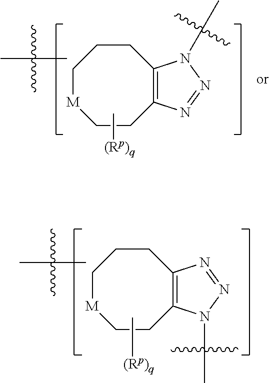

or a mixture thereof. In other embodiments, the modified reactive cap may be a triazole regioisomer:

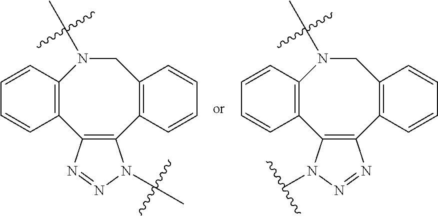

or a mixture thereof.

In certain embodiments of Formulas (I), R^{b} is fluoro, chloro, bromo, iodo, methanesulfonate, p-toluenesulfonate, trifluoromethanesulfonate, or acetate. In other embodiments, R^{b} is chloro or bromo. In still other embodiments, R^{b} is bromo. In still other embodiments, R^{b} is iodo.

In certain embodiments of Formulas (I) and (IA), R² is H and R¹ is a chemical moiety of Formula (B) and Formula (B-1).

In certain embodiments of Formulas (I) and (IA), alkyl spacer B in Formula (B) and Formula (B-1), respectively, is —C₁₋₂₀alkylene-, wherein one or more carbon units within the alkylene is replaced with C₃₋₇cycloalkylene, —C(O)NH—, —NHC(O)—, —C(O)O—, —OC(O)—, —C(O)—, —NH—, or —O—. In other embodiments, alkyl spacer B is —C₆₋₁₂alkylene-, wherein one or more carbon units within the alkylene is replaced with C₃₋₇cycloalkylene, —C(O)NH—, —NHC(O)—, or —C(O)—. In other embodiments, alkyl spacer B is -hexyl-NHC(O)-pentyl-, pentyl-NHC(O)-cyclohexyl-methyl-, -methyl-C(O)-hexyl-, —C(O)NH-hexyl-, or —C(O)NH-hexyl-NHC(O)-cyclohexyl-methyl-.

In certain embodiments of Formulas (II) and (IIA), X is S. In other embodiments, X is SO. In still other embodiments, X is SO₂.

In certain embodiments of Formulas (II) and (IIA), R¹ is H and R² is

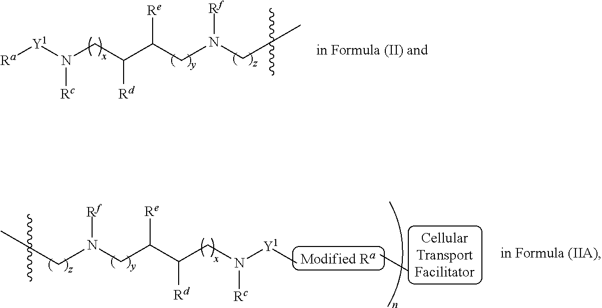

respectively.

In certain embodiments of Formulas (II) and (IIA), x is 0. In other embodiments, x is 1. In still other embodiments, x is 2. In some embodiments, y is 0. In other embodiments, y is 1. In some embodiments, the sum of x and y is 0. In other embodiments, the sum of x and y is 1. In still other embodiments, the sum of x and y is 2. In still other embodiments, the sum of x and y is 3.

In certain embodiments of Formulas (II) and (IIA), z is 0. In other embodiments, z is 1.

In certain embodiments of Formulas (II) and (IIA), R^{c} is H. In other embodiments, R^{c} is methyl.

In certain embodiments of Formulas (II) and (IIA), R^{f} is H. In other embodiments, R^{f} is methyl. In certain embodiments, R^{d} and R^{f} taken together form a bond. In other embodiments, R^{d} and R^{f} taken together form —CH₂—. In still other embodiments, R^{d} and R^{f} taken together form —CH₂CH₂—. In some embodiments, R^{e} and R^{f} taken together form a bond. In some embodiments, R^{c} and R^{f} taken together form —CH₂CH₂—.

In certain embodiments of Formulas (II) and (IIA), Y¹ is —C(O)C₁₋₁₆alkylene in which one or more carbon units are optionally replaced with C₃₋₇cycloalkylene, —C(O)NH—, —NHC(O)—, —C(O)O—, —OC(O)—, —C(O)—, NH, or O. In other embodiments, Y¹ is —C(O)C₁₋₁₃alkylene-, wherein one or more carbon units within the alkylene is optionally replaced with C₃₋₇cycloalkylene, —C(O)NH—, —NHC(O)—, —OC(O)—, or O. In other embodiments, Y¹ is absent or is —C(O)-methylene, —C(O)-ethylene, —C(O)-propylene, —C(O)-pentylene, —C(O)-pentyl-NHC(O)-pentyl-, —C(O)-cyclohexyl-methyl-, —C(O)-pentyl-OC(O)-cyclohexylmethyl-, —C(O)-pentyl-NHC(O)-cyclohexyl-methyl-, or —C(O)—CH₂—(OCH₂CH₂)₄—.

In certain embodiments of Formulas (II) and (IIA), R² is H and R¹ is

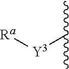

### in Formula (II) and

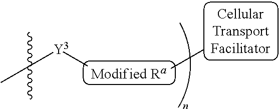

in Formula (IIA), respectively.

In certain embodiments of Formulas (II) and (IIA), Y³ is —C₆₋₁₂alkylene-, wherein one or more carbon units within the alkylene is replaced with C₃₋₇cycloalkylene, —C(O)NH—, —NHC(O)—, or —C(O)—. In other embodiments, Y³ is -hexyl-NHC(O)-pentyl-, -pentyl-C(O)NH— hexyl-, pentyl-NHC(O)-cyclohexyl-methyl-, -methyl-C(O)-hexyl-, —C(O)NH-hexyl-, or —C(O)NH-hexyl-NHC(O)-cyclohexyl-methyl-.

In certain embodiments of Formula (II), R^{a} is —N₃, —C≡CH, —CO₂H, —ONH₂,

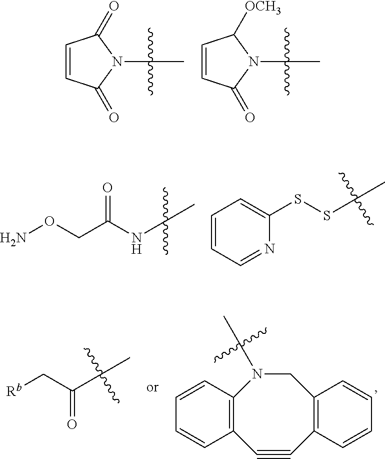

wherein R^{b} is a leaving group. In other embodiments, R^{a} is —CO₂H, or is

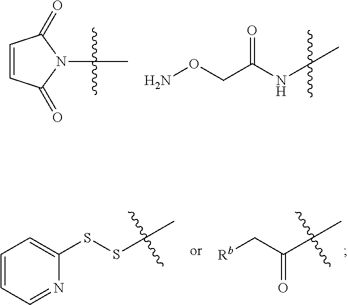

wherein R^{b} is a leaving group. In other embodiments, R^{a} is —ONH₂. In certain embodiments, R^{b} is iodo, chloro, bromo, or para-toluenesulfonate. In other embodiments, R^{b} is chloro, bromo, or para-toluenesulfonate. In other embodiments, R^{b} is chloro or bromo. In other embodiments, R^{b} is iodo. In other embodiments, R^{a} is

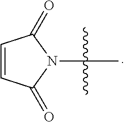 [SMILES: *N1C(=O)C=CC1=O]

In still other embodiments, R^{a} is —N₃ or —C≡CH. In other embodiments, R^{a} is

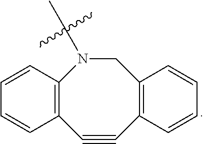 [SMILES: *N1Cc2ccccc2C#Cc2ccccc21]

In still other embodiments, R^{a} is:

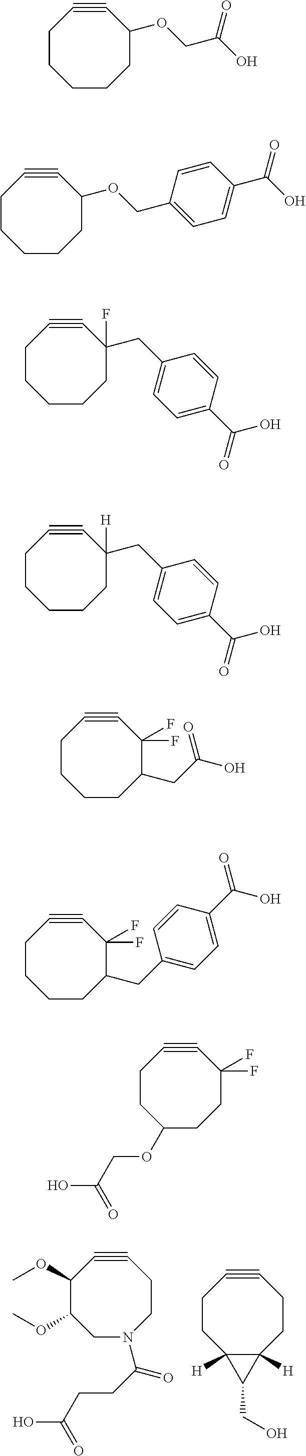

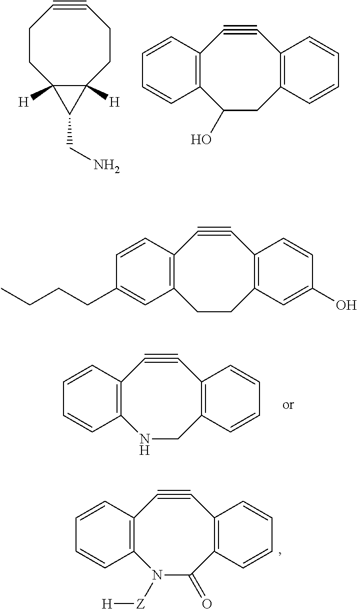

wherein Z is a bond, —C₁₋₄alkylene-O—, or —C₁₋₄alkylene-NH—, or a triazole regioisomer thereof, or a mixture of triazole regioisomers.

In certain embodiments of Formula (IIA), modified R^{a} is —C(O)NH—, or is:

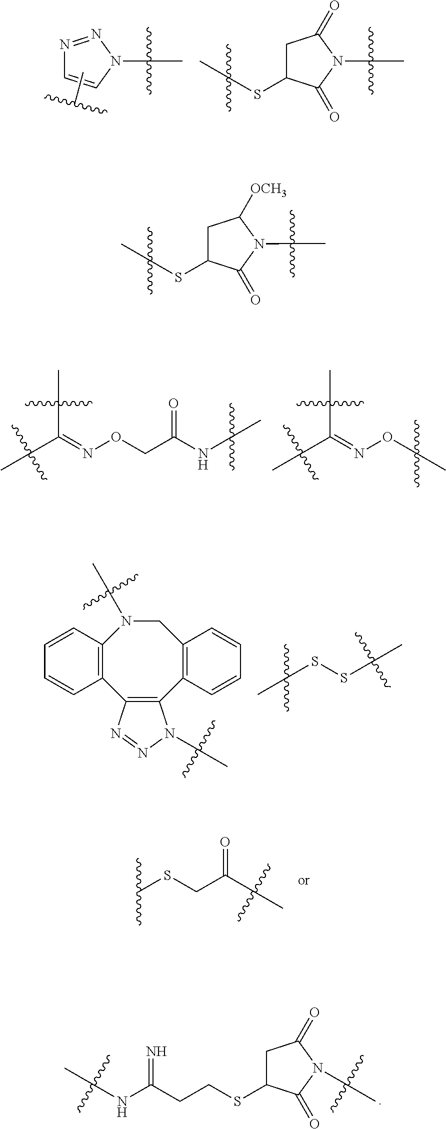

In other embodiments, modified R^{a} is —C(O)NH—, or is

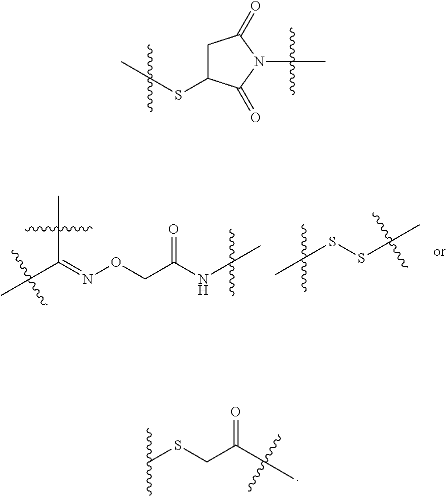

In other embodiments, modified R^{a} is

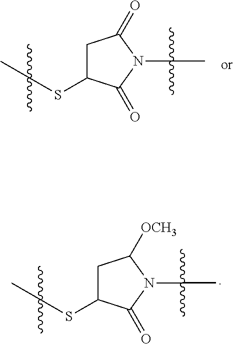 [SMILES: *SC1CC(=O)N(*)C1=O ; *SC1CC(OC)N(*)C1=O]

In still other embodiments, modified R^{a} is

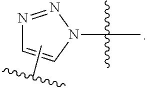

In still other embodiments, modified R^{a} is

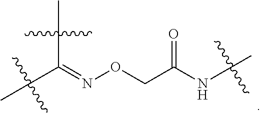 [SMILES: *NC(=O)CON=C(*)*]

In still other embodiments, modified R^{a} is

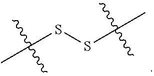 [SMILES: *SS*]

In still other embodiments, modified R^{a} is

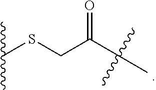 [SMILES: *SCC(*)=O]

In still other embodiments, modified R^{a} is

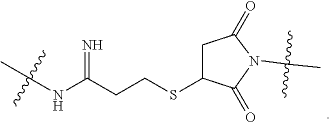 [SMILES: *NC(=N)CCSC1CC(=O)N(*)C1=O]

In still other embodiments, modified R^{a} is

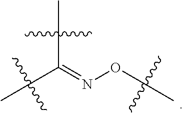 [SMILES: *ON=C(*)*]

In still other embodiments, modified R^{a} is

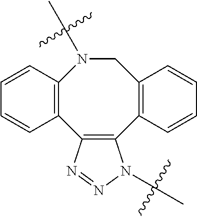 [SMILES: *N1Cc2ccccc2-c2c(nnn2*)-c2ccccc21]

or a triazole regioisomer thereof. In still other embodiments, the reactive cap is:

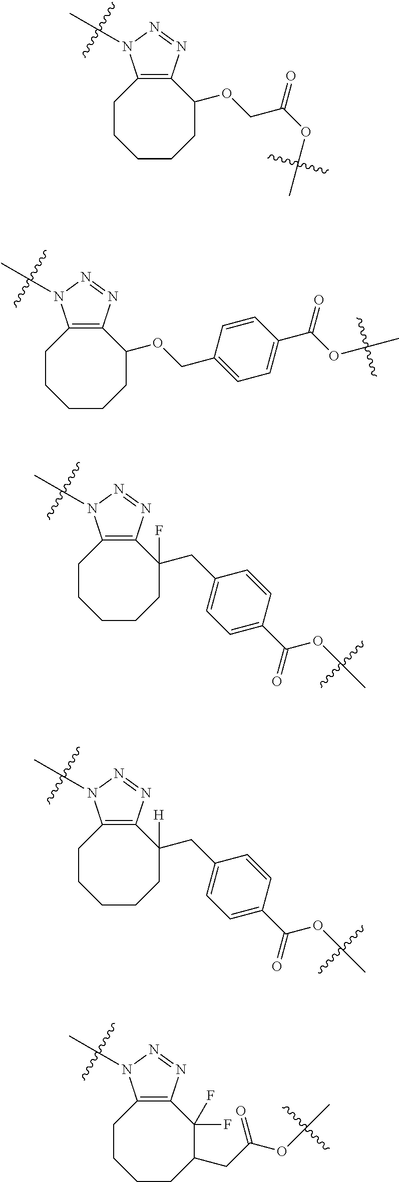

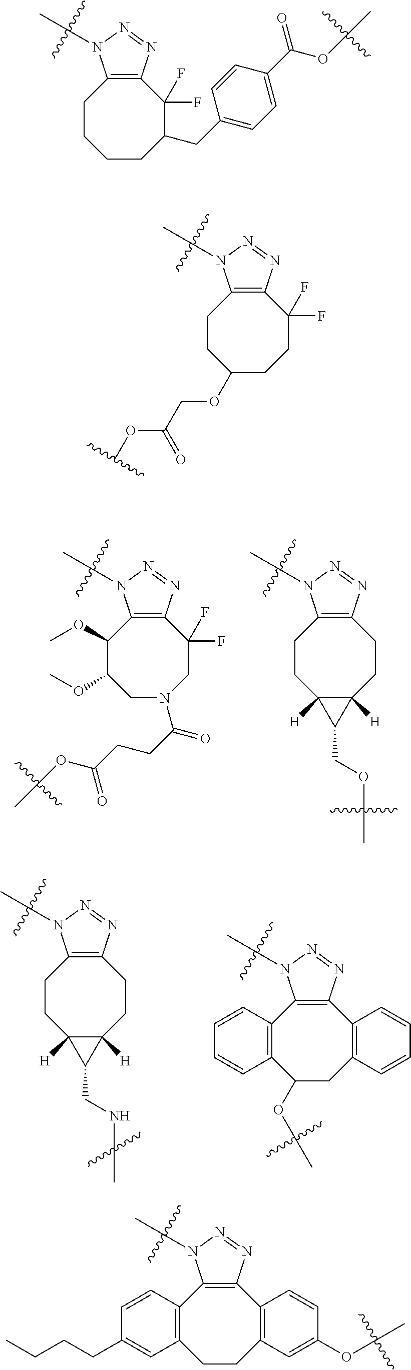

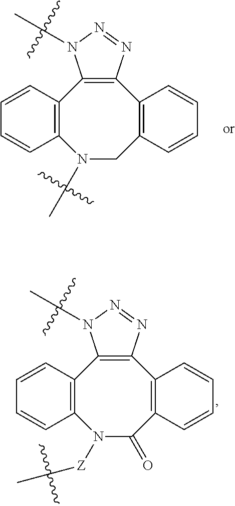

wherein Z is a bond, —C₁₋₄alkylene-O—, or —C₁₋₄alkylene-NH—, or a triazole regioisomer thereof, or a mixture of triazole regioisomers as described above for Formula (IA).

In certain embodiments of Formulas (IA) and (IIA), the cellular transport facilitator is an antibody or a peptide. In some embodiments, the antibody or peptide comprises a linker with a functional group suitable for coupling with a reactive cap moiety to form a covalent bond between the cellular transport facilitator and the remainder of the molecule.

In some embodiments, the compounds of the invention are compounds of Formula (III):

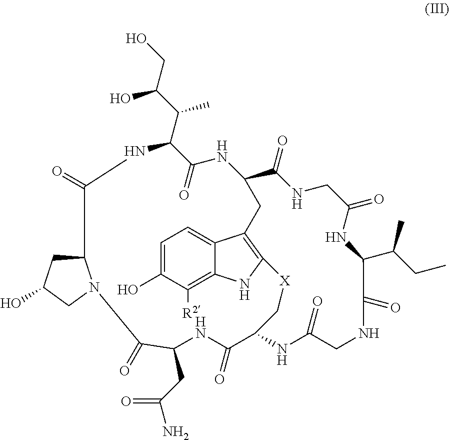

wherein

### X is S, SO, or SO₂; and

### R^{2′} is

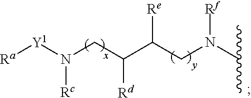

wherein Y¹, R^{a}, R^{c}, R^{d}, R^{e}, R^{f}, x, and y are defined as for Formula (II);

and pharmaceutically acceptable salts thereof.

In other embodiments, the compounds of the invention are compounds of Formula (IV):

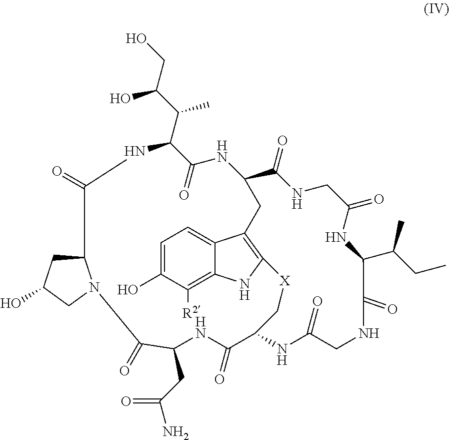

wherein

### X is S, SO, or SO₂; and

### R^{2′} is

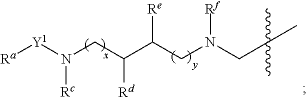

wherein Y¹, R^{a}, R^{c}, R^{d}, R^{e}, R^{f}, x, and y are defined as for Formula (II);

and pharmaceutically acceptable salts thereof.

In still other embodiments, the compounds of the invention are compounds of Formula (V):

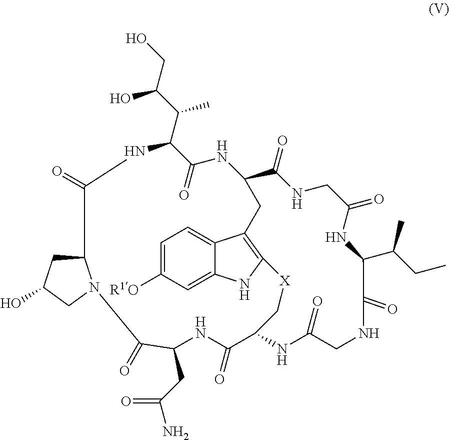

wherein

X is S, SO, or SO₂; and

R^{1′} is

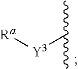

wherein R^{a} and Y³ are defined as for Formula (II);
   and pharmaceutically acceptable salts thereof.

In still other embodiments, the variables shown in Formula (III), Formula (IV), or Formula (V) may be defined, individually or collectively, as described above for Formula (I), (II), (A), or (B).

In still other embodiments, the invention is directed to conjugates between compounds of Formulas (III), (IV), and (V) and a cellular transport facilitator as shown in Formulas (IIA) through the modified R^{a} defined as for Formula (IIA). For example, the invention is directed to compounds of Formula (IIIA), Formula (IVA), or Formula (VA):

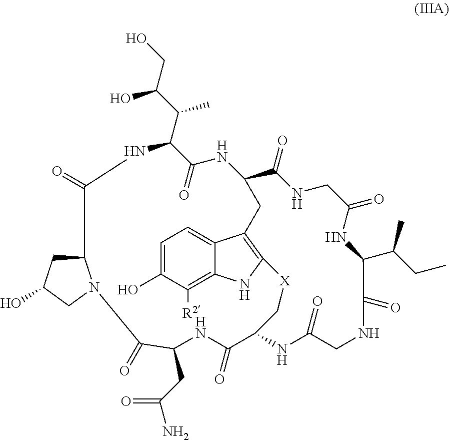

wherein
X is S, SO, or SO₂; and
R^{2′} is

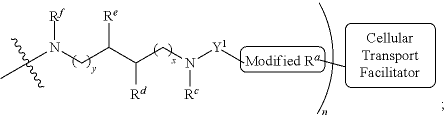

wherein Y¹, R^{a}, R^{c}, R^{d}, R^{e}, R^{f}, x, and y are defined as for Formula (II), and the modified R^{a}, the cellular transport facilitator, and n are defined as for Formula (IIA);
   and pharmaceutically acceptable salts thereof;

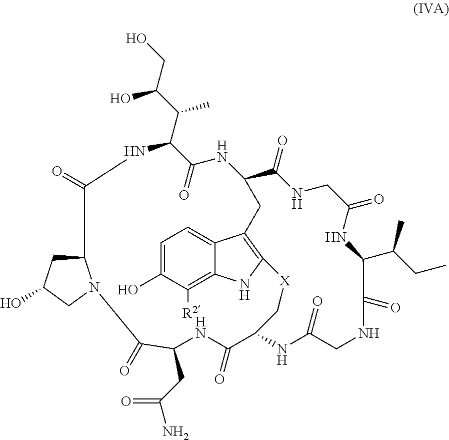

wherein

X is S, SO, or SO₂; and

R^{2′} is

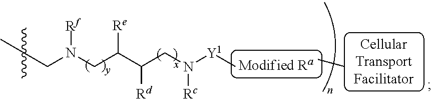

wherein Y¹, R^{a}, R^{c}, R^{d}, R^{e}, R^{f}, x, and y are defined as for Formula (II), and the modified R^{a}, the cellular transport facilitator, and n are defined as for Formula (IIA);
   and pharmaceutically acceptable salts thereof, or

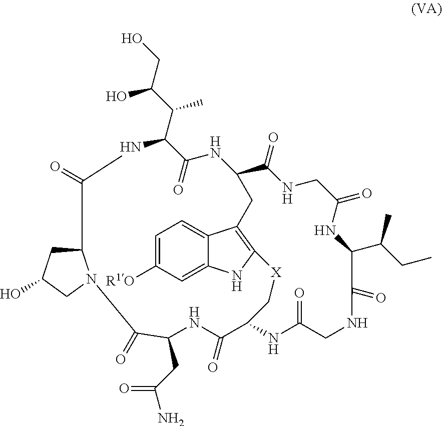

wherein

X is S, SO, or SO₂; and

R^{1′} is

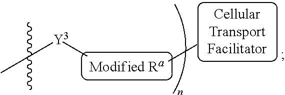

wherein Y³ are defined as for Formula (II), and the modified R^{a}, the cellular transport facilitator, and n are defined as for Formula (IIA);
   and pharmaceutically acceptable salts thereof. In certain embodiments of each definitions of the Formulas (III), (IIIA), (IV), (IVA), (V), and (VA), and the variables included therein, can be referred to the ones as exemplified as for Formula (II) and (IIA) above.

In certain embodiments of Formula (III), X is SO; (i) Y¹ is pentyl-(CO)—, R^{c} is H, R^{d} and R^{f} are taken together to form —CH₂CH₂—, R^{e} is H, x is 0, and y is 1, or (ii) Y¹ is pentyl-(CO)—, R^{d} is H, R^{c} and R^{f} are taken together to form —CH₂CH₂—, R^{e} is H, x is 0, and y is 0; and R^{a} is —N₃, —C≡CH, —CO₂H, —ONH₂,

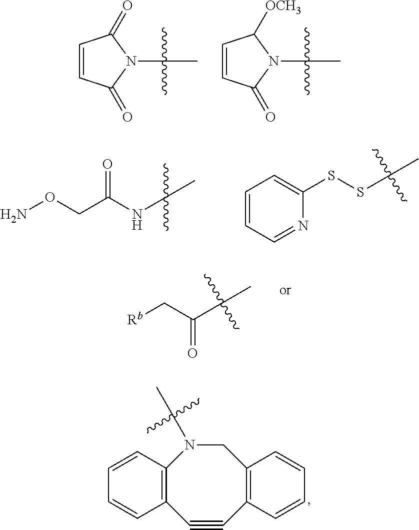

wherein R^{b} is a leaving group.

In certain embodiments of Formula (IIIA), X is SO; (i) Y¹ is pentyl-(CO)—, R^{c} is H, R^{d} and R^{f} are taken together to form —CH₂CH₂—, R^{e} is H, x is 0, and y is 1, or (ii) Y¹ is pentyl-(CO)—, R^{d} is H, R^{c} and R^{f} are taken together to form —CH₂CH₂—, R^{e} is H, x is 0, and y is 0; the modified R^{a} is —C(O)NH—, or is:

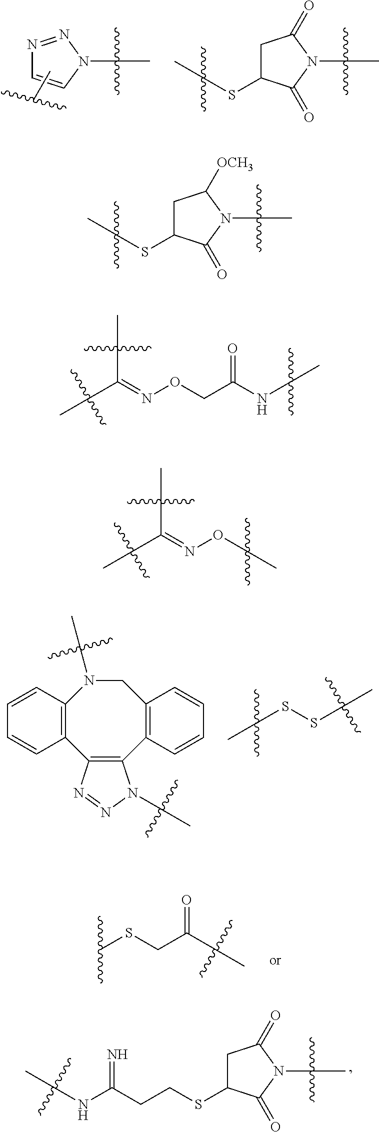

and the cellular transport facilitator is an antibody.

In certain embodiments of Formula (V), X is SO, Y³ is -hexyl-NHC(O)-pentyl- or -pentyl-C(O)NH-hexyl-, and R^{a} is —N₃, —C≡CH, —CO₂H, —ONH₂,

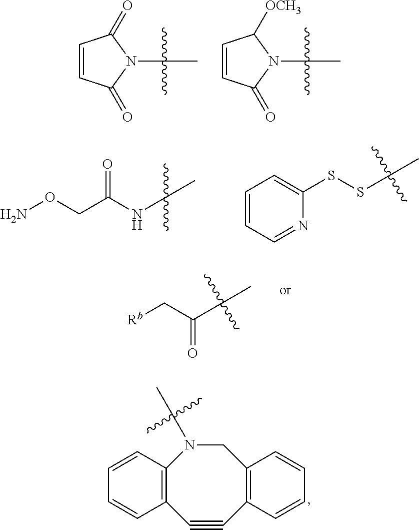

wherein R^{b} is a leaving group.

In certain embodiments of Formula (VA), X is SO, Y³ is -hexyl-NHC(O)-pentyl- or -pentyl-C(O)NH-hexyl-, and the modified R^{a} is —C(O)NH—, or is:

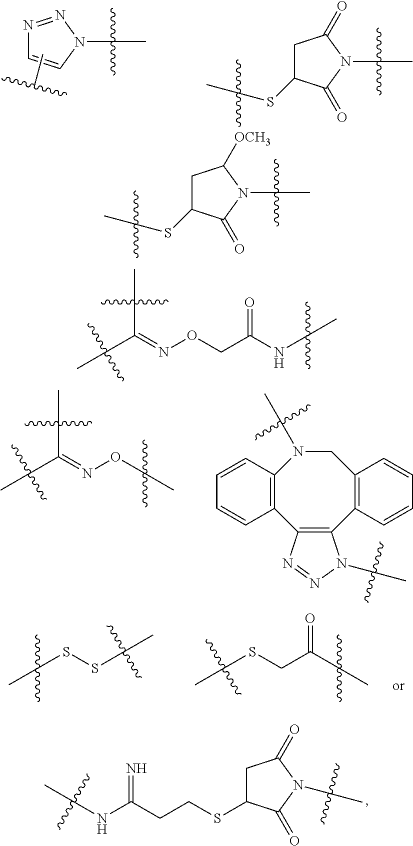

and the cellular transport facilitator is an antibody.

The compounds of Formulas (III), (IV) and (V) are included in the compounds of Formula (II), and the description on the compounds of Formula (II) is also understood as the description on the compounds of Formulas (III), (IV) and (V) in the specification and the claims, unless otherwise indicated.

The compounds of Formulas (IIIA), (IVA) and (VA) are included in the compounds of Formula (II), and the description on the compounds of Formula (IIA) is also understood as the description on the compounds of Formulas (IIIA), (IVA) and (VA) in the specification and the claims, unless otherwise indicated.

In other embodiments, compounds of Formula (I) and (II) are selected from those presented in Table 1:

TABLE 1

| Ex. | Chemical Name |
|---|---|
| 1 | 7′C-(4-(6-(maleimido)hexanoyl)piperazin-1-yl)-α-amanitin; |
| 2 | 7′C-(4-(6-(maleimido)hexanamido)piperidin-1-yl)-α-amanitin; |
| 3 | 7′C-(4-(6-(6-(maleimido)hexanamido)hexanoyl)piperazin-1-yl)-α-amanitin; |
| 4 | 7′C-(4-(4-((maleimido)methyl)cyclohexanecarbonyl)piperazin-1-yl)-α-amanitin; |
| 5 | 7′C-(4-(6-(4-((maleimido)methyl)cyclohexanecarboxamido)hexanoyl)piperazin-1- |
| | yl)-α-amanitin; |
| 6 | 7′C-(4-(2-(6-(maleimido)hexanamido)ethyl)piperidin-1-yl)-α-amanitin; |
| 7 | 7′C-(4-(2-(6-(6-(maleimido)hexanamido)hexanamido)ethyl)piperidin-1-yl)-α- |
| | amanitin; |
| 8 | 7′C-(4-(2-(4-((maleimido)methyl)cyclohexanecarboxamido)ethyl)piperidin-1-yl)- |
| | α-amanitin; |
| | 7′C-(4-(2-(6-(4- |
| 9 | ((maleimido)methyl)cyclohexanecarboxamido)hexanamido)ethyl)piperidin-1-yl)-α- |
| | amanitin; |
| 10 | 7′C-(4-(2-(3-carboxypropanamido)ethyl)piperidin-1-yl)-α-amanitin; |
| 11 | 7′C-(4-(2-(2-bromoacetamido)ethyl)piperidin-1-yl)-α-amanitin; |
| 12 | 7′C-(4-(2-(3-(pyridin-2-yldisulfanyl)propanamido)ethyl)piperidin-1-yl)-α- |
| | amanitin; |
| 13 | 7′C-(4-(2-(4-(maleimido)butanamido)ethyl)piperidin-1-yl)-α-amanitin; |
| 14 | 7′C-(4-(2-(maleimido)acetyl)piperazin-1-yl)-α-amanitin; |
| 15 | 7′C-(4-(3-(maleimido)propanoyl)piperazin-1-yl)-α-amanitin; |
| 16 | 7′C-(4-(4-(maleimido)butanoyl)piperazin-1-yl)-α-amanitin; |
| 17 | 7′C-(4-(2-(6-(4-((maleimido)methyl)cyclohexanecarboxamido)hexanamido) |
| | ethyl)piperidin-1-yl)-α-amanitin; |
| 18 | 7′C-(3-((6-(maleimido)hexanamido)methyl)pyrrolidin-1-yl)-α-amanitin; |
| 19 | 7′C-(3-((6-(6-(maleimido)hexanamido)hexanamido)methyl)pyrrolidin-1-yl)-α- |
| | amanitin; |
| 20 | 7′C-(3-((4-((maleimido)methyl)cyclohexanecarboxamido)methyl)pyrrolidin-1-yl)- |
| | α-amanitin; |
| 21 | 7′C-(3-((6-(4-((maleimido)methyl)cyclohexanecarboxamido)hexanamido) |
| | methyl)pyrrolidin-1-yl)-α-amanitin; |
| 22 | 7′C-(4-(2-(6-(2-(aminooxy)acetamido)hexanamido)ethyl)piperidin-1-yl)-α- |
| | amanitin; |
| 23 | 7′C-(4-(2-(4-(2-(aminooxy)acetamido)butanamido)ethyl)piperidin-1-yl)-α- |
| | amanitin; |
| 24 | 7′C-(4-(4-(2-(aminooxy)acetamido)butanoyl)piperazin-1-yl)-α-amanitin; |
| 25 | 7′C-(4-(6-(2-(aminooxy)acetamido)hexanoyl)piperazin-1-yl)-α-amanitin; |
| 26 | 7′C-((4-(6-(maleimido)hexanamido)piperidin-1-yl)methyl)-α-amanitin; |
| 27 | 7′C-((4-(2-(6-(maleimido)hexanamido)ethyl)piperidin-1-yl)methyl)-α-amanitin; |
| 28 | 7′C-((4-(6-(maleimido)hexanoyl)piperazin-1-yl)methyl)-α-amanitin; |
| 29 | (R)-7′C-((3-((6-(maleimido)hexanamido)methyl)pyrrolidin-1-yl)methyl)-α- |
| | amanitin; |
| 30 | (S)-7′C-((3-((6-(maleimido)hexanamido)methyl)pyrrolidin-1-yl)methyl)-α- |
| | amanitin; |
| 31 | 7′C-((4-(2-(6-(6-(maleimido)hexanamido)hexanamido)ethyl)piperidin-1- |
| | yl)methyl)-α-amanitin; |
| 32 | 7′C-((4-(2-(4-((maleimido)methyl)cyclohexanecarboxamido)ethyl)piperidin-1- |
| | yl)methyl)-α-amanitin; |
| 33 | 7′C-((4-(2-(6-(4- |
| | ((maleimido)methyl)cyclohexanecarboxamido)hexanamido)ethyl)piperidin-1- |
| | yl)methyl)-α-amanitin; |
| 34 | 7′C-((4-(2-(6-(maleimido)hexanamido)ethyl)piperazin-1-yl)methyl)-α-amanitin; |
| 35 | 7′C-((4-(2-(6-(6-(maleimido)hexanamido)hexanamido)ethyl)piperazin-1- |
| | yl)methyl)-α-amanitin; |
| 36 | 7′C-((4-(2-(4-((maleimido)methyl)cyclohexanecarboxamido)ethyl)piperazin-1- |
| | yl)methyl)-α-amanitin; |
| 37 | 7′C-((4-(2-(6-(4- |
| | ((maleimido)methyl)cyclohexanecarboxamido)hexanamido)ethyl)piperazin-1- |
| | yl)methyl)-α-amanitin; |
| 38 | 7′C-((3-((6-(6-(maleimido)hexanamido)hexanamido)-S-methyl)pyrrolidin-1- |
| | yl)methyl)-α-amanitin; |
| 39 | 7′C-((3-((6-(6-(maleimido)hexanamido)hexanamido)-R-methyl)pyrrolidin-1- |
| | yl)methyl)-α-amanitin; |
| 40a | 7′C-((3-((4-((maleimido)methyl)cyclohexanecarboxamido)-S-methyl)pyrrolidin-1- |
| | yl)methyl)-α-amanitin; |
| 40b | 7′C-((3-((4-((maleimido)methyl)cyclohexanecarboxamido)-R-methyl)pyrrolidin-1- |
| | yl)methyl)-α-amanitin; |
| 41 | 7′C-((3-((6-(4- |
| | ((maleimido)methyl)cyclohexanecarboxamido)hexanamido)methyl)pyrrolidin-1- |
| | yl)methyl)-α-amanitin; |
| 42 | 7′C-((4-(2-(3-carboxypropanamido)ethyl)piperazin-1-yl)methyl)-α-amanitin; |
| 43 | 7′C-((4-(6-(6-(maleimido)hexanamido)hexanoyl)piperazin-1-yl)methyl)-α- |
| | amanitin; |
| 44 | 7′C-((4-(6-(4-((maleimido)methyl)cyclohexanecarboxamido)hexanoyl)piperazin-1- |
| | yl)methyl)-α-amanitin; |
| 45 | 7′C-((4-(2-(maleimido)acetyl)piperazin-1-yl)methyl)-α-amanitin; |
| 46 | 7′C-((4-(3-(maleimido)propanoyl)piperazin-1-yl)methyl)-α-amanitin; |
| 47 | 7′C-((4-(4-(maleimido)butanoyl)piperazin-1-yl)methyl)-α-amanitin; |
| 48 | 7′C-((4-(2-(2-(maleimido)acetamido)ethyl)piperidin-1-yl)methyl)-α-amanitin; |
| 49 | 7′C-((4-(2-(4-(maleimido)butanamido)ethyl)piperidin-1-yl)methyl)-α-amanitin; |
| 50 | 7′C-((4-(2-(6-(4- |
| | ((maleimido)methyl)cyclohexanecarboxamido)hexanamido)ethyl)piperidin-1- |
| | yl)methyl)-α-amanitin; |
| 51 | 7′C-((3-((6-(maleimido)hexanamido)methyl)azetidin-1-yl)methyl)-α-amanitin; |
| 52 | 7′C-((3-(2-(6-(maleimido)hexanamido)ethyl)azetidin-1-yl)methyl)-α-amanitin; |
| 53 | 7′C-((3-((4-((maleimido)methyl)cyclohexanecarboxamido)methyl)azetidin-1- |
| | yl)methyl)-α-amanitin; |
| 54 | 7′C-((3-(2-(4-((maleimido)methyl)cyclohexanecarboxamido)ethyl)azetidin-1- |
| | yl)methyl)-α-amanitin; |
| 55 | 7′C-((3-(2-(6-(4- |
| | ((maleimido)methyl)cyclohexanecarboxamido)hexanamido)ethyl)azetidin-1- |
| | yl)methyl)-α-amanitin; |
| 56 | 7′C-(((2-(6-(maleimido)-N-methylhexanamido)ethyl)(methyl)amino)methyl)-α- |
| | amanitin; |
| 57 | 7′C-(((4-(6-(maleimido)-N-methylhexanamido)butyl(methyl)amino)methyl)-α- |
| | amanitin; |
| 58 | 7′C-((2-(2-(6-(maleimido)hexanamido)ethyl)aziridin-1-yl)methyl)-α-amanitin; |
| 59 | 7′C-((2-(2-(6-(4- |
| | ((maleimido)methyl)cyclohexanecarboxamido)hexanamido)ethyl)aziridin-1- |
| | yl)methyl)-α-amanitin; |
| 60 | 7′C-((4-(6-(6-(2-(aminooxy)acetamido)hexanamido)hexanoyl)piperazin-1- |
| | yl)methyl)-α-amanitin; |
| 61 | 7′C-((4-(1-(aminooxy)-2-oxo-6,9,12,15-tetraoxa-3-azaheptadecan-17- |
| | oyl)piperazin-1-yl)methyl)-α-amanitin; |
| 62 | 7′C-((4-(2-(2-(aminooxy)acetamido)acetyl)piperazin-1-yl)methyl)-α-amanitin; |
| 63 | 7′C-((4-(3-(2-(aminooxy)acetamido)propanoyl)piperazin-1-yl)methyl)-α-amanitin; |
| 64 | 7′C-((4-(4-(2-(aminooxy)acetamido)butanoyl)piperazin-1-yl)methyl)-α-amanitin; |
| 65 | 7′C-((4-(2-(6-(2-(aminooxy)acetamido)hexanamido)ethyl)piperidin-1-yl)methyl)- |
| | α-amanitin; |
| 66 | 7′C-((4-(2-(2-(2-(aminooxy)acetamido)acetamido)ethyl)piperidin-1-yl)methyl)-α- |
| | amanitin; |
| 67 | 7′C-((4-(2-(4-(2-(aminooxy)acetamido)butanamido)ethyl)piperidin-1-yl)methyl)-α- |
| | amanitin; |
| 68 | 7′C-((4-(20-(aminooxy)-4,19-dioxo-6,9,12,15-tetraoxa-3,18-diazaicosyl)piperidin- |
| | 1-yl)methyl)-α-amanitin; |
| 69 | 7′C-(((2-(6-(2-(aminooxy)acetamido)-N- |
| | methylhexanamido)ethyl)(methyl)amino)methyl)-α-amanitin; |
| 70 | 7′C-(((4-(6-(2-(aminooxy)acetamido)-N- |
| | methylhexanamido)butyl)(methyl)amino)methyl)-α-amanitin; |
| 71 | 7′C-((3-((6-(4- |
| | ((maleimido)methyl)cyclohexanecarboxamido)hexanamido)methyl)pyrrolidin-1- |
| | yl)-S-methyl)-α-amanitin; |
| 72 | 7′C-((3-((6-(4-((maleimido)methyl)cyclohexanecarboxamido)hexanamido)-R- |
| | methyl)pyrrolidin-1-yl)methyl)-α-amanitin; |
| 73 | 7′C-((4-(2-(2-bromoacetamido)ethyl)piperazin-1-yl)methyl)-α-amanitin; |
| 74 | 7′C-((4-(2-(2-bromoacetamido)ethyl)piperidin-1-yl)methyl)-α-amanitin; |
| 75 | 7′C-((4-(2-(3-(pyridine-2-yldisulfanyl)propanamido)ethyl)piperidin-1-yl)methyl)- |
| | α-amanitin; |
| 76 | 6′O-(6-(6-(maleimido)hexanamido)hexyl)-α-amanitin; |
| 77 | 6′O-(5-(4-((maleimido)methyl)cyclohexanecarboxamido)pentyl)-α-amanitin; |
| 78 | 6′O-(2-((6-(maleimido)hexyl)oxy)-2-oxoethyl)-α-amanitin; |
| 79 | 6′O-((6-(maleimido)hexyl)carbamoyl)-α-amanitin; |
| 80 | 6′O-((6-(4-((maleimido)methyl)cyclohexanecarboxamido)hexyl)carbamoyl)-α- |
| | amanitin; |
| 81 | 6′O-(6-(2-bromoacetamido)hexyl)-α-amanitin; |
| 82 | 7′C-(4-(6-(azido)hexanamido)piperidin-1-yl)-α-amanitin; |
| 83 | 7′C-(4-(hex-5-ynoylamino)piperidin-1-yl)-α-amanitin; |
| 84 | 7′C-(4-(2-(6-(maleimido)hexanamido)ethyl)piperazin-1-yl)-α-amanitin; |
| 85 | 7′C-(4-(2-(6-(6-(maleimido)hexanamido)hexanamido)ethyl)piperazin-1-yl)-α- |
| | amanitin; |
| 86 | 6′O-(6-(6-(11,12-didehydro-5,6-dihydro-dibenz[b,f]azocin-5-yl)-6- |
| | oxohexanamido)hexyl)-α-amanitin; |
| 87 | 6′O-(6-(hex-5-ynoylamino)hexyl)-α-amanitin; |
| 88 | 6′O-(6-(2-(aminooxy)acetylamido)hexyl)-α-amanitin; |
| 89 | 6′O-((6-aminooxy)hexyl)-α-amanitin; and |
| 90 | 6′O-(6-(2-iodoacetamido)hexyl)-α-amanitin; |

and pharmaceutically acceptable salts thereof.

In other embodiments, compounds of Formula (I) and (II) are selected from those presented in Table 2:

TABLE 2

| Ex. | Chemical Name |
|---|---|
| 1 | 7′C-(4-(6-(maleimido)hexanoyl)piperazin-1-yl)-α-amanitin; |
| 2 | 7′C-(4-(6-(maleimido)hexanamido)piperidin-1-yl)-α-amanitin; |
| 3 | 7′C-(4-(6-(6-(maleimido)hexanamido)hexanoyl)piperazin-1-yl)-α-amanitin; |
| 4 | 7′C-(4-(4-((maleimido)methyl)cyclohexanecarbonyl)piperazin-1-yl)-α-amanitin; |
| 5 | 7′C-(4-(6-(4-((maleimido)methyl)cyclohexanecarboxamido)hexanoyl)piperazin-1- |
| | yl)-α-amanitin; |
| 6 | 7′C-(4-(2-(6-(maleimido)hexanamido)ethyl)piperidin-1-yl)-α-amanitin; |
| 7 | 7′C-(4-(2-(6-(6-(maleimido)hexanamido)hexanamido)ethyl)piperidin-1-yl)-α- |
| | amanitin; |
| 8 | 7′C-(4-(2-(4-((maleimido)methyl)cyclohexanecarboxamido)ethyl)piperidin-1-yl)- |
| | α-amanitin; |
| 9 | 7′C-(4-(2-(6-(4- |
| | ((maleimido)methyl)cyclohexanecarboxamido)hexanamido)ethyl)piperidin-1-yl)-α- |
| | amanitin; |
| 26 | 7′C-((4-(6-(maleimido)hexanamido)piperidin-1-yl)methyl)-α-amanitin; |
| 27 | 7′C-((4-(2-(6-(maleimido)hexanamido)ethyl)piperidin-1-yl)methyl)-α-amanitin; |
| 28 | 7′C-((4-(6-(maleimido)hexanoyl)piperazin-1-yl)methyl)-α-amanitin; |
| 29 | 7′C-((3-((6-(maleimido)hexanamido)-R-methyl)pyrrolidin-1-yl)methyl)-α- |
| | amanitin; |
| 30 | 7′C-((3-((6-(maleimido)hexanamido)-S-methyl)pyrrolidin-1-yl)methyl)-α- |
| | amanitin; |
| 31 | 7′C-((4-(2-(6-(6-(maleimido)hexanamido)hexanamido)ethyl)piperidin-1- |
| | yl)methyl)-α-amanitin; |
| 32 | 7′C-((4-(2-(4-((maleimido)methyl)cyclohexanecarboxamido)ethyl)piperidin-1- |
| | yl)methyl)-α-amanitin; |
| 33 | 7′C-((4-(2-(6-(4- |
| | ((maleimido)methyl)cyclohexanecarboxamido)hexanamido)ethyl)piperidin-1- |
| | yl)methyl)-α-amanitin; |
| 34 | 7′C-((4-(2-(6-(maleimido)hexanamido)ethyl)piperazin-1-yl)methyl)-α-amanitin; |
| 35 | 7′C-((4-(2-(6-(6-(maleimido)hexanamido)hexanamido)ethyl)piperazin-1- |
| | yl)methyl)-α-amanitin; |
| 36 | 7′C-((4-(2-(4-((maleimido)methyl)cyclohexanecarboxamido)ethyl)piperazin-1- |
| | yl)methyl)-α-amanitin; |
| 37 | 7′C-((4-(2-(6-(4- |
| | ((maleimido)methyl)cyclohexanecarboxamido)hexanamido)ethyl)piperazin-1- |
| | yl)methyl)-α-amanitin; |
| 38 | 7′C-((3-((6-(6-(maleimido)hexanamido)hexanamido)-S-methyl)pyrrolidin-1- |
| | yl)methyl)-α-amanitin; |
| 39 | 7′C-((3-((6-(6-(maleimido)hexanamido)hexanamido)-R-methyl)pyrrolidin-1- |
| | yl)methyl)-α-amanitin; |
| 40a | 7′C-((3-((4-((maleimido)methyl)cyclohexanecarboxamido)-S-methyl)pyrrolidin-1- |
| | yl)methyl)-α-amanitin; |
| 40b | 7′C-((3-((4-((maleimido)methyl)cyclohexanecarboxamido)-R-methyl)pyrrolidin-1- |
| | yl)methyl)-α-amanitin; |
| 71 | 7′C-((3-((6-(4- |
| | ((maleimido)methyl)cyclohexanecarboxamido)hexanamido)methyl)pyrrolidin-1- |
| | yl)-S-methyl)-α-amanitin; |
| 72 | 7′C-((3-((6-(4-((maleimido)methyl)cyclohexanecarboxamido)hexanamido)-R- |
| | methyl)pyrrolidin-1-yl)methyl)-α-amanitin; |
| 76 | 6′O-(6-(6-(maleimido)hexanamido)hexyl)-α-amanitin; |
| 77 | 6′O-(5-(4-((maleimido)methyl)cyclohexanecarboxamido)pentyl)-α-amanitin; |
| 81 | 6′O-(6-(2-bromoacetamido)hexyl)-α-amanitin; |
| 85 | 7′C-(4-(2-(6-(6-(maleimido)hexanamido)hexanamido)ethyl)piperazin-1-yl)-α- |
| | amanitin; and |
| 86 | 6′O-(6-(6-(11,12-didehydro-5,6-dihydro-dibenz[b,f]azocin-5-yl)-6- |
| | oxohexanamido)hexyl)-α-amanitin |

and pharmaceutically acceptable salts thereof.

In other embodiments, compounds of Formula (I) and (II) are selected from those presented in Table 3:

TABLE 3

| Ex. | Chemical Name |
|---|---|
| 1 | 7′C-(4-(6-(maleimido)hexanoyl)piperazin-1-yl)-α-amanitin; |
| 2 | 7′C-(4-(6-(maleimido)hexanamido)piperidin-1-yl)-α-amanitin; |
| 3 | 7′C-(4-(6-(6-(maleimido)hexanamido)hexanoyl)piperazin-1-yl)-α-amanitin; |
| 4 | 7′C-(4-(4-((maleimido)methyl)cyclohexanecarbonyl)piperazin-1-yl)-α-amanitin; |
| 5 | 7′C-(4-(6-(4-((maleimido)methyl)cyclohexanecarboxamido)hexanoyl)piperazin-1- |
| | yl)-α-amanitin; |
| 6 | 7′C-(4-(2-(6-(maleimido)hexanamido)ethyl)piperidin-1-yl)-α-amanitin; |
| 7 | 7′C-(4-(2-(6-(6-(maleimido)hexanamido)hexanamido)ethyl)piperidin-1-yl)-α- |
| | amanitin; |
| 8 | 7′C-(4-(2-(4-((maleimido)methyl)cyclohexanecarboxamido)ethyl)piperidin-1-yl)- |
| | α-amanitin; |
| 9 | 7′C-(4-(2-(6-(4- |
| | ((maleimido)methyl)cyclohexanecarboxamido)hexanamido)ethyl)piperidin-1-yl)-α- |
| | amanitin; |
| 10 | 7′C-(4-(2-(3-carboxypropanamido)ethyl)piperidin-1-yl)-α-amanitin; |
| 26 | 7′C-((4-(6-(maleimido)hexanamido)piperidin-1-yl)methyl)-α-amanitin; |
| 27 | 7′C-((4-(2-(6-(maleimido)hexanamido)ethyl)piperidin-1-yl)methyl)-α-amanitin; |
| 28 | 7′C-((4-(6-(maleimido)hexanoyl)piperazin-1-yl)methyl)-α-amanitin; |
| 29 | (R)-7′C-((3-((6-(maleimido)hexanamido)methyl)pyrrolidin-1-yl)methyl)-α- |
| | amanitin; |
| 30 | (S)-7′C-((3-((6-(maleimido)hexanamido)methyl)pyrrolidin-1-yl)methyl)-α- |
| | amanitin; |
| 31 | 7′C-((4-(2-(6-(6-(maleimido)hexanamido)hexanamido)ethyl)piperidin-1- |
| | yl)methyl)-α-amanitin; |
| 32 | 7′C-((4-(2-(4-((maleimido)methyl)cyclohexanecarboxamido)ethyl)piperidin-1- |
| | yl)methyl)-α-amanitin; |
| 33 | 7′C-((4-(2-(6-(4- |
| | ((maleimido)methyl)cyclohexanecarboxamido)hexanamido)ethyl)piperidin-1- |
| | yl)methyl)-α-amanitin; |
| 34 | 7′C-((4-(2-(6-(maleimido)hexanamido)ethyl)piperazin-1-yl)methyl)-α-amanitin; |
| 35 | 7′C-((4-(2-(6-(6-(maleimido)hexanamido)hexanamido)ethyl)piperazin-1- |
| | yl)methyl)-α-amanitin; |
| 36 | 7′C-((4-(2-(4-((maleimido)methyl)cyclohexanecarboxamido)ethyl)piperazin-1- |
| | yl)methyl)-α-amanitin; |
| 37 | 7′C-((4-(2-(6-(4- |
| | ((maleimido)methyl)cyclohexanecarboxamido)hexanamido)ethyl)piperazin-1- |
| | yl)methyl)-α-amanitin; |
| 38 | 7′C-((3-((6-(6-(maleimido)hexanamido)hexanamido)-S-methyl)pyrrolidin-1- |
| | yl)methyl)-α-amanitin; |
| 39 | 7′C-((3-((6-(6-(maleimido)hexanamido)hexanamido)-R-methyl)pyrrolidin-1- |
| | yl)methyl)-α-amanitin; |
| 40a | 7′C-((3-((4-((maleimido)methyl)cyclohexanecarboxamido)-S-methyl)pyrrolidin-1- |
| | yl)methyl)-α-amanitin; |
| 40b | 7′C-((3-((4-((maleimido)methyl)cyclohexanecarboxamido)-R-methyl)pyrrolidin-1- |
| | yl)methyl)-α-amanitin; |
| 42 | 7′C-((4-(2-(3-carboxypropanamido)ethyl)piperazin-1-yl)methyl)-α-amanitin; |
| 71 | 7′C-((3-((6-(4- |
| | ((maleimido)methyl)cyclohexanecarboxamido)hexanamido)methyl)pyrrolidin-1- |
| | yl)-S-methyl)-α-amanitin; |
| 79 | 7′C-((3-((6-(4-((maleimido)methyl)cyclohexanecarboxamido)hexanamido)-R- |
| | methyl)pyrrolidin-1-yl)methyl)-α-amanitin; |
| 76 | 6′O-(6-(6-(maleimido)hexanamido)hexyl)-α-amanitin; |
| 77 | 6′O-(5-(4-((maleimido)methyl)cyclohexanecarboxamido)pentyl)-α-amanitin; |
| 81 | 6′O-(6-(2-bromoacetamido)hexyl)-α-amanitin; |
| 82 | 7′C-(4-(6-(azido)hexanamido)piperidin-1-yl)-α-amanitin; |
| 83 | 7′C-(4-(hex-5-ynoylamino)piperidin-1-yl)-α-amanitin; |
| 84 | 7′C-(4-(2-(6-(maleimido)hexanamido)ethyl)piperazin-1-yl)-α-amanitin; |
| 85 | 7′C-(4-(2-(6-(6-(maleimido)hexanamido)hexanamido)ethyl)piperazin-1-yl)-α- |
| | amanitin; |
| 86 | 6′O-(6-(6-(11,12-didehydro-5,6-dihydro-dibenz[b,f]azocin-5-yl)-6- |
| | oxohexanamido)hexyl)-α-amanitin; |
| 87 | 6′O-(6-(hex-5-ynoylamino)hexyl)-α-amanitin; |
| 88 | 6′O-(6-(2-(aminooxy)acetylamido)hexyl)-α-amanitin; and |
| 89 | 6′O-((6-aminooxy)hexyl)-α-amanitin; |

and pharmaceutically acceptable salts thereof.

In further embodiments, compounds of the invention are compounds of Formula (IA) and (IIA) in which a compound from Table 1 is covalently bound to the cellular transport facilitator. In still further embodiments, compounds of the invention are compounds of Formula (IA) and (IIA) in which a compound from Table 2 is covalently bound to the cellular transport facilitator. In still other embodiments, compounds of the invention are compounds of Formula (IA) and (IIA) in which a compound from Table 3 is covalently bound to the cellular transport facilitator. In other embodiments, compounds of the invention are compounds of Formula (IA) and (IIA) in which the compounds listed in Table 3 have been covalently bound to a cellular transport facilitator. In other embodiments, the compounds of Formula (IA) and (IIA) are selected from those described in Example 91 below and/or in the Figure descriptions.

The invention includes pharmaceutically acceptable salts of the compounds of Formula (I), Formula (IA), Formula (II), and Formula (IIA), including of those described above and the specific compounds exemplified herein, pharmaceutical compositions comprising such salts, and methods of using such salts. In some embodiments, compounds of Formula (I) and (II) are selected from the group consisting of those listed in Table 1 or Table 2, and pharmaceutically acceptable salts thereof. In some embodiments, compounds of Formula (I) and (II) are selected from the group consisting of those listed in Table 3, and pharmaceutically acceptable salts thereof.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

In the context of methods of inhibition, compositions comprising compounds of the invention, including those comprising conjugates as described herein, may further comprise one or more additives. Such additives may be pharmaceutically-acceptable excipients, as described further below, or may be additives that are compatible with in vitro or ex vivo assay conditions.

A “cellular transport facilitator” as used herein is any molecule that, when covalently bound to the toxin, promotes entry of the toxin into a cell, but does not substantially alter the cytotoxicity of the toxin. Transport into the cell may be, for example, through active transport, passive transport, facilitated diffusion, or endocytosis. Such facilitators include antibodies, antibody fragments, enzymes, polypeptides, synthetic polymers, and vesicles such as liposomes. Polypeptides may include, for example, poly(amino acid)s such as poly(lysine) and poly(valine) and mixed-sequence polypeptides. Polypeptides may further include pseudopeptides which comprise linkages other than amide linkages, such as CH₂NH₂ linkages as well as peptidomimetics. Synthetic polymers may include, for example, poly(ethylene glycol) (PEG), poly(ethylene oxide) (PEO), poly(ethylene imine) (PEI), and co-polymers thereof; and polysaccharides such as dextrans. The facilitator will comprise at least one functional group suitable for conjugation to the toxin, either natively or after chemical transformation, such as an amine, carboxylic acid, alcohol, thiol, alkyne, azide, maleimide, or other chemical group.

The term “antibody” is used in the broadest sense unless clearly indicated otherwise. Therefore, an “antibody” can be naturally occurring or man-made such as monoclonal antibodies produced by conventional hybridoma technology. Suitable antibodies comprise monoclonal and polyclonal antibodies as well as fragments containing the antigen-binding domain and/or one or more complementarity determining regions of these antibodies. As used herein, the term “antibody” refers to any form of antibody or fragment thereof that specifically binds to a target antigen and/or exhibits the desired biological activity and specifically covers monoclonal antibodies (including full length monoclonal antibodies), polyclonal antibodies, multispecific antibodies (e.g., bispecific antibodies), and antibody fragments so long as they specifically bind to the target antigen and/or exhibit the desired biological activity.

Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. In one embodiment, the antibody is an IgG antibody. For example, the antibody is an IgG1, IgG2, IgG3, or IgG4 antibody.

The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, and apes. Therefore, in one embodiment, an antibody useful in the present invention is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard et al. Monoclonal Antibodies (Oxford University Press, 2000); Goding, Monoclonal Antibodies: Principles and Practice (Academic Press, 1993); and Current Protocols in Immunology (John Wiley & Sons, most recent edition). An antibody useful in the present invention can be modified by recombinant means to increase efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. No. 5,624,821, U.S. Pat. No. 6,194,551, PCT Appl. Publ. No. WO 99/58572; and Angal et al. (1993) Mol. Immunol. 30:105-08. Suitable amino acid modifications include deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to normal or defective targets. See e.g., Antibody Engineering: A Practical Approach (Oxford University Press, 1996).

In one embodiment, an antibody useful in the present invention is a “human antibody.” As used herein, the term “human antibody” refers to an antibody in which essentially the entire sequences of the light chain and heavy chain sequences, including the complementary determining regions (CDRs), are from human genes. In one embodiment, human monoclonal antibodies are prepared by the trioma technique, the human B-cell technique (see, e.g., Kozbor, et al. (1983) Immunol. Today 4:72), EBV transformation technique (see, e.g., Cole et al. (1985) Monoclonal Antibodies and Cancer Therapy, UCLA Symposia on Molecular and Cellular Biology, Vol. 27, New Series (R. A. Reisfeld and S. Sell, eds.), pp. 77-96), or using phage display (see, e.g., Marks et al. (1991) J. Mol. Biol. 222:581). In a specific embodiment, the human antibody is generated in a transgenic mouse. Techniques for making such partially to fully human antibodies are known in the art and any such techniques can be used. According to one particularly preferred embodiment, fully human antibody sequences are made in a transgenic mouse engineered to express human heavy and light chain antibody genes. An exemplary description of preparing transgenic mice that produce human antibodies found in Application No. WO 02/43478 and U.S. Pat. No. 6,657,103 (Abgenix) and its progeny. B cells from transgenic mice that produce the desired antibody can then be fused to make hybridoma cell lines for continuous production of the antibody. See, e.g., U.S. Pat. Nos. 5,569,825, 5,625,126, 5,633,425, 5,661,016, and 5,545,806; Jakobovits (1998) Adv. Drug Del. Rev. 31:33-42; and Green et al. (1998) J. Exp. Med. 188:483-95.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. See e.g., Cabilly, U.S. Pat. No. 4,816,567; Queen et al. (1989) Proc. Natl. Acad. Sci. USA 86:10029-10033; and Antibody Engineering: A Practical Approach (Oxford University Press 1996).

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. In one embodiment, the polyclonal antibody contains a plurality of monoclonal antibodies with different epitope specificities, affinities, or avidities within a single antigen that contains multiple antigenic epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. These monoclonal antibodies will usually bind with at least a K_{d} of about 1 μM, more usually at least about 300 nM, typically at least about 30 nM, preferably at least about 10 nM, more preferably at least about 3 nM or better, usually determined by ELISA.

In a preferred embodiment, the antibody is a fully human antibody.

Cellular transport facilitators comprise a functional group or may be modified to comprise a functional group that allows for conjugation with one or more molecules of toxin. The cellular transport facilitator may be modified to include a spacer or linker group that itself contains a suitable conjugation handle. For example, an antibody or other peptide or amino-containing cellular transport facilitator, may be modified with 2-iminothiolane (Traut's reagent) to append a spacer group that terminates with a thiol moiety and thereby provides a handle for conjugation with a toxin that is suitably reactive (e.g., a maleimido, α-haloketo, or disulfide group). As used herein, the terms “spacer” or “linker” refer to a bifunctional compound that can be used to link a compound of Formula (I) or (II) to cellular transport facilitator to form a compound of Formula (IA) or (IIA). A variety of linkers can be used with the present compositions. For example, exemplary linkers, including their structure and synthesis, are described in PCT Appl. Publ. No. WO 2004/010957, and U.S. Pat. Publ. Nos. 2006/0074008, 2005/0238649, 2006/0024317, 2003/0083263, 2005/0238649, and 2005/0009751, each of which is incorporated herein by reference in its entirety and for all purposes.

In some embodiments, the linker is cleavable under intracellular conditions, such that cleavage of the linker releases the drug unit from the cellular transport facilitator in the intracellular environment. In yet other embodiments, the linker unit is not cleavable and the drug is released, for example, by degradation of the cellular transport facilitator. (See U.S. Pat. Publ. No. 2005/0238649 incorporated by reference herein in its entirety and for all purposes).

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker (1999) Pharm. Therapeutics 83:67-123). Most typical are peptidyl linkers that are cleavable by enzymes that are present in tumor cells expressing the target antigen. For example, a peptidyl linker that is cleavable by the thiol-dependent protease cathepsin-B, which is highly expressed in cancerous tissue, can be used (e.g., a Phe-Leu containing linker). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive and is cleaved for example, by hydrolysis, at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. See, e.g., U.S. Pat. Nos. 5,122,368, 5,824,805, and 5,622,929; Dubowchik and Walker (1999), supra; Neville et al. (1989) Biol. Chem. 264:14653-14661. Such linkers are relatively stable under neutral pH conditions, such as those in the blood, but are unstable at below pH 5.5 or 5.0, the approximate pH of the lysosome. In certain embodiments, the hydrolyzable linker is a thioether linker such as, e.g., a thioether attached to the therapeutic agent via an acylhydrazone bond. See, e.g., U.S. Pat. No. 5,622,929.

In yet other specific embodiments, the linker is a malonate linker (Johnson et al. (1995) Anticancer Res. 15:1387-93), a maleimidobenzoyl linker (Lau et al. (1995) Bioorg. Med. Chem. 3(10):1299-1304), or a 3′-N-amide analog (Lau et al. (1995) Bioorg. Med. Chem. 3(10):1305-12).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of drug conjugate compound, are cleaved when the drug conjugate compound presents in an extracellular environment (e.g., in plasma). Whether a linker is not substantially sensitive to the extracellular environment can be determined, for example, by incubating the drug conjugate with plasma for a predetermined time period (e.g., 2, 4, 8, 16, or 24 hours) and then quantitating the amount of free drug present in the plasma.

In other embodiments, conjugates of the cellular transport facilitator and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described by Vitetta et al. (1987) Science 238:1098. Carbon-14-labeled 1-isothiocyanatobenzyl-3-methyldiethylene triaminepentaacetic acid (MX-DTPA) is an exemplary chelating agent for conjugation of radionucleotide to the antibody (WO94/11026).

A number of different reactions are available for covalent attachment of drugs and/or linkers to cellular transport facilitators. This is often accomplished by reaction of the amino acid residues of the cellular transport facilitator, e.g., antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to binding agents is the Schiff base reaction. This method involves the periodate oxidation of a drug that contains glycol or hydroxy groups, thus forming an aldehyde which is then reacted with the cellular transport facilitator. Attachment occurs via formation of a Schiff base with amino groups of the binding agent. Isothiocyanates can also be used as coupling agents for covalently attaching drugs to cellular transport facilitators. Compounds with carboxylic acid termini may be activated with amide coupling reagents well-known in the art, for example, N-hydroxysuccinimide, and reacted with the terminal amino group of a lysine residue to form an amide linkage. Other techniques are known to the skilled artisan and within the scope of the present invention.

For conjugates with cellular transport facilitators, multiple equivalents of the toxin derivative may be appended to the facilitator. Drug loading may range from 1 to 20 toxin molecules per facilitator molecule. The average number of toxin molecules per facilitator molecule in preparation of conjugation reactions may be characterized by conventional means such as mass spectroscopy, ELISA assay, and HPLC. The quantitative distribution of Facilitator-Toxin-Conjugates may also be determined. In some instances, separation, purification, and characterization of homogeneous conjugates where drug loading is a defined value may be achieved by means such as reverse phase HPLC or electrophoresis. In exemplary embodiments, drug loading is 2 to 8 toxin molecules per facilitator molecule.

In the context of methods of treating, pharmaceutical compositions comprising compounds of the invention, including those comprising conjugates as described herein, may further comprise one or more pharmaceutically-acceptable excipients. A pharmaceutically-acceptable excipient is a substance that is non-toxic and otherwise biologically suitable for administration to a subject. Such excipients facilitate formulation and administration of a compound of the invention and are compatible with the active ingredient. Examples of pharmaceutically-acceptable excipients include stabilizers, lubricants, surfactants, diluents, anti-oxidants, binders, coloring agents, emulsifiers, or taste-modifying agents. In preferred embodiments, pharmaceutical compositions are sterile compositions. For antibody-toxin conjugates, suitable excipients include those described above, as well as Tween, sorbitol, sugars such as trehalose or sucrose, acetate buffers, and phosphate buffers.

The pharmaceutical compositions described herein may be formulated as solutions, emulsions, suspensions, or dispersions in suitable pharmaceutical solvents or carriers, or as pills, tablets, lozenges, suppositories, powders for reconstitution, or capsules along with solid carriers according to conventional methods known in the art for preparation of various dosage forms. For topical applications, the compounds described herein are preferably formulated as creams or ointments or a similar vehicle suitable for topical administration. The pharmaceutical compositions and compounds described herein may be administered in the inventive methods by a suitable route of delivery, e.g., oral, nasal, parenteral, rectal, topical, ocular, or by inhalation.

The term “treat” or “treating” as used herein is intended to refer to administration of a compound of the present invention to a subject for the purpose of creating a therapeutic benefit. Treating includes reversing, ameliorating, alleviating, inhibiting the progress of, or lessening the severity of, a disease, disorder, or condition, or one or more symptoms of that disease, disorder, or condition. The term “subject” refers to a mammalian patient in need of such treatment, such as a human.

In some embodiments the presently described compounds and conjugates are useful in treating cancer. Non-limiting embodiments include cancer(s) selected from bladder, lung, ovarian, kidney, breast or prostate cancer. Additionally, liquid tumor cancers such as leukemia are contemplated. In other embodiments, the cancer is breast or prostate cancer.

In treatment methods according to the invention, “an effective amount” means an amount or dose sufficient to generally bring about the desired therapeutic benefit in a subject needing such treatment. Effective amounts or doses of the compounds described herein may be ascertained by routine methods, such as modeling, dose escalation or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 2 mg of active compound per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID). In the context of drug-facilitator conjugates, a suitable dose is in the range of 1 to 10 mg per kilogram of the subject's body weight per dose, or from 3 to 8 mg per kilogram, or about 5 mg per kilogram, with administration of from 1 to 7 doses per day. For conjugates as described herein, determination of suitable doses is within the skill in the art.

When referring to modulating the target receptor, an “effective amount” means an amount sufficient to affect the activity of such receptor. Measuring the activity of the target receptor may be performed by routine analytical methods. Target receptor modulation is useful in a variety of settings, including assays. “Modulators” include both inhibitors and activators, where “inhibitors” refer to compounds that decrease, prevent, inactivate, desensitize or down-regulate target receptor expression or activity, and “activators” are compounds that increase, activate, facilitate, sensitize, or up-regulate target receptor expression or activity.

The compounds described herein may be used in the pharmaceutical compositions or methods described herein in combination with additional active ingredients. The additional active ingredients may be administered separately from a described compound of the invention or may be included with a compound or conjugate of the invention in a pharmaceutical composition according to the invention. For example, additional active ingredients are those that are known or discovered to be effective in treating cancer, including those active against another target associated with cancer, such as but not limited to, Velcade, Rituximab, Methotrexate, Herceptin, Vincristine, Prednisone, and Irinotecan, or a combination thereof. Such a combination may serve to increase efficacy, decrease one or more side effects, to promote internalization of the administered compound into cells, or decrease the required dose of a disclosed compound.

Compounds of Formula (I), Formula (IA), Formula (II), and Formula (IIA) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (e.g., amino, carboxy, hydroxyl, indole nitrogen, and other groups) from reaction conditions, and that such groups are removed under standard conditions when appropriate. Each of the reactions depicted in the following schemes is preferably run at a temperature from about room temperature to the reflux temperature of the organic solvent used. Unless otherwise specified, the variables are as defined above in reference to Formula (I).

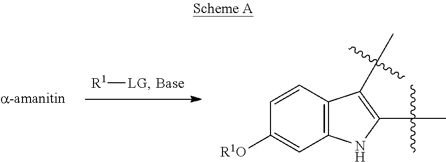

Referring to Scheme A, compounds of Formula (I) where R² is H may be prepared by alkylation of the 6-hydroxyindole group of α-amanitin with suitable alkylating agent, R¹-LG, wherein LG is a leaving group such as bromo, chloro, iodo, mesylate, or tosylate, in the presence of a base such as potassium tert-butoxide.

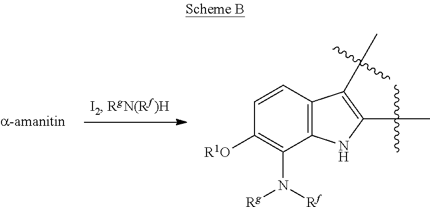

Referring to Scheme B, compounds of Formula (I) where R¹ is H, and compounds of Formula (II) wherein R¹ is H and z is 0 may be prepared by activation of the 7-position of the indole group of α-amanitin with a reagent such as iodine, followed by coupling with a suitably substituted amino reagent, R^{g}N(R^{f})H, which corresponds to an amino group at the diamine spacer in Formula (I), or where R^{f} is as defined in Formula (II), and R^{g} is the remainder of the R² group shown in Formula (II).

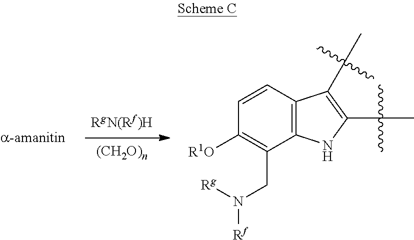

Referring to Scheme C, compounds of Formula (II) wherein R¹ is H and z is 1 may be prepared by reaction the indole group of α-amanitin with a suitably substituted amino reagent, R^{g}N(R^{f})H, where R^{f} is as defined in Formula (II), and R^{g} is the remainder of the R² group shown in Formula (II), in the presence of formaldehyde or a formaldehyde equivalent.

Amines R^{g}N(R^{f})H and alkylating agents R¹-LG may be prepared using methods known to one of skill in the art, including the particular methods described in the examples, as well as alkylation, protection/deprotection, amide coupling, reductive amination, halogenation, and the like. Alternatively, a portion of amines R^{g}N(R^{f})H and alkylating agents R¹-LG may be coupled to α-amanitin using methods such as those described above, and the remaining sections of the molecule built on after coupling is accomplished. One of skill in the art will recognize that the sequence of addition reactions may be chosen in a manner that is compatible with the functionalities of the subunits involved.

A compound of Formula (IA) or Formula (IIA) can be prepared by reacting a compound of Formula (I) or Formula (II) with a cellular transport facilitator. The generation of conjugates of compound of Formula (I) or Formula (II) with suitable cellular transport facilitators to obtain compound of Formula (IA) or Formula (IIA) can be accomplished by any technique known to the skilled artisan as exemplified in working examples in the specification. Briefly, the reactive cap or the R^{a} group in compounds of Formula (I) or Formula (II) may be reacted with an amino, thiol, carboxy, carbonyl, azide, or alkynyl group in the cellular transport facilitator, for example, a peptide, an antibody, a liposome, or a polymer, or in a linker attached thereto or capable of being attached thereto, to form a covalent bond. Such cellular transport facilitators can be treated with other reagents, such as 2-iminothiolane (Traut's reagent), to introduce a functional group which is reactive with the reactive cap or the R^{a} group in compounds of Formula (I) or Formula (II), before reacting with compounds of Formula (I) or Formula (II). Other techniques are known to the skilled artisan and within the scope of the present invention.

The following examples are offered to illustrate but not to limit the invention. The compounds are prepared using the general methods described above and the specific methods described below. Due to the size and complexity of the product compounds, ¹H NMR data was not a meaningful method for assessing compound identity or purity and therefore, mass spectrometry data was used for compound identification. Unless otherwise specified, compounds that were purified by preparative reverse-phase high performance liquid chromatography (RP-HPLC) were purified with a Phenomenex Synergi 10μ Max-R^{p} 80 Å column (150×30 mm) using 10% to 90% MeCN in 0.05% aqueous TFA as the eluent. These conditions were expected to yield TFA salt forms of the intermediate and target compounds.

Where the structures drawn herein are drawn as substructures, it is understood that the substructure shown is connected to the remainder of the α-amanitin structure at the 2- or 3-position of the central indole ring, consistent with Formulae (I) and (II). Example 1, below, shows the full structure of the example compound as well as the substructure according to the drawing convention used herein.

### Example 1

### 7′C-(4-(6-(maleimido)hexanoyl)piperazin-1-yl)-α-amanitin

### Full Structure:

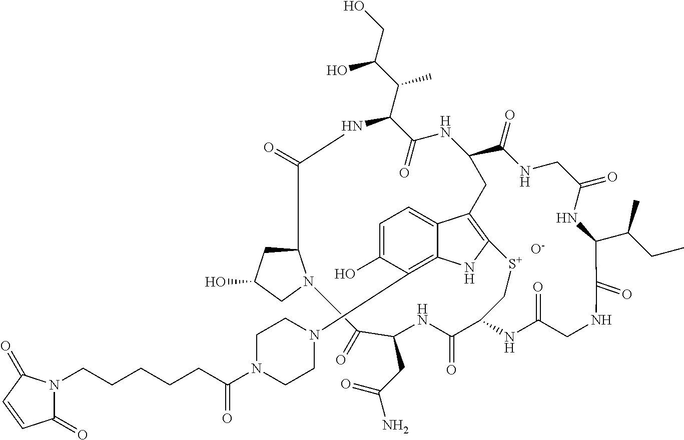

### Substructure According to Drawing Convention:

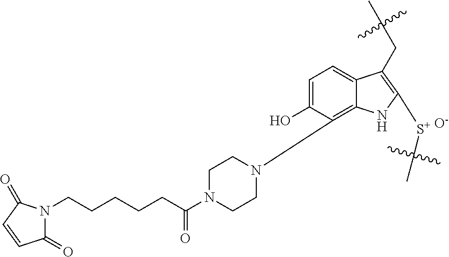

A solution of α-amanitin (69.3 mg, 75.4 μmol) in methanol (15 mL) was treated with a pre-mixed solution of 10 mM iodine/30 mM Boc-piperazine in methanol (7.54 mL) under argon atmosphere. The reaction mixture was stirred for 16 h at ambient temperature. The solution was concentrated in vacuo to approximately 3 mL and the resulting solution was added dropwise to a flask containing diethyl ether (45 mL) to precipitate the desired product. The supernatant was decanted and discarded. The precipitate was purified by preparative RP-HPLC to obtain 7′C-(4-N-Boc-piperazine-1-yl)-α-amanitin (Intermediate 1.1; 50 mg) as a white powder; [M+H]⁺=1104.50).

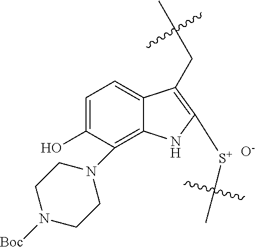

To a portion of purified 7′C-(4-N-Boc-piperazine-1-yl)-α-amanitin (15 mg) was added trifluoroacetic acid (TFA; 2 mL), methylene chloride (0.5 mL), and water (25 μL), and the reaction mixture was stirred for 1 h. The reaction mixture was concentrated under reduced pressure, and the residue was further dried under high vacuum pump to give 7′C-piperazine-1-yl-α-amanitin (Intermediate 1.2) as the TFA salt a gummy amorphous solid ([M+H]⁺=1004.40).

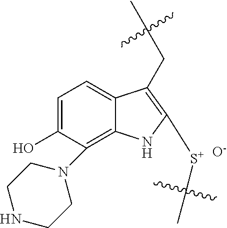

Intermediate 1.2 (15 mg) was immediately dissolved in tetrahydrofuran (THF; 2 mL) and N,N-dimethylsulfoxide (DMSO; 0.2 mL). To this solution was added N-(6-maleimideocaproyloxy)succinimide (4.6 mg) and pyridine (0.2 mL). The solution was stirred for 16 h at ambient temperature under argon atmosphere. The solution was concentrated under reduced pressure, and the residue was purified by preparative RP-HPLC to yield 2.6 mg of 7′C-(4-(6-(maleimido)hexanoyl)piperazin-1-yl)-α-amanitin ([M+H]⁺=1197.5; HRMS-ESI+ (m/z): [M+H]⁺ calcd for C₅₃H₇₄N₁₃O₁₇S, 1196.50411. found, 1196.50500). The composition of Example 1 was conjugated to H16-7.8 MAb in the same manner set forth in Example 76.

### Example 2

### 7′C-(4-(6-(maleimido)hexanamido)piperidin-1-yl)-α-amanitin

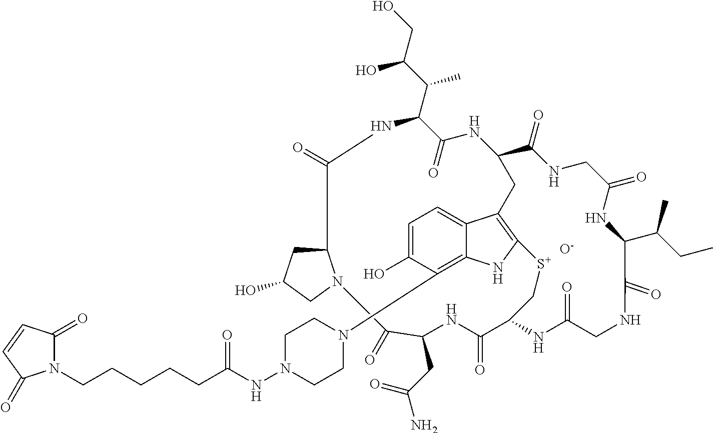

To α-amanitin (25 mg, 0.027 mmol) in methanol (10 mL) was added 4.1 mL of a pre-mixed solution of 10 mM iodine and 30 mM 4-(N-Boc-amino)piperidine in methanol under an argon atmosphere. The reaction mixture was stirred overnight at room temperature. The reaction mixture was then concentrated under reduced pressure to 2 mL and was added dropwise into diethyl ether (45 mL) and the resulting precipitate was separated from the supernatant. The precipitate was purified by preparative RP-HPLC. A total of 15 mg of 7′C-(4-N-Boc-aminopiperidin-1-yl)-α-amanitin (Intermediate 2.1) was obtained as white solid. [M+H]⁺=1118.60.

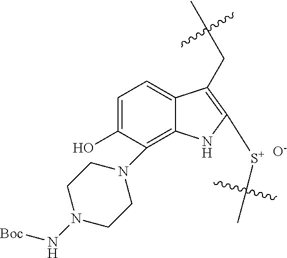

To the above Intermediate 2.1 (15 mg) was added a mixture of TFA (2 mL), methylene chloride (0.5 mL) and water (25 μL) and the reaction mixture was stirred for 1 h. The reaction mixture was concentrated under reduced pressure and the resulting residue was further dried under high vacuum. The residue was used without further purification. A total of 15 mg of 7′C-(4-aminopiperidin-1-yl)-α-amanitin (Intermediate 2.2) was obtained as the TFA salt as a gummy amorphous solid. [M+H]⁺=1017.60.

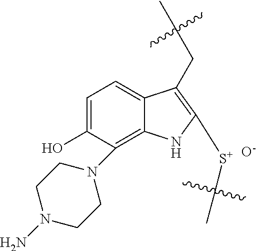

To a solution of Intermediate 2.2 (15 mg) in THF (2 mL), DMSO (0.2 mL), and pyridine (0.2 mL) was added N-(6-maleimideocaproyloxy)succinimide (4.6 mg, 0.015 mmol). The reaction mixture was stirred at reflux under an argon atmosphere. The reaction mixture was evaporated under reduced pressure and the residue was purified by preparative RP-HPLC. A total of 2.6 mg of 7′C-(4-(6-(maleimido)hexanamido)piperidin-1-yl)-α-amanitin was obtained as a gray-colored solid. HRMS-ESI+ (m/z): [M+H]⁺ calcd for C₅₄H₇₆N₁₃O₁₇S, 1210.51976. found, 1210.52431. [M+H]⁺=1210.7.

The composition of Example 2 was conjugated to H16-7.8 MAb in the same manner set forth in Example 76.

### Example 3

### 7′C-(4-(6-(6-(maleimido)hexanamido)hexanoyl)piperazin-1-yl)-α-amanitin

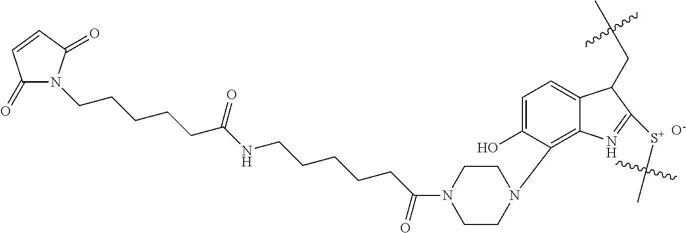

To a solution of Intermediate 1.2 (11 mg) in THF (2 mL) and DMSO (0.2 mL) was added 2,5-dioxopyrrolidin-1-yl 6-(6-(2,5-dioxo-2,5-dihydro-1H-pyrrol-1-yl)hexanamido)hexanoate (5 mg, 0.012 mmol) and pyridine (0.2 mL). After 1 h, the reaction mixture was concentrated under reduced pressure and the residue was purified by preparative RP-HPLC. A total of 5 mg of Example 3 was obtained as a white solid. HRMS-ESI+ (m/z): [M+H]⁺ calcd for C₅₉H₈₅N₁₄O₁₈S, 1309.58817. found, 1309.58179.

### Example 4

### 7′C-(4-(4-((maleimido)methyl)cyclohexanecarbonyl)piperazin-1-yl)-α-amanitin

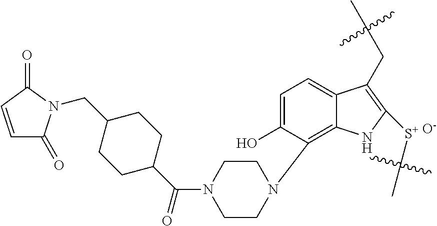

To a solution of Intermediate 1.2 (11 mg) in THF (2 mL) and DMSO (0.5 mL) was added 2,5-dioxopyrrolidin-1-yl 4-((2,5-dioxo-2,5-dihydro-1H-pyrrol-1-yl)methyl)cyclohexanecarboxylate (3.8 mg, 0.011 mmol), diisopropylethylamine (12.5 μL) and pyridine (0.2 mL). After 4 h, the reaction mixture was concentrated under reduced pressure and the resulting residue was purified by preparative RP-HPLC. A total of 5 mg of Example 4 was obtained as a white solid. HRMS-ESI+ (m/z): [M+H]⁺ calcd for C₅₅H₇₆N₁₃O₁₇S, 1222.51976. found, 1222.52063.

### Example 5

### 7′C-(4-(6-(4-((maleimido)methyl)cyclohexanecarboxamido)hexanoyl)piperazin-1-yl)-α-amanitin

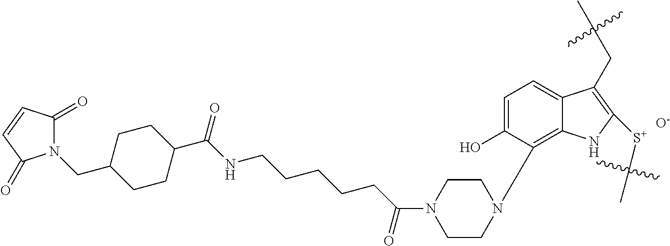

To a solution of Intermediate 1.2 (11 mg) in THF (2 mL) and DMSO (0.5 mL) was added 2,5-dioxopyrrolidin-1-yl 6-(4-((2,5-dioxo-2,5-dihydro-1H-pyrrol-1-yl)methyl)cyclohexanecarboxamido)hexanoate (5 mg, 0.011 mmol), diisopropyethylamine (10 μL), and pyridine (0.2 mL). After 1 h, the reaction mixture was concentrated under reduced pressure and the resulting residue was purified by preparative RP-HPLC. A total of 5 mg of Example 5 was obtained as a white solid. HRMS-ESI+ (m/z): [M+H]⁺ calcd for C₆₁H₈₇N₁₄O₁₈S, 1335.60382. found, 1335.6038.

### Example 6

### 7′C-(4-(2-(6-(maleimido)hexanamido)ethyl)piperidin-1-yl)-α-amanitin

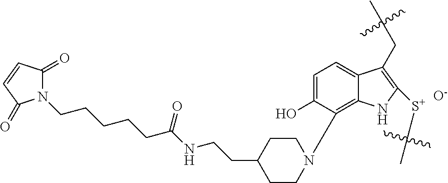

To α-amanitin (27.2 mg, 0.030 mmol) in methanol (3 mL) was added a 2.96 mL of a pre-mixed solution of 10 mM of iodine and 30 mM of 4-(N-Boc-aminoethyl)piperidine in methanol under an argon atmosphere. The reaction mixture was stirred overnight at room temperature. The reaction mixture was concentrated under reduced pressure to 2 mL and was added dropwise into diethyl ether (45 mL) and the precipitate was separated from the supernatant. The precipitate was purified by preparative RP-HPLC. A total of 12 mg of Intermediate 6.1 was obtained as a white solid. [M+H]⁺=1145.45.

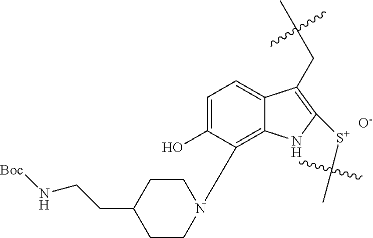

To Intermediate 6.1 (12 mg) was added a mixture of TFA (2 mL), methylene chloride (0.5 mL), and anisole (25 μL). After 1 h, the reaction mixture was concentrated under reduced pressure and the residue was further dried under high vacuum. The residue was used without further purification. A total of 15 mg of Intermediate 6.2 was obtained as the TFA salt as a gummy amorphous solid. [M+H]⁺=1045.20.

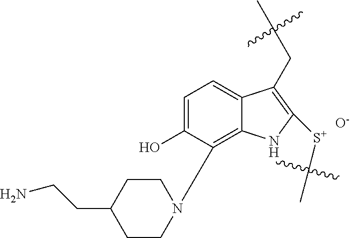

To a solution of Intermediate 6.2 (6 mg) in THF (2 mL), DMSO (0.5 mL), and pyridine (0.2 mL) was added N-(6-maleimideocaproyloxy)succinimide (2.4 mg, 0.008 mmol). After 1 h of stirring at 50° C. under an argon atmosphere, the reaction mixture was concentrated under reduced pressure and the residue was purified by preparative RP-HPLC. A total of 2 mg of Example 6 was obtained as a gray-colored solid. HRMS-ESI+ (m/z): [M+H]⁺ calcd for C₅₆H₈₀N₁₃O₁₇S, 1238.55106. found, 1238.55528.

### Example 7

### 7′C-(4-(2-(6-(6-(maleimido)hexanamido)hexanamido)ethyl)piperidin-1-yl)-α-amanitin

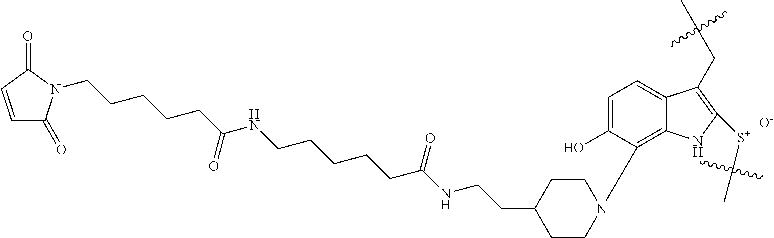

To a solution of Intermediate 6.2 (6 mg) in THF (2 mL), DMSO (0.5 mL), and pyridine (0.2 mL) was added 2,5-dioxopyrrolidin-1-yl 6-(6-(2,5-dioxo-2,5-dihydro-1H-pyrrol-1-yl)hexanamido)hexanoate (2.4 mg, 0.008 mmol). After 1 h of stirring at 50° C. under an argon atmosphere, the solution was concentrated under reduced pressure and the residue was purified by preparative RP-HPLC. A total of 2 mg of Example 7 was obtained as gray-colored solid. HRMS-ESI+ (m/z): [M+2H]²⁺ calcd for (C₆₂H₉₂N₁₄O₁₈S)/2, 676.32121. found, 676.32068.

### Example 8

### 7′C-(4-(2-(4-((maleimido)methyl)cyclohexanecarboxamido)ethyl)piperidin-1-yl)-α-amanitin

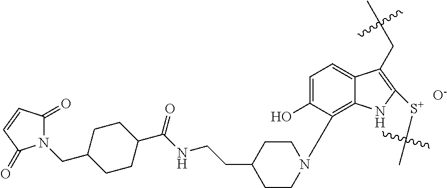

To a solution of Intermediate 6.2 (7 mg) in THF (2 mL), DMSO (0.5 mL), and pyridine (0.2 mL) was added 2,5-dioxopyrrolidin-1-yl 4-((2,5-dioxo-2,5-dihydro-1H-pyrrol-1-yl)methyl)cyclohexanecarboxylate (2.4 mg, 0.007 mmol). After 1 h of stirring at room temperature under an argon atmosphere, the solution was concentrated under reduced pressure and the residue was purified by preparative RP-HPLC. A total of 2 mg of Example 8 was obtained as a gray-colored solid. HRMS-ESI+ (m/z): [M+2H]²⁺ calcd for (C₅₈H₈₃N₁₃O₁₇S)/2, 632.78701. found, 632.78726.

### Example 9

### 7′C-(4-(2-(6-(4-((maleimido)methyl)cyclohexanecarboxamido)hexanamido)ethyl)piperidin-1-yl)-α-amanitin

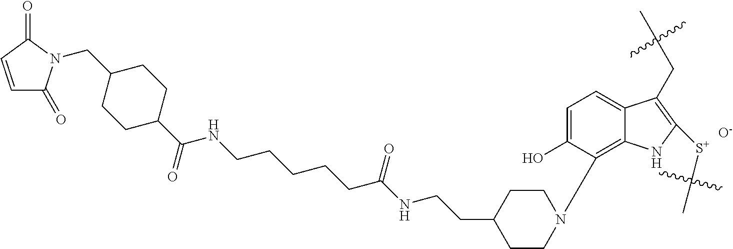

To a solution of Intermediate 6.2 (7 mg) in THF (2 mL), DMSO (0.5 mL), and pyridine (0.2 mL) was added 2,5-dioxopyrrolidin-1-yl 6-(4-((2,5-dioxo-2,5-dihydro-1H-pyrrol-1-yl)methyl)cyclohexanecarboxamido)hexanoate (3.2 mg, 0.007 mmol). After 1 h of stirring at room temperature under an argon atmosphere, the solution was concentrated under reduced pressure and the residue was purified by preparative RP-HPLC. A total of 2 mg of Example 9 was obtained as a gray-colored solid. HRMS-ESI+ (m/z): [M+2H]²⁺ calcd for (C₆₄H₉₄N₁₄O₁₈S)/2, 689.32917. found, 689.32917.

### Example 10

### 7′C-(4-(2-(3-carboxypropanamido)ethyl)piperidin-1-yl)-α-amanitin

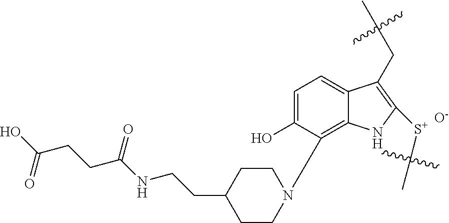

To a solution of Intermediate 6.2 (5.5 mg) in DMSO (1 mL) was added succinic anhydride (0.6 mg, 0.011 mmol) and diisopropylethylamine (1.8 μL). After stirring for 1 h at room temperature under an argon atmosphere, the reaction mixture was concentrated under reduced pressure, and the resulting residue was purified by preparative RP-HPLC. A total of 3 mg of Example 10 was obtained as a gray-colored solid. HRMS-ESI+ (m/z): [M+H]⁺ calcd for C₅₀H₇₃N₁₂O₁₇S, 1145.49321. found, 1145.49928.

The compounds in Examples 11-25 may be prepared using methods analogous to those described above, starting from Intermediate 1.2 or 6.2, and reacting as described above with suitable, commercially available acylating reagents; or reacting α-amanitin with 1-N-Boc-3-R-(aminomethyl)pyrrolidine, removing the Boc protecting group, and acylating as described in the preceding examples.

### Example 11

### 7′C-(4-(2-(2-bromoacetamido)ethyl)piperidin-1-yl)-α-amanitin

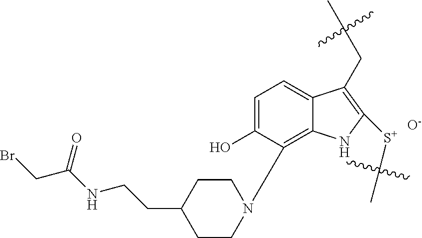

### Example 12

### 7′C-(4-(2-(3-(pyridin-2-yldisulfanyl)propanamido)ethyl)piperidin-1-yl)-α-amanitin

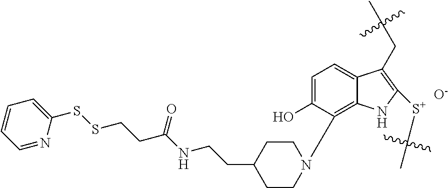

### Example 13

### 7′C-(4-(2-(4-(maleimido)butanamido)ethyl)piperidin-1-yl)-α-amanitin

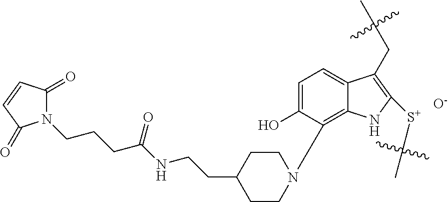

### Example 14

### 7′C-(4-(2-(maleimido)acetyl)piperazin-1-yl)-α-amanitin

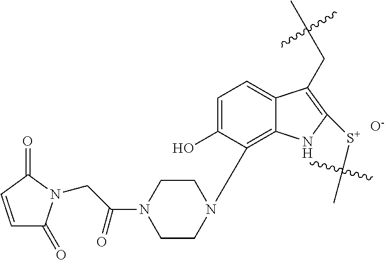

### Example 15

### 7′C-(4-(3-(maleimido)propanoyl)piperazin-1-yl)-α-amanitin

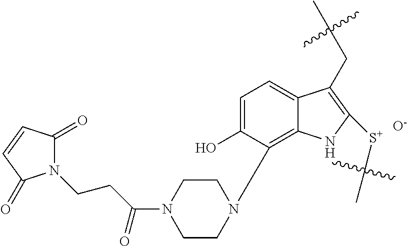

### Example 16

### 7′C-(4-(4-(maleimido)butanoyl)piperazin-1-yl)-α-amanitin

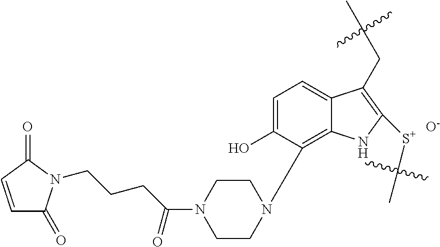

### Example 17

### 7′C-(4-(2-(6-(4-((maleimido)methyl)cyclohexanecarboxamido)hexanamido)ethyl)piperidin-1-yl)-α-amanitin

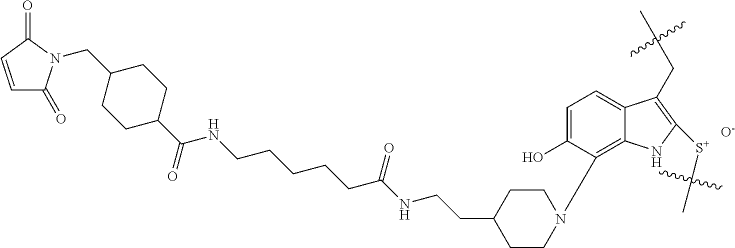

### Example 18

### 7′C-(3-((6-(maleimido)hexanamido)methyl)pyrrolidin-1-yl)-α-amanitin

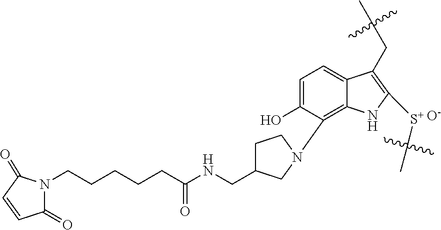

### Example 19

### 7′C-(3-((6-(6-(maleimido)hexanamido)hexanamido)methyl)pyrrolidin-1-yl)-α-amanitin

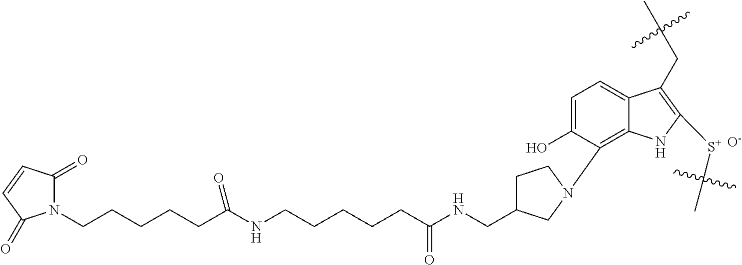

### Example 20

### 7′C-(3-((4-((maleimido)methyl)cyclohexanecarboxamido)methyl)pyrrolidin-1-yl)-α-amanitin

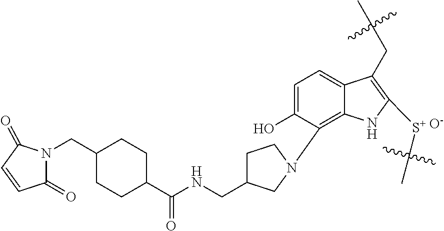

### Example 21

### 7′C-(3-((6-((4-(maleimido)methyl)cyclohexanecarboxamido)hexanamido)methyl)pyrrolidin-1-yl)-α-amanitin

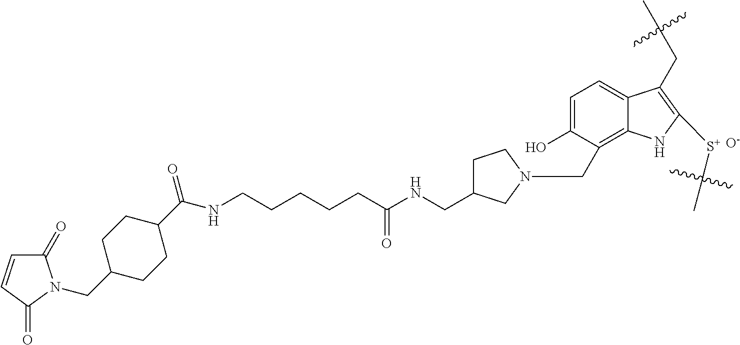

### Example 22

### 7′C-(4-(2-(6-(2-(aminooxy)acetamido)hexanamido)ethyl)piperidin-1-yl)-α-amanitin

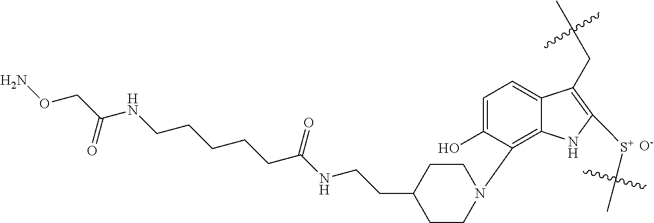

### Example 23

### 7′C-(4-(2-(4-(2-(aminooxy)acetamido)butanamido)ethyl)piperidin-1-yl)-α-amanitin

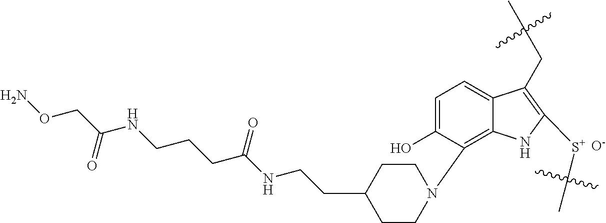

### Example 24

### 7′C-(4-(4-(2-(aminooxy)acetamido)butanoyl)piperazin-1-yl)-α-amanitin

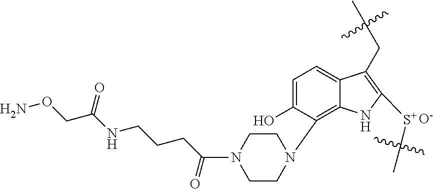

### Example 25

### 7′C-(4-(6-(2-(aminooxy)acetamido)hexanoyl)piperazin-1-yl)-α-amanitin

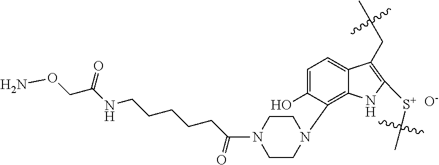

### Example 26

### 7′C-((4-(6-(maleimido)hexanamido)piperidin-1-yl)methyl)-α-amanitin

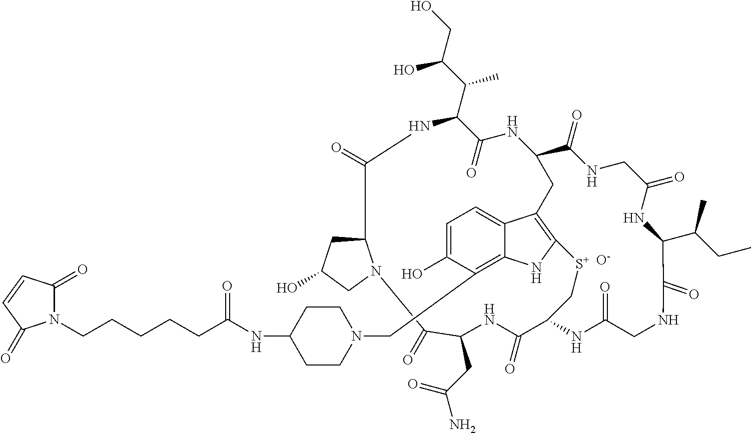

To a solution of α-amanitin (26 mg) and 4-(Boc-amino)piperidine (45 mg) in ethanol (10 mL) was added paraformaldehyde (23 mg), and the reaction mixture was heated at reflux for 7 h. The mixture was concentrated under reduced pressure, and the crude residue was dissolved in methanol (1 mL) and was added dropwise to diethyl ether (40 mL). The resulting precipitate was collected and purified by preparative RP-HPLC. The resulting 7′C—N-4-Boc-aminopiperidin-1-yl-methyl derivative (Intermediate 26.1; 23 mg) ([M+H]⁺=1131.7) was recovered as a white powder.

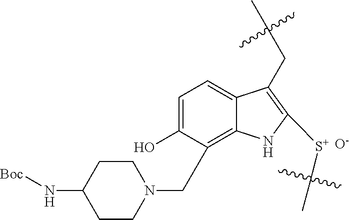

To Intermediate 26.1 (23 mg) was added TFA (2 mL), methylene chloride (0.5 mL), and anisole (25 μL), and the reaction mixture was stirred for 1 h. The reaction mixture was concentrated under reduced pressure, and the residue was further dried under high vacuum. The compound 7′C-aminopiperidine-1-yl-methyl derivative (Intermediate 26.2; [M+H]⁺=1031.5) was isolated as a TFA salt without further purification.

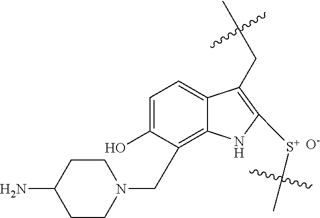

After isolation, Intermediate 26.2 was immediately dissolved in THF (2 mL) and DMSO (0.4 mL). To this solution was added N-(6-maleimideocaproyloxy)succinimide (7.5 mg), N,N-dimethylaminopyridine (1 mg), and pyridine (0.2 mL). The solution was stirred for 1.5 h at 50° C. under argon atmosphere. The solution was concentrated under reduced pressure, and the residue was purified by preparative RP-HPLC to yield 2.62 mg of 7′C-((4-(6-(maleimido)hexanamido)piperidin-1-yl)methyl)-α-amanitin ([M+H]⁺=1224.8; HRMS-ESI+(m/z): [M+H]⁺ calcd for C₅₅H₇₈N₁₃O₁₇S, 1224.53541. found, 1224.53988).

### Example 27

### 7′C-((4-(2-(6-(maleimido)hexanamido)ethyl)piperidin-1-yl)methyl)-α-amanitin

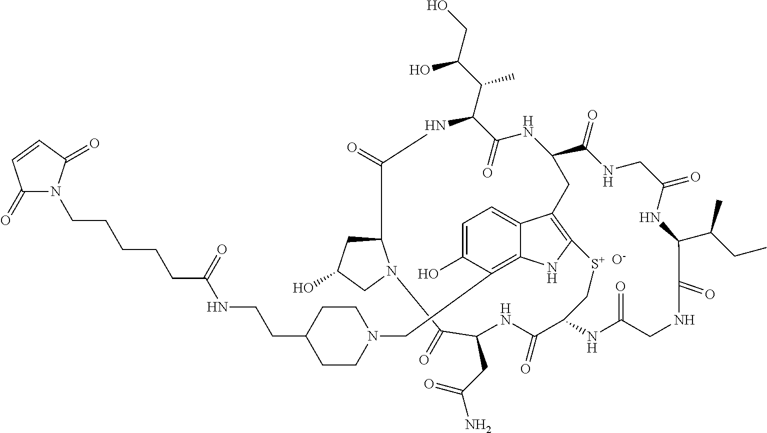

To a solution of α-amanitin (25 mg, 0.027 mmol) and 4-(N-Boc-aminoethyl)piperidine (49.7 mg, 0.22 mmol) in ethanol (2 mL) was added paraformaldehyde (22 mg). After 3 h, the reaction mixture was concentrated under reduced pressure and the resulting residue was purified by preparative RP-HPLC. A total of 25 mg of Intermediate 27.1 was obtained as a white solid. [M+H]⁺=1159.38.

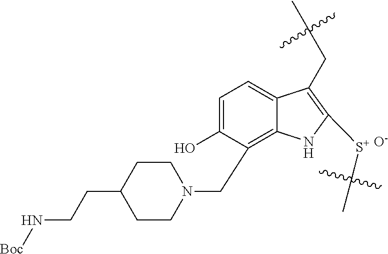

To Intermediate 27.1 (25 mg) was added a mixture of TFA (2 mL), methylene chloride (0.5 mL) and anisole (25 μL). After 1 h of stirring at room temperature, the reaction mixture was concentrated under reduced pressure and the resulting residue was further dried under high vacuum. The residue was used without further purification. Intermediate 27.2 (38 mg) was obtained as a TFA salt as a gummy amorphous solid. [M+H]⁺=1059.51.

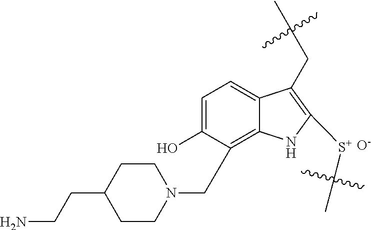

To a solution of Intermediate 27.2 (38 mg) in THF (2 mL), DMSO (0.2 mL), and pyridine (0.4 mL) was added N-(6-maleimideocaproyloxy)succinimide (12 mg, 0.039 mmol) and diisopropylethylamine (10 μL). After 4 h of stirring at 50° C. under an argon atmosphere, the reaction mixture was concentrated under reduced pressure, and the resulting residue was purified by preparative RP-HPLC. A total of 11 mg of Example 27 was obtained as gray-colored solid. HRMS-ESI+ (m/z): [M+H]⁺ calcd for C₅₇H₈₂N₁₃O₁₇S, 1252.56671. found, 1252.5739; [M+H]⁺=1252.39.

The composition of Example 27 was conjugated to H16-7.8 MAb in the same manner set forth in Example 76.

### Example 28

### 7′C-((4-(6-(maleimido)hexanoyl)piperazin-1-yl)methyl)-α-amanitin

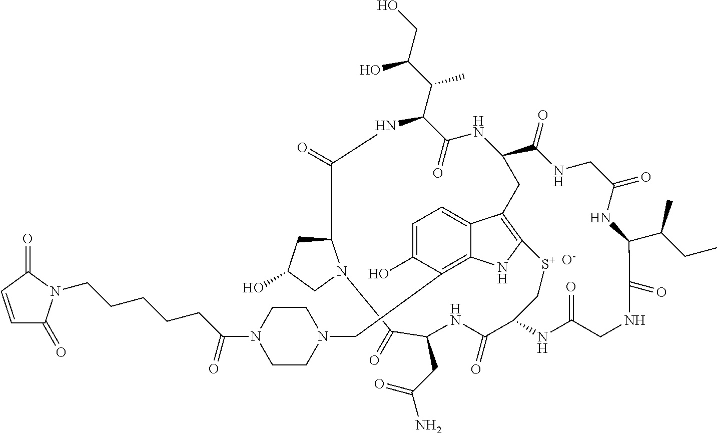

To a solution of Boc-piperazine (200 mg, 1.074 mmol) and maleimidocaproic acid (249.5 mg, 1.181 mmol) in methylene chloride (10 mL) was added 1-ethyl-3-(3-dimethylaminopropyl)carbodiimide hydrochloride (226.4 mg, 1.181 mmol) and N,N-dimethylaminopyridine (13.1 mg, 0.017 mmol). After stirring at room temperature for 2 h, the reaction mixture was diluted with 100 mL of ethyl acetate and this organic solution was washed with saturated sodium bicarbonate (100 mL), 0.1 N HCl (100 mL), and saturated brine (100 mL). The combined organic layers were concentrated under reduced pressure and the resulting residue was purified by preparative RP-HPLC. A total of 100 mg of Intermediate 28.1 was obtained as gray-colored solid. [M+H]⁺=380.30.

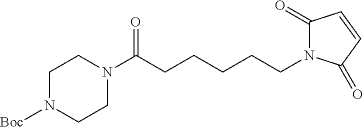 [SMILES: CC(C)(C)OC(=O)N1CCN(C(=O)CCCCCN2C(=O)C=CC2=O)CC1]

To Intermediate 28.1 (100 mg) was added a mixture of TFA (2 mL), methylene chloride (0.5 mL), and anisole (25 μL). After 1 h of stirring at room temperature, the reaction mixture was concentrated under reduced pressure, and the resulting residue was further dried under high vacuum. The residue, Intermediate 28.2, was obtained as the TFA salt, and used further without purification. [M+H]⁺=280.30.

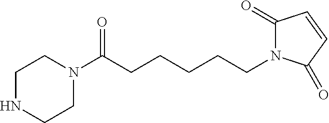 [SMILES: O=C(CCCCCN1C(=O)C=CC1=O)N1CCNCC1]

α-Amanitin (25 mg, 0.027 mmol) and paraformaldehyde (15 mg) were added to a solution of Intermediate 28.2 in ethanol (1.5 mL, ˜0.033 mM) and this mixture was further diluted with additional ethanol (2 mL). After stirring overnight at 50° C., the reaction mixture was concentrated under reduced pressure, and the resulting residue was purified by preparative RP-HPLC. A total of 20 mg of Example 28 was obtained as white solid. HRMS-ESI+ (m/z): [M+H]⁺ calcd for C₅₄H₇₆N₁₃O₁₇S, 1210.51976. found, 1210.52531; [M+H]⁺=1210.8.

### Example 29

### (R)-7′C-((3-((6-(maleimido)hexanamido)methyl)pyrrolidin-1-yl)methyl)-α-amanitin

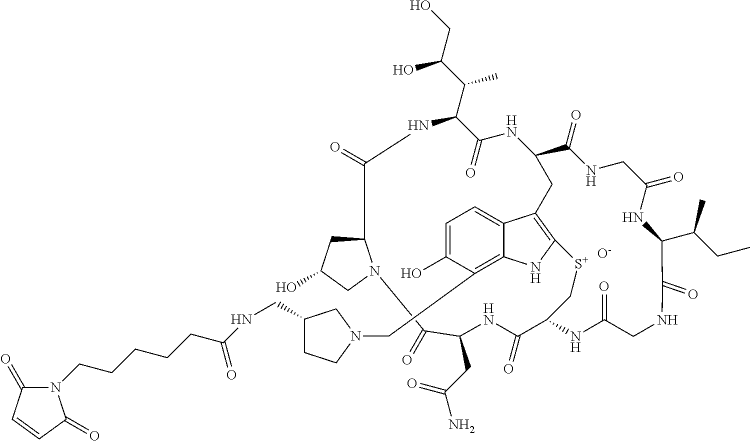

To a solution of 1-N-Boc-3-R-(aminomethyl)pyrrolidine (200 mg, 0.999 mmol) and maleimidocaproic acid (232 mg, 1.098 mmol) in methylene chloride (4 mL) was added 1-ethyl-3-(3-dimethylaminopropyl)carbodiimide hydrochloride (229 mg, 1.198 mmol). After stirring for 2 h at room temperature, the mixture was diluted with 50 mL of ethyl acetate, washed with saturated sodium bicarbonate (100 mL), 0.1 N HCl (100 mL), and saturated brine (100 mL). The combined organic layers were concentrated under reduced pressure, and the resulting residue purified by preparative RP-HPLC. A total of 360 mg of Intermediate 29.1 was obtained as a colorless oil. [M+H]⁺=394.39.

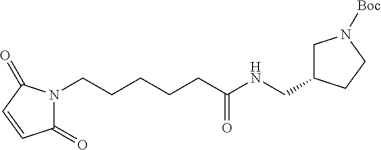 [SMILES: CC(C)(C)OC(=O)N1CC[C@H](CNC(=O)CCCCCN2C(=O)C=CC2=O)C1]

To Intermediate 29.1 (360 mg) was added a mixture of TFA (2 mL), methylene chloride (0.5 mL) and anisole (25 μL). After stirring for 1 h at room temperature, the reaction mixture was concentrated under reduced pressure and the resulting residue then further dried under high vacuum. The residue was obtained as a TFA salt (Intermediate 29.2) and was used subsequently without further purification. [M+H]⁺=294.12.

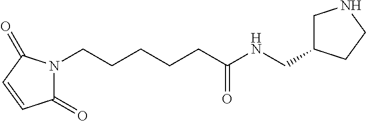 [SMILES: O=C(CCCCCN1C(=O)C=CC1=O)NC[C@H]1CCNC1]

α-Amanitin (9 mg, 0.009 mmol), paraformaldehyde (5 mg), and Intermediate 29.2 (18 mg) were dissolved in ethanol (2.5 mL). After stirring overnight at 75° C., the reaction mixture was concentrated under reduced pressure, and the resulting residue was purified by preparative RP-HPLC. A total of 8 mg of Example 29 was obtained as gray-colored solid. HRMS-ESI+ (m/z): [M+H]⁺ calcd for C₅₅H₇₈N₁₃O₁₇S, 1224.53541. found, 1224.54296; [M+H]⁺=1224.8.

### Example 30

### (S)-7′C-((3-((6-(maleimido)hexanamido)methyl)pyrrolidin-1-yl)methyl)-α-amanitin

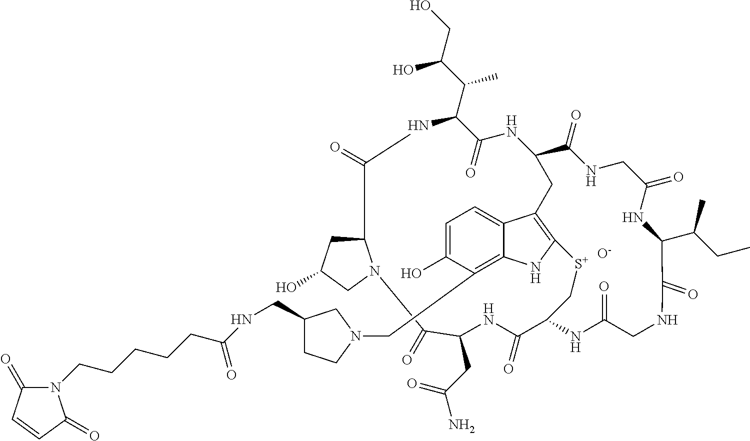

To a solution of 1-N-Boc-3-S-(aminomethyl)pyrrolidine (200 mg, 0.999 mmol) and maleimidocaproic acid (232 mg, 1.098 mmol) in methylene chloride (4 mL) was added 1-ethyl-3-(3-dimethylaminopropyl)carbodiimide hydrochloride (229 mg, 1.198 mmol). After stirring for 2 h at room temperature, the mixture was diluted with 50 mL of ethyl acetate, and the organic solution was washed with saturated sodium bicarbonate (100 mL), 0.1 N HCl (100 mL), and saturated brine (100 mL). The combined organic layers were evaporated, and the resulting residue purified by preparative RP-HPLC. A total of 360 mg of Intermediate 30.1 was obtained as colorless oil. [M+H]⁺=394.39.

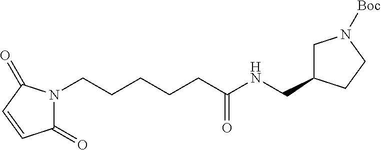 [SMILES: CC(C)(C)OC(=O)N1CC[C@@H](CNC(=O)CCCCCN2C(=O)C=CC2=O)C1]

To Intermediate 30.1 (360 mg) was added a mixture of TFA (2 mL), methylene chloride (0.5 mL), and anisole (25 μL). After stirring for 1 h at room temperature, the reaction mixture was concentrated under reduced pressure and the resulting residue then further dried under high vacuum. The residue (containing Intermediate 30.2 and TFA salt) was used subsequently without further purification. [M+H]⁺=294.12.

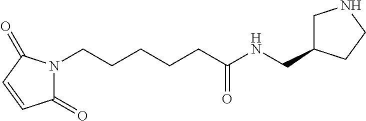 [SMILES: O=C(CCCCCN1C(=O)C=CC1=O)NC[C@@H]1CCNC1]

To a mixture of α-amanitin (23.5 mg, 0.026 mmol) and Intermediate 30.2 (60 mg) in ethanol (3 mL) was added paraformaldehyde (12 mg) under an anhydrous nitrogen atmosphere at 75° C. After stirring for 18 h, the reaction mixture was concentrated under reduced pressure, and the resulting residue was purified by preparative RP-HPLC with a Phenomenex Synergi 10μ Max-R^{p} 80 Å column (150×30 mm) using 10% to 90% MeCN in 0.1% formic acid as the eluent. A total of 20 mg of Example 30 was obtained as gray-colored solid. HRMS-ESI+ (m/z): [M+H]⁺ calcd for C₅₅H₇₈N₁₃O₁₇S, 1224.53541. found, 1224.54226; [M+H]⁺=1224.8.

### Example 31

### 7′C-((4-(2-(6-(6-(maleimido)hexanamido)hexanamido)ethyl)piperidin-1-yl)methyl)-α-amanitin

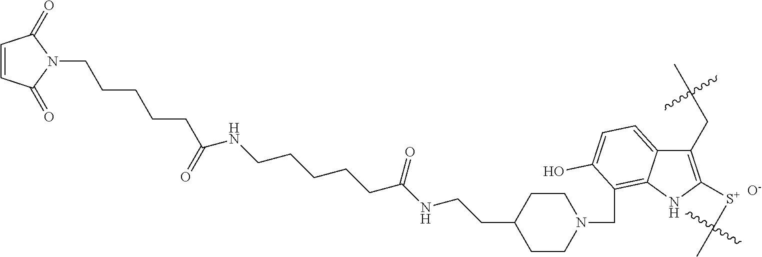

To a solution of Intermediate 27.2 (11 mg) in THF (2 mL), DMSO (0.5 mL), and pyridine (0.2 mL) was added 2,5-dioxopyrrolidin-1-yl 6-(6-(2,5-dioxo-2,5-dihydro-1H-pyrrol-1-yl)hexanamido)hexanoate (4.7 mg, 0.011 mmol), and diisopropylethylamine (12 pt). After 16 h of stirring at room temperature under an argon atmosphere, the reaction mixture was concentrated under reduced pressure, and the resulting residue was purified by preparative RP-HPLC. A total of 5 mg of Example 31 was obtained as gray-colored solid. HRMS-ESI+ (m/z): [M+H]⁺ calcd for C₆₃H₉₃N₁₄O₁₈S, 1365.65077. found, 1365.65387.

### Example 32

### 7′C-((4-(2-(4-((maleimido)methyl)cyclohexanecarboxamido)ethyl)piperidin-1-yl)methyl)-α-amanitin

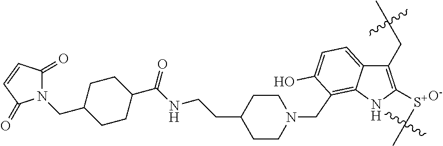

To a solution of Intermediate 27.2 (11 mg) in THF (2 mL), DMSO (0.5 mL), and pyridine (0.2 mL) was added 2,5-dioxopyrrolidin-1-yl 4-((2,5-dioxo-2,5-dihydro-1H-pyrrol-1-yl)methyl)cyclohexanecarboxylate (3.8 mg, 0.011 mmol), and diisopropylethylamine (12 μL). After 2 h of stirring at room temperature under an argon atmosphere, the reaction mixture was concentrated under reduced pressure, and the resulting residue was purified by preparative RP-HPLC. A total of 5 mg of Example 32 was obtained as gray-colored solid. HRMS-ESI+ (m/z): [M+H]⁺ calcd for C₅₉H₈₄N₁₃O₁₇S, 1278.58236. found, 1278.58616.

### Example 33

### 7′C-((4-(2-(6-(4-((maleimido)methyl)cyclohexanecarboxamido)hexanamido)ethyl)piperidin-1-yl)methyl)-α-amanitin

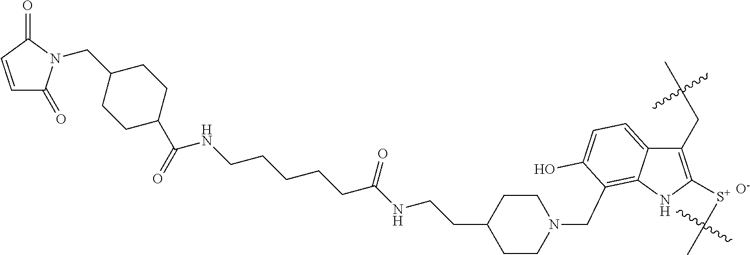

To a solution of Intermediate 27.2 (11 mg) in THF (2 mL), DMSO (0.5 mL), and pyridine (0.2 mL) was added 2,5-dioxopyrrolidin-1-yl 6-(4-((2,5-dioxo-2,5-dihydro-1H-pyrrol-1-yl)methyl)cyclohexanecarboxamido)hexanoate (5 mg, 0.011 mmol), and diisopropylethylamine (10 μL). After 1 h of stirring at room temperature under an argon atmosphere, the reaction mixture was concentrated under reduced pressure, and the residue was purified by preparative RP-HPLC. A total of 2 mg of Example 33 was obtained as gray-colored solid. HRMS-ESI+ (m/z): [M+2H]²⁺ calcd for (C₆₅H₉₆N₁₄O₁₈S)/2, 696.33686. found, 696.33687.

### Example 34

### 7′C-((4-(2-(6-(maleimido)hexanamido)ethyl)piperazin-1-yl)methyl)-α-amanitin

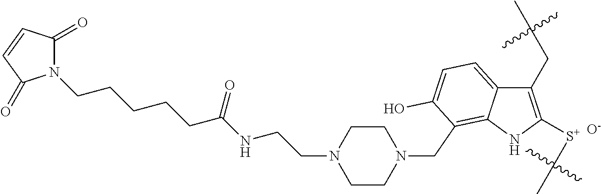

To a solution of 4-N-(2-aminoethyl)-1-N-Boc-piperazine (35 mg, 0.153 mmol) in methylene chloride (4 mL) was added maleimidocaproic acid-N-hydroxysuccinimide (51.8 mg, 0.168 mmol). After 1 h of stirring at room temperature, the reaction mixture was diluted with 50 mL of ethyl acetate, and this organic solution was washed with saturated sodium bicarbonate (100 mL), 0.5 N HCl (100 mL), and saturated brine (100 mL). The combined organic layers were concentrated under reduced pressure, and the resulting residue was purified by preparative RP-HPLC. A total of 80 mg of Intermediate 34.1 as a TFA salt was obtained as a white solid. [M+H]⁺=422.80.

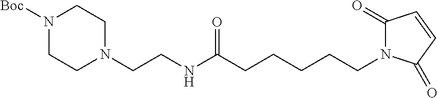 [SMILES: CC(C)(C)OC(=O)N1CCN(CCNC(=O)CCCCCN2C(=O)C=CC2=O)CC1]

To Intermediate 34.1 (77 mg) was added a mixture of TFA (2 mL), methylene chloride (0.5 mL), and anisole (10 μL). After 30 min of stirring at room temperature, the reaction mixture was concentrated under reduced pressure, and the resulting residue was then further dried under high vacuum. Intermediate 34.2 was obtained as TFA salt and used further without additional purification. [M+H]⁺=323.18.

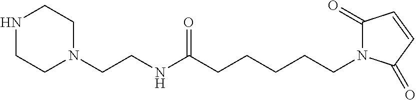 [SMILES: O=C(CCCCCN1C(=O)C=CC1=O)NCCN1CCNCC1]

To α-amanitin (8.1 mg, 0.009 mmol), paraformaldehyde (5 mg), and crude Intermediate 34.2 (10 mg) was added ethanol (3 mL). After 18 h of stirring at 50° C., the reaction mixture was concentrated under reduced pressure and the resulting residue was purified by preparative RP-HPLC. A total of 6 mg of Example 34 was obtained as gray-colored solid. HRMS-ESI+ (m/z): [M+H]⁺ calcd for C₅₆H₈₁N₁₄O₁₇S, 1253.56196. found, 1253.56525.

### Example 35

### 7′C-((4-(2-(6-(6-(maleimido)hexanamido)hexanamido)ethyl)piperazin-1-yl)methyl)-α-amanitin

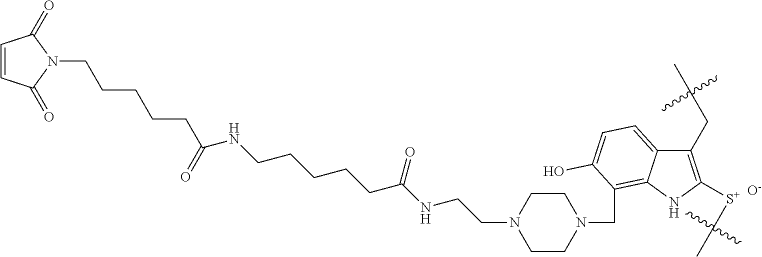

To a solution of 4-N-(2-aminoethyl)-1-N-Boc-piperazine (15 mg, 0.065 mmol) in methylene chloride (4 mL) was added 2,5-dioxopyrrolidin-1-yl 6-(6-(2,5-dioxo-2,5-dihydro-1H-pyrrol-1-yl)hexanamido)hexanoate (30.3 mg, 0.072 mmol). After 1 h stirring at room temperature, the mixture was diluted with 50 mL of ethyl acetate and the organic solution was washed with saturated sodium bicarbonate (100 mL), 0.5 N HCl (100 mL), and saturated brine (100 mL). The combined organic layers were concentrated under reduced pressure, and the resulting residue purified by preparative RP-HPLC. A total of 23 mg of Intermediate 35.1 was obtained as a white solid. [M+H]⁺=537.40.

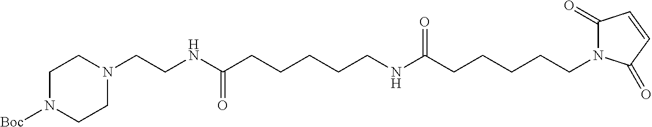 [SMILES: CC(C)(C)OC(=O)N1CCN(CCNC(=O)CCCCCNC(=O)CCCCCN2C(=O)C=CC2=O)CC1]

To Intermediate 35.1 (23 mg) was added a mixture of TFA (2 mL), methylene chloride (0.5 mL), and anisole (10 μL). After 30 min of stirring at room temperature, the reaction mixture was concentrated under reduced pressure and the resulting residue was then further dried under high vacuum. The residue (Intermediate 35.2) was obtained as the TFA salt (20 mg) and was used immediately in the next step without further purification. [M+H]+=436.25.

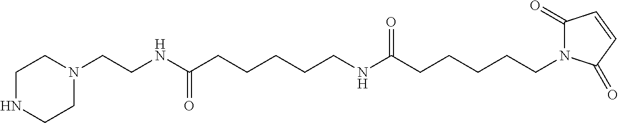 [SMILES: O=C(CCCCCN1C(=O)C=CC1=O)NCCCCCC(=O)NCCN1CCNCC1]

α-Amanitin (7.9 mg, 0.009 mmol), paraformaldehyde (5 mg), and crude Intermediate 35.2 (10 mg) were dissolved in ethanol (3 mL). After 18 h of stirring at 50° C., the reaction mixture was concentrated under reduced pressure, and the resulting residue was purified by preparative RP-HPLC. A total of 8 mg of Example 35 was obtained as a gray-colored solid. HRMS-ESI+ (m/z): [M+H]⁺ calcd for C₆₂H₉₂N₁₅O₁₈S, 1366.64602. found, 1366.64836.

### Example 36

### 7′C-((4-(2-(4-((maleimido)methyl)cyclohexanecarboxamido)ethyl)piperazin-1-yl)methyl)-α-amanitin

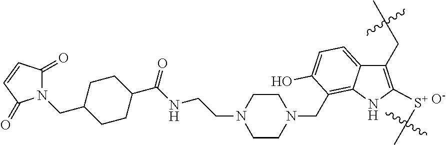

To a solution of 4-N-(2-aminoethyl)-1-N-Boc-piperazine (40 mg, 0.174 mmol) in methylene chloride (4 mL) was added 2,5-dioxopyrrolidin-1-yl 4-((2,5-dioxo-2,5-dihydro-1H-pyrrol-1-yl)methyl)cyclohexanecarboxylate (64 mg, 0.192 mmol). After 1 h of stirring at room temperature, the reaction mixture was diluted with 50 mL of ethyl acetate, and this organic solution was washed with saturated sodium bicarbonate (100 mL), 0.5 N HCl (100 mL), and saturated brine (100 mL). The combined organic layers were concentrated under reduced pressure and the resulting residue purified by preparative RP-HPLC. A total of 80 mg of Intermediate 36.1 (as the TFA salt) was obtained as a white solid and immediately used without further purification. [M+H]⁺=450.50.

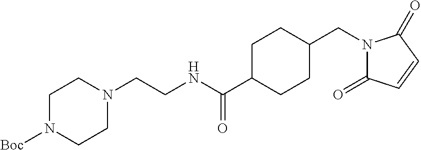 [SMILES: CC(C)(C)OC(=O)N1CCN(CCNC(=O)C2CCC(CN3C(=O)C=CC3=O)CC2)CC1]

To Intermediate 36.1 (80 mg) was added a mixture of TFA (2 mL), methylene chloride (0.5 mL), and anisole (10 μL). After 30 min of stirring at room temperature, the reaction mixture was concentrated under reduced pressure, and the resulting residue further dried under high vacuum. The crude Intermediate 36.2 was obtained as a TFA salt and was used subsequently without further purification. [M+H]⁺=349.18.

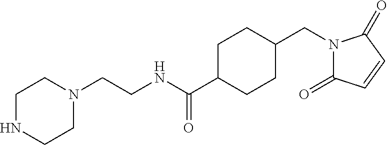 [SMILES: O=C(NCCN1CCNCC1)C1CCC(CN2C(=O)C=CC2=O)CC1]

To α-amanitin (8.9 mg, 0.010 mmol), paraformaldehyde (5 mg), and Intermediate 36.2 (10 mg) was added ethanol (3 mL). After 18 h of stirring at 50° C., the reaction mixture was concentrated under reduced pressure and the resulting residue was purified by preparative RP-HPLC. A total of 10 mg of Example 36 was obtained as gray-colored solid. HRMS-ESI+ (m/z): [M+H]⁺ calcd for C₅₈H₈₃N₁₄O₁₇S, 1279.57761. found, 1279.58071.

### Example 37

### 7′C-((4-(2-(6-(4-((maleimido)methyl)cyclohexanecarboxamido)hexanamido)ethyl)piperazin-1-yl)methyl)-α-amanitin

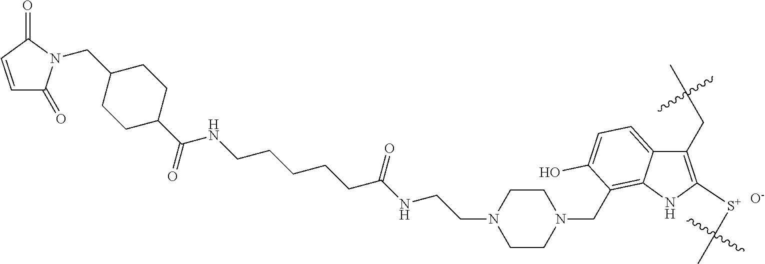

To a solution of 4-N-(2-aminoethyl)-1-N-Boc-piperazine (30 mg, 0.131 mmol) in methylene chloride (4 mL) was added 2,5-dioxopyrrolidin-1-yl 6-(4-((2,5-dioxo-2,5-dihydro-1H-pyrrol-1-yl)methyl)cyclohexanecarboxamido)hexanoate (15 mg, 0.0335 mmol). After 1.5 h of stirring at room temperature, the mixture was diluted with 50 mL of ethyl acetate, washed with saturated sodium bicarbonate (100 mL), 0.5 N HCl (100 mL), and saturated brine (100 mL). The combined organic layers were evaporated, and the resulting residue purified by preparative RP-HPLC. A total of 10 mg of Intermediate 37.1 was obtained as a white solid. [M+H]⁺=562.75.

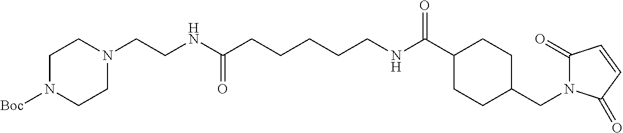 [SMILES: CC(C)(C)OC(=O)N1CCN(CCNC(=O)CCCCCNC(=O)C2CCC(CN3C(=O)C=CC3=O)CC2)CC1]

To Intermediate 37.1 (7 mg) was added a mixture of TFA (2 mL), methylene chloride (0.5 mL), and anisole (10 μL). After stirring for 30 min at room temperature, the reaction mixture was concentrated under reduced pressure, and the resulting residue was further dried under high vacuum. The resulting residue was used subsequently without further purification. Intermediate 37.2 was obtained as TFA salt. [M+H]⁺=462.25.

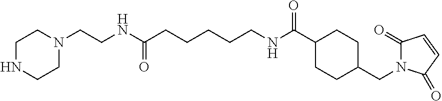 [SMILES: O=C(CCCCCNC(=O)C1CCC(CN2C(=O)C=CC2=O)CC1)NCCN1CCNCC1]

α-Amanitin (8.8 mg, 0.008 mmol), paraformaldehyde (5 mg), and Intermediate 37.2 (7 mg) were dissolved in ethanol (3 mL). After 18 h of stirring at 50° C., the reaction mixture was concentrated under reduced pressure, and the resulting residue was purified by preparative RP-HPLC. A total of 4 mg of Example 37 was obtained as gray-colored solid. HRMS-ESI+(m/z): [M+H]⁺ calcd for C₆₄H₉₄N₁₅O₁₈S, 1392.66167. found, 1392.66155.

### Example 38

### 7′C-((3-((6-(6-(maleimido)hexanamido)hexanamido)-S-methyl)pyrrolidin-1-yl)methyl)-α-amanitin

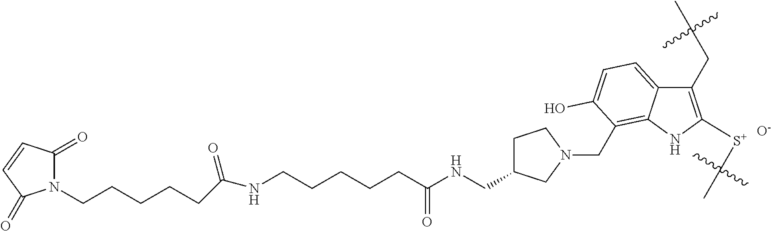

To a solution of 1-N-Boc-3-S-(aminomethyl)pyrrolidine (15 mg, 0.077 mmol) in methylene chloride (4 mL) was added 2,5-dioxopyrrolidin-1-yl 6-(6-(2,5-dioxo-2,5-dihydro-1H-pyrrol-1-yl)hexanamido)hexanoate (34.7 mg, 0.082 mmol). After stirring for 1.5 h at room temperature, the mixture was diluted with 50 mL of ethyl acetate, and the organic solution was washed with saturated sodium bicarbonate (100 mL), 0.1 N HCl (100 mL), and saturated brine (100 mL). The combined organic layers were evaporated, and the residue purified by preparative RP-HPLC. A total of 41 mg of Intermediate 38.1 was obtained as a white solid. [M+H]⁺=507.30.

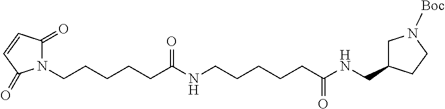 [SMILES: CC(C)(C)OC(=O)N1CC[C@@H](CNC(=O)CCCCCNC(=O)CCCCCN2C(=O)C=CC2=O)C1]

To Intermediate 38.1 (41 mg) was added a mixture of TFA (2 mL), methylene chloride (0.5 mL), and anisole (10 μL). After stirring for 30 min at room temperature, the reaction mixture was concentrated under reduced pressure and the resulting residue then further dried under high vacuum. The residue (containing Intermediate 38.2 and TFA salt) was used subsequently without further purification. [M+H]⁺=407.19.

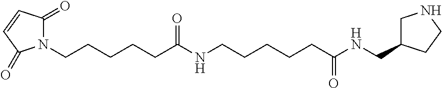 [SMILES: O=C(CCCCCN1C(=O)C=CC1=O)NCCCCCC(=O)NC[C@@H]1CCNC1]

α-Amanitin (8.8 mg, 0.010 mmol), paraformaldehyde (5 mg), and Intermediate 38.2 (10 mg) were dissolved in ethanol (3 mL). After stirring overnight at 75° C., the reaction mixture was concentrated under reduced pressure, and the resulting residue was purified by preparative RP-HPLC. A total of 8 mg of Example 38 was obtained as gray-colored solid. HRMS-ESI+(m/z): [M+H]⁺ calcd for C₆₁H₈₉N₁₄O₁₈S, 1338.62677. found, 1338.62974.

### Example 39

### 7′C-((3-((6-(6-(maleimido)hexanamido)hexanamido)-R-methyl)pyrrolidin-1-yl)methyl)-α-amanitin

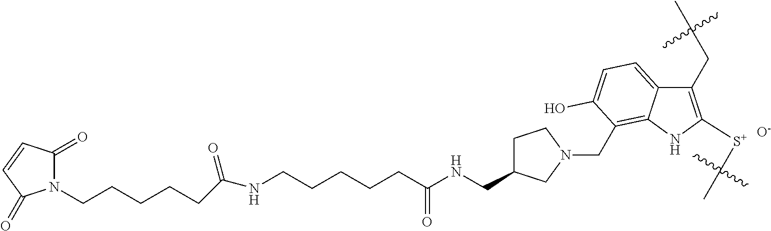

To a solution of 1-N-Boc-3-R-(aminomethyl)pyrrolidine (15 mg, 0.077 mmol) in methylene chloride (4 mL) was added 2,5-dioxopyrrolidin-1-yl 6-(6-(2,5-dioxo-2,5-dihydro-1H-pyrrol-1-yl)hexanamido)hexanoate (35.9 mg, 0.085 mmol). After stirring for 1.5 h at room temperature, the mixture was diluted with 50 mL of ethyl acetate, washed with saturated sodium bicarbonate (100 mL), 0.1 N HCl (100 mL), and saturated brine (100 mL). The combined organic layers were concentrated under reduced pressure, and the resulting residue purified by preparative RP-HPLC. A total of 32 mg of Intermediate 39.1 was obtained as a white solid. [M+H]⁺=508.25.

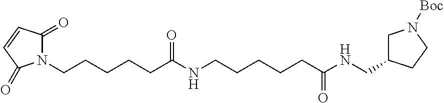 [SMILES: CC(C)(C)OC(=O)N1CC[C@H](CNC(=O)CCCCCNC(=O)CCCCCN2C(=O)C=CC2=O)C1]

To Intermediate 39.1 (32 mg) was added a mixture of TFA (2 mL), methylene chloride (0.5 mL), and anisole (10 μL). After stirring for 30 min at room temperature, the reaction mixture was concentrated under reduced pressure and the resulting residue then further dried under high vacuum. The residue (containing Intermediate 39.2 and TFA salt) was used subsequently without further purification. [M+H]⁺=407.19.

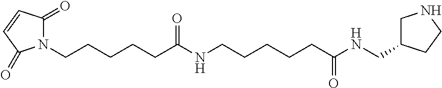 [SMILES: O=C(CCCCCN1C(=O)C=CC1=O)NCCCCCC(=O)NC[C@H]1CCNC1]

α-Amanitin (10 mg, 0.011 mmol), paraformaldehyde (5 mg), and Intermediate 39.2 (10 mg) were dissolved in ethanol (3 mL). After stirring overnight at 70° C., the reaction mixture was concentrated under reduced pressure, and the resulting residue was purified by preparative RP-HPLC. A total of 8 mg of Example 39 was obtained as gray-colored solid. HRMS-ESI+(m/z): [M+H]⁺ calcd for C₆₁H₈₉N₁₄O₁₈S, 1337.61947. found, 1337.62267.

### Example 40a

### 7′C-((3-((4-((maleimido)methyl)cyclohexanecarboxamido)-S-methyl)pyrrolidin-1-yl)methyl)-α-amanitin

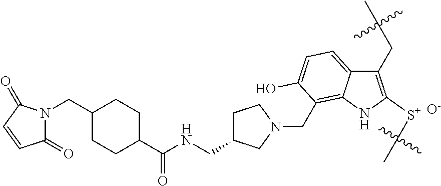

To a solution of 1-N-Boc-3-S-(aminomethyl)pyrrolidine (17.8 mg, 0.089 mmol) in methylene chloride (4 mL) was added 2,5-dioxopyrrolidin-1-yl 4-((2,5-dioxo-2,5-dihydro-1H-pyrrol-1-yl)methyl)cyclohexanecarboxylate (32.7 mg, 0.098 mmol). After stirring for 1.5 h at room temperature, the mixture was diluted with 50 mL of ethyl acetate, and the organic solution was washed with saturated sodium bicarbonate (100 mL), 0.1 N HCl (100 mL), and saturated brine (100 mL). The combined organic layers were evaporated, and the resulting residue purified by preparative RP-HPLC. A total of 34 mg of Intermediate 40a.1 was obtained as a white solid. [M+H]⁺=420.15.

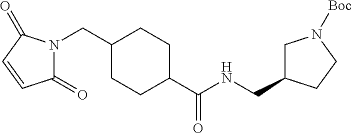 [SMILES: CC(C)(C)OC(=O)N1CC[C@@H](CNC(=O)C2CCC(CN3C(=O)C=CC3=O)CC2)C1]

To Intermediate 40a.1 (34 mg) was added a mixture of TFA (2 mL), methylene chloride (0.5 mL), and anisole (10 μL). After stirring 30 min at room temperature, the reaction mixture was concentrated under reduced pressure and the resulting residue then further dried under high vacuum. The residue (containing Intermediate 40a.2 and TFA salt) was used subsequently without further purification. [M+H]⁺=320.08.

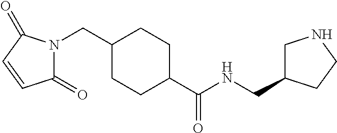 [SMILES: O=C(NC[C@@H]1CCNC1)C1CCC(CN2C(=O)C=CC2=O)CC1]

α-Amanitin (9.6 mg, 0.010 mmol), paraformaldehyde (10 mg), and Intermediate 40a.2 (10 mg) were dissolved in ethanol (3 mL). After stirring overnight at 75° C., the reaction mixture was concentrated under reduced pressure, and the resulting residue was purified by preparative RP-HPLC. A total of 9 mg of Example 40a was obtained as gray-colored solid. HRMS-ESI+ (m/z): [M+2H]²⁺ calcd for (C₅₇H₈₁N₁₃O₁₇S)/2, 625.77918. found, 625.78047.

### Example 40b

### 7′C-((3-((4-((maleimido)methyl)cyclohexanecarboxamido)-R-methyl)pyrrolidin-1-yl)methyl)-α-amanitin

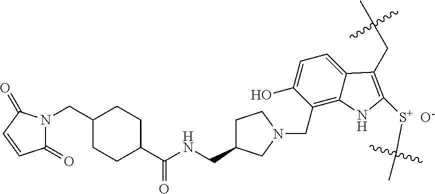

To a solution of 1-N-Boc-3-R-(aminomethyl)pyrrolidine (35 mg, 0.175 mmol) in methylene chloride (4 mL) was added 2,5-dioxopyrrolidin-1-yl 4-((2,5-dioxo-2,5-dihydro-1H-pyrrol-1-yl)methyl)cyclohexanecarboxylate (64 mg, 0.192 mmol). After stirring for 1.5 h at room temperature, the mixture was diluted with 50 mL of ethyl acetate, washed with saturated sodium bicarbonate (100 mL), 0.1 N HCl (100 mL), and saturated brine (100 mL). The combined organic layers were evaporated, and the resulting residue purified by preparative RP-HPLC. A total of 80 mg of Intermediate 40b.1 was obtained as white solid. [M+H]⁺=420.20.

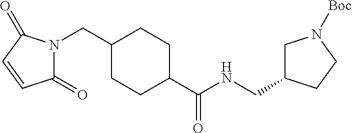 [SMILES: CC(C)(C)OC(=O)N1CC[C@H](CNC(=O)C2CCC(CN3C(=O)C=CC3=O)CC2)C1]

To Intermediate 40b.1 (80 mg) was added a mixture of TFA (2 mL), methylene chloride (0.5 mL), and anisole (10 μL). After stirring for 30 min at room temperature, the reaction mixture was concentrated under reduced pressure and the resulting residue then further dried under high vacuum. The residue (containing Intermediate 40b.2 and TFA salt) was used subsequently without further purification. [M+H]⁺=310.13.

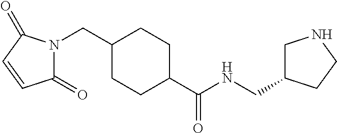 [SMILES: O=C(NC[C@H]1CCNC1)C1CCC(CN2C(=O)C=CC2=O)CC1]

α-Amanitin (10 mg, 0.011 mmol), paraformaldehyde (10 mg), and Intermediate 40b.2 (38 mg) were dissolved in ethanol (2 mL). After stirring overnight at 75° C., the reaction mixture was concentrated under reduced pressure, and the resulting residue was purified by preparative RP-HPLC. A total of 8 mg of Example 40b was obtained as gray-colored solid. HRMS-ESI+ (m/z): [M+H]⁺ calcd for C₅₇H₈₀N₁₃O₁₇S, 1250.55106. found, 1250.55676.

### Example 41

### 7′C-((3-((6-(4-((maleimido)methyl)cyclohexanecarboxamido)hexanamido)methyl)pyrrolidin-1-yl)methyl)-α-amanitin

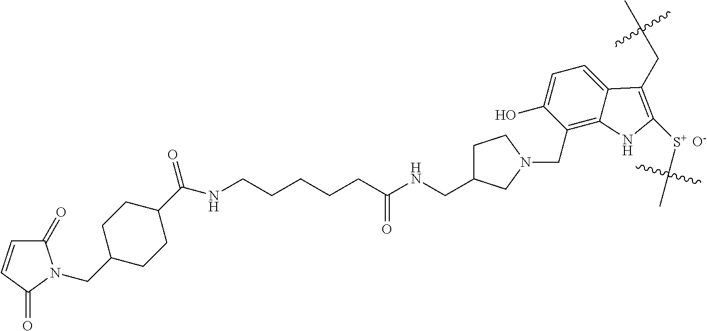

Example 41 may be prepared using methods analogous to those described for the preceding examples.

### Example 42

### 7′C-((4-(2-(3-carboxypropanamido)ethyl)piperazin-1-yl)methyl)-α-amanitin

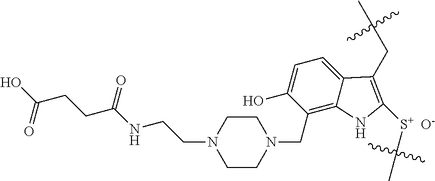

α-Amanitin (10 mg, 0.011 mmol), paraformaldehyde (2.6 mg), and 4-(2-Boc-aminoethyl)piperazine (10 mg, 0.04 mmol) were dissolved in ethanol (3 mL). After stirring for 18 h at 65° C., the reaction mixture was concentrated under reduced pressure, and the resulting residue was purified by preparative RP-HPLC. A total of 10 mg of Intermediate 42.1 was obtained as gray-colored solid. [M+H]⁺=1161.59.

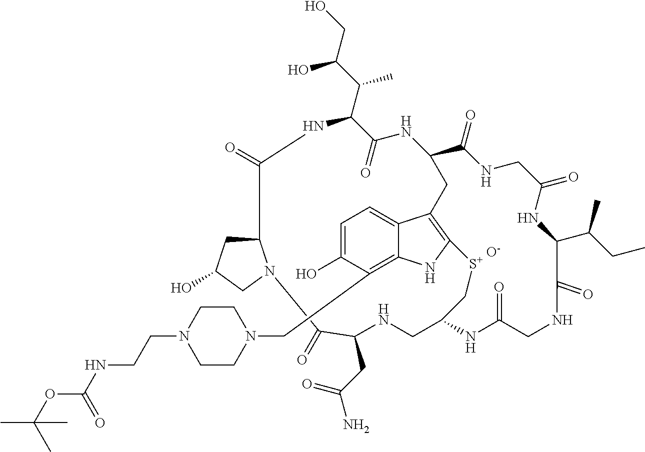

To Intermediate 42.1 (10 mg) was added a mixture of TFA (2 mL), methylene chloride (0.5 mL), and anisole (20 μL). After stirring for 1 h at room temperature, the reaction mixture was concentrated under reduced pressure and the resulting residue was then further dried under high vacuum. The residue (containing Intermediate 42.2 and TFA salt) was used subsequently without further purification. [M+H]⁺=1060.85.

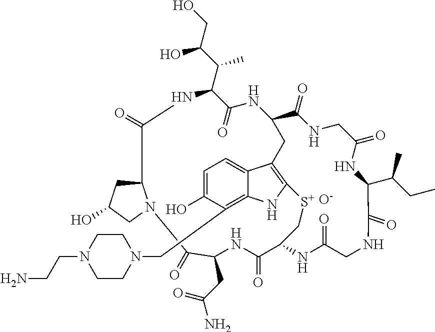

To a solution of Intermediate 42.2 (10 mg) in pyridine (1 mL) was added succinic anhydride (0.9 mg, 0.01 mmol). After stirring for 24 h, 1 mg of additional succinic anhydride was added to the reaction mixture. After further stirring, the reaction mixture was concentrated under reduced pressure, and the resulting residue was purified by preparative RP-HPLC. A total of 3 mg of Example 42 was obtained as a gray-colored solid. [M+H]⁺=1160.68.

The compounds in Examples 43-70 may be prepared using methods analogous to those described above.

### Example 43

### 7′C-((4-(6-(6-(maleimido)hexanamido)hexanoyl)piperazin-1-yl)methyl)-α-amanitin

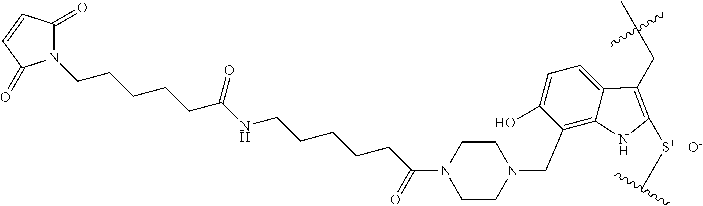

### Example 44

### 7′C-((4-(6-(4-((maleimido)methyl)cyclohexanecarboxamido)hexanoyl)piperazin-1-yl)methyl)-α-amanitin

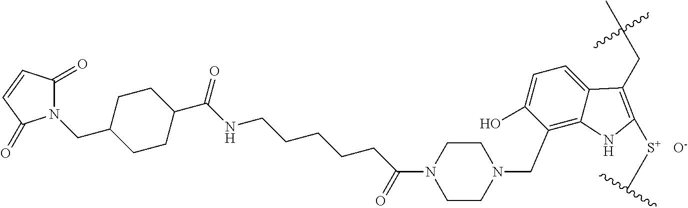

### Example 45

### 7′C-((4-(2-(maleimido)acetyl)piperazin-1-yl)methyl)-α-amanitin

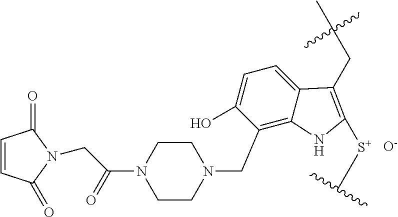

### Example 46

### 7′C-((4-(3-(maleimido)propanoyl)piperazin-1-yl)methyl)-α-amanitin

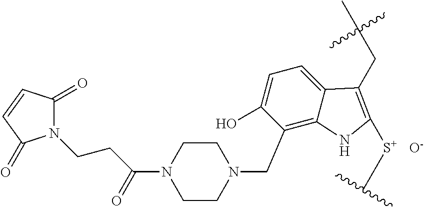

### Example 47

### 7′C-((4-(4-(maleimido)butanoyl)piperazin-1-yl)methyl)-α-amanitin

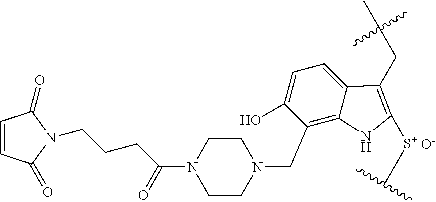

### Example 48

### 7′C-((4-(2-(2-(maleimido)acetamido)ethyl)piperidin-1-yl)methyl)-α-amanitin

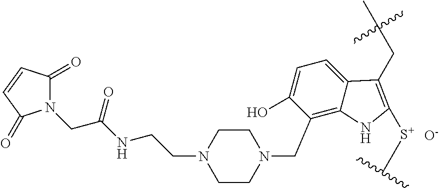

### Example 49

### 7′C-((4-(2-(4-(maleimido)butanamido)ethyl)piperidin-1-yl)methyl)-α-amanitin

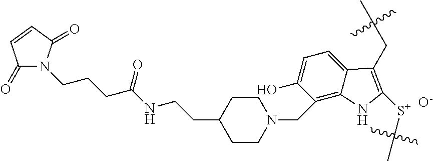

### Example 50

### 7′C-((4-(2-(6-(4-((maleimido)methyl)cyclohexanecarboxamido)hexanamido)ethyl)piperidin-1-yl)methyl)-α-amanitin

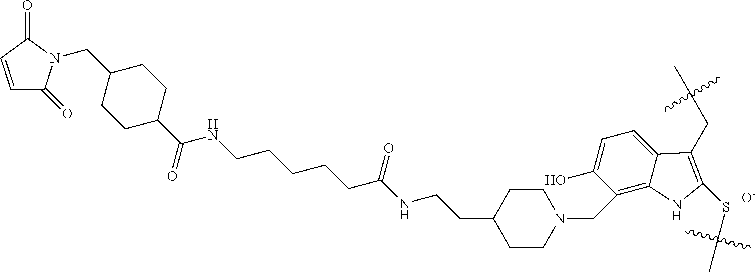

### Example 51

### 7′C-((3-((6-(maleimido)hexanamido)methyl)azetidin-1-yl)methyl)-α-amanitin

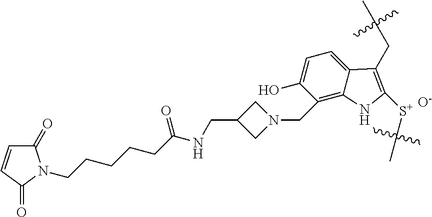

### Example 52

### 7′C-((3-(2-(6-(maleimido)hexanamido)ethyl)azetidin-1-yl)methyl)-α-amanitin

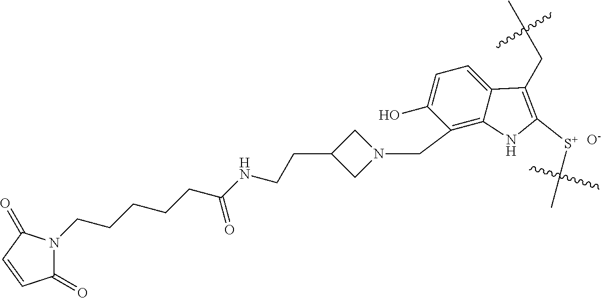

### Example 53

### 7′C-((3-((4-((maleimido)methyl)cyclohexanecarboxamido)methyl)azetidin-1-yl)methyl)-α-amanitin

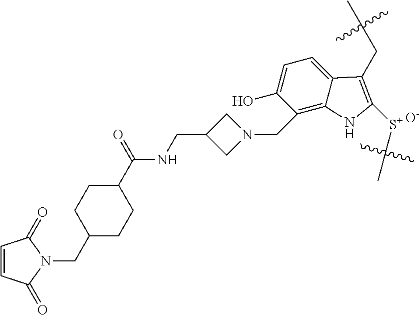

### Example 54

### 7′C-((3-(2-(4-((maleimido)methyl)cyclohexanecarboxamido)ethyl)azetidin-1-yl)methyl)-α-amanitin

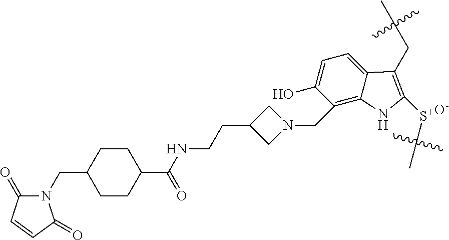

### Example 55

### 7′C-((3-(2-(6-(4-((maleimido)methyl)cyclohexanecarboxamido)hexanamido)ethyl)azetidin-1-yl)methyl)-α-amanitin

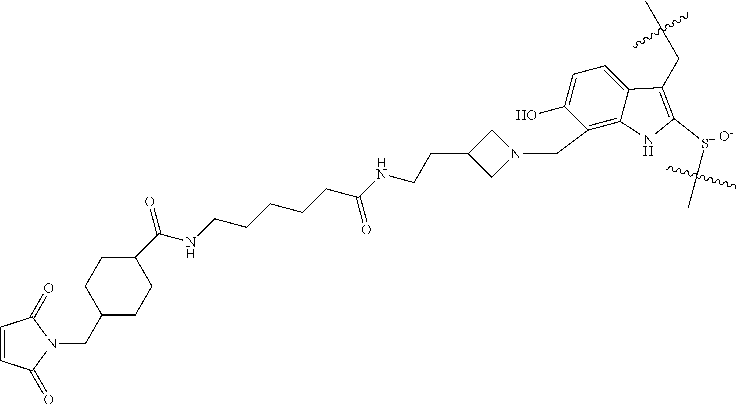

### Example 56

### 7′C-(((2-(6-(maleimido)-N-methylhexanamido)ethyl)(methyl)amino)methyl)-α-amanitin

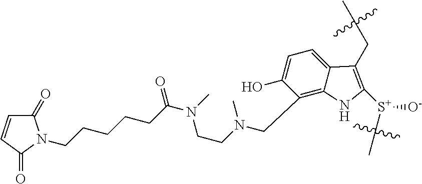 [SMILES: *Cc1c([S@@+](*)[O-])[nH]c2c(CN(C)CCN(C)C(=O)CCCCCN3C(=O)C=CC3=O)c(O)ccc12]

### Example 57

### 7′C-(((4-(6-(maleimido)-N-methylhexanamido)butyl(methyl)amino)methyl)-α-amanitin

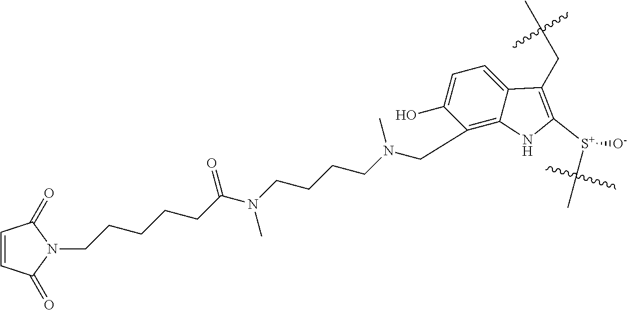 [SMILES: *Cc1c([S@@+](*)[O-])[nH]c2c(CN(C)CCCCN(C)C(=O)CCCCCN3C(=O)C=CC3=O)c(O)ccc12]

### Example 58

### 7′C-((2-(2-(6-(maleimido)hexanamido)ethyl)aziridin-1-yl)methyl)-α-amanitin

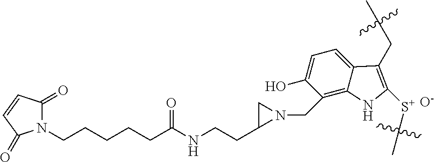

### Example 59

### 7′C-((2-(2-(6-(4-((maleimido)methyl)cyclohexanecarboxamido)hexanamido)ethyl)aziridin-1-yl)methyl)-α-amanitin

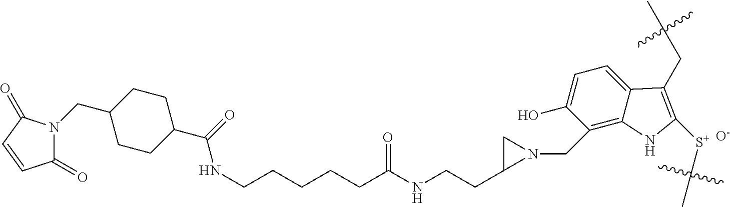

### Example 60

### 7′C-((4-(6-(6-(2-(aminooxy)acetamido)hexanamido)hexanoyl)piperazin-1-yl)methyl)-α-amanitin

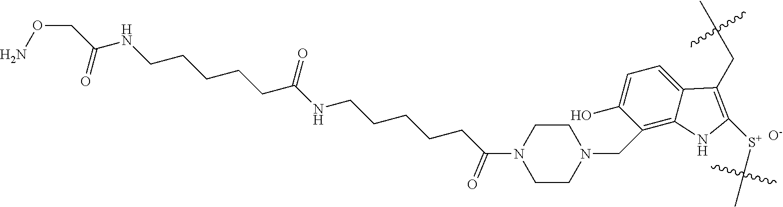

### Example 61

### 7′C-((4-(1-(aminooxy)-2-oxo-6,9,12,15-tetraoxa-3-azaheptadecan-17-oyl)piperazin-1-yl)methyl)-α-amanitin

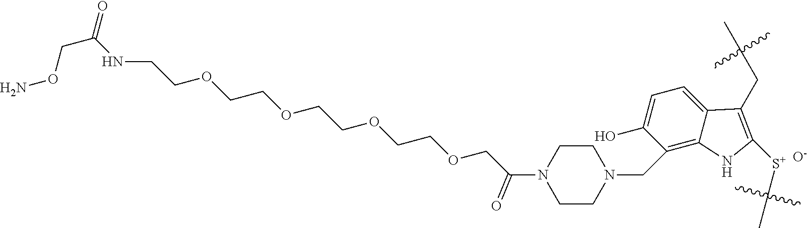

### Example 62

### 7′C-((4-(2-(2-(aminooxy)acetamido)acetyl)piperazin-1-yl)methyl)-α-amanitin

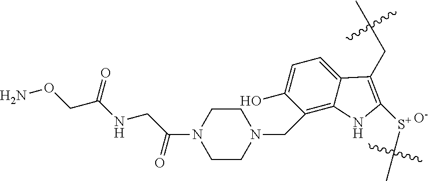

### Example 63

### 7′C-((4-(3-(2-(aminooxy)acetamido)propanoyl)piperazin-1-yl)methyl)-α-amanitin

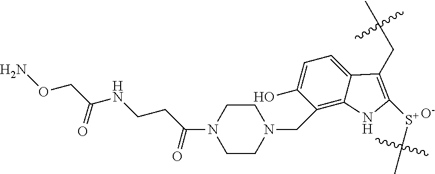

### Example 64

### 7′C-((4-(4-(2-(aminooxy)acetamido)butanoyl)piperazin-1-yl)methyl)-α-amanitin

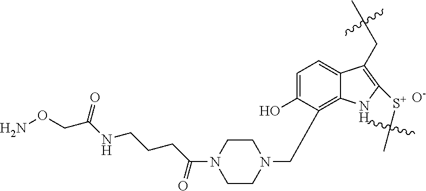

### Example 65

### 7′C-((4-(2-(6-(2-(aminooxy)acetamido)hexanamido)ethyl)piperidin-1-yl)methyl)-α-amanitin

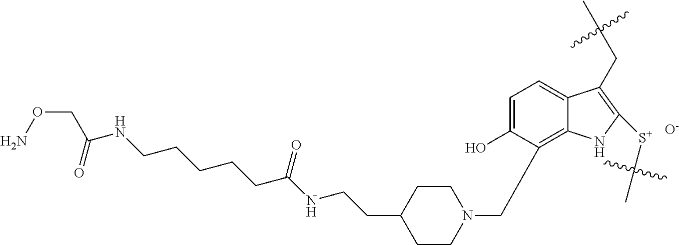

### Example 66

### 7′C-((4-(2-(2-(2-(aminooxy)acetamido)acetamido)ethyl)piperidin-1-yl)methyl)-α-amanitin

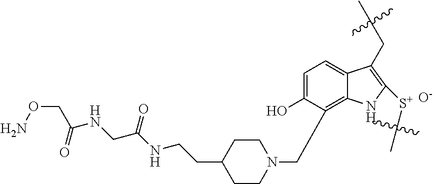

### Example 67

### 7′C-((4-(2-(4-(2-(aminooxy)acetamido)butanamido)ethyl)piperidin-1-yl)methyl)-α-amanitin

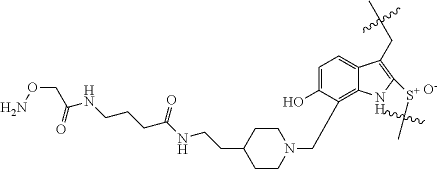

### Example 68

### 7′C-((4-(20-(aminooxy)-4,19-dioxo-6,9,12,15-tetraoxa-3,18-diazaicosyl)piperidin-1-yl)methyl)-α-amanitin

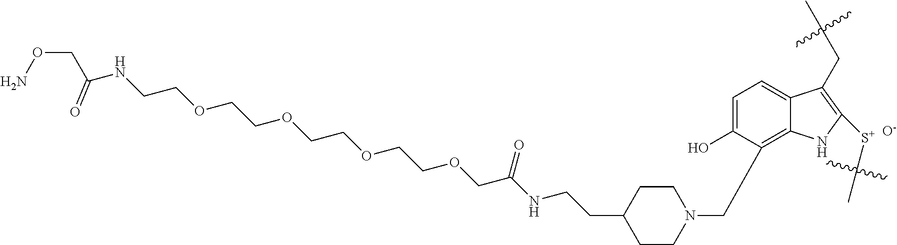

### Example 69

### 7′C-(((2-(6-(2-(aminooxy)acetamido)-N-methylhexanamido)ethyl)(methyl)amino)methyl)-α-amanitin

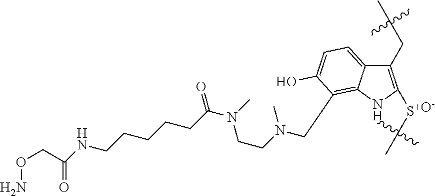

### Example 70

### 7′C-(((4-(6-(2-(aminooxy)acetamido)-N-methylhexanamido)butyl)(methyl)amino)methyl)-α-amanitin

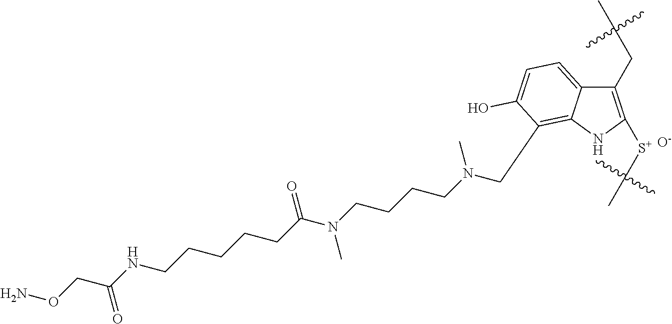

### Example 71

### 7′C-((3-((6-(4-((maleimido)methyl)cyclohexanecarboxamido)hexanamido)methyl)pyrrolidin-1-yl)-S-methyl)-α-amanitin

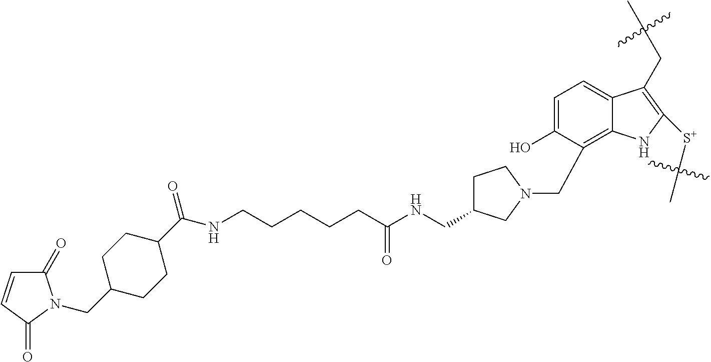

To a solution of 1-N-Boc-3-S-(aminomethyl)pyrrolidine (20 mg, 0.100 mmol) in methylene chloride (4 mL) was added 2,5-dioxopyrrolidin-1-yl 6-(4-((2,5-dioxo-2,5-dihydro-1H-pyrrol-1-yl)methyl)cyclohexanecarboxamido)hexanoate (49 mg, 0.11 mmol). After stirring for 1.5 h at room temperature, the mixture was diluted with 50 mL of ethyl acetate and the organic solution was washed with saturated sodium bicarbonate (100 mL), 0.1 N HCl (100 mL), and saturated brine (100 mL). The combined organic layers were evaporated, and the resulting residue purified by preparative RP-HPLC. A total of 51 mg of Intermediate 71.1 was obtained as a white solid. [M+H]⁺=533.25.

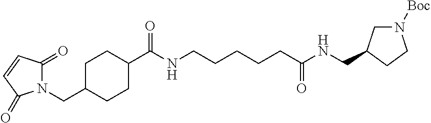 [SMILES: CC(C)(C)OC(=O)N1CC[C@@H](CNC(=O)CCCCCNC(=O)C2CCC(CN3C(=O)C=CC3=O)CC2)C1]

To Intermediate 71.1 (51 mg) was added a mixture of TFA (2 mL), methylene chloride (0.5 mL), and anisole (10 μL). After stirring for 30 min at room temperature, the reaction mixture was concentrated under reduced pressure and the resulting residue then further dried under high vacuum. The residue (containing Intermediate 71.2 and TFA salt) was used subsequently without further purification. [M+H]⁺=433.60.

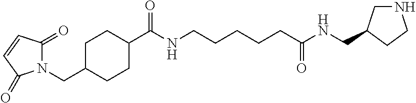 [SMILES: O=C(CCCCCNC(=O)C1CCC(CN2C(=O)C=CC2=O)CC1)NC[C@@H]1CCNC1]

α-Amanitin (9.3 mg, 0.010 mmol), paraformaldehyde (5 mg), and Intermediate 71.2 (10 mg) were dissolved in ethanol (3 mL). After stirring for 18 h at 75° C., the reaction mixture was concentrated under reduced pressure, and the resulting residue was purified by preparative RP-HPLC. A total of 8.2 mg of Example 71 was obtained as gray-colored solid. HRMS-ESI+(m/z): [M+H]⁺ calcd for C₆₃H₉₁N₁₄O₁₈S, 1363.63512. found, 1363.63416.

### Example 72

### 7′C-((3-((6-(4-((maleimido)methyl)cyclohexanecarboxamido)hexanamido)-R-methyl)pyrrolidin-1-yl)methyl)-α-amanitin

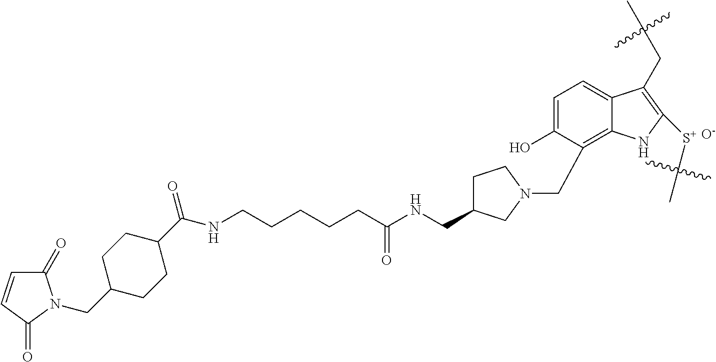

To a solution of 1-N-Boc-3-R-(aminomethyl)pyrrolidine (15.8 mg, 0.079 mmol) in methylene chloride (4 mL) was added 2,5-dioxopyrrolidin-1-yl 6-(4-((2,5-dioxo-2,5-dihydro-1H-pyrrol-1-yl)methyl)cyclohexanecarboxamido)hexanoate (38.8 mg, 0.087 mmol). After stirring for 1.5 h at room temperature, the reaction mixture was diluted with 50 mL of ethyl acetate, and this organic solution was washed with saturated sodium bicarbonate (100 mL), 0.1 N HCl (100 mL), and saturated brine (100 mL). The combined organic layers were concentrated under reduced pressure, and the resulting residue purified by preparative RP-HPLC. A total of 28 mg of Intermediate 72.1 was obtained as a white solid. [M+H]⁺=533.8.

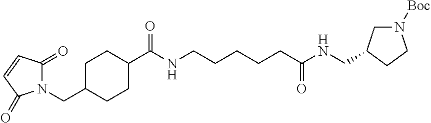 [SMILES: CC(C)(C)OC(=O)N1CC[C@H](CNC(=O)CCCCCNC(=O)C2CCC(CN3C(=O)C=CC3=O)CC2)C1]

To Intermediate 72.1 (28 mg) was added a mixture of TFA (2 mL), methylene chloride (0.5 mL), and anisole (10 μL). After stirring for 30 min at room temperature, the reaction mixture was concentrated under reduced pressure and the resulting residue then further dried under high vacuum. The residue (containing Intermediate 72.2 and TFA salt) was used subsequently without further purification. [M+H]⁺=433.60.

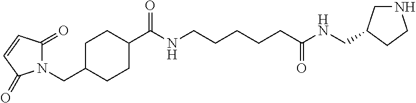 [SMILES: O=C(CCCCCNC(=O)C1CCC(CN2C(=O)C=CC2=O)CC1)NC[C@H]1CCNC1]

α-Amanitin (9.6 mg, 0.010 mmol), paraformaldehyde (5 mg), and Intermediate 72.2 (10 mg) were dissolved in ethanol (3 mL). After stirring for 18 h at 75° C., the reaction mixture was concentrated under reduced pressure, and the resulting residue was purified by preparative RP-HPLC. A total of 7 mg of Example 72 was obtained as gray-colored solid. HRMS-ESI+(m/z): [M+H]⁺ calcd for C₆₃H₉₁N₁₄O₁₈S, 1363.63512. found, 1363.63418.

The compounds in Examples 73-75 may be prepared using methods analogous to those described above.

### Example 73

### 7′C-((4-(2-(2-bromoacetamido)ethyl)piperazin-1-yl)methyl)-α-amanitin

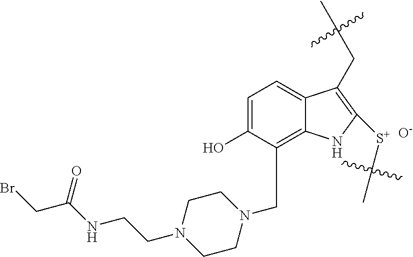

### Example 74

### 7′C-((4-(2-(2-bromoacetamido)ethyl)piperidin-1-yl)methyl)-α-amanitin

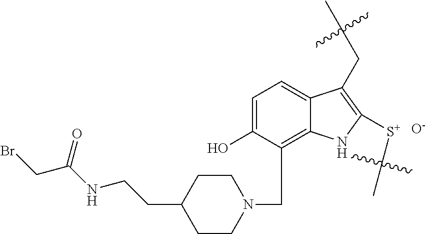

### Example 75

### 7′C-((4-(2-(3-(pyridin-2-yldisulfanyl)propanamido)ethyl)piperidin-1-yl)methyl)-α-amanitin

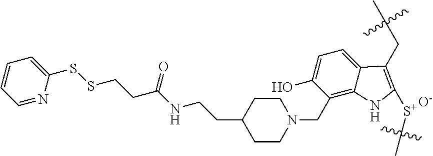

### Example 76

### 6′O-(6-(6-(maleimido)hexanamido)hexyl)-α-amanitin

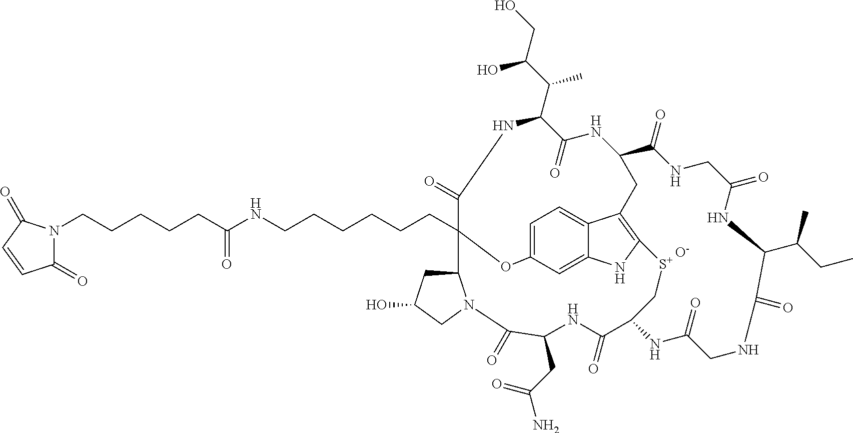

A solution of α-amanitin (20 mg) and 6-(Boc-amino)hexyl bromide (30.7 mg) in DMSO (1 mL) was treated with potassium tert-butoxide (2.4 mg) under argon atmosphere. After stirring at ambient temperature for 1.5 h, the reaction mixture was acidified by addition of acetic acid (100 μL) and then the mixture was added dropwise to a flask containing diethyl ether (40 mL) in order to precipitate the desired ether intermediate. Then the supernatant was decanted and discarded. The precipitate was purified by preparative RP-HPLC to provide 6′O-(6-(Boc-amino)hexyl)-α-amanitin ([M+H]⁺=1118.5, 10 mg) as a white powder. To this material was added TFA (2 mL), methylene chloride (0.5 mL), and anisole (25 μL), and the reaction mixture was stirred for 1 h at ambient temperature. The reaction mixture was concentrated under reduced pressure, and the residue was further dried under high vacuum. The 6′O-(6-amino-hexyl) derivative ([M+H]⁺=1018.5) was recovered as a TFA salt (15 mg) and immediately dissolved in THF (1.5 mL) and DMSO (0.4 mL). To this solution was added N-(6-maleimideocaproyloxy)succinimide (2 mg) and pyridine (0.2 mL). The solution was stirred for 1 h at 50° C. under argon atmosphere. The solution was concentrated under reduced pressure, and the residue was purified by preparative RP-HPLC to yield 2.3 mg of 6′O-(6-(6-(maleimido)hexanamido)hexyl)-α-amanitin ([M+H]⁺=1211.8).

The composition of Example 76 was then conjugated to H16-7.8 in the following manner. To a solution of 6 mg of H16-7.8 dissolved in 898 μL of 50 mM of sodium borate, 200 mM of NaCl, pH 9.0 buffer was added 15.9 μL of 10 mM tris(2-carboxyethyl)phosphine (TCEP) solution, and 9.1 μL of 0.5 M EDTA. The reaction mixture was incubated in a 37° C. water bath for 2 h. To this mixture was added 18 μL of a 10.2 mM solution of Example 76 in DMSO. The reaction was performed for 1 h at rt. To this solution was added 8.9 μL of 0.1 M N-acetyl-L-cysteine. The isolation of the H16-7.8-Example 76 conjugate was performed by separation of the macromolecular component on a G-25 gel filtration column and yielded 2.7 mg of drug-antibody conjugate. The drug-antibody ratio was calculated by measuring the absorbance at 310 nm and 280 nm of AGS16C-Example 76 conjugate, using the extinction coefficient for α-amanitin of 13500 cm⁻¹M⁻¹. The drug-antibody ratio of this conjugate was 4.8.

### Alternative Synthesis

To a mixture of α-amanitin (0.115 g, 0.125 mmol) and 6-(Boc-amino)hexyl bromide (0.210 g, 0.751 mmol) in DMSO (2 mL) was added potassium tert-butoxide (0.018 g, 0.163 mmol) under an anhydrous nitrogen atmosphere at room temperature. After 1 h stirring at room temperature, the reaction mixture was treated with glacial acetic acid (0.1 mL). The reaction mixture was added dropwise to 40 mL of diethyl ether. A dark precipitate was separated from the supernatant by centrifugation and purified by preparative RP-HPLC. A total of 74 mg of Intermediate 76.1 was obtained as gray-colored solid. [M+H]⁺=1118.5.

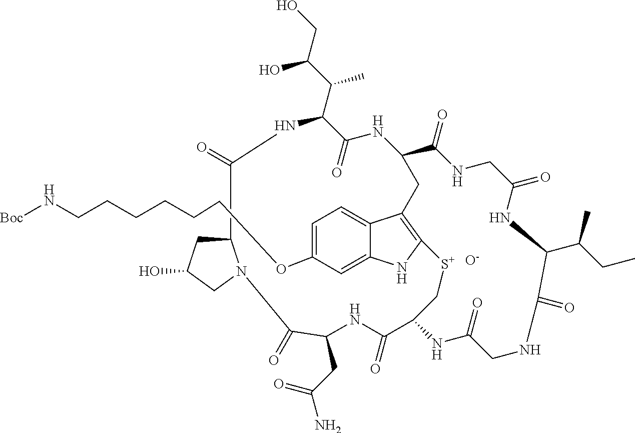

To Intermediate 76.1 (74 mg, 0.066 mmol) was added the mixture of TFA (0.5 mL), methylene chloride (3 mL), water (5 μL), and anisole (5 μL) at room temperature. After 30 min of stirring at room temperature, the reaction mixture was concentrated under reduced pressure and the resulting residue was dried under high vacuum. The resulting yellow-colored oil was used subsequently without further purification. A total of 85 mg of Intermediate 76.2 was obtained as a TFA salt.

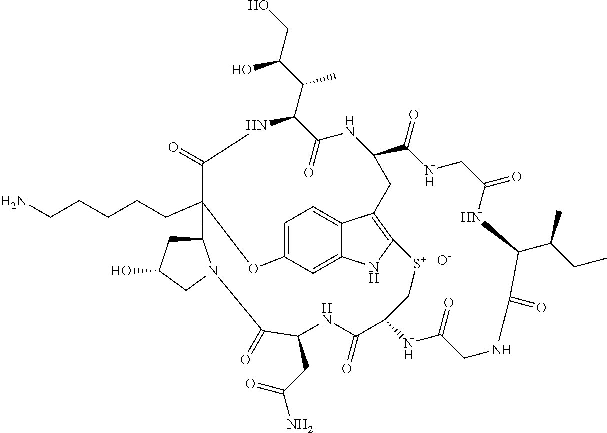

To a solution of Intermediate 76.2 (50 mg, 0.049 mmol) in N,N-dimethylformamide (DMF; 3 mL) was added maleimidocaproic acid (11 mg, 0.052 mmol), 0-(7-azabenzotriazol-1-yl)-N,N,N′,N′-tetramethyluronium hexafluorophosphate (HATU; 17 mg, 0.074 mmol) and diisopropylethyl amine (0.026 mL, 0.147 mmol) at room temperature under an anhydrous nitrogen atmosphere. After 1 h of stirring at room temperature, the reaction mixture was concentrated and the crude mixture was purified by preparative RP-HPLC. A total of 82 mg of 6′O-(6-(6-(maleimido)hexanamido)hexyl)-α-amanitin was obtained as a pale brown-colored solid. HRMS-ESI+ (m/z): [M+H]⁺ calcd for C₅₅H₇₉N₁₂O₁₇S, 1211.54016. found, 1211.54555.

### Example 77

### 6′O-(5-(4-((maleimido)methyl)cyclohexanecarboxamido)pentyl)-α-amanitin

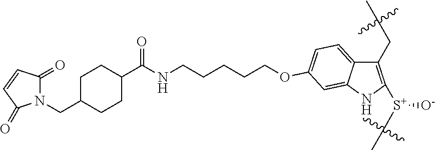 [SMILES: *Cc1c([S@@+](*)[O-])[nH]c2cc(OCCCCCNC(=O)C3CCC(CN4C(=O)C=CC4=O)CC3)ccc12]

To a solution of Intermediate 76.2 (4 mg) in THF (1 mL), DMSO (0.4 mL), and pyridine (0.2 mL) was added 2,5-dioxopyrrolidin-1-yl 4-((2,5-dioxo-2,5-dihydro-1H-pyrrol-1-yl)methyl)cyclohexanecarboxylate (1.8 mg, 0.005 mmol). After 1 h of stirring at 50° C. under an argon atmosphere, the reaction mixture was concentrated under reduced pressure, and the resulting residue was purified by preparative RP-HPLC. A total of 1 mg of Example 77 was obtained as gray-colored solid. HRMS-ESI+ (m/z): [M+H]⁺ calcd for C₅₇H₈₁N₁₂O₁₇S, 1237.55581. found, 1237.56190.

The compounds in Examples 78-80 may be prepared using methods analogous to those described in the preceding examples.

### Example 78

### 6′O-(2-((6-(maleimido)hexyl)oxy)-2-oxoethyl)-α-amanitin

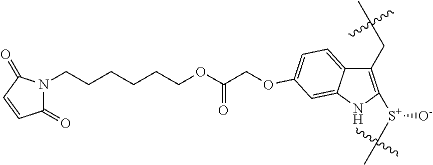 [SMILES: *Cc1c([S@@+](*)[O-])[nH]c2cc(OCC(=O)OCCCCCCN3C(=O)C=CC3=O)ccc12]

### Example 79

### 6′O-((6-(maleimido)hexyl)carbamoyl)-α-amanitin

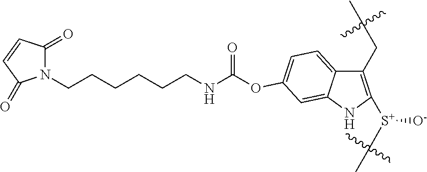 [SMILES: *Cc1c([S@@+](*)[O-])[nH]c2cc(OC(=O)NCCCCCCN3C(=O)C=CC3=O)ccc12]

### Example 80

### 6′O-((6-(4-((maleimido)methyl)cyclohexanecarboxamido)hexyl)carbamoyl)-α-amanitin

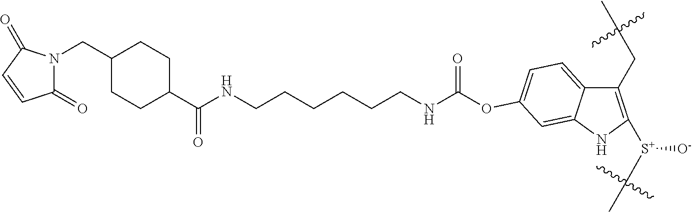 [SMILES: *Cc1c([S@@+](*)[O-])[nH]c2cc(OC(=O)NCCCCCCNC(=O)C3CCC(CN4C(=O)C=CC4=O)CC3)ccc12]

### Example 81

### 6′O-(6-(2-bromoacetamido)hexyl)-α-amanitin

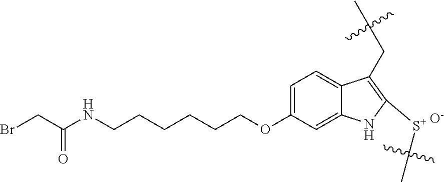

To a mixture of Intermediate 76.2 (8.8 mg) and bromoacetyl bromide (0.75 μL, 0.009 mmol) in DMF (3 mL) was added diisopropylethyl amine (4.1 μL, 0.023 mmol) under an anhydrous nitrogen atmosphere at room temperature. After 1 h stirring at room temperature, the reaction mixture was concentrated under reduced pressure, and the resulting residue was purified by preparative RP-HPLC with a Phenomenex Synergi 10μ Max-RP 80 Å column (150×30 mm) using 10% to 90% MeCN in 0.1% formic acid as the eluent. A total of 3 mg of Example 81 was obtained as a white solid. [M+H]⁺=1140.44.

### Example 82

### 7′C-(4-(6-(azido)hexanamido)piperidin-1-yl)-α-amanitin

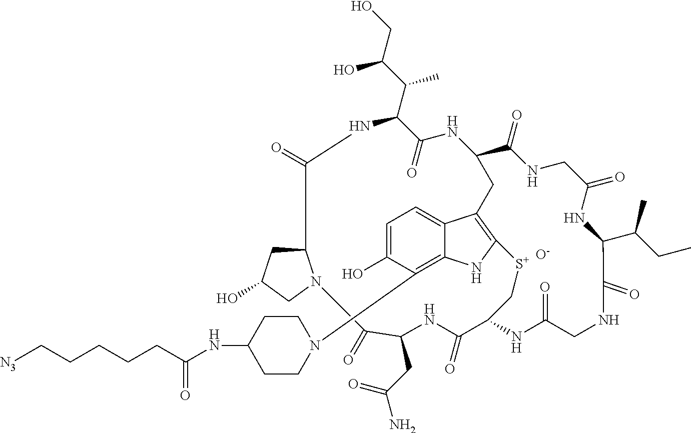

To a mixture of Intermediate 2.2 (12 mg), HATU (6 mg, 0.016 mmol), and 6-azidohexanoic acid (2 mg, 0.013 mmol) in DMF (2 mL) was added diisopropylethyl amine (9 μL) under an anhydrous nitrogen atmosphere at room temperature. After stirring 30 min at room temperature, the reaction mixture was concentrated under reduced pressure, and the resulting residue was purified by preparative RP-HPLC with a Phenomenex Synergi 10μ Max-RP 80 Å column (150×30 mm) using 10% to 90% MeCN in 0.1% formic acid as the eluent. A total of 8 mg of Example 82 was obtained as white a solid. HRMS-ESI+ (m/z): [M+2H]²⁺ calcd for (C₅₀H₇₅N₁₅O₁₅S)/2, 578.76386. found, 578.76375.

### Example 83

### 7′C-(4-(hex-5-ynoylamino)piperidin-1-yl)-α-amanitin

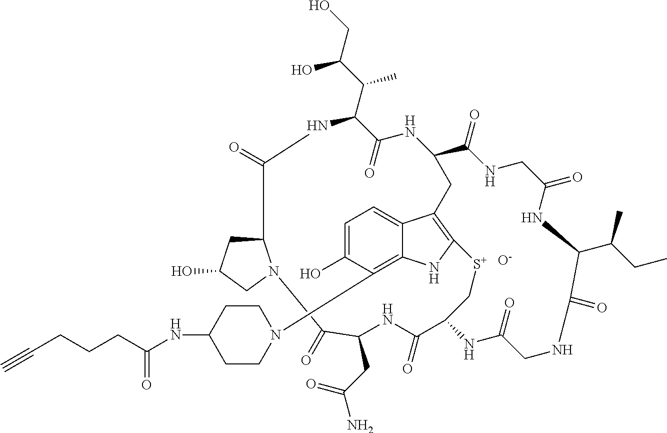

To a solution of Intermediate 2.2 (8 mg) and hex-5-ynoic acid (0.9 mg, 0.008 mmol) in DMF (2 mL) was added HATU (3.9 mg, 0.01 mmol) and diisopropylethyl amine (3.7 μL). After stirring for 30 min, another 3.7 μL of diisopropylethyl amine was added to the reaction mixture. After stirring at room temperature for 1 h, 1% aqueous formic acid solution (1 mL) was added to the reaction mixture. The reaction mixture was purified by preparative RP-HPLC with a Phenomenex Gemeni-NX 10μ C18 110 Å column (150×30 mm) using 10% to 90% MeCN in 0.1% formic acid as the eluent. A total of 3 mg of Example 83 was obtained as a white solid. [M+H]⁺=1111.6. HRMS-ESI+ (m/z): [M+H]⁺ calcd for C₅₀H₇₁N₁₂O₁₅S, 1111.48826. found, 1111.48824

### Example 84

### 7′C-(4-(2-(6-(maleimido)hexanamido)ethyl)piperazin-1-yl)-α-amanitin

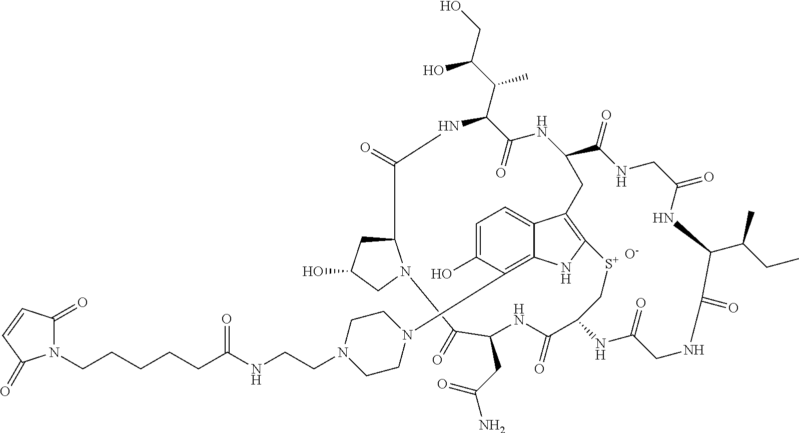

To α-amanitin (48 mg, 0.052 mmol) in methanol (15 mL) was added 5.2 mL of a pre-mixed solution of 10 mM iodine/30 mM tert-butyl (2-(piperazin-1-yl)ethyl)carbamate in methanol under an argon atmosphere. After stirring overnight at room temperature, the reaction mixture was concentrated under reduced pressure to 3 mL and then added dropwise to diethyl ether (45 mL) and the resulting precipitate separated from the supernatant. The precipitate was purified by preparative RP-HPLC. A total of 32 mg of Intermediate 84.1 was obtained as a white solid. [M+H]⁺=1147.63.

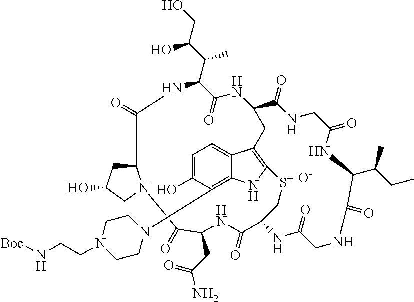

To Intermediate 84.1 (32 mg) was added a mixture of TFA (2 mL), methylene chloride (0.5 mL), and water (50 μL). After stirring for 30 min, the reaction mixture was concentrated under reduced pressure and the resulting residue was then further dried under high vacuum. Intermediate 84.2 was obtained as the TFA salt as a gummy solid, was used subsequently without further purification. [M+H]⁺=1047.82.

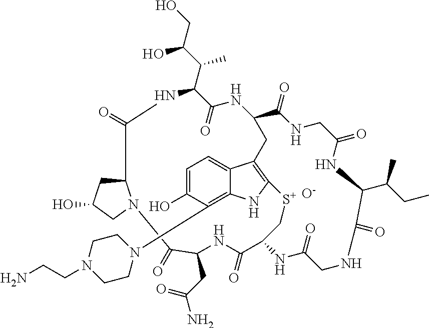

To a solution of intermediate 84.2 (16 mg) in DMSO (2 mL) was added N-(6-maleimideocaproyloxy)succinimide (5.6 mg, 0.018 mmol). After stirring for 1 h, the reaction mixture was purified by preparative RP-HPLC. A total of 6 mg of Example 84 was obtained as a white solid. HRMS-ESI+ (m/z): [M+H]⁺ calcd for C₅₅H₇₉N₁₄O₁₇S, 1239.54631. found, 1239.55071.

### Example 85

### 7′C-(4-(2-(6-(6-(maleimido)hexanamido)hexanamido)ethyl)piperazin-1-yl)-α-amanitin

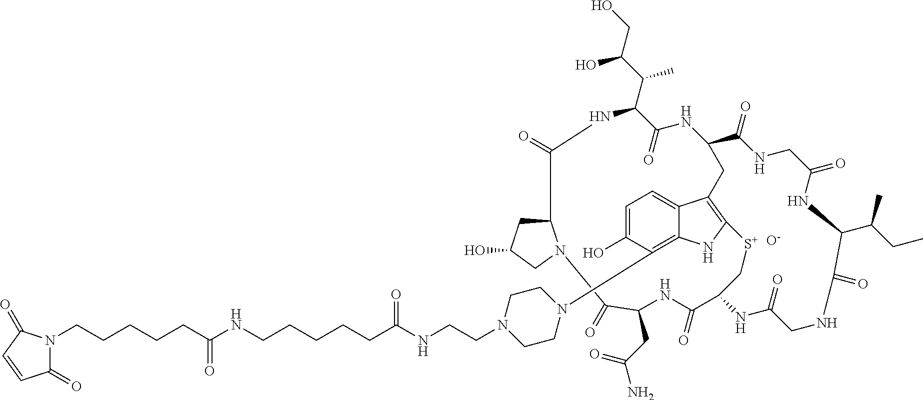

To a solution of intermediate 84.2 (16 mg) in DMSO (2 mL) was added 2,5-dioxopyrrolidin-1-yl 6-(6-(2,5-dioxo-2,5-dihydro-1H-pyrrol-1-yl)hexanamido)hexanoate (7.7 mg, 0.018 mmol). After stirring for 1 h, the reaction mixture was purified by preparative RP-HPLC. A total of 5 mg of Example 85 was obtained as a white solid. HRMS-ESI+ (m/z): [M+H]⁺ calcd for C₆₁H₉₀N₁₅O₁₈S, 1352.63037. found, 1352.63498.

### Example 86

### 6′O-(6-(6-(11,12-didehydro-5,6-dihydro-dibenz[b,f]azocin-5-yl)-6-oxohexanamido)hexyl)-α-amanitin

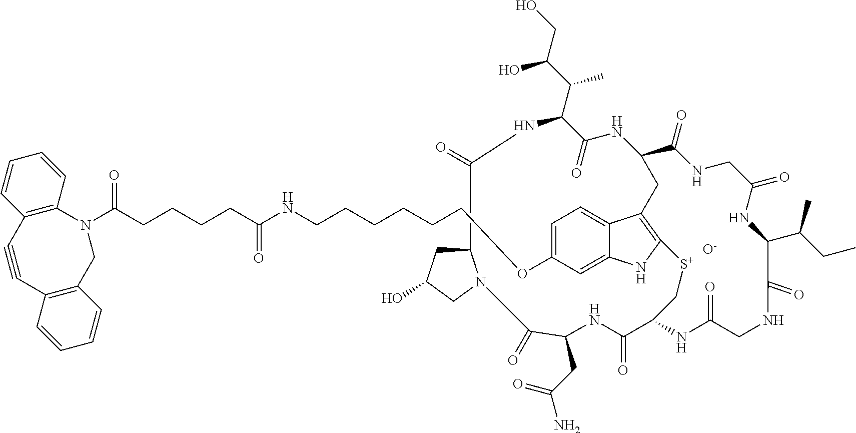

To a mixture of intermediate 76.2 (20 mg) and N-(6-(11,12-didehydro-5,6-dihydro-dibenz[b,f]azocin-5-yl)-6-oxohexanoyloxy)succinimide:

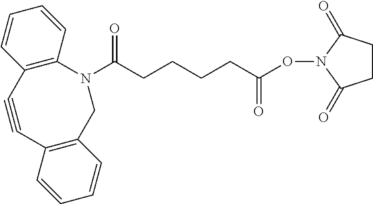 [SMILES: O=C(CCCCC(=O)N1Cc2ccccc2C#Cc2ccccc21)ON1C(=O)CCC1=O]

(10 mg, 0.023 mmol) in DMSO (3 mL) was added diisopropylethyl amine (6.8 μL) under an anhydrous nitrogen atmosphere at room temperature. After stirring for 1 h, the reaction mixture was concentrated under reduced pressure, and the resulting residue was purified by preparative RP-HPLC with a Phenomenex Synergi 10μ Max-RP 80 Å column (150×30 mm) using 10% to 90% MeCN in 0.1% formic acid as the eluent. A total of 7 mg of Example 86 was obtained as a white solid. HRMS-ESI+ (m/z): [M+2H]²⁺ calcd for (C₆₆H₈₆N₁₂O₁₆S)/2, 667.29975. found, 667.30161.

### Example 87

### 6′O-(6-(hex-5-ynoylamino)hexyl)-α-amanitin

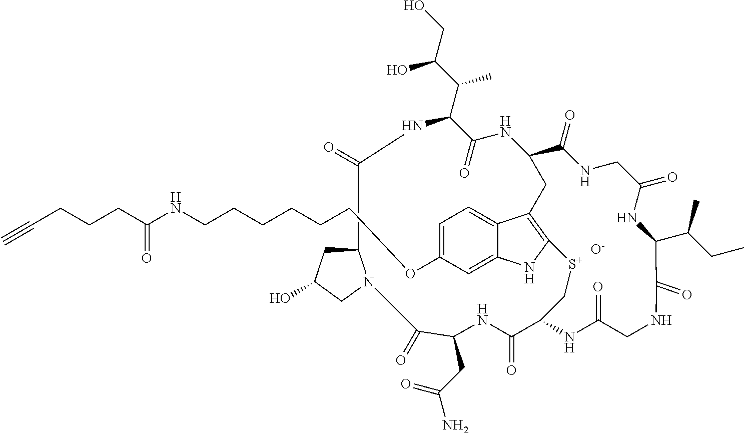

To a mixture of intermediate 76.2 (25 mg) and hex-5-ynoic acid (3 mg, 0.027 mmol) in DMF (1 mL) was added diisopropylethyl amine (9 μL) under an anhydrous nitrogen atmosphere at room temperature. After stirring for 3 h, the reaction mixture was concentrated under reduced pressure, and the resulting residue was purified by preparative RP-HPLC RP-HPLC with a Phenomenex Synergi 10μ Max-RP 80 Å column (150×30 mm) using 10% to 90% MeCN in 0.1% formic acid as the eluent. A total of 10 mg of Example 87 was obtained as a white solid. HRMS-ESI+ (m/z): [M+H]⁺ calcd for C₅₁H₇₄N₁₁O₁₅S, 1112.50813. found, 1112.51098.

### Example 88

### 6′O-(6-(2-(aminooxy)acetylamido)hexyl)-α-amanitin

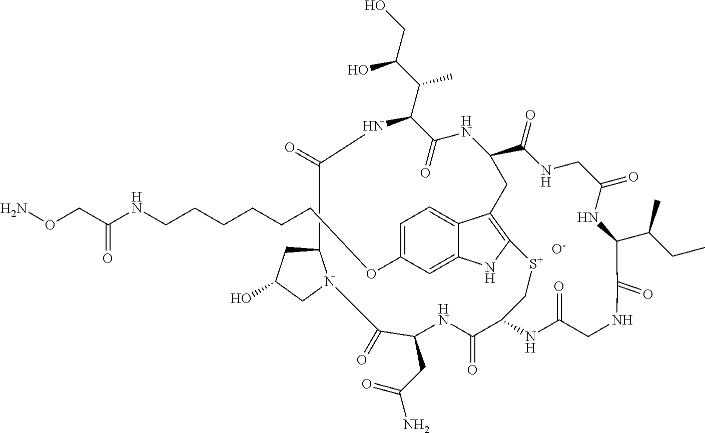

To a solution of intermediate 76.2 (20 mg) in DMF (2 mL) was added 2-(Boc-aminooxy)acetic acid (4.5 mg, 0.024 mmol), diisopropylethyl amine (18 μL), and HATU (9.2 mg, 0.039 mmol). After stirring for 30 min at room temperature, the reaction mixture was purified by preparative RP-HPLC with a Phenomenex Gemeni-NX 10μ C18 110 Å column (150×30 mm) using 10% to 90% MeCN in 5 mM aqueous NH₄OH as the eluent. Intermediate 88.1 (18 mg) was obtained as a white powder. [M+H]⁺=1192.01.

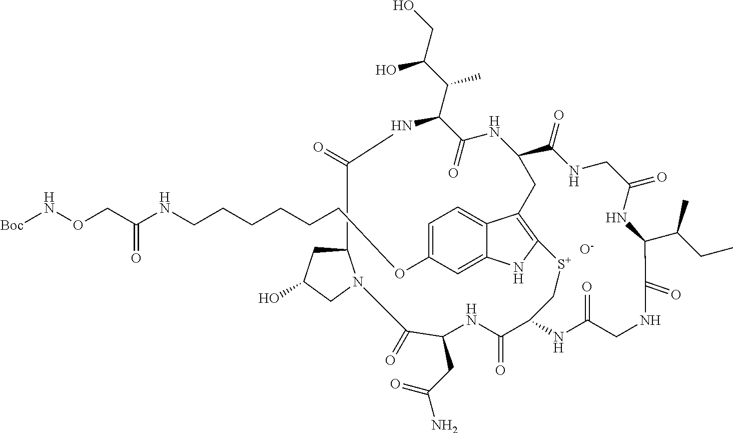

To intermediate 5.5 (18 mg) was added a mixture of TFA (1 mL), dichloromethane (2 mL), water (10 μL), anisole (10 μL) at room temperature. After stirring for 0.3 h, the reaction mixture was concentrated under reduced pressure and the resulting residue was purified by preparative RP-HPLC with a Phenomenex Synergi 10μ Max-RP 80 Å column (150×30 mm) using 10% to 90% MeCN in 0.1% TFA as the eluent. A total of 18 mg of the TFA salt of Example 88 was obtained as a white powder. HRMS-ESI+ (m/z): [M+H]⁺ calcd for C₄₇H₇₁N₁₂O₁₆S, 1091.48264. found, 1091.4799.

### Example 89

### 6′O-((6-aminooxy)hexyl)-α-amanitin

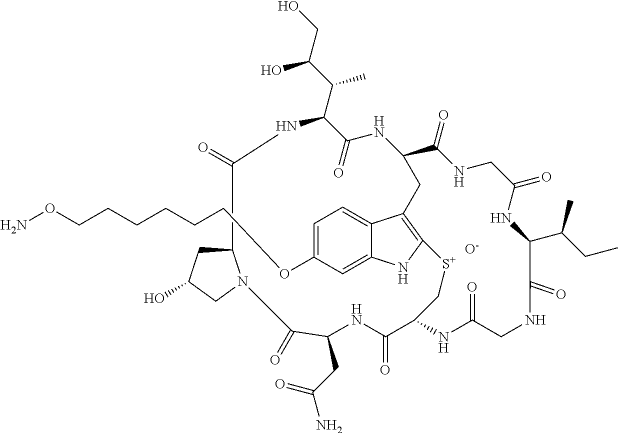

To a stirred 23° C. solution of N-hydroxyphthalimide (1.0 g, 6.13 mmol) and 1,6-dibromohexane (5 mL, 32.5 mmol) in DMF (20 mL) was added K₂CO₃ (0.85 g, 6.13 mmol). After stirring for 72 h, the reaction mixture was diluted with ethyl acetate (150 mL). A white precipitate (inorganic salt) was separated from the supernatant by filtration. The supernatant was concentrated under reduced pressure, and the resulting residue was purified by silica gel column chromatography with 10 to 30% ethyl acetate/hexane to give 1.76 g (5.389 mmol) of Intermediate 89.1 as a colorless oil. [M+H]⁺=328.0.

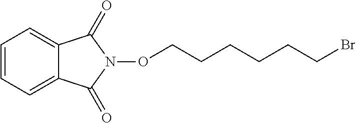 [SMILES: O=C1c2ccccc2C(=O)N1OCCCCCCBr]

To a mixture of α-amanitin (66 mg, 0.072 mmol) and Intermediate 89.1 (117 mg, 0.359 mmol) in DMSO (3 mL) was added potassium tert-butoxide (10 mg, 0.09 mmol) under an anhydrous nitrogen atmosphere at room temperature. After stirring for 18 h, the reaction mixture was treated with glacial acetic acid (0.1 mL) and the mixture was purified by preparative RP-HPLC with a Phenomenex Gemeni-NX 10μ C18 110 Å column (150×30 mm) using on 10% to 90% MeCN in 5 mM aqueous NH₄OH as the eluent. A total of 14 mg of Intermediate 89.2 was obtained as a white solid. [M+H]⁺=1164.51.

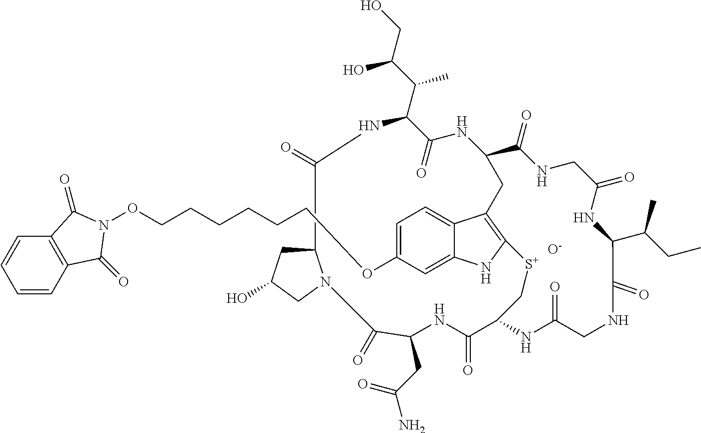

To a solution of Intermediate 89.2 (14 mg) in DMF (3 mL) was added hydrazine monohydrate (100 μL) at room temperature. After stirring for 30 min, the reaction mixture was purified by preparative RP-HPLC with a Phenomenex Gemeni-NX 10μ C18 110 Å column (150×30 mm) using on 10% to 90% MeCN in 0.1% formic acid as the eluent. A total of 5 mg of Example 89 was obtained as a white solid. HRMS-ESI+ (m/z): [M+H]⁺ calcd for C₄₅H₆₈N₁₁O₁₅S, 1034.46118. found, 1034.45990.

### Example 90

### 6′O-(6-(2-iodoacetamido)hexyl)-α-amanitin

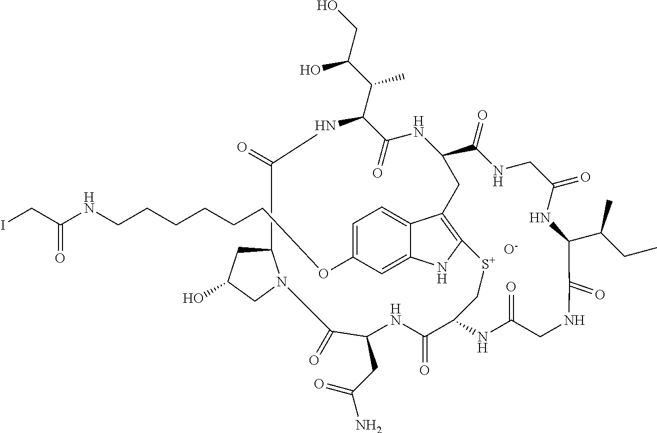

Example 90 may be prepared using the method described for Example 81, using iodoacetyl bromide in place of bromoacetyl bromide.

### Example 91

### Conjugation of amanitin derivatives to cellular transport facilitators

The following procedures provide examples of conjugation of a drug moiety to an antibody via a cysteine residue, with a low drug-antibody ratio for the conjugate.

(A) Herceptin-Example 1. To a solution of 5 mg of Herceptin dissolved in 234 μL of water was added 12.5 μL of 1 M Tris pH 7.4 solution, 364 μL of water, 7.9 μL of 10 mM tris(2-carboxyethyl)phosphine (TCEP) solution, and 6.3 μL of 0.5 M EDTA. The reaction mixture was incubated in a 37° C. water bath for 2 h. To this mixture was added 34.9 μL of a 2.9 mM solution of Example 1 in DMSO. The reaction was performed for 1 h at rt. To this solution was added 2 μL of 0.1 M N-acetyl-L-cysteine. The isolation of the Herceptin-Example 1 conjugate was performed by separation of the macromolecular component on a G-25 gel filtration column and yielded 3.5 mg of drug-antibody conjugate. The drug-antibody ratio was calculated by measuring the absorbance at 310 nm and 280 nm of Herceptin-Example 1 conjugate, using the extinction coefficient for α-amanitin of 13500 cm⁻¹M⁻¹. The drug-antibody ratio of this conjugate was 3.1.

(B) Herceptin-Example 2. To a solution of 5 mg of Herceptin dissolved in 234 μL of water was added 12.5 μL of 1 M Tris pH 7.4 solution, 364 μL of water, 7.9 μL of 10 mM TCEP solution, and 6.3 μL of 0.5 M EDTA. The reaction mixture was incubated in a 37° C. water bath for 2 h. To this mixture was added 33.7 μL of a 3.0 mM solution of Example 2 in DMSO. The reaction was performed for 1 h at rt. To this solution was added 2 μL of 0.1 M N-acetyl-L-cysteine. The isolation of Herceptin-Example 2 conjugate was performed by separation of the macromolecular component on a G-25 gel filtration column and yielded 3.2 mg of drug-antibody conjugate. The drug-antibody ratio was calculated by measuring the absorbance at 310 nm and 280 nm of Herceptin-Example 2 conjugate, using the extinction coefficient for α-amanitin of 13500 cm⁻¹M⁻¹. The drug-antibody ratio of this conjugate was 3.3.

(C) Herceptin-Example 76. To a solution of 5 mg of Herceptin dissolved in 234 μL of water was added 12.5 μL of 1 M Tris pH 7.4 solution, 364 μL of water, 7.9 μL of 10 mM TCEP solution, and 6.3 μL of 0.5 M EDTA. The reaction mixture was incubated in a 37° C. water bath for 2 h. To this mixture was added 18.4 μL of a 5.5 mM solution of Example 76 in DMSO. The reaction was performed for 1 h at rt. To this solution was added 2 μL of 0.1 M N-acetyl-L-cysteine. The isolation of Herceptin-Example 76 conjugate was performed by separation of the macromolecular component on a G-25 gel filtration column and yielded 3.4 mg of antibody-drug conjugate. The drug-antibody ratio was calculated by measuring the absorbance at 310 nm and 280 nm of Herceptin-Example 76 conjugate, using the extinction coefficient for α-amanitin of 13500 cm⁻¹M⁻¹. The drug-antibody ratio of this conjugate was 3.5.

The following procedures provide examples of conjugation of a drug moiety to an antibody via a cysteine residue, with a high drug-antibody ratio for the conjugate.

(D) Herceptin-Example 1. To a solution of 7 mg of Herceptin dissolved in 327 μL of water was added 18 μL of 1 M Tris pH 7.4 solution, 493 μL of water, 28 μL of 10 mM TCEP solution, and 9 μL of 0.5 M EDTA. The reaction mixture was incubated in a 37° C. water bath for 2 h. To this mixture was added 135 μL of a 2.9 mM solution of Example 1 in DMSO. The reaction was performed for 1 h at rt. To this solution was added 2 μL of 0.1 M N-acetyl-L-cysteine. The isolation of Herceptin-Example 1 conjugate was performed by separation of the macromolecular component on a G-25 gel filtration column and yielded 5.5 mg of antibody-drug conjugate. The drug-antibody ratio was calculated by measuring the absorbance at 310 nm and 280 nm of Herceptin-example 1 conjugate, using the extinction coefficient for α-amanitin of 13500 cm⁻¹M⁻¹. The drug-antibody ratio of this conjugate was 6.3.

(E) Herceptin-Example 2. To a solution of 5 mg of Herceptin dissolved in 234 μL of water was added 12.5 μL of 1 M Tris pH 7.4 solution, 351 μL of water, 20 μL of 10 mM TCEP solution, and 6.2 μL of 0.5 M EDTA. The reaction mixture was incubated in a 37° C. water bath for 2 h. To this mixture was added 89 μL of a 3.0 mM solution of Example 2 in DMSO. The reaction was performed for 1 h at rt. To this solution was added 2 μL of 0.1 M N-acetyl-L-cysteine. The isolation of Herceptin-Example 2 conjugate was performed by separation of the macromolecular component on a G-25 gel filtration column and gave yielded 3.0 mg of antibody-drug conjugate. The drug-antibody ratio was calculated by measuring the absorbance at 310 nm and 280 nm of Herceptin-Example 2 conjugate, using the extinction coefficient for α-amanitin of 13500 cm⁻¹M⁻¹. The drug-antibody ratio of this conjugate was 8.4.

(F) Herceptin-Example 76. To a solution of 5 mg of Herceptin dissolved in 234 μL of water was added 10 μL of 1 M Tris pH 7.4 solution, 231 μL of water, 20 μL of 10 mM TCEP solution, and 5 μL of 0.5 M EDTA. The reaction mixture was incubated in a 37° C. water bath for 2 h. To this mixture was added 51 μL of a 5.5 mM solution of Example 76 in DMSO. The reaction was performed for 1 h at RT. To this solution was added 2 μL of 0.1 M N-acetyl-L-cysteine. The isolation of Herceptin-Example 76 conjugate was performed by separation of the macromolecular component on a G-25 gel filtration column and yielded 3.5 mg of antibody-drug conjugate. The drug-antibody ratio was calculated by measuring the absorbance at 310 nm and 280 nm of Herceptin-Example 76 conjugate, using the extinction coefficient for α-amanitin of 13500 cm⁻¹M⁻¹. The drug-antibody ratio of this conjugate was 7.8.

(G) H3-1.4.1.2-Example 1. To a solution of 7 mg of H3-1.4.1.2 (also referred to herein as IgG1) dissolved in 2 mL of PBS was added 42 μL of 1 M Tris pH 7.4 solution, 29 μL of water, 28 μL of 10 mM TCEP solution, and 30 μL of 0.5 M EDTA. The reaction mixture was incubated in a 37° C. water bath for 2 h. To the eluted solution was added 135 μL of a 2.9 mM solution of Example 1 in DMSO. The reaction was performed for 1 h at rt. To this solution was added 2 μL of 0.1 M N-acetyl-L-cysteine. The isolation of H3-1.4.1.2-Example 1 conjugate (also referred to herein as IgG1-Example 1) was performed by separation of the macromolecular component on a G-25 gel filtration column and yielded 5 mg of antibody-drug conjugate. The drug-antibody ratio was calculated by measuring the absorbance at 310 nm and 280 nm of H3-1.4.1.2-Example 1 conjugate, using the extinction coefficient for α-amanitin of 13500 cm⁻¹M⁻¹. The drug-antibody ratio of this conjugate was 6.6.

(H) H16-7.8-Example 76. To a solution of 6 mg of H16-7.8 dissolved in 898 μL of 50 mM of sodium borate, 200 mM of NaCl, pH 9.0 buffer was added 15.9 μL of 10 mM tris(2-carboxyethyl)phosphine (TCEP) solution, and 9.1 μL of 0.5 M EDTA. The reaction mixture was incubated in a 37° C. water bath for 2 h. To this mixture was added 18 μL of a 10.2 mM solution of Example 76 in DMSO. The reaction was performed for 1 h at rt. To this solution was added 8.9 μL of 0.1 M N-acetyl-L-cysteine. The isolation of the H16-7.8-Example 76 conjugate was performed by separation of the macromolecular component on a G-25 gel filtration column and yielded 2.7 mg of drug-antibody conjugate. The drug-antibody ratio was calculated by measuring the absorbance at 310 nm and 280 nm of AGS16C-Example 76 conjugate, using the extinction coefficient for α-amanitin of 13500 cm⁻¹M⁻¹. The drug-antibody ratio of this conjugate was 4.8.

(I) Anti-CD33-antibody-Example 1. All anti-CD33 conjugations used a mouse derived IgG1K monoclonal antibody derived from VelocImmune mice (Regeneron, Tarrytown, N.Y.) that comprise fully human variable regions and mouse constant regions. To a solution of 9.1 mg of Anti-CD33-antibody dissolved in 5.5 ml of PBS was added 616 μL of 0.5 M of sodium borate pH 9.0 buffer, 246 μL of 5M NaCl, 22.6 μL of 10 mM tris(2-carboxyethyl)phosphine (TCEP) solution, and 61.6 μL of 0.5 M EDTA. The reaction mixture was incubated in a 37 oC water bath for 2 h. To the 1.149 ml of this mixture was added 53 μL of a 1.3 mM solution of Example 1 in DMSO. The reaction was performed for 1 h at rt. Anti-CD33-antibody-Example 1 conjugate was performed by separation of the macromolecular component on a G-25 gel filtration column and yielded 1.6 mg of drug-antibody conjugate. The drug-antibody ratio was calculated by measuring the absorbance at 310 nm and 280 nm of Anti-CD33-antibody-Example 1 conjugate, using the extinction coefficient for Example 1 of 14996 cm⁻¹M⁻¹. The drug-antibody ratio of this conjugate was 5.1.

(J) Anti-CD33-antibody-Example 2. To a solution of 9.1 mg of Anti-CD33-antibody dissolved in 5.5 ml of PBS was added 616 μL of 0.5 M of sodium borate pH 9.0 buffer, 246 μL of 5M NaCl, 22.6 μL of 10 mM tris(2-carboxyethyl)phosphine (TCEP) solution, and 61.6 μL of 0.5 M EDTA. The reaction mixture was incubated in a 37 oC water bath for 2 h. To the 1.149 ml of this mixture was added 23 μL of a 3 mM solution of Example 2 in DMSO. The reaction was performed for 1 h at rt. Anti-CD33-antibody-Example 2 conjugate was performed by separation of the macromolecular component on a G-25 gel filtration column and yielded 1.7 mg of drug-antibody conjugate. The drug-antibody ratio was calculated by measuring the absorbance at 310 nm and 280 nm of Anti-CD33-antibody-Example 2 conjugate, using the extinction coefficient for Example 2 of 14996 cm⁻¹M⁻¹. The drug-antibody ratio of this conjugate was 5.6.

(K) Anti-CD33-antibody-Example 27. To a solution of 9.1 mg of Anti-CD33-antibody dissolved in 5.5 ml of PBS was added 616 μL of 0.5 M of sodium borate pH 9.0 buffer, 246 μL of 5M NaCl, 22.6 μL of 10 mM tris(2-carboxyethyl)phosphine (TCEP) solution, and 61.6 μL of 0.5 M EDTA. The reaction mixture was incubated in a 37 oC water bath for 2 h. To the 1.149 ml of this mixture was added 11 μL of a 6.2 mM solution of Example 27 in DMSO. The reaction was performed for 1 h at rt. Anti-CD33-antibody-Example 27 conjugate was performed by separation of the macromolecular component on a G-25 gel filtration column and yielded 1.6 mg of drug-antibody conjugate. The drug-antibody ratio was calculated by measuring the absorbance at 310 nm and 280 nm of Anti-CD33-antibody-Example 27 conjugate, using the extinction coefficient for Example 27 of 14996 cm⁻¹M⁻¹. The drug-antibody ratio of this conjugate was 6.6.

(L) Anti-CD33-antibody-Example 76. To a solution of 9.1 mg of Anti-CD33-antibody dissolved in 5.5 ml of PBS was added 616 μL of 0.5 M of sodium borate pH 9.0 buffer, 246 μL of 5M NaCl, 22.6 μL of 10 mM tris(2-carboxyethyl)phosphine (TCEP) solution, and 61.6 μL of 0.5 M EDTA. The reaction mixture was incubated in a 37 oC water bath for 2 h. To the 1.149 ml of this mixture was added 7 μL of a 10.2 mM solution of Example 76 in DMSO. The reaction was performed for 1 h at rt. Anti-CD33-antibody-Example 76 conjugate was performed by separation of the macromolecular component on a G-25 gel filtration column and yielded 1.7 mg of drug-antibody conjugate. The drug-antibody ratio was calculated by measuring the absorbance at 310 nm and 280 nm of Anti-CD33-antibody-Example 76 conjugate, using the extinction coefficient for Example 76 of 16708 cm⁻¹M⁻¹. The drug-antibody ratio of this conjugate was 4.0.

(M) Anti-CD33-antibody-Example 76. To a solution of 12 mg of Anti-CD33-antibody dissolved in 2.891 ml of PBS was added 333 μL of 0.5 M of sodium borate pH 9.0 buffer, 63.5 μL of 10 mM tris(2-carboxyethyl)phosphine (TCEP) solution, 33.3 μL of 0.5 M EDTA and 12 μL of water. The reaction mixture was incubated in a 37° C. water bath for 2 h. To this mixture was added 75 μL of a 10.6 mM solution of Example 76 in DMSO. The reaction was performed for 1 h at rt. To this mixture was added 5 μL of 0.1 M N-acetyl-L-cysteine. Anti-CD33-antibody-Example 76 conjugate was performed by separation of the macromolecular component on a G-25 gel filtration column and yielded 10.6 mg of drug-antibody conjugate. The drug-antibody ratio was calculated by measuring the absorbance at 310 nm and 280 nm of Anti-CD33-antibody-Example 76 conjugate, using the extinction coefficient for Example 76 of 16708 cm⁻¹M⁻¹. The drug-antibody ratio of this conjugate was 8.6.

(N) Anti-CD71-antibody-Example 1. All anti-CD71 conjugations used a mouse derived murine IgG1 monoclonal antibody (a.k.a. v56-1e7.1) derived from VelocImmune mice (Regeneron, Tarrytown, N.Y.) that comprise fully human variable regions and mouse constant regions. To a solution of 16.1 mg of Anti-CD71-antibody dissolved in 3.8 ml of PBS was added 434 μL of 0.5 M of sodium borate pH 9.0 buffer, 174 μL of 5M NaCl, 39.8 μL of 10 mM tris(2-carboxyethyl)phosphine (TCEP) solution, 43.4 μL of 0.5 M EDTA and 25 μL of water. The reaction mixture was incubated in a 37 oC water bath for 2 h. To the 1.047 ml of this mixture was added 99 μL of a 1.3 mM solution of Example 1 in DMSO. The reaction was performed for 1 h at rt. Anti-CD71-antibody-Example 1 conjugate was performed by separation of the macromolecular component on a G-25 gel filtration column and yielded 3.3 mg of drug-antibody conjugate. The drug-antibody ratio was calculated by measuring the absorbance at 310 nm and 280 nm of Anti-CD71-antibody-Example 1 conjugate, using the extinction coefficient for Example 1 of 14996 cm⁻¹M⁻¹. The drug-antibody ratio of this conjugate was 5.3.

(O) Anti-CD71-antibody-Example 2. To a solution of 16.1 mg of Anti-CD71-antibody dissolved in 3.8 ml of PBS was added 434 μL of 0.5 M of sodium borate pH 9.0 buffer, 174 μL of 5M NaCl, 39.8 μL of 10 mM tris(2-carboxyethyl)phosphine (TCEP) solution, 43.4 μL of 0.5 M EDTA and 25 μL of water. The reaction mixture was incubated in a 37° C. water bath for 2 h. To the 1.047 ml of this mixture was added 43 μL of a 3 mM solution of Example 2 in DMSO. The reaction was performed for 1 h at rt. Anti-CD71-antibody-Example 2 conjugate was performed by separation of the macromolecular component on a G-25 gel filtration column and yielded 3.2 mg of drug-antibody conjugate. The drug-antibody ratio was calculated by measuring the absorbance at 310 nm and 280 nm of Anti-CD71-antibody-Example 2 conjugate, using the extinction coefficient for Example 2 of 14996 cm⁻¹M⁻¹. The drug-antibody ratio of this conjugate was 3.1

(P) Anti-CD71-antibody-Example 27. To a solution of 16.1 mg of Anti-CD71-antibody dissolved in 3.8 ml of PBS was added 434 μL of 0.5 M of sodium borate pH 9.0 buffer, 174 μL of 5M NaCl, 39.8 μL of 10 mM tris(2-carboxyethyl)phosphine (TCEP) solution, 43.4 μL of 0.5 M EDTA and 25 μL of water. The reaction mixture was incubated in a 37 oC water bath for 2 h. To the 1.047 ml of this mixture was added 21 μL of a 6.2 mM solution of Example 27 in DMSO. The reaction was performed for 1 h at rt. Anti-CD71-antibody-Example 27 conjugate was performed by separation of the macromolecular component on a G-25 gel filtration column and yielded 3.0 mg of drug-antibody conjugate. The drug-antibody ratio was calculated by measuring the absorbance at 310 nm and 280 nm of Anti-CD71-antibody-Example 27 conjugate, using the extinction coefficient for Example 27 of 14996 cm⁻¹M⁻¹. The drug-antibody ratio of this conjugate was 6.7.

(Q) Anti-CD71-antibody-Example 76. To a solution of 16.1 mg of Anti-CD71-antibody dissolved in 3.8 ml of PBS was added 434 μL of 0.5 M of sodium borate pH 9.0 buffer, 174 μL of 5M NaCl, 39.8 μL of 10 mM tris(2-carboxyethyl)phosphine (TCEP) solution, 43.4 μL of 0.5 M EDTA and 25 μL of water. The reaction mixture was incubated in a 37 oC water bath for 2 h. To the 1.047 ml of this mixture was added 13 μL of a 10.2 mM solution of Example 76 in DMSO. The reaction was performed for 1 h at rt. Anti-CD71-antibody-Example 76 conjugate was performed by separation of the macromolecular component on a G-25 gel filtration column and yielded 2.9 mg of drug-antibody conjugate. The drug-antibody ratio was calculated by measuring the absorbance at 310 nm and 280 nm of Anti-CD71-antibody-Example 76 conjugate, using the extinction coefficient for Example 76 of 16708 cm⁻¹M⁻¹. The drug-antibody ratio of this conjugate was 4.2.

(R) Anti-FLT3-antibody-Example 1. All anti-FLT3 conjugations used a mouse derived IgG2a monoclonal antibody derived from VelocImmune mice (Regeneron, Tarrytown, N.Y.) that comprise fully human variable regions and mouse constant regions. To a solution of 11.3 mg of Anti-FLT3-antibody dissolved in 1.849 ml of PBS was added 222 μL of 0.5 M of sodium borate pH 9.0 buffer, 89 μL of 5M NaCl, 22.5 μL of 10 mM tris(2-carboxyethyl)phosphine (TCEP) solution, 22.2 μL of 0.5 M EDTA and 18 μL of water. The reaction mixture was incubated in a 37 oC water bath for 2 h. To the 558 μL of this mixture was added 69 μL of a 1.3 mM solution of Example 1 in DMSO. The reaction was performed for 1 h at rt. Anti-FLT3-antibody-Example 1 conjugate was performed by separation of the macromolecular component on a G-25 gel filtration column and yielded 2.2 mg of drug-antibody conjugate. The drug-antibody ratio was calculated by measuring the absorbance at 310 nm and 280 nm of Anti-FLT3-antibody-Example 1 conjugate, using the extinction coefficient for Example 1 of 14996 cm⁻¹M⁻¹. The drug-antibody ratio of this conjugate was 4.6.

(S) Anti-FLT3-antibody-Example 2. To a solution of 11.3 mg of Anti-FLT3-antibody dissolved in 1.849 ml of PBS was added 222 μL of 0.5 M of sodium borate pH 9.0 buffer, 89 μL of 5M NaCl, 22.5 μL of 10 mM tris(2-carboxyethyl)phosphine (TCEP) solution, 22.2 μL of 0.5 M EDTA and 18 μL of water. The reaction mixture was incubated in a 37 oC water bath for 2 h. To the 558 μL of this mixture was added 29 μL of a 3.04 mM solution of Example 2 in DMSO. The reaction was performed for 1 h at rt. Anti-FLT3-antibody-Example 2 conjugate was performed by separation of the macromolecular component on a G-25 gel filtration column and yielded 2.3 mg of drug-antibody conjugate. The drug-antibody ratio was calculated by measuring the absorbance at 310 nm and 280 nm of Anti-FLT3-antibody-Example 2 conjugate, using the extinction coefficient for Example 2 of 14996 cm⁻¹M⁻¹. The drug-antibody ratio of this conjugate was 4.6.

(T) Anti-FLT3-antibody-Example 27. To a solution of 11.3 mg of Anti-FLT3-antibody dissolved in 1.849 ml of PBS was added 222 μL of 0.5 M of sodium borate pH 9.0 buffer, 89 μL of 5M NaCl, 22.5 μL of 10 mM tris(2-carboxyethyl)phosphine (TCEP) solution, 22.2 μL of 0.5 M EDTA and 18 μL of water. The reaction mixture was incubated in a 37 oC water bath for 2 h. To the 558 μL of this mixture was added 14 μL of a 6.2 mM solution of Example 27 in DMSO. The reaction was performed for 1 h at rt. Anti-FLT3-antibody-Example 27 conjugate was performed by separation of the macromolecular component on a G-25 gel filtration column and yielded 2.2 mg of drug-antibody conjugate. The drug-antibody ratio was calculated by measuring the absorbance at 310 nm and 280 nm of Anti-FLT3-antibody-Example 27 conjugate, using the extinction coefficient for Example 27 of 14996 cm⁻¹M⁻¹. The drug-antibody ratio of this conjugate was 5.7.

(U) Anti-FLT3-antibody-Example 76. To a solution of 9 mg of Anti-FLT3-antibody dissolved in 1.468 ml of PBS was added 176 μL of 0.5 M of sodium borate pH 9.0 buffer, 71 μL of 5M NaCl, 17.9 μL of 10 mM tris(2-carboxyethyl)phosphine (TCEP) solution, 17.6 μL of 0.5 M EDTA and 14 μL of water. The reaction mixture was incubated in a 37° C. water bath for 2 h. To this mixture was added 26 μL of a 10.2 mM solution of Example 76 in DMSO. The reaction was performed for 1 h at rt. Anti-FLT3-antibody-Example 76 conjugate was performed by separation of the macromolecular component on a G-25 gel filtration column and yielded 7.4 mg of drug-antibody conjugate. The drug-antibody ratio was calculated by measuring the absorbance at 310 nm and 280 nm of Anti-FLT3-antibody-Example 76 conjugate, using the extinction coefficient for Example 76 of 16708 cm⁻¹M⁻¹. The drug-antibody ratio of this conjugate was 4.0.

(V) Anti-PSCA-antibody-Example 1. To a solution of 15 mg of Anti-PSCA-antibody dissolved in 625 μL of 10 mM Histidine, 150 mM NaCl, 0.1% PS80 was added 31 μL of 1 M of Tris pH 7.4 buffer, 82 μL of 10 mM tris(2-carboxyethyl)phosphine (TCEP) solution, 15 μL of 0.5 M EDTA and 746 μL of water. The reaction mixture was incubated in a 37° C. water bath for 2 h. To the 715 μL of this mixture was added 176 μL of a 2.9 mM solution of Example 1 in DMSO. The reaction was performed for 1 h at rt. Anti-PSAC-antibody-Example 1 conjugate was performed by separation of the macromolecular component on a G-25 gel filtration column and yielded 3.9 mg of drug-antibody conjugate. The drug-antibody ratio was calculated by measuring the absorbance at 310 nm and 280 nm of Anti-PACS-antibody-Example 1 conjugate, using the extinction coefficient for alpha-amanitin of 13500 cm⁻¹M⁻¹. The drug-antibody ratio of this conjugate was 7.7.

(W) Anti-PSCA-antibody-Example 76. To a solution of 15 mg of Anti-PSCA-antibody dissolved in 625 μL of 10 mM Histidine, 150 mM NaCl, 0.1% PS80 was added 31 μL of 1 M of Tris pH 7.4 buffer, 82 μL of 10 mM tris(2-carboxyethyl)phosphine (TCEP) solution, 15 μL of 0.5 M EDTA and 746 μL of water. The reaction mixture was incubated in a 37° C. water bath for 2 h. To the 715 μL of this mixture was added 93 μL of a 5.5 mM solution of Example 76 in DMSO. The reaction was performed for 1 h at rt. Anti-PSAC-antibody-Example 76 conjugate was performed by separation of the macromolecular component on a G-25 gel filtration column and yielded 3.5 mg of drug-antibody conjugate. The drug-antibody ratio was calculated by measuring the absorbance at 310 nm and 280 nm of Anti-PACS-antibody-Example 76 conjugate, using the extinction coefficient for alpha-amanitin of 13500 cm⁻¹M⁻¹. The drug-antibody ratio of this conjugate was 8.8.

The following procedures provide examples of conjugation of a drug moiety to an antibody via lysine conjugation with 2-iminothiolane.

(H) Herceptin-Example 1. To a solution of 5 mg of Herceptin dissolved in 233 μL of water was added 25 μL of 0.5 M sodium borate pH 8.45 solution, 291 μL of water, 70 μL of 10 mM 2-iminothiolane solution, and 6 μL of 0.5 M EDTA. The reaction mixture was incubated in a 37° C. water bath for 1 h. Excess 2-iminothiolane was removed by a G-25 gel filtration column with PBS elution. To the eluted solution was added 56 μL of a 2.9 mM solution of Example 1 in DMSO. The reaction was performed for 1 h at rt. The isolation of Herceptin-Example 1 conjugate was performed by separation of the macromolecular component on a G-25 gel filtration column. The drug-antibody ratio was calculated by measuring the absorbance at 310 nm and 280 nm of Herceptin-Example 1 conjugate, using the extinction coefficient for α-amanitin of 13500 cm⁻¹M⁻¹. The drug-antibody ratio of this conjugate was 4.1.

(I) Herceptin-Example 2. To a solution of 5 mg of Herceptin dissolved in 233 μL of water was added 25 μL of 0.5 M sodium borate pH 8.45 solution, 291 μL of water, 70 μL of 10 mM 2-iminothiolane solution, and 6 μL of 0.5 M EDTA. The reaction mixture was incubated in a 37° C. water bath for 1 h. Excess 2-iminothiolane was removed by a G-25 gel filtration column with PBS elution. To the eluted solution was added 56 μL of a 2.6 mM solution of Example 2 in DMSO. The reaction was performed for 1 h at rt. The isolation of Herceptin-Example 2 conjugate was performed by separation of the macromolecular component on a G-25 gel filtration column and yielded 3.7 mg of antibody-drug conjugate. The drug-antibody ratio was calculated by measuring the absorbance at 310 nm and 280 nm of Herceptin-Example 2 conjugate, using the extinction coefficient for α-amanitin of 13500 cm⁻¹M⁻¹. The drug-antibody ratio of this conjugate was 4.5.

(J) Herceptin-Example 76. To a solution of 5 mg of Herceptin dissolved in 233 μL of water was added 25 μL of 0.5 M sodium borate pH 8.45 solution, 291 μL of water, 70 μL of 10 mM 2-iminothiolane solution, and 6 μL of 0.5 M EDTA. The reaction mixture was incubated in a 37° C. water bath for 1 h. Excess 2-iminothiolane was removed by a G-25 gel filtration column with PBS elution. To the eluted solution was added 56 μL of a 2.6 mM solution of Example 76 in DMSO. The reaction was performed for 1 h at rt. The isolation of Herceptin-Example 76 conjugate was performed by separation of the macromolecular component on a G-25 gel filtration column and yielded 2.5 mg of antibody-drug conjugate. The drug-antibody ratio was calculated by measuring the absorbance at 310 nm and 280 nm of Herceptin-Example 76 conjugate, using the extinction coefficient for α-amanitin of 13500 cm⁻¹M⁻¹. The drug-antibody ratio of this conjugate was 4.6.

The following references provide examples of conjugation of a prior art drug moiety to an antibody used to compare against compounds of the present invention.

“Prior Art ADC” refers to an α-amanitin-glutarate-IgG1 ADC (prepared from α-amanitin-glutaric acid N-hydroxysuccinimidate as described in WO2010/115629 (published 14 Oct. 2010), page 42, Ex. 1.11.2 and IgG1, as in Ex. 1.11.3 of the same reference).

“Prior Art ADC 2” refers to the ADC prepared in accordance with WO2012/041504 (published 5 Apr. 2012) denoted Her-DSC-30.0134.

### Biological Example 1

### In Vitro Cytotoxicity Assessment

The in vitro efficacy of the antibody-drug conjugates are measured by evaluating their cytotoxic activity on various cancer cell lines. Origin and descriptions of the cell line(s) used in the cytotoxicity assays are as follows:

| Name | Type of Cancer | Source |
|---|---|---|
| MOLM-13 | Acute myeloid leukemia | DSMZ |
| RS4-11 | Acute lymphoblastic leukemia | ATCC |
| EOL-1 | Eosinophilic leukemia | Sigma/HPA |
| Hel92.1.7 | Erythroleukemia | ATCC |
| Pfeiffer | Diffuse large cell lymphoma | ATCC |
| PC3 | human prostate cancer, HER2(−) | ATCC |
| HCC-1954 | human mammary ductal | ATCC |
| | carcinoma, HER2(+) | |
| MDA-MB-468 | Human mammary | ATCC |
| | adenocarcinoma, HER2(−) | |
| UG-K3 | Kidney | Patient derived |

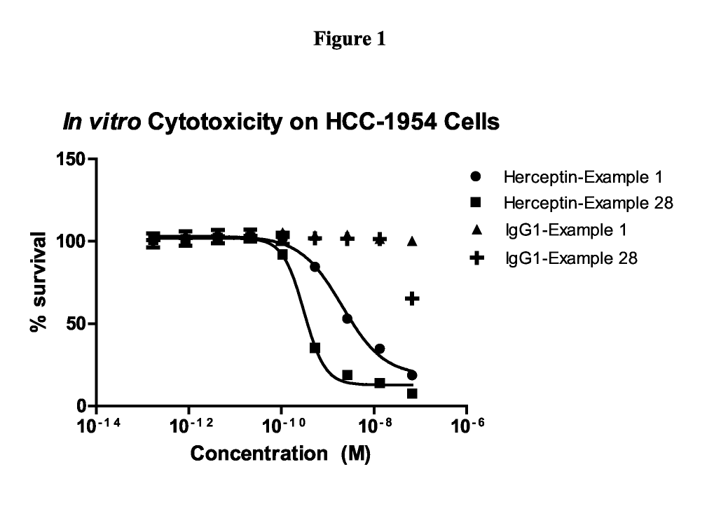

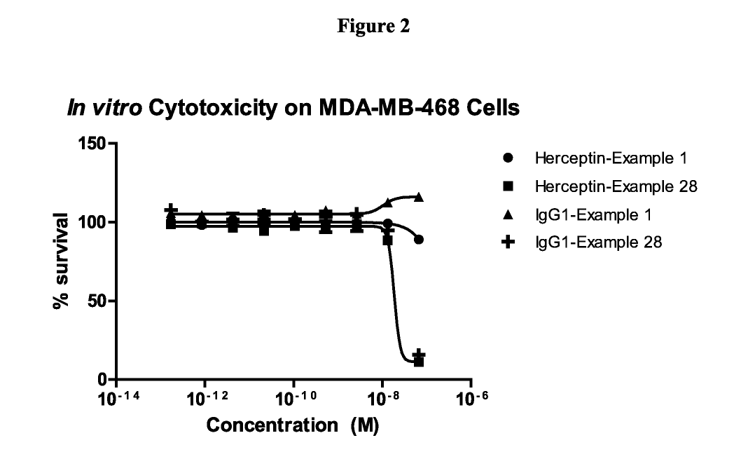

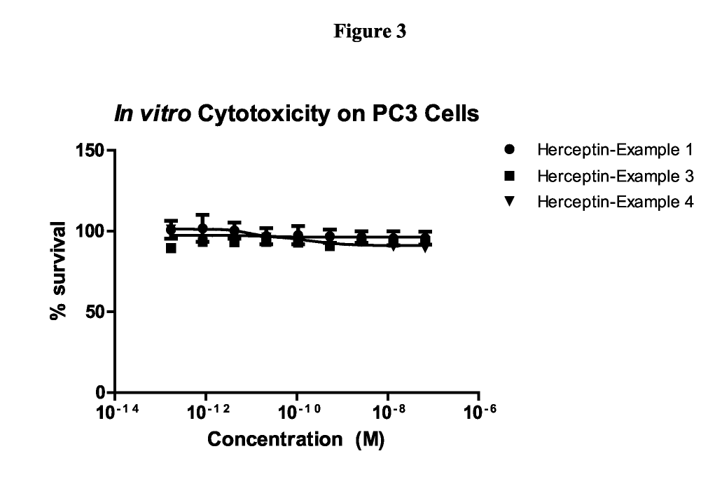

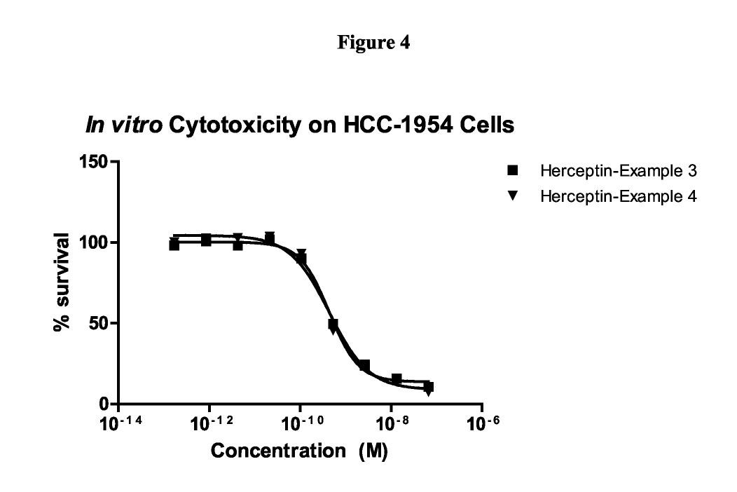

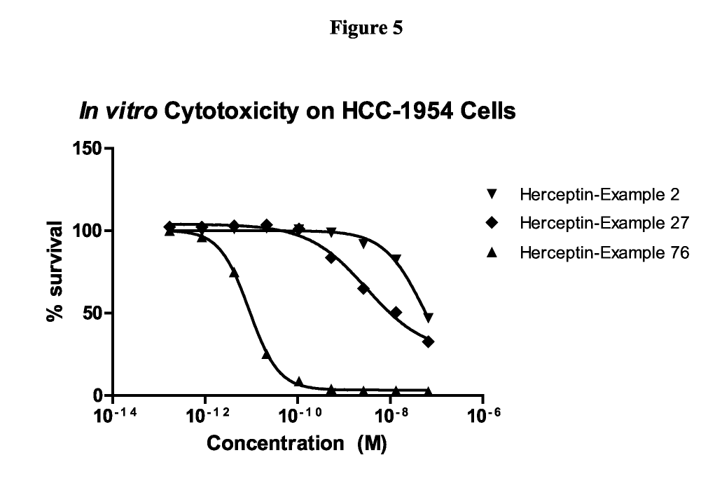

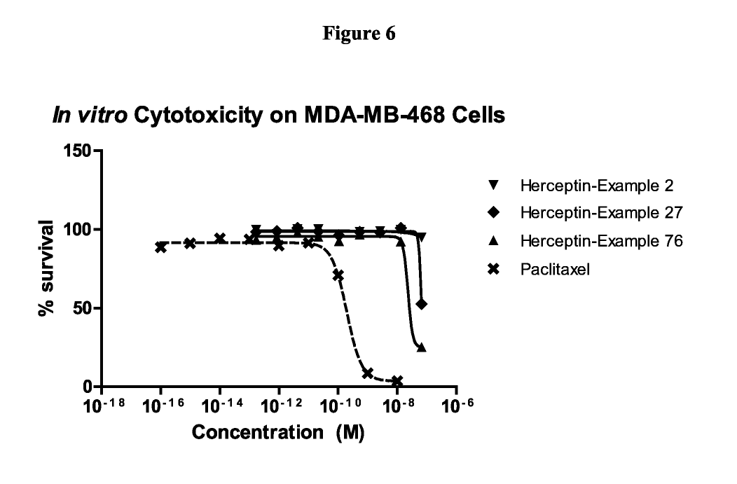

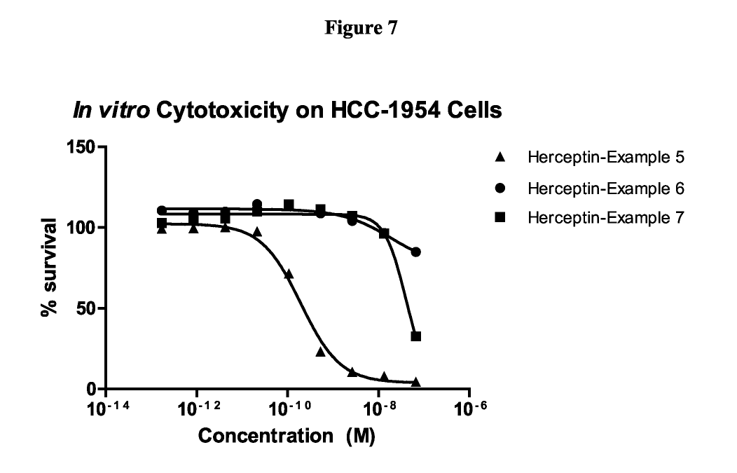

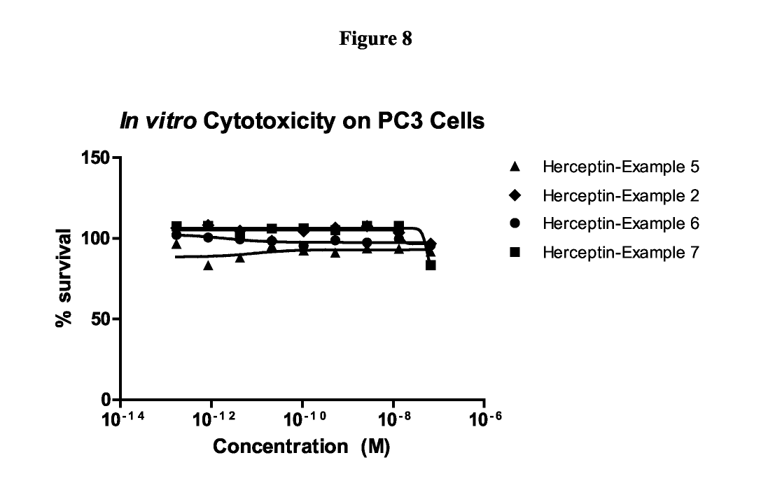

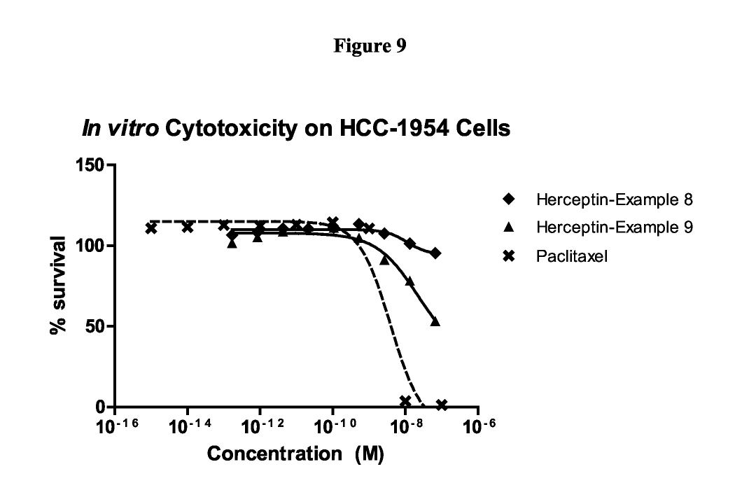

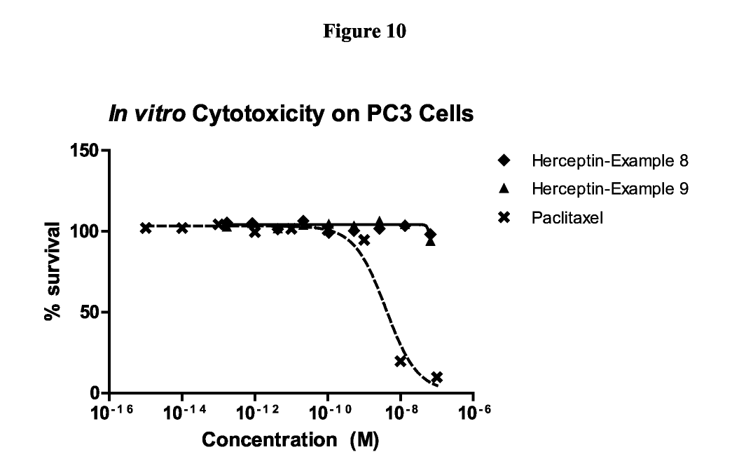

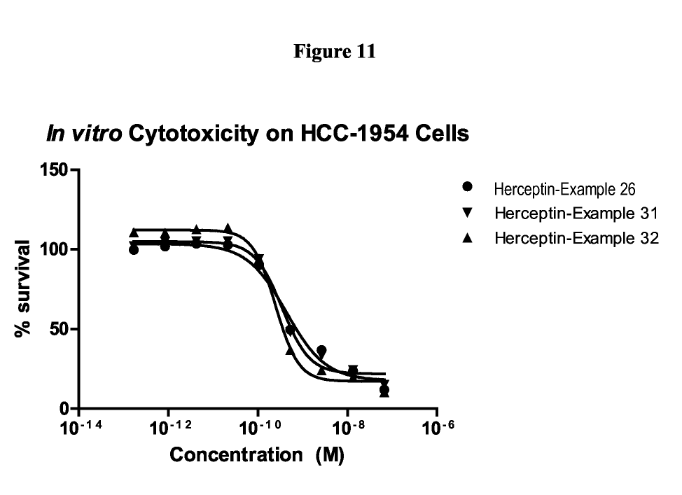

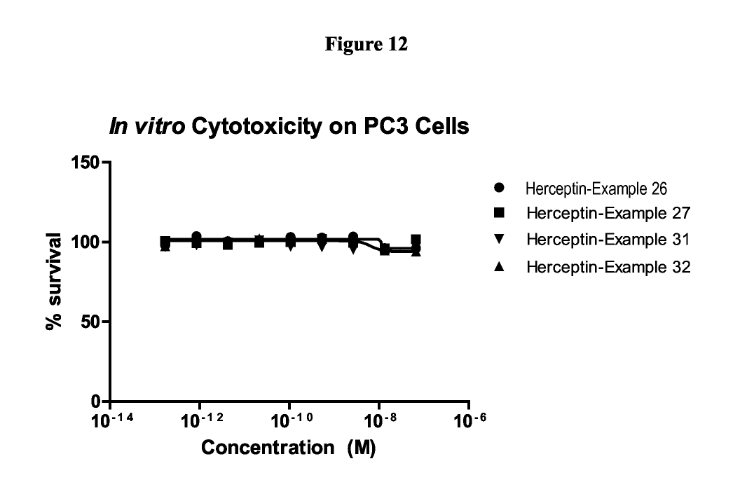

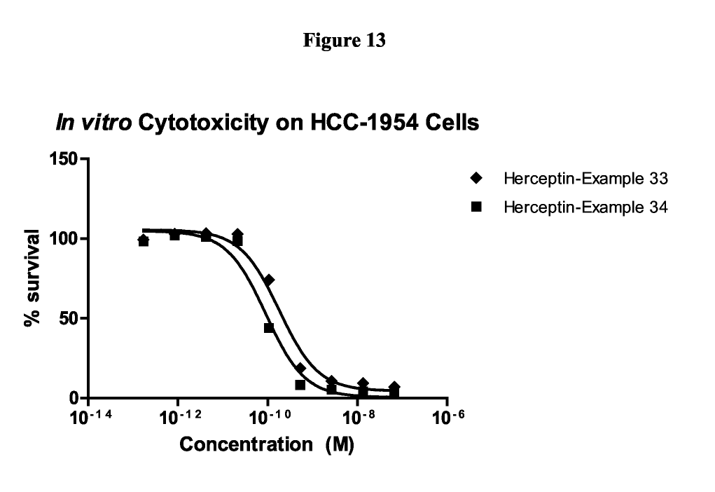

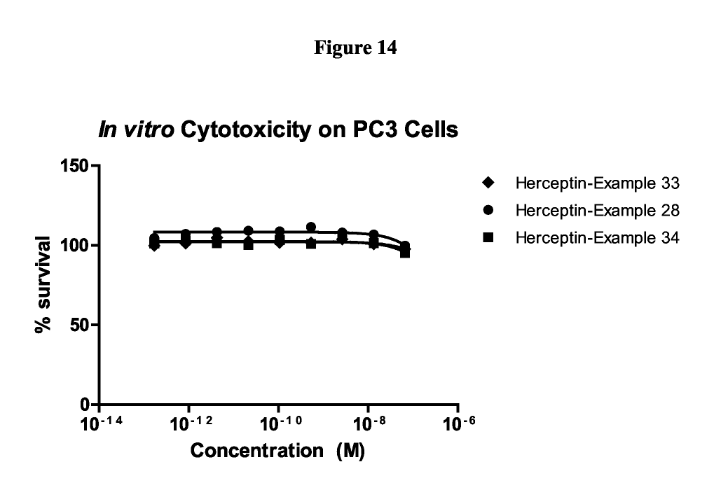

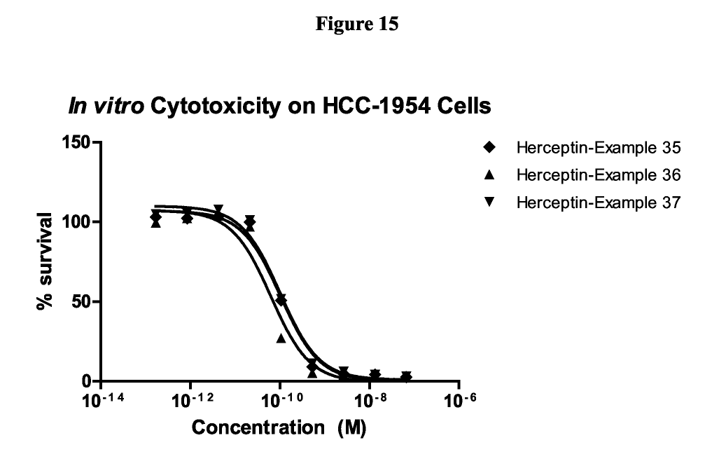

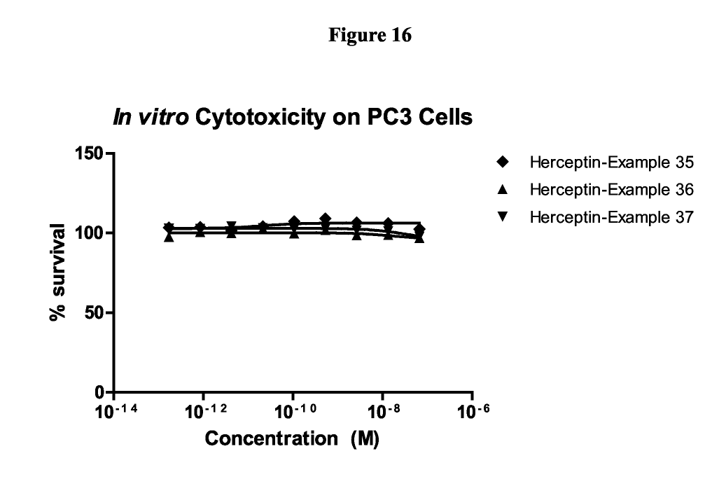

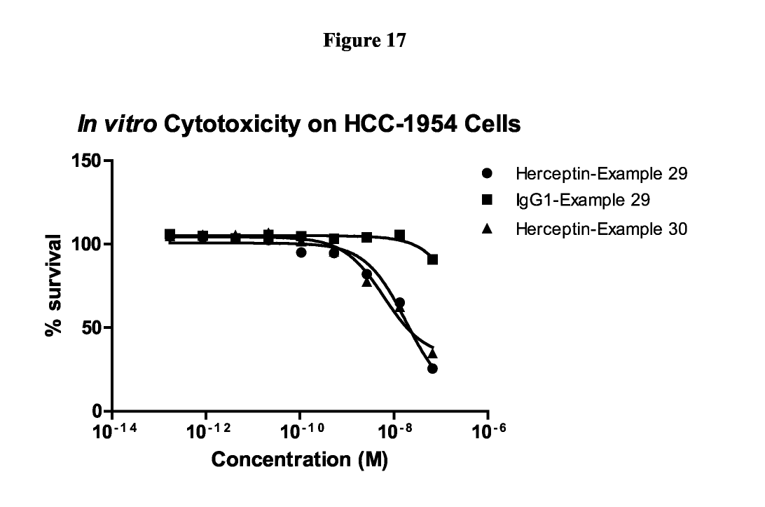

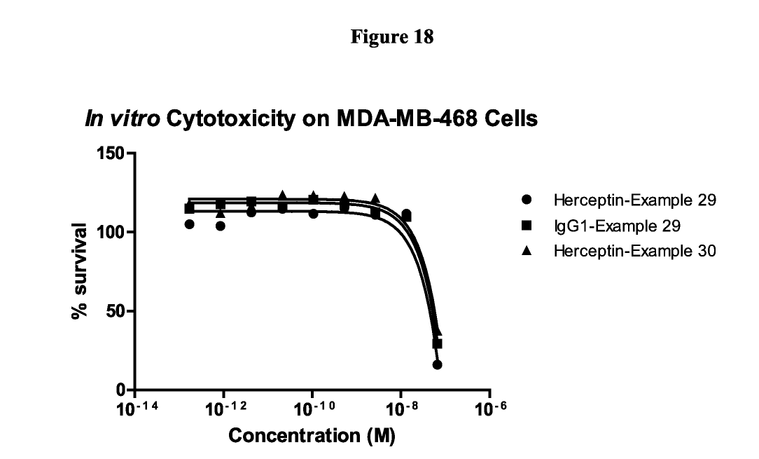

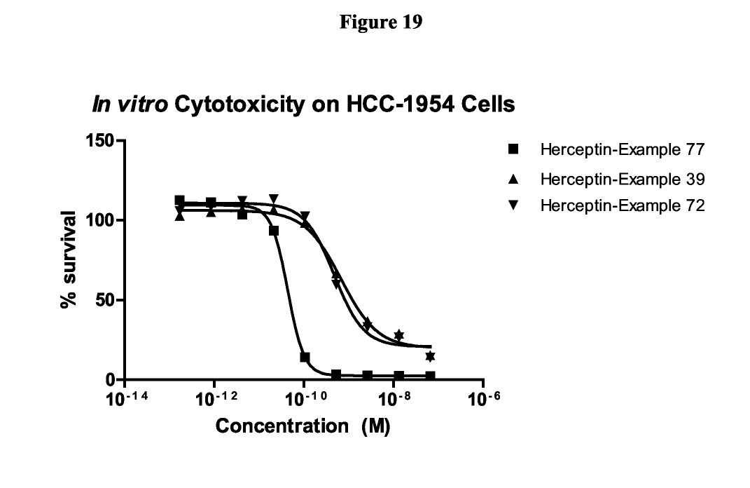

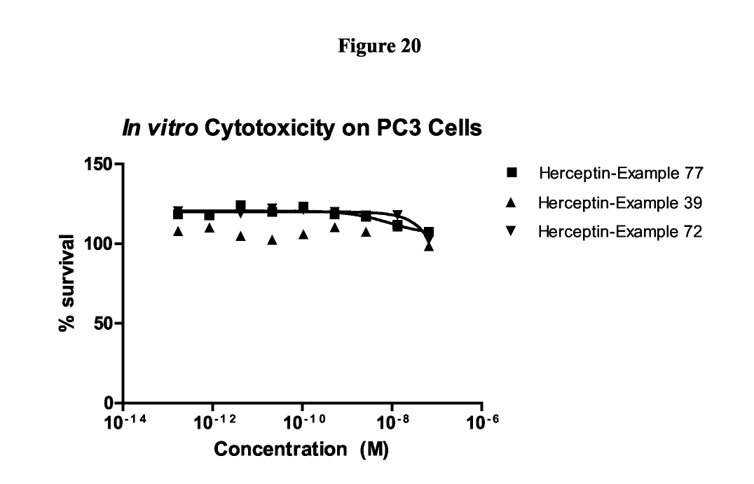

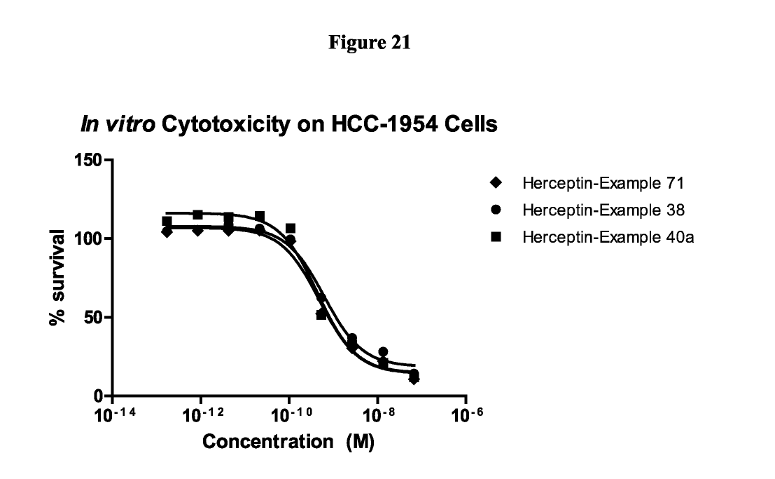

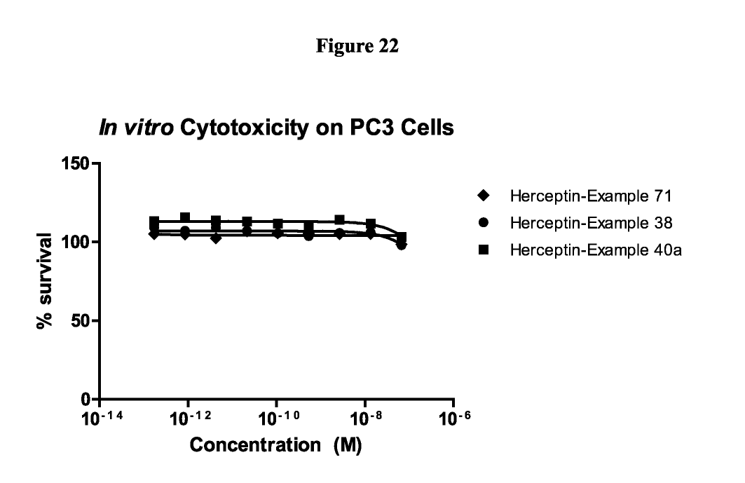

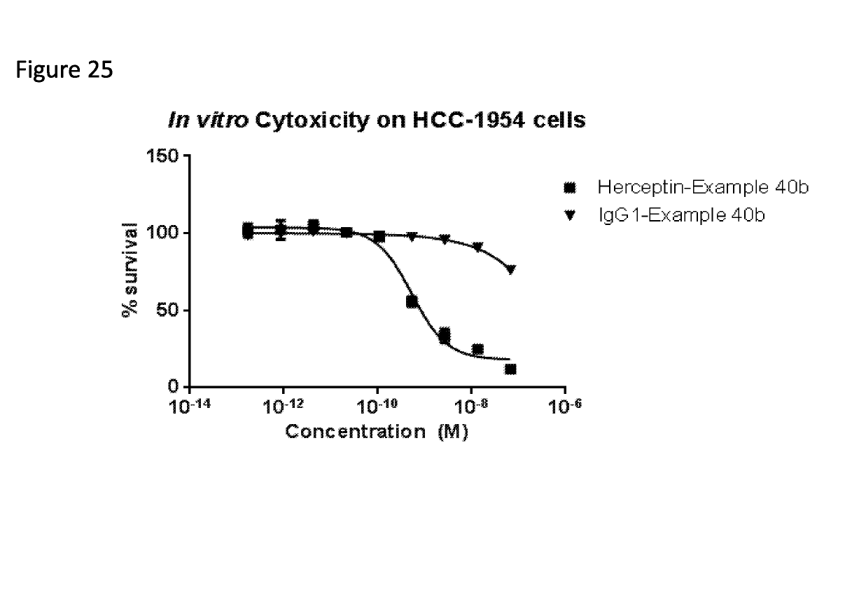

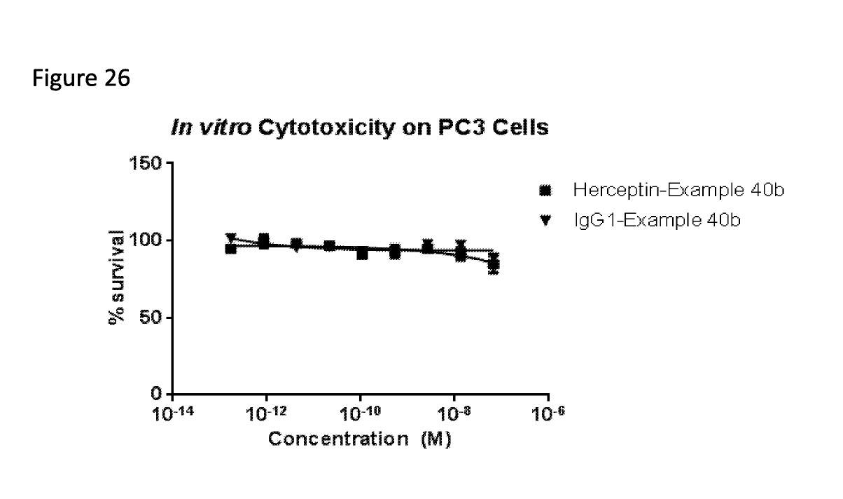

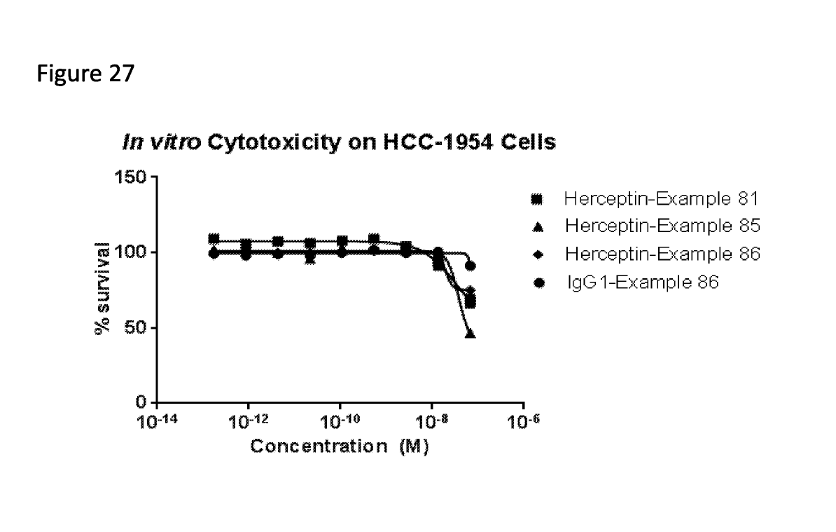

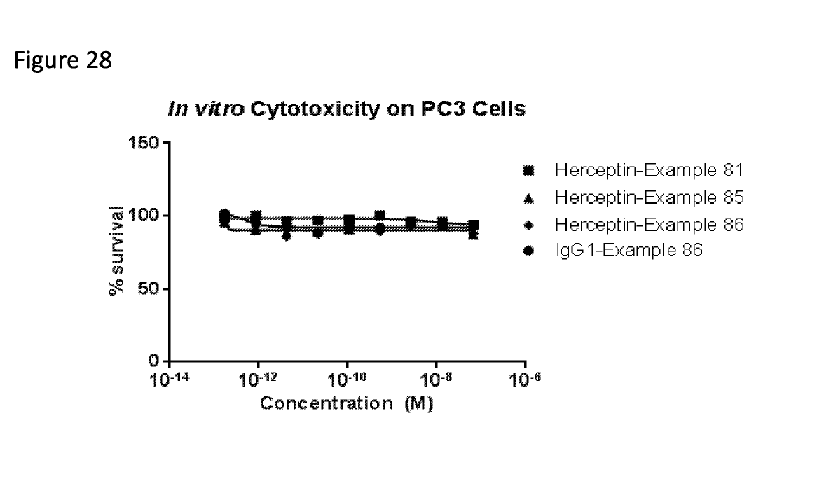

The assay(s) were conducted in clear tissue-culture treated 96-well plates, using high drug-antibody ratio conjugates which were prepared as described in Example 91(D)-(G). The cell lines used were PC3 (human prostate carcinoma, HER2(−)), HCC-1954 (human mammary ductal carcinoma, HER2(+)), and MDA-MB-468 (human mammary adenocarcinoma, HER2(−)). Briefly, Cells were seeded at approximately 1,000-2,000 cells per well in 50 μL of growth media (RPMI-1640+10% heat-inactivated fetal bovine serum or Leibovitz's L-15+10% heat-inactivated serum) and incubated overnight at 37° C. with 5% CO2 to allow them to attach. The next day, 50 μL of test articles at varying concentrations were diluted in growth media and added to each well in triplicate. In addition, control wells with no cells or untreated cells alone were used. The plates were incubated in the humidified tissue culture incubator with 5% CO2 at 37° C. for 4 to 6 days after addition of test articles. After 4 or 6 days, 20 μL of PrestoBlue™ Cell Viability Reagent (Life Technologies, Carlsbad, Calif.) was added per well. Plates were incubated at 37° C. for 1 to 2 hours. Fluorescence was recorded at 540ex/590em using the Biotek Synergy™ H4 plate reader. Data was graphed as percent survival compared to untreated control wells, and is presented in FIGS. 1-22 and 25-28. The data show that certain example compounds conjugated to herceptin as described herein exhibit cytotoxicity in various cancer cell lines of less than 50% survival at picomolar to nanomolar concentrations. The data additionally show that certain example compounds conjugated to herceptin as described herein exhibit increased cytotoxicity in various cancer cell lines relative to example compounds conjugated to IgG1 that were used as controls.

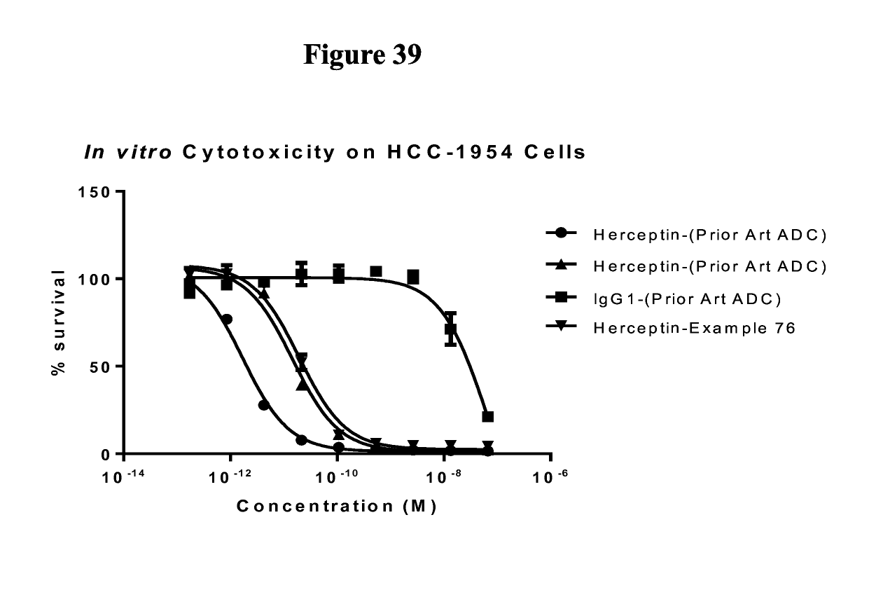

In another set of assay(s), The in vitro efficacy of the antibody-drug conjugates were measured by evaluating their cytotoxic activity on additional cancer cell lines. This assay is conducted in clear tissue-culture treated 96-well plates. The cell lines used are MOLM-13 (human acute myeloid leukemia), RS4-11 (human acute lymphoblastic leukemia), Hel92.1.7 (human erythroleukemia), EOL-1 (human eosinophilic leukemia), and Pfeiffer (human diffuse large cell lymphoma). Cells were seeded at approximately 1,000-6,000 cells per well in 50 μl of growth media (RPMI-1640+10% heat-inactivated fetal bovine serum or Leibovitz's L-15+10% heat-inactivated serum) and incubated overnight at 37° C. with 5% CO2. The next day, 50 μl of test articles at varying concentrations are diluted in growth media and added to each well in triplicate. In addition, control wells with no cells or untreated cells alone are used. The plates are incubated in the humidified tissue culture incubator with 5% CO2 at 37° C. for 4 to 6 days after addition of test articles to measure cytotoxicity. After 4-6 days, 20 μl of PrestoBlue™ Cell Viability Reagent is added per well. Plates are incubated at 37° C. for 1 to 2 hours. Fluorescence is recorded at 540ex/590em using the Biotek Synergy™ H4 plate reader. Representative data is graphed as percent survival compared to untreated control wells. The data are presented in FIGS. 39-51. The data show that certain example compounds conjugated to herceptin as described herein exhibit cytotoxicity in various cancer cell lines of less than 50% survival at picomolar to nanomolar concentrations. The data additionally show that certain example compounds conjugated to herceptin as well as anti-CD33, anti-CD71, and anti-FLT3 as described herein exhibit increased cytotoxicity in various cancer cell lines relative to example compounds conjugated to IgG1 that were used as controls.

Specifically, FIG. 39 shows The Herceptin-(Prior Art ADC) antibody-drug conjugates of DAR=5.7 () and DAR=1.7 (▴) both demonstrate cytotoxicity at sub nM concentrations after a 6 day treatment with greater potency observed using the DAR=5.7 conjugate. Both are more efficacious than Herceptin-Example 76 with a DAR=5.2, which is also cytotoxic at sub nM concentrations. IgG1-(Prior Art ADC) at DAR=1.1 also demonstrates cytotoxicity at the highest concentration after a 6 day treatment.

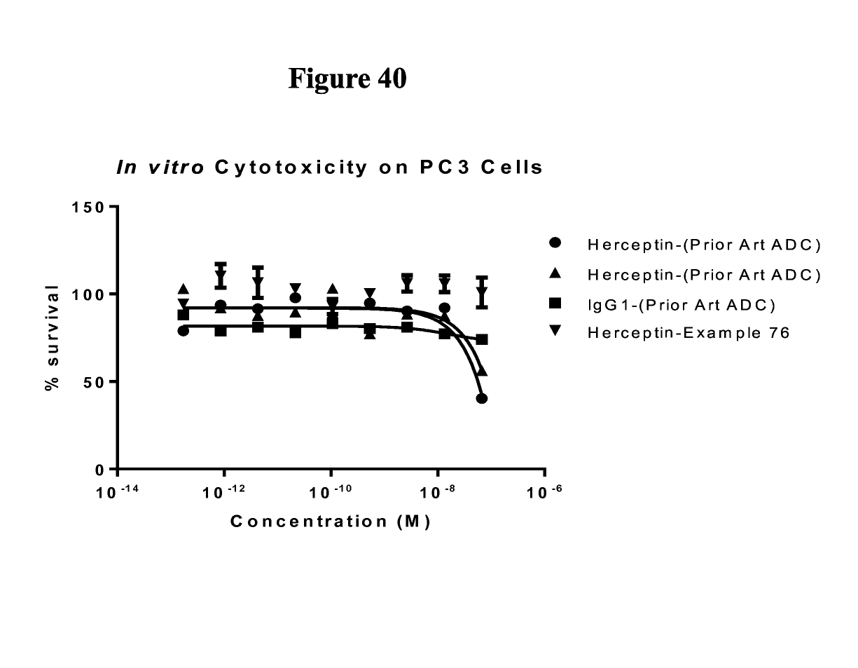

FIG. 40 shows the Herceptin-(Prior Art ADC) antibody-drug conjugates of DAR=5.7 () and DAR=1.7 (▴) both demonstrate cytotoxicity at the highest concentration, whereas IgG1-(Prior Art ADC) at DAR=1.1 does not demonstrate cytotoxicity after a 4 day treatment. In addition, Herceptin-Example 76 of DAR=5.2 does not demonstrate cytotoxicity after a 4 day treatment.

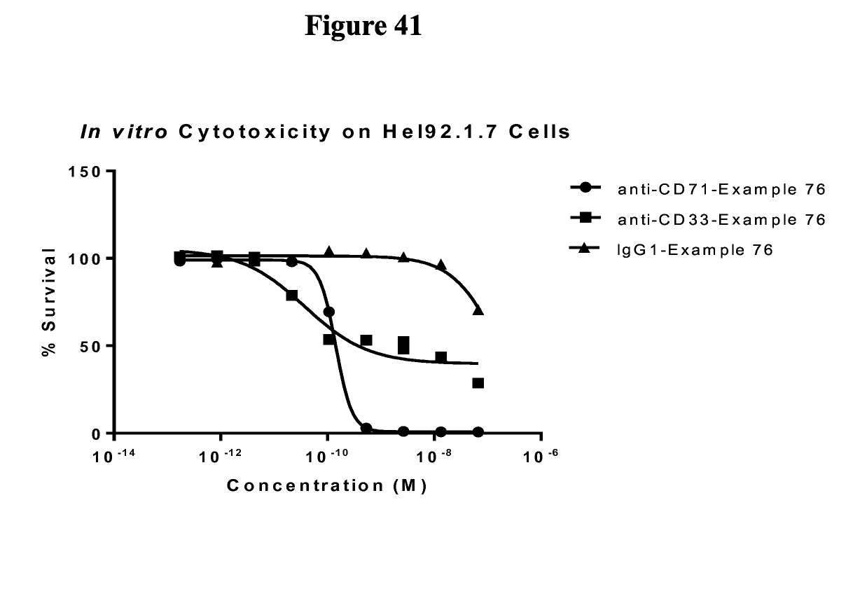

FIG. 41 shows both antibody-drug conjugates to anti-CD71 & CD33 demonstrate cytotoxicity at sub nM concentrations after a 5 day treatment. Anti-CD71-Example 76 shows complete killing to 0% survival, whereas anti-CD33-Example 76 demonstrates cytotoxicity to 30% survival at the highest concentration. IgG1-Example 76 does not demonstrate cytotoxicity after a 5 day treatment.

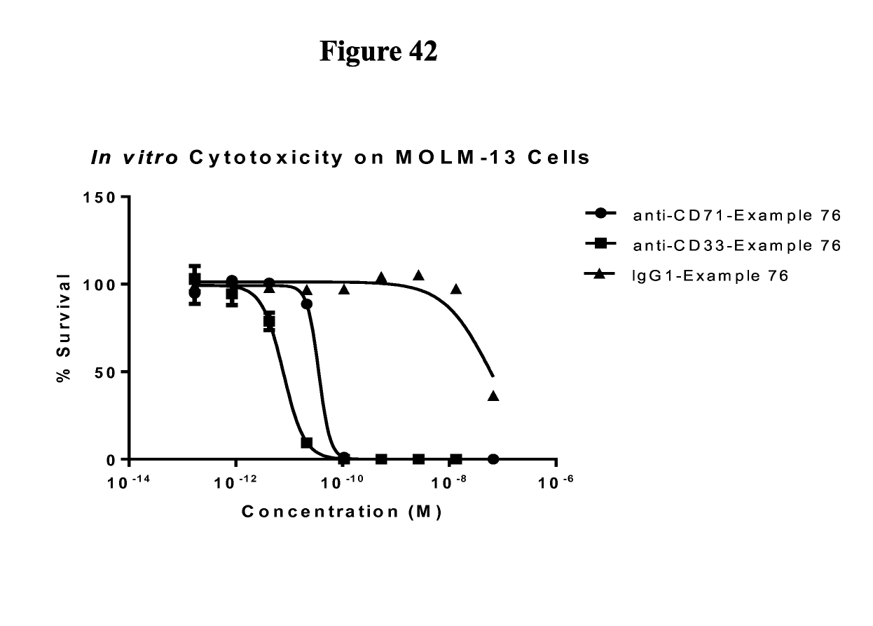

FIG. 42 shows Both antibody-drug conjugates to anti-CD71 & CD33 demonstrate cytotoxicity at sub nM concentrations after a 5 day treatment where anti-CD33-Example 76 shows greater cytotoxicity than anti-CD71-Example 76. IgG1-Example 76 exhibits cytotoxicity only at the highest concentration after a 5 day treatment.

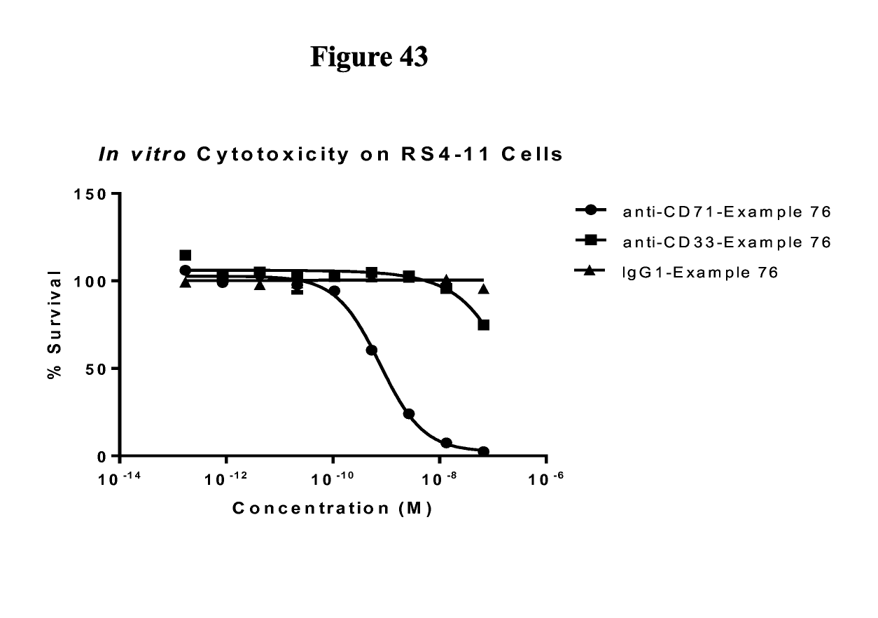

FIG. 43 shows only anti-CD71-Example 76 demonstrates cytotoxicity at sub nM concentrations after a 5 day treatment. Anti-CD33-Example 76 & IgG1-Example 76 do not demonstrate cytotoxicity after a 5 day treatment.

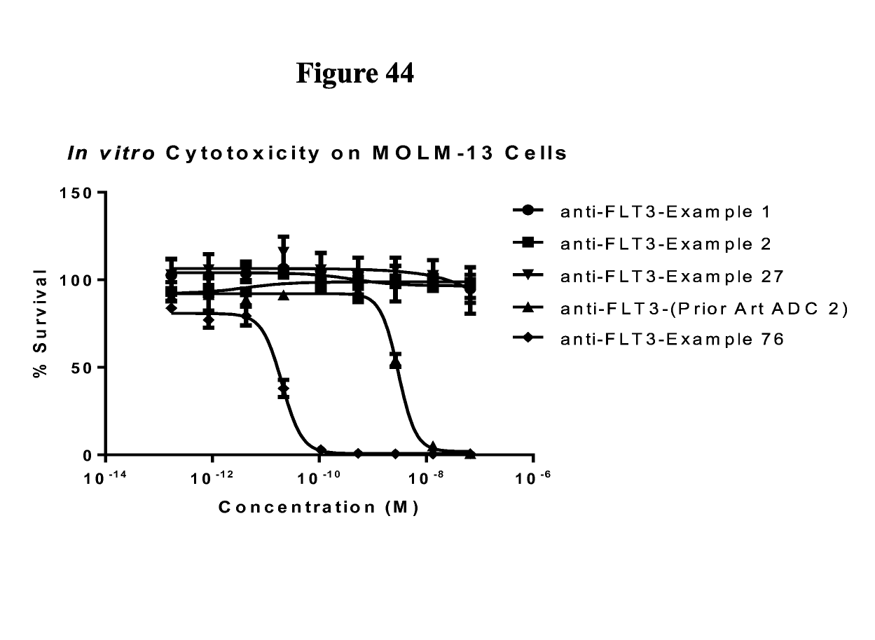

FIG. 44 shows anti-FLT3-(Prior Art ADC 2) is cytotoxic at nM concentrations, whereas anti-FLT3-Example 76 is cytotoxic at sub nM concentrations exhibiting greater potency after a 5 day treatment. However, the anti-FLT3 antibody conjugated to Examples 1, 2, and 27 does not demonstrate cytotoxicity after a 5 day treatment.

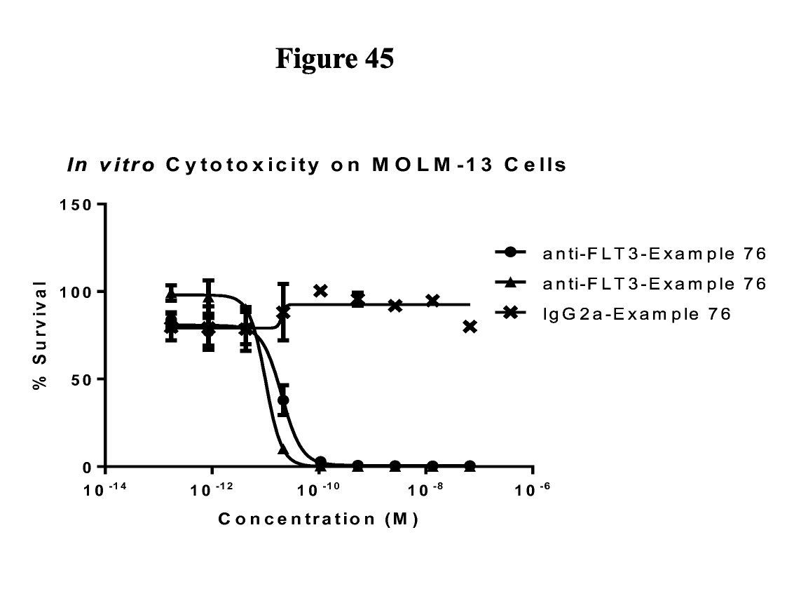

FIG. 45 shows both anti-FLT3-Example 76 at DAR=4 () and anti-FLT3-Example 76 at DAR=7.6 (▴) demonstrate cytotoxicity at sub nM concentrations after a 5 day treatment, with anti-FLT3-Example 76 at DAR=7.6 being the more potent antibody-drug conjugate. IgG2a-Example 76 does not demonstrate cytotoxicity after a 5 day treatment.

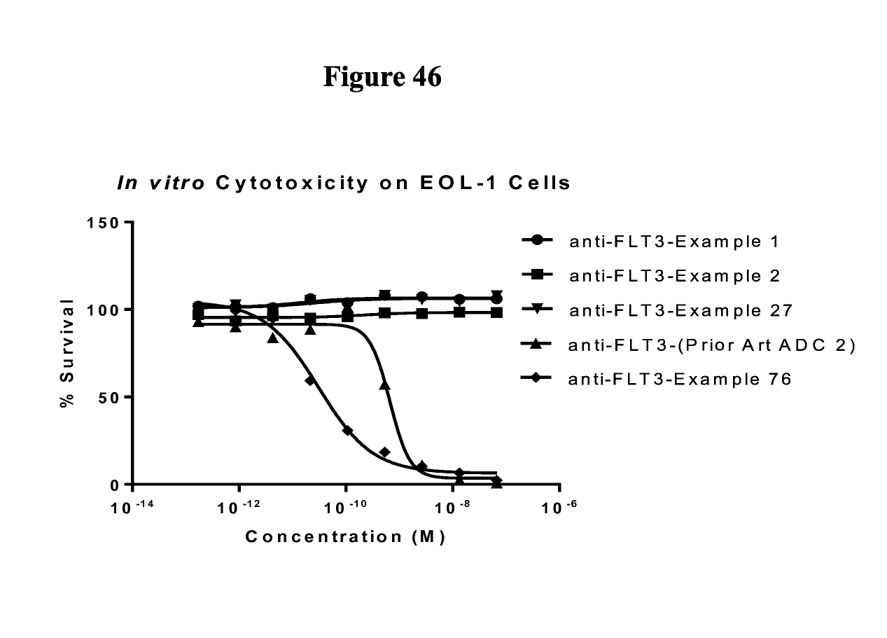

FIG. 46 shows anti-FLT3-(Prior Art ADC 2) is cytotoxic at nM concentrations, whereas anti-FLT3-Example 76 is cytotoxic at sub nM concentrations, exhibiting greater potency after a 5 day treatment. However, the anti-FLT3 antibody conjugated to Examples 1, 2, and 27 does not demonstrate cytotoxicity after a 5 day treatment.

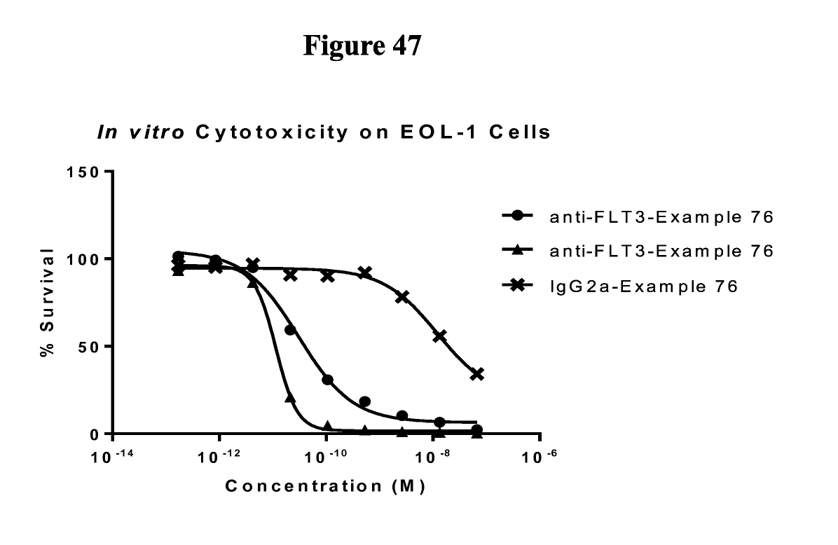

FIG. 47 shows Both anti-FLT3-Example 76 at DAR=4 () and anti-FLT3-Example 76 at DAR=7.6 (▴) demonstrate cytotoxicity at sub nM concentrations after a 5 day treatment, with anti-FLT3-Example 76 at DAR=7.6 being the more potent antibody-drug conjugate. IgG2a-Example 76 also demonstrates cytotoxicity, but only at high concentrations after a 5 day treatment.

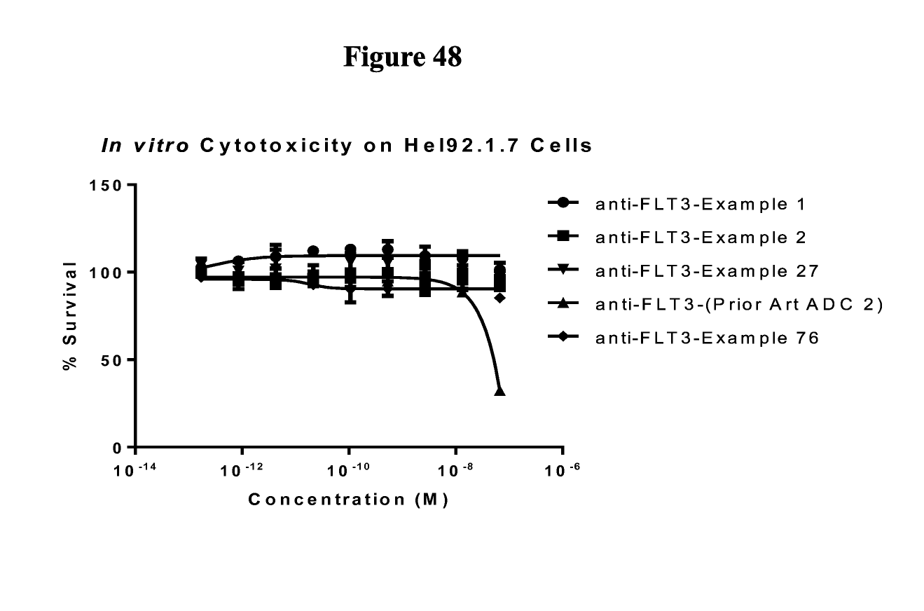

FIG. 48 shows only anti-FLT3-(Prior Art ADC 2) is cytotoxic at the highest concentration after a 5 day treatment. Anti-FLT3 conjugated to Examples 1, 2, 27, and 76 does not demonstrate cytotoxicity after a 5 day treatment.

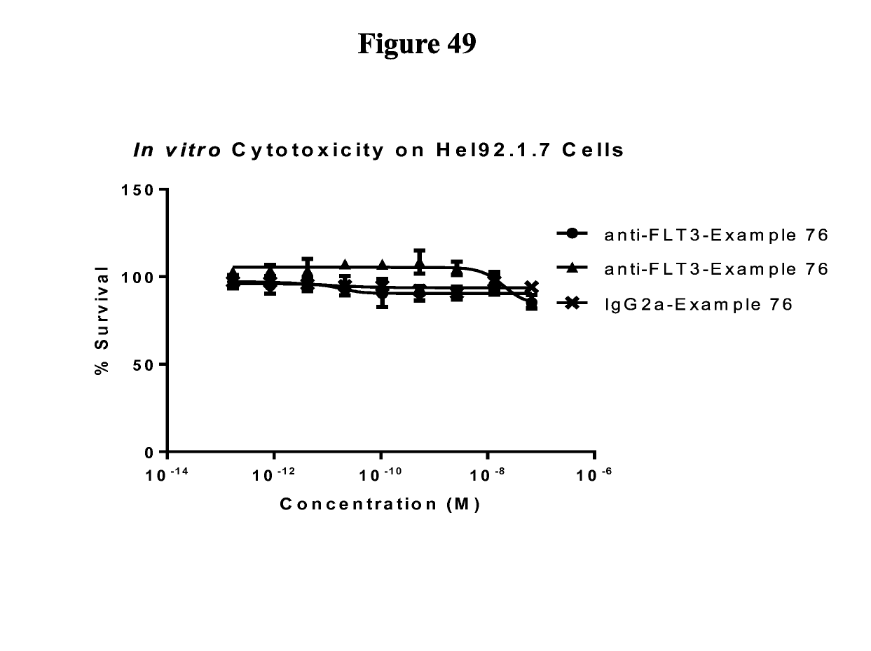

FIG. 49 shows both anti-FLT3-Example 76 at DAR=4 () and anti-FLT3-Example 76 at DAR=7.6 (▴) do not demonstrate cytotoxicity after a 5 day treatment. IgG2a-Example 76 also does not demonstrate cytotoxicity after a 5 day treatment.

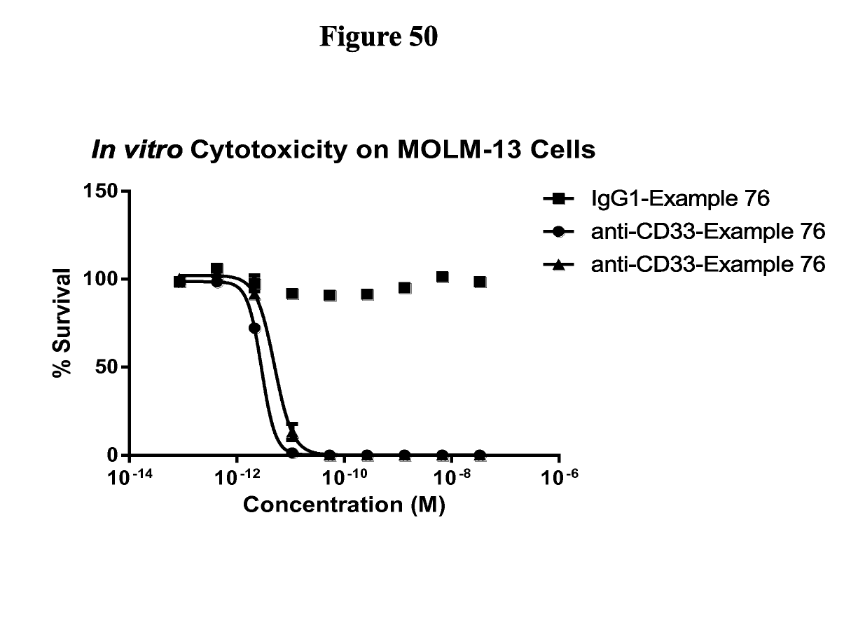

FIG. 50 shows that Example 76 demonstrates cytotoxicity when conjugated to the anti-CD33 antibody, but has no cytotoxicity when conjugated to the IgG1 control antibody. Both anti-CD33-Example 76 at DAR=8.6 () and anti-CD33-Example 76 at DAR=4.0 (▴) demonstrate cytotoxicity at sub nM concentrations after a 6 day treatment with anti-CD33-Example 76 at DAR=8.6 being the more potent antibody-drug conjugate than the one at DAR=4.0.

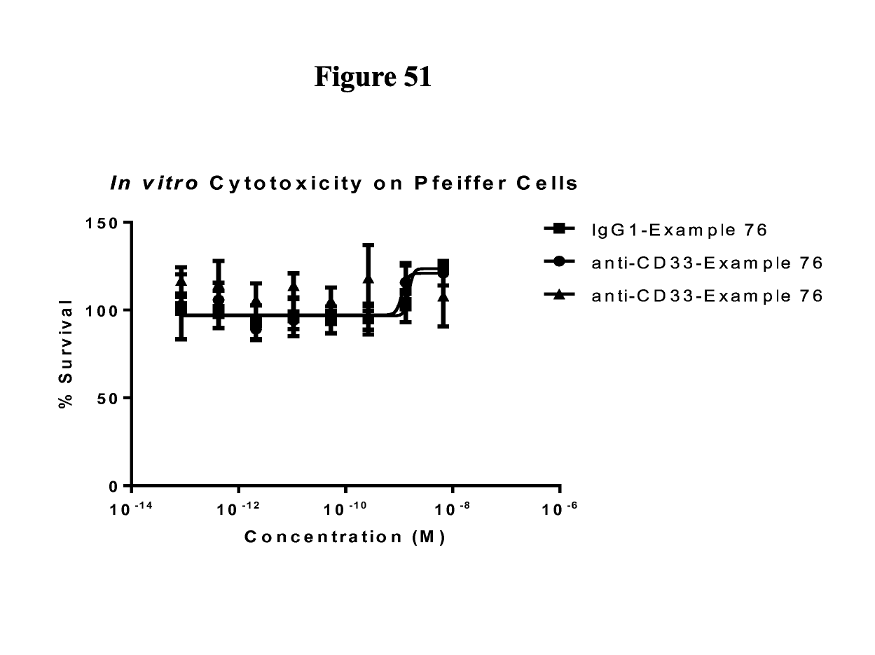

FIG. 51 shows the cytotoxicity of Example 76 conjugated to the anti-CD33 and IgG1 isotype control antibody on Pfeiffer, human diffuse large cell lymphoma (seeded 6,000 cells per well). Note, no antibody-drug conjugates demonstrated cytotoxicity after a 6 day treatment.

### Biological Example 2

### In Vivo Xenograph Model in Breast Cancer Cell Lines

Five to six week old ICR SCID female mice (Taconic Farms, Hudson, N.Y.) were housed in ventilated cage racks, with food and water provided ad libitum. Routine husbandry and handling of experimental animals complied with regulations and guidelines governing the use of animals in research. Mice were acclimated for 72 hours before beginning the study. Experimental animals were tested in compliance with IACUC protocols #002. Mice were injected with HCC1954 human breast cancer cells (3×10⁶ cells/mouse) into the mammary fatpads and tumor growth rate was monitored. After study start, tumor growth was monitored using caliper measurements every three to four days until the end of the study. Tumor volume was calculated as Width x Length/2, where width is the smallest dimension and length is the largest. When the average tumor volume reached ˜200 mm³, tumors were size matched and mice were randomized to treatment groups (n=10) to ensure similar mean tumor size and variation in each group using Study Director Software (v.1.7; Studylog Systems, Inc., South San Francisco, Calif.). The tumor-bearing mice were treated with a single i.v. bolus of vehicle or test agent on day zero. The amount of test agent administered was based on the individual body weight of each animal obtained immediately prior to dosing. Test agents were example compounds conjugated to Herceptin, at high drug-antibody ratios, prepared as described in Example 91(D)-(G).

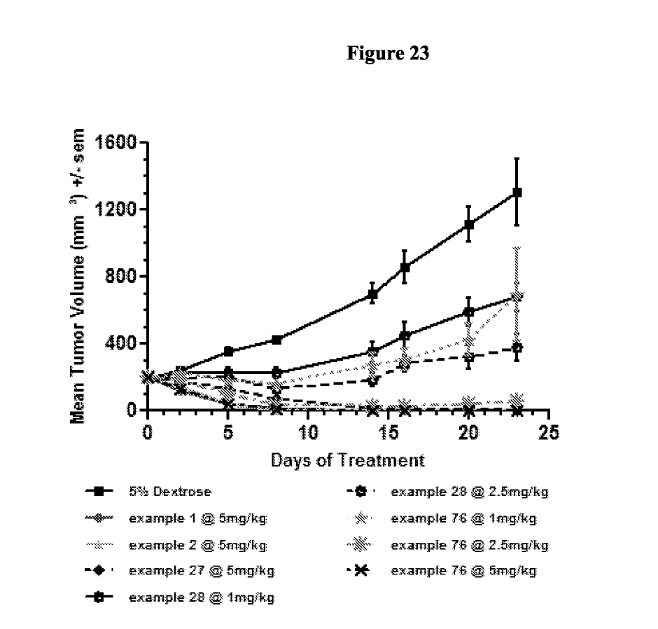

FIG. 23 shows that Example 28 conjugate and Example 76 conjugate at 1 mg/kg caused tumor growth inhibition. Example 28 conjugate at 2.5 mg/kg caused tumor growth inhibition while Example 76 conjugate at the same dose caused tumor regression. Conjugates of Examples 1, 2, 27 and 76 dosed at 5 mg/kg caused tumor regression. Other than the Example 27 conjugate, the regression caused by the 5 mg/kg dose groups was maintained for a prolonged length of time.

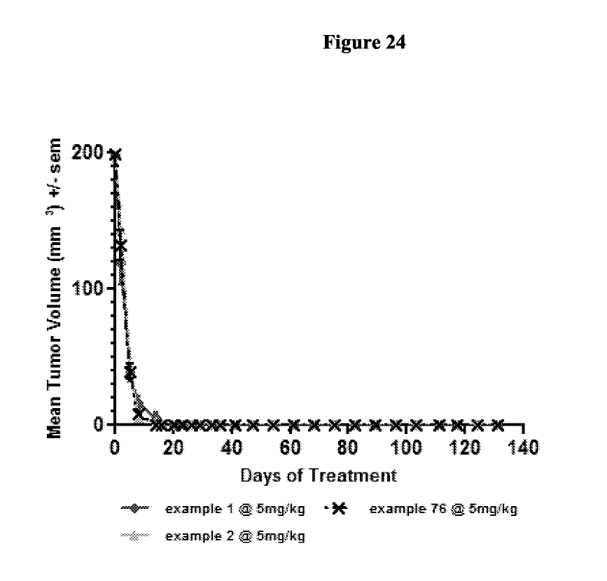

FIG. 24 shows that Examples 1, 2, and 76 conjugates, when dosed at 5 mg/kg, maintained prolonged anti-tumor efficacy with no tumor re-growth up to 131 days after initial treatment.

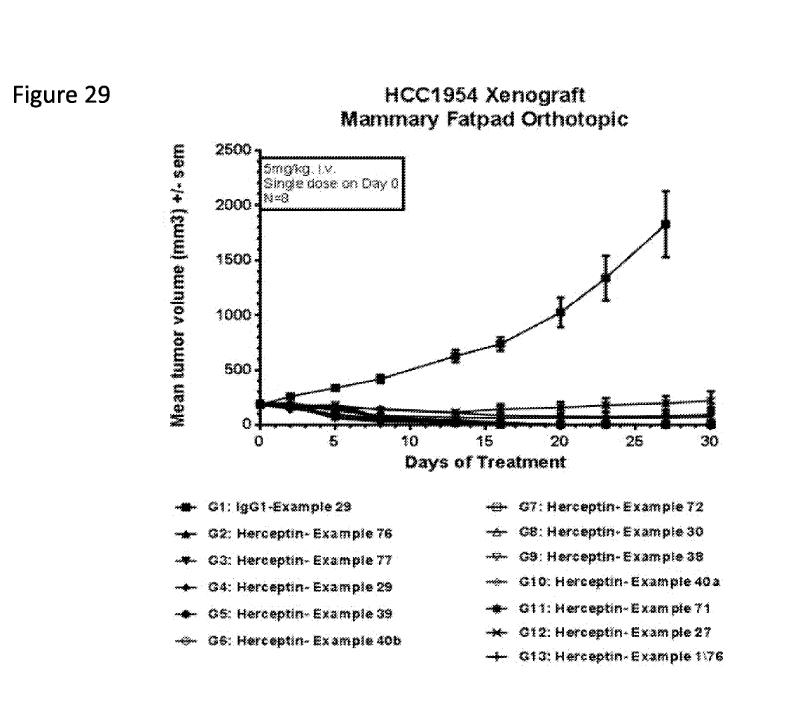

FIG. 29 shows the results for Herceptin ADC conjugates of Examples 27, 29, 30, 38, 39, 40a, 40b, 71, 72, 76, and 77 at a dose of 5 mg/kg. The conjugate of IgG1 with Example 29 was used as the control agent. All test conjugates caused tumor stasis at the 5 mg/kg dose level, and Herceptin conjugates of Examples 76, 77, 39, 72, 38, 71, 40a and 40b showed tumor regression.

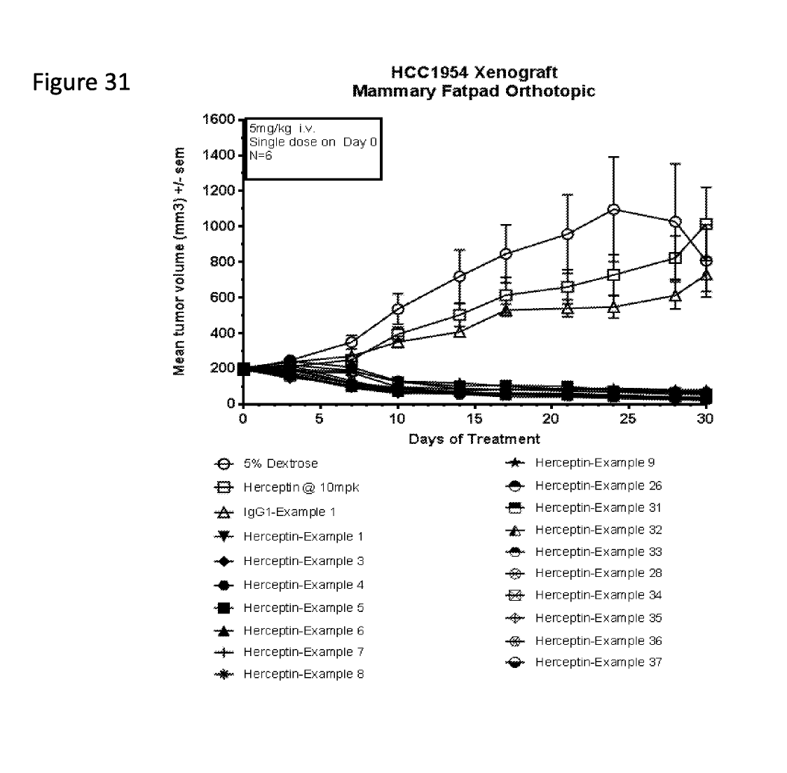

FIG. 31 shows the results for Herceptin conjugates of Examples 1, 3-9, 26, 28, and 31-37 at a dose of 5 mg/kg. The conjugate of IgG1 with Example 29 was used as the control agent. Herceptin conjugates of Examples 1, 3, 4, 5, 6, 7, 8, 9, 26, 31, and 32 caused tumor regression at the 5 mg/kg dose level. Herceptin conjugates with Examples 28, 34, 35, 36, and 37 were terminated on Day 3 due to non-tolerability.

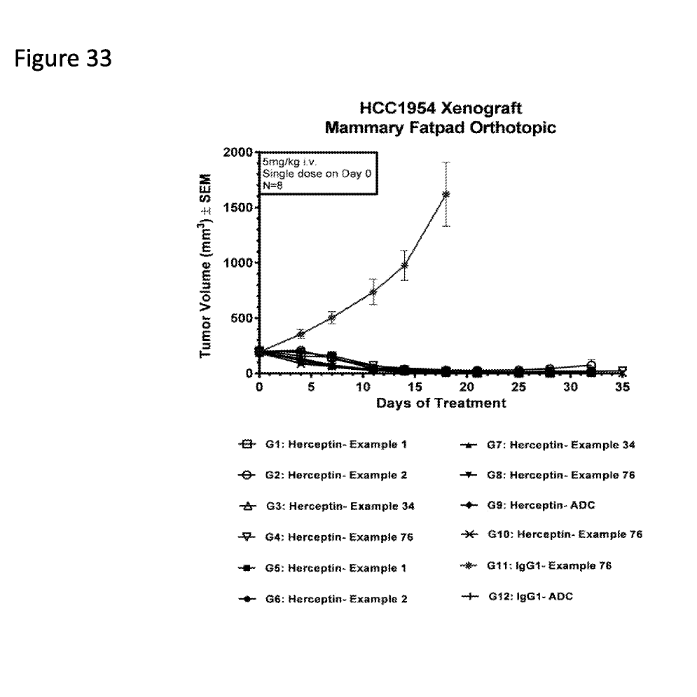

FIG. 33 shows the results for Herceptin conjugates of Examples 1, 2, 34, and 76 at a dose of 5 mg/kg as compared to control groups for an α-amanitin-glutarate-Herceptin ADC (see WO2010/115629, page 43, Ex. 1.11.3), IgG1-Example 76 conjugate, and an α-amanitin-glutarate-IgG1 ADC (prepared from α-amanitin-glutaric acid N-hydroxysuccinimidate as described in WO2010/115629, page 42, Ex. 1.11.2 and IgG1, as in Ex. 1.11.3 of the same reference). All animals in the two Prior Art ADC groups were found dead a couple of days after treatment. One week after treatment, four mice in the Herceptin-Example 34 treated group were found dead. Eighteen days after treatment initiation the remaining control group, IgG1-Example 76, was humanely euthanized due to tumor burden. FIG. 33 shows that all Herceptin-Example conjugates caused tumor regression at the 5 mg/kg dose level.

### Biological Example 3

### Subcutaneous Xenograft Tumor Studies in Breast Cancer Cell Lines

This study employed the same protocol as Biological Example 2, with the exception that Human breast cancer HCC1954 cells (3×10⁶ cells per mouse) were injected subcutaneously into the flanks of individual SCID mice.

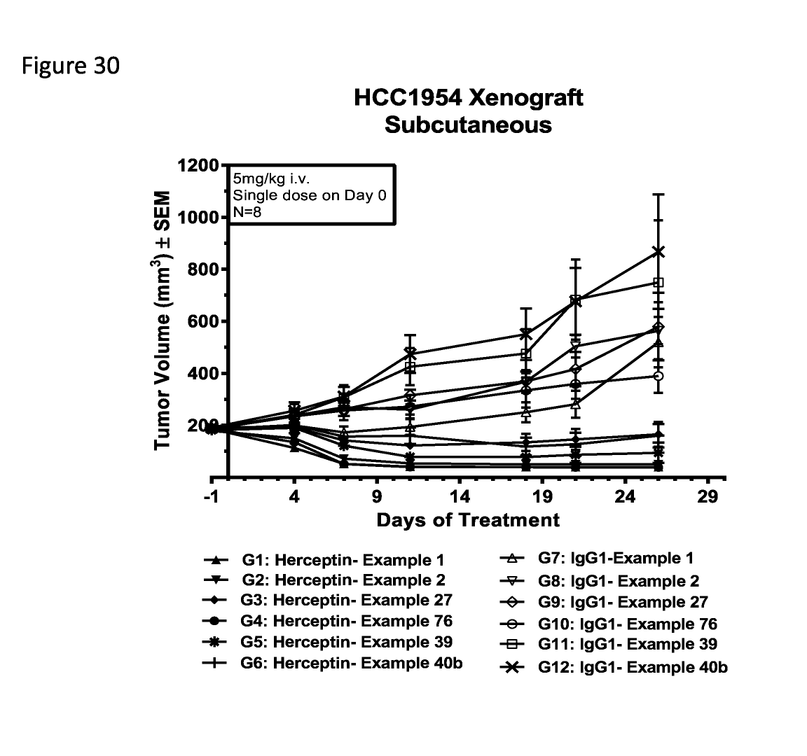

FIG. 30 shows the results for Herceptin conjugates of Examples 1, 2, 27, 76, 39, and 40b at a dose of 5 mg/kg as compared to IgG1 conjugates with the same compounds. All Herceptin Examples caused tumor stasis at the 5 mg/kg dose level. In addition, Examples 1, 2, 76 and 39 showed tumor regression.

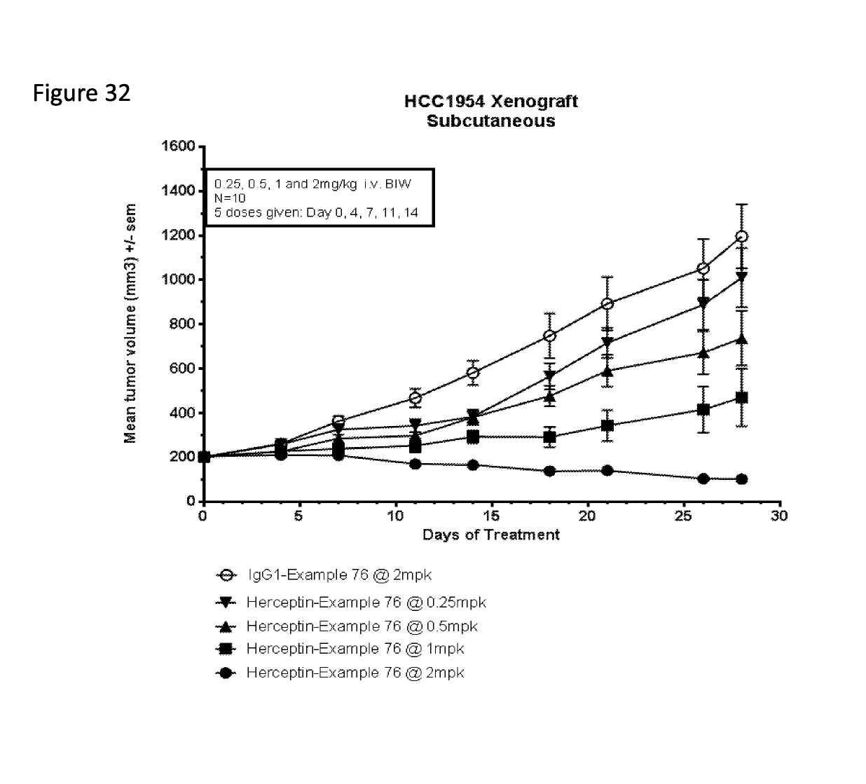

FIG. 32 shows the results for a Herceptin conjugate of Example 76 at twice weekly doses of 0.25, 0.5, 1, and 2 mg/kg as compared to an IgG1-Example 76 control conjugate at 2 mg/kg. All groups received five doses in total. The amount of each ADC administered was based on the individual body weight of each animal obtained immediately prior to dosing. The data show a dose-dependent anti-tumor effect for the Herceptin-Example 76 conjugate. Tumor regression was observed at a dose level of 2 mg/kg.

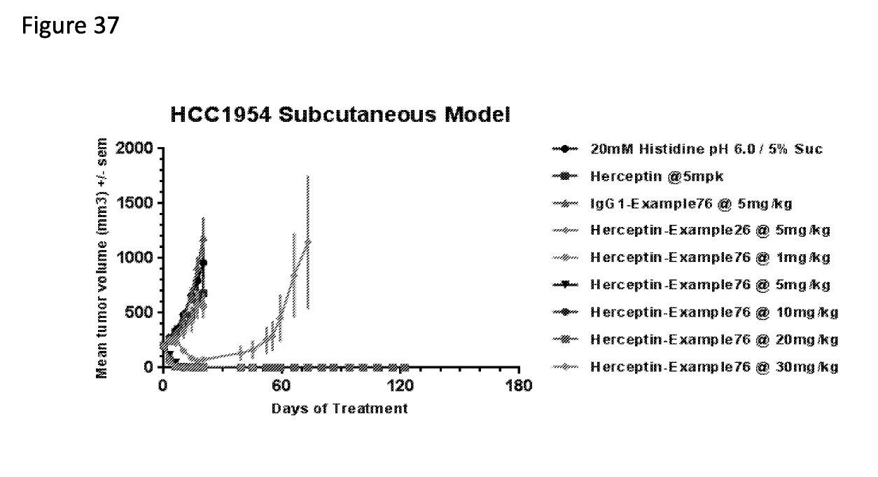

FIG. 37 shows the results for Herceptin conjugates of Examples 26 (5 mg/kg) and 76 (1, 5, 10, 20, and 30 mg/kg) as compared to 20 mM histidine, Herceptin (5 mg/kg), and IgG1-Example 76 ADC (5 mg/kg) controls. The data show that the Herceptin conjugate of Example 76 at 1 mg/kg caused tumor growth inhibition. The Herceptin-Example 76 ADC caused tumor growth regression at 5, 10, 20, and 30 mg/kg. The regression caused by the 5, 10, 20, and 30 mg/kg dose groups was maintained for a prolonged length of time.

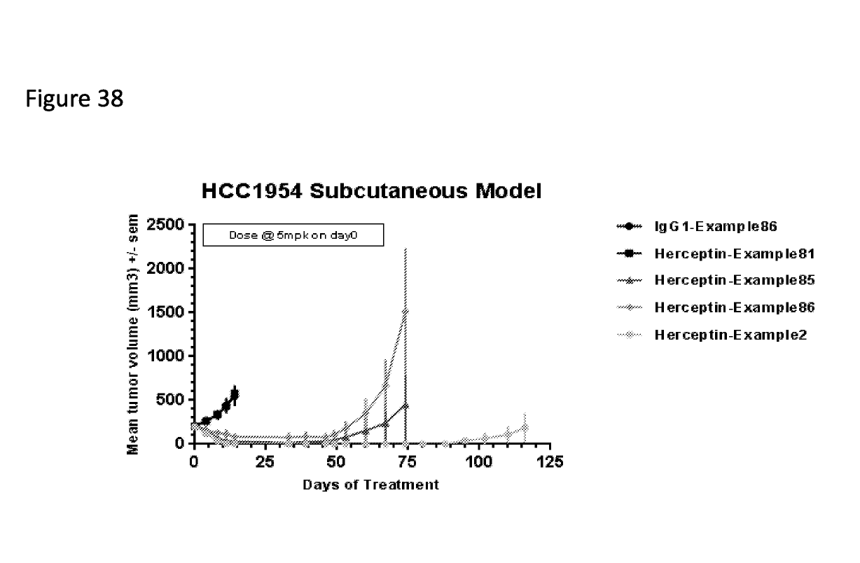

FIG. 38 shows the results for Herceptin conjugates of Examples 2, 81, 85, and 86 at 5 mg/kg as compared to a IgG1-Example 86 ADC control. The data show that Herceptin conjugates of Examples 85 and 86 caused tumor regression followed by delayed regrowth around day 50. The Herceptin-Example 2 ADC caused tumor growth regression, which was maintained for a prolonged length of time.

### Biological Example 4

### In Vivo Studies in UG-K3 Subcutaneous Xenograft Model

UG-K3 is a human renal clear cell carcinoma xenograft derived from a patient tumor specimen. The xenograft was maintained by cryopreservation and serial passages in immunodeficient mice since its establishment. Immunohistochemistry analysis showed strong to moderate expression of ENPP3 in more than 90% of the tumor cells. UG-K3 stock tumors were harvested under sterile conditions and minced into small pieces. The tumor pieces were enzymatically digested to single cell suspension using Liberase Blendzyme (Roche Applied Science, Indianapolis, Ind.). Cells (1.5 million) were injected subcutaneously into the flanks of individual SCID mice and tumors were allowed to grow. Tumor growth was monitored. When the average tumor volumes reached approximately 200 mm³, animals were size matched and randomized into treatment and control groups to ensure similar mean tumor size and variation in each group using Study Director Software (v.1.7; Studylog Systems, Inc., South San Francisco, Calif.). All groups received a single dose of test agent at 5 mg/kg by intravenous bolus injection on day 0. The amount of each ADC administered was based on the individual body weight of each animal obtained immediately prior to dosing.

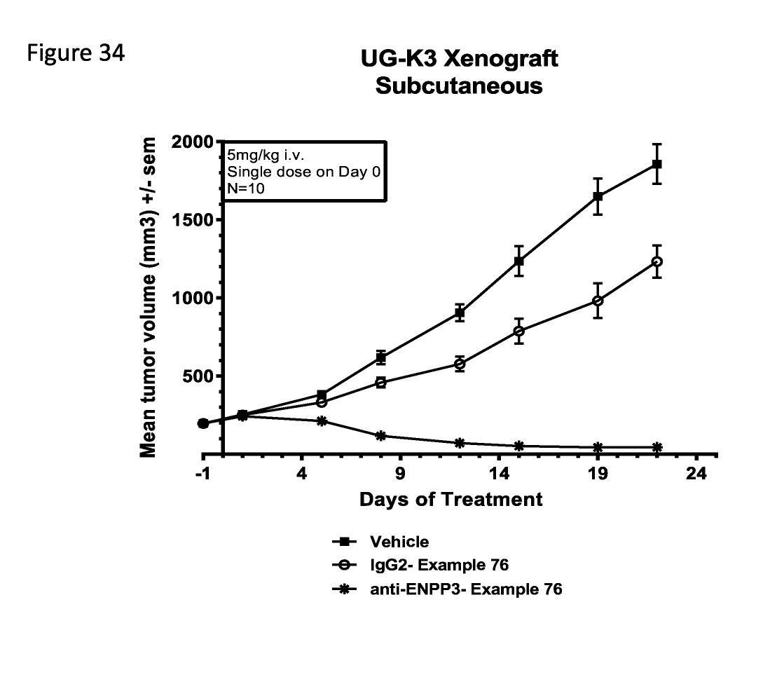

FIG. 34 shows the results for an anti-ENPP3-Example 76 conjugate as compared to vehicle and a IgG1-Example 76 conjugate at 5 mg/kg. Anti-ENPP3 is a fully human IgG2K derived monoclonal antibody (also known as clone H16-7.8) to ENPP3 antigen (expressed by the ENPP3 gene, NCBI Gene I.D. No. 5169), a ectonucleotide pyrophosphatase/phosphodiesterase 3, an 875 amino acid type II single transmembrane antigen that is up-regulated in the majority of renal cancers and in a subset of hepatocellular cancers (also known as 161P2F10B) (See, U.S. Pat. No. 7,279,556 (Agensys, Inc., Santa Monica, Calif.), U.S. Pat. No. 7,405,290 (Agensys, Inc., Santa Monica, Calif.), U.S. Pat. No. 7,067,130 (Agensys, Inc., Santa Monica, Calif.), and U.S. Pat. No. 7,226,594 (Agensys, Inc., Santa Monica, Calif.)).

In this study, the Vehicle and IgG1-Example 76 were used as controls. The data show that anti-ENPP3-Example 76 ADC caused tumor regression at the 5 mg/kg dose level.

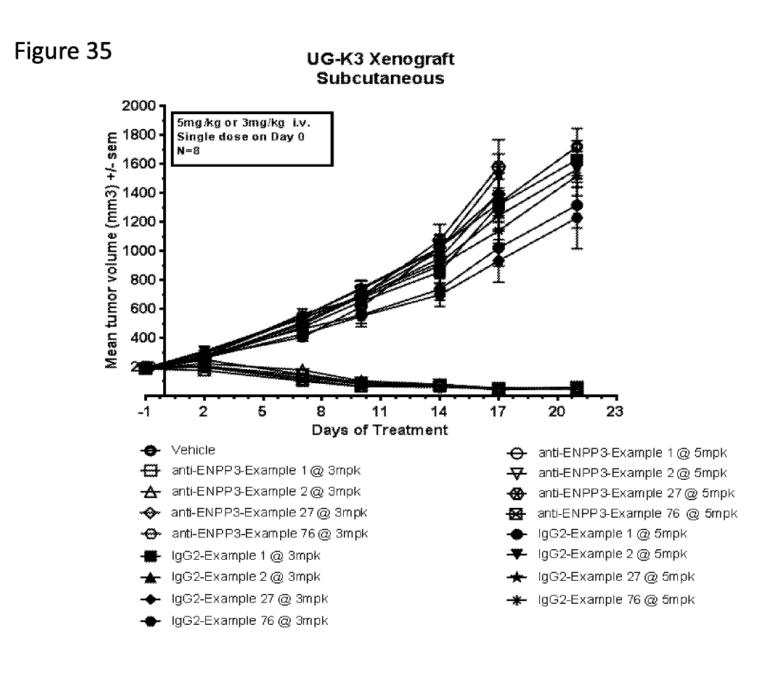

FIG. 35 shows the results for anti-ENPP3 conjugates of Examples 1, 2, 27, and 76 as compared to vehicle and control IgG2 conjugates of the same examples, at 3 and 5 mg/kg doses. The data show that anti-ENPP3 conjugates of Examples 1, 2 and 76 caused tumor regression at the 3 and 5 mg/kg dose levels. The anti-ENPP3-Example 27 ADC did not have any significant effect. The IgG2-Example 27 at 3 mg/kg group was terminated on Day 14 due to tumor burden. The following groups were terminated on Day 17 also due to tumor burden: anti-ENPP3-Example 27 at 3 and 5 mg/kg, IgG2-Example 2 at 3 mg/kg, and IgG2-Example 27 at 5 mg/kg.

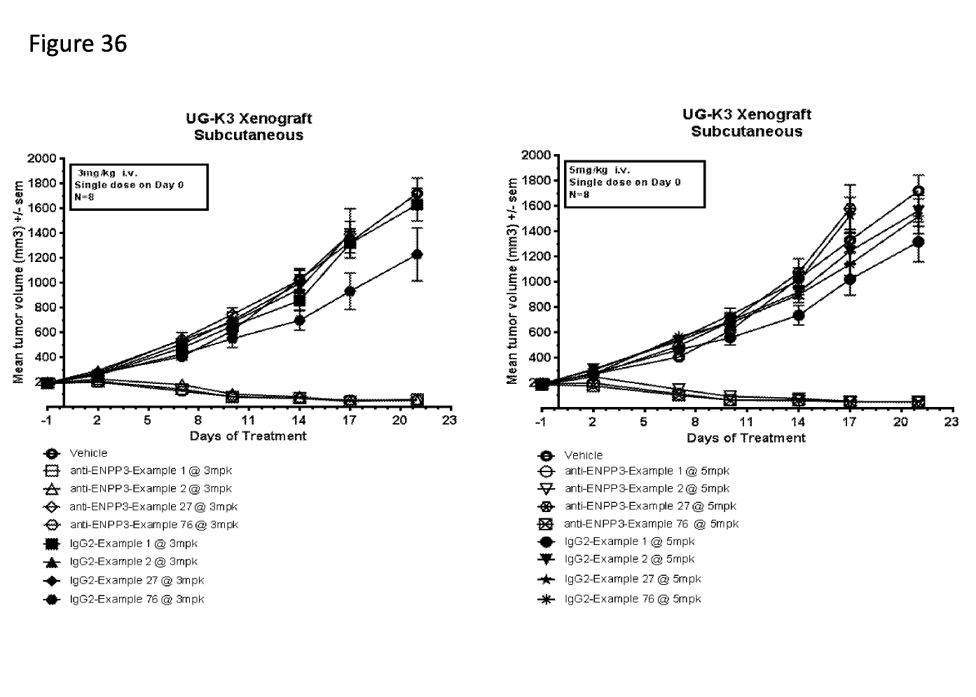

FIG. 36 shows the results for anti-ENPP3 conjugates of Examples 1, 2, 27, and 76 at 3 and 5 mg/kg, as compared to vehicle and IgG2 control conjugates of the same Example compounds at the same doses. The data show that anti-ENPP3 conjugates of Examples 1, 2 and 76 caused tumor regression at the 3 and 5 mg/kg dose levels. The anti-ENPP3-Example 27 ADC did not have any significant effect. The IgG2-Example 27 at 3 mg/kg group was terminated on Day 14 due to tumor burden. The following groups were terminated on Day 17 also due to tumor burden: anti-ENPP3-Example 27 at 3 and 5 mg/kg, IgG2-Example 2 at 3 mg/kg, and IgG2-Example 27 at 5 mg/kg.

### Biological Example 5

### In Vivo Studies in MOLM-13 Subcutaneous Xenograft Model

MOLM-13 is a cell line derived from acute myeloid leukemia. Briefly, MOLM-13 cells (1×106) were injected into the flanks of individual SCID mice and tumors were allowed to grow. Generally, after the start of the study tumor growth was monitored using caliper measurements every three (3) to four (4) days until the end of the study. Tumor volume was calculated as (Width2× Length)/2, where width is the smallest dimension and length is the largest.

In one experiment, eight to nine week old CB 17/SCID female mice (Charles River Laboratories, Wilmington, Mass.) were used. Upon arrival at the facility mice were housed in ventilated cage racks, with food and water provided ad libitum. Routine husbandry and handling was performed with experimental animals for compliance with regulations and guidelines governing the use of animals in research. Mice were acclimated for 72 hours before initiating the study. Experimental animals were tested in compliance with IACUC protocols #002. When the average tumor volumes reached a predetermined size (200 mm³), animals were size matched and randomized into treatment and control groups to ensure similar mean tumor size and variation in each group using Study Director Software (v.2.1; Studylog Systems, Inc., South San Francisco, Calif.). All groups received a single dose at 2 mg/kg by intravenous bolus injection on day 0. The amount of each ADC administered was based on the individual body weight of each animal obtained immediately prior to dosing. A vehicle control of 20 mM Histidine, pH 6.0/5% Sucrose (formulation buffer) was used.

Anti-CD71 conjugates of Example 1, 2, 27, and 76 were administered a single dose at 2 mg/kg. Anti-CD71 MAbs were IgG1 antibodies against CD71 antigen, a human transferrin receptor I (expressed by the TFRC gene, NCBI Gene I.D. No. 7037), a 760 amino acid type II transmembrane antigen found in most cells. Transferrin receptor and its ligand, transferrin, mediate cellular iron uptake required for cell metabolism and proliferation.

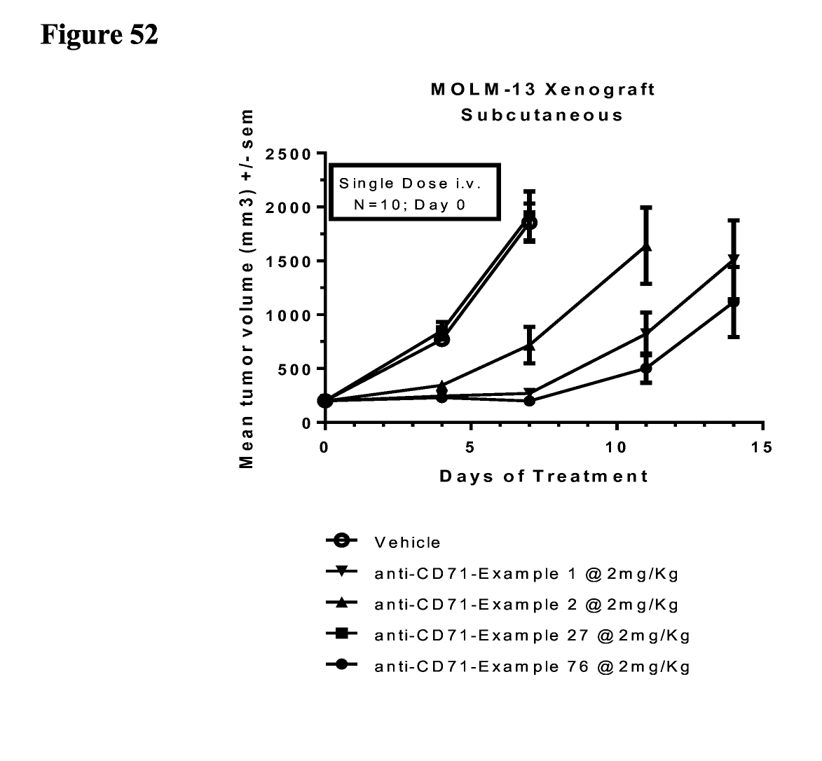

The results show that CD71 conjugates to Example 1, Example 2, and Example 76 caused tumor growth inhibition. In addition, Example 76 caused tumor regression on Day 7 post treatment by statistical analysis. (FIG. 52).

In another experiment, five to six week old IRC SCID female mice (Taconic Farms, Hudson, N.Y.) were used. Upon arrival at the facility mice were housed in ventilated cage racks, with food and water provided ad libitum. Routine husbandry and handling was performed with experimental animals for compliance with regulations and guidelines governing the use of animals in research. Mice were acclimated for 72 hours before initiating the study. Experimental animals were tested in compliance with IACUC protocols #002. When the average tumor volumes reached a predetermined size (200 mm³), animals were size matched and randomized into treatment and control groups to ensure similar mean tumor size and variation in each group using Study Director Software (v.2.1; Studylog Systems, Inc., South San Francisco, Calif.). All groups received a single dose at 1 mg/kg by intravenous bolus injection on day 0. The amount of each ADC administered was based on the individual body weight of each animal obtained immediately prior to dosing. A vehicle control of 20 mM Histidine, pH 6.0/5% Sucrose (formulation buffer) was used.

Anti-CD33 conjugates of Example 1, 2, 27, and 76 were administered a single dose at 1 mg/kg. Anti-CD33 MAbs were generated to CD33 antigen (expressed by the CD33 gene, NCBI Gene I.D. No. 945), which is a 364 amino acid type I transmembrane glycoprotein that is expressed on malignant cells in the majority of patients with acute myeloid leukemia.

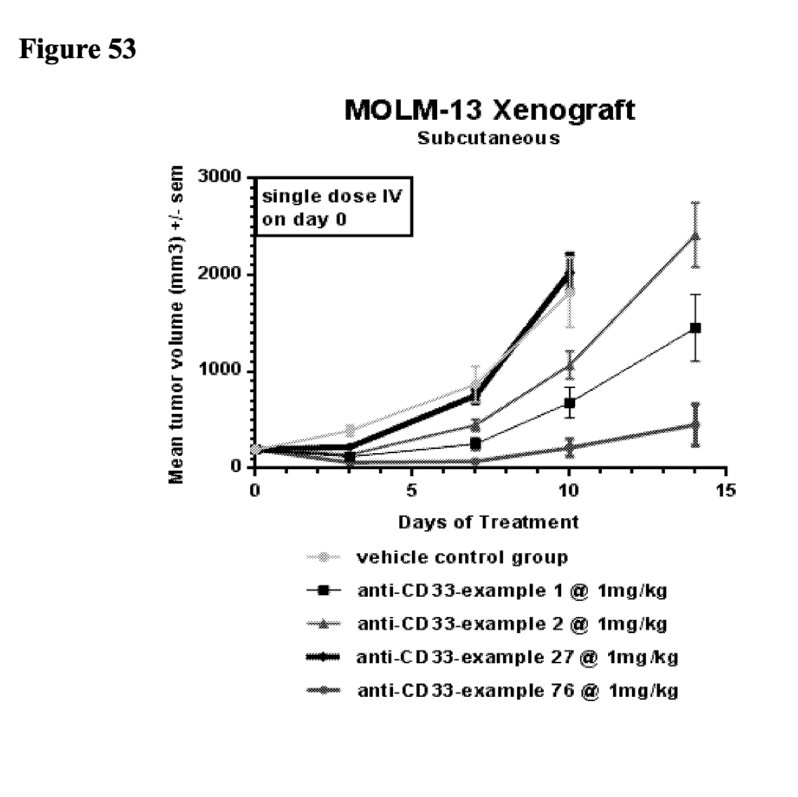

The results show that CD33 conjugates of Examples 1 and 76 caused tumor inhibition. (FIG. 53).

In another experiment, eight to nine week old CB 17/SCID female mice (Charles River Laboratories, Wilmington, Mass.) were used. Upon arrival at the facility mice were housed in ventilated cage racks, with food and water provided ad libitum. Routine husbandry and handling was performed with experimental animals for compliance with regulations and guidelines governing the use of animals in research. Mice were acclimated for 72 hours before initiating the study. Experimental animals were tested in compliance with IACUC protocols #002. When the average tumor volumes reached a predetermined size (200 mm³), animals were size matched and randomized into treatment and control groups to ensure similar mean tumor size and variation in each group using Study Director Software (v.2.1; Studylog Systems, Inc., South San Francisco, Calif.). All groups received a single dose at 2 mg/kg by intravenous bolus injection on day 0. The amount of each ADC administered was based on the individual body weight of each animal obtained immediately prior to dosing. A vehicle control of 20 mM Histidine, pH 6.0/5% Sucrose (formulation buffer) was used.

Anti-FLT3 conjugates of Examples 1, 2, 27, and 76 were administered a single dose at 2 mg/kg. Anti-FLT3 MAbs were generated to FLT3 antigen (expressed by the FLT3 gene, NCBI Gene I.D. No. 2322), otherwise known as fms-like tyrosine kinase 3, an antigen which is highly expressed in hematological malignancies like acute myeloid leukemia and acute lymphoblastic leukemia.

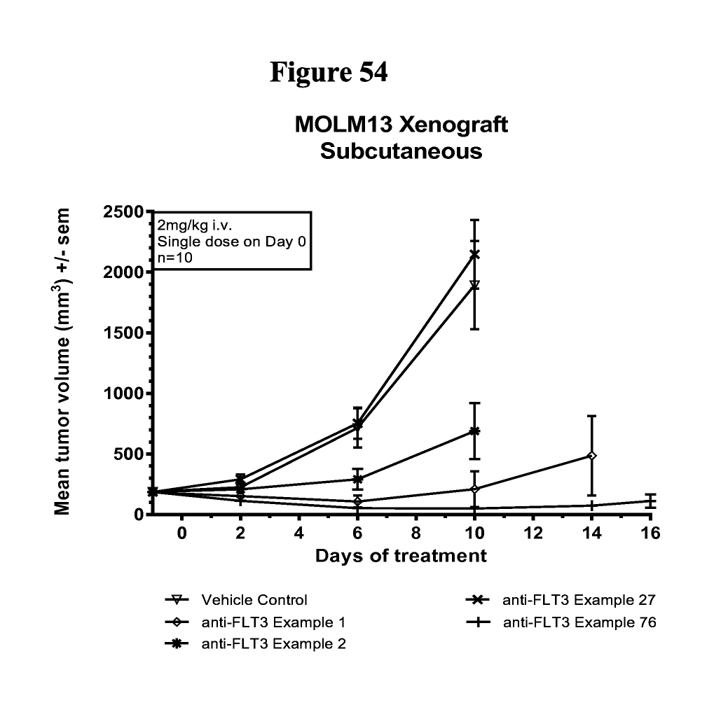

The results show that anti-FLT3 conjugated to Example 1 and Example 76 inhibited tumor growth and Example 76 caused sustained tumor regression. (FIG. 54)

In another experiment, eight to nine week old CB 17/SCID female mice (Charles River Laboratories, Wilmington, Mass.) were used. Upon arrival at the facility mice were housed in ventilated cage racks, with food and water provided ad libitum. Routine husbandry and handling was performed with experimental animals for compliance with regulations and guidelines governing the use of animals in research. Mice were acclimated for 72 hours before initiating the study. Experimental animals were tested in compliance with IACUC protocols #002. When the average tumor volumes reached a predetermined size (200 mm³), animals were size matched and randomized into treatment and control groups to ensure similar mean tumor size and variation in each group using Study Director Software (v.2.1; Studylog Systems, Inc., South San Francisco, Calif.). All groups received a single dose at 2 mg/kg by intravenous bolus injection on day 0. The amount of each ADC administered was based on the individual body weight of each animal obtained immediately prior to dosing. A vehicle control of 5% Dextrose in water (formulation buffer) was used.

Anti-FLT3 conjugates of Examples 1, 2, 27, and 76 and Prior Art ADC 2 (supra, WO2012/041504) were administered a single dose at 2 mg/kg. Anti-FLT3 MAbs were generated to FLT3 antigen (expressed by the FLT3 gene, NCBI Gene I.D. No. 2322), otherwise known as fms-like tyrosine kinase 3, an antigen which is highly expressed in hematological malignancies like acute myeloid leukemia and acute lymphoblastic leukemia.

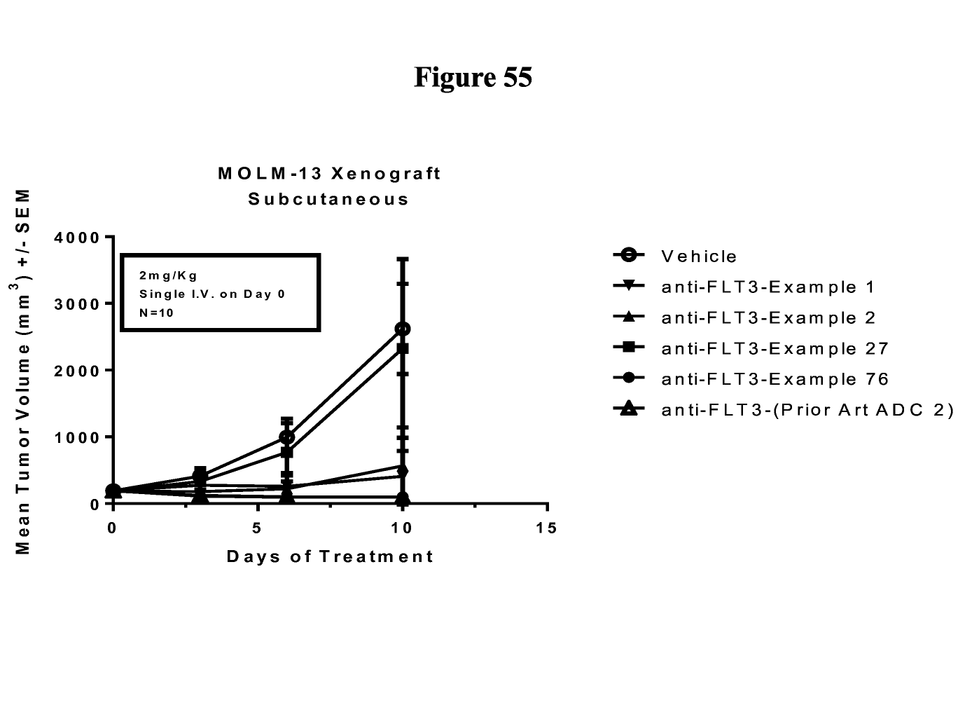

The results show that anti-FLT3-Example 1 and anti-FLT3-Example 2 caused tumor growth inhibition while anti-FLT3-Example 76 and anti-FLT3-(Prior Art ADC 2) caused tumor regression on Day 10 post treatment. (FIG. 55).

Conclusion. The results of the in vivo experiments show that one of ordinary skill in that art will recognize and be enabled to utilize compounds of the invention in a plurality of tumor models. Specifically, Example 76 is shown to inhibit tumor growth in several cancer models, including, renal cancer, breast cancer, and leukemia. Accordingly, compounds of the present invention can be used for therapeutic purposes to treat human cancers.

### Biological Example 6

### Stability Assays of Compounds In Vitro

The stability of drug-linker in ADC can be measured by tracing the quantity of released free drug or drug-linker from ADC by using HPLC. Briefly, the ADC in PBS (3 mg/mL) was incubated at 37° C., and 100 mg of ADC was inject on HPLC at each time point to determine free drug. The quantity of released free drug was determined by integration between 5-6 minutes retention time at 1=310 nm.

For free drug quantification, LC Hisep column manufactured by Supelco (Sigma-Aldrich Group, St. Louis, Mo.) was used with mobile phase A of 0.1% TFA in water and mobile phase B of the mixture of 90% of acetonitrile, 10% of water and 0.1% TFA. 100% of mobile phase A was eluted from 0 min to 2 min, and mobile phase B was linearly increase from 0% to 100% for next 8 min with 1 mL/min flow rate. The result is a calculated half-life (t_{1/2}) denoted in hours by applying first order kinetics using the Prism6 for Windows v. 6.02 (GraphPad Software, Inc. La Jolla, Calif.).

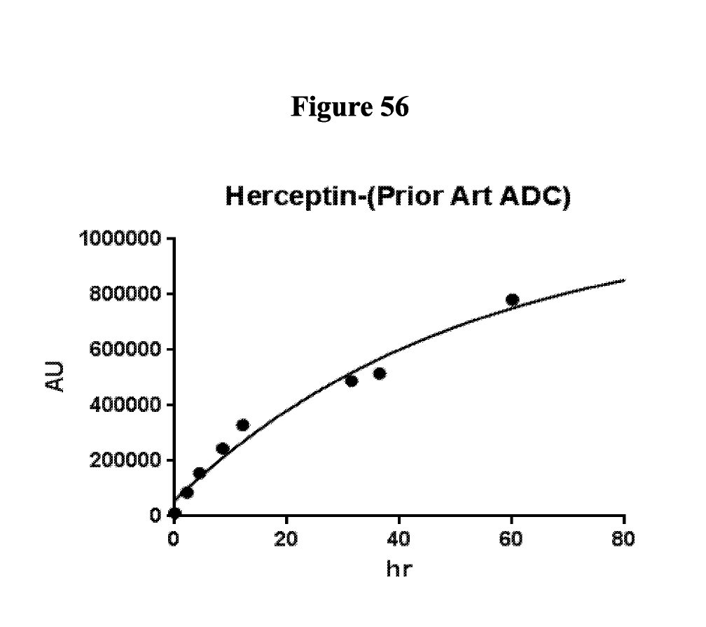

In one experiment, the release of free drug from the ADC of Herceptin-(Prior Art ADC) in PBS at 37° C. The calculated t_{1/2} is 35.7 hours. (FIG. 56).

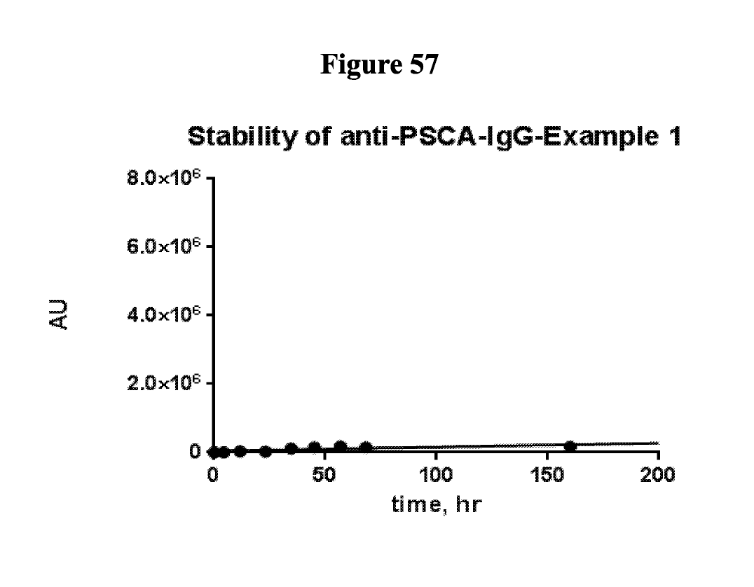

In another experiment, the release of free drug from the ADC of anti-PSCA-Example 1 in PBS at 37° C. The calculated t_{1/2} is 3480 hours. (FIG. 57).

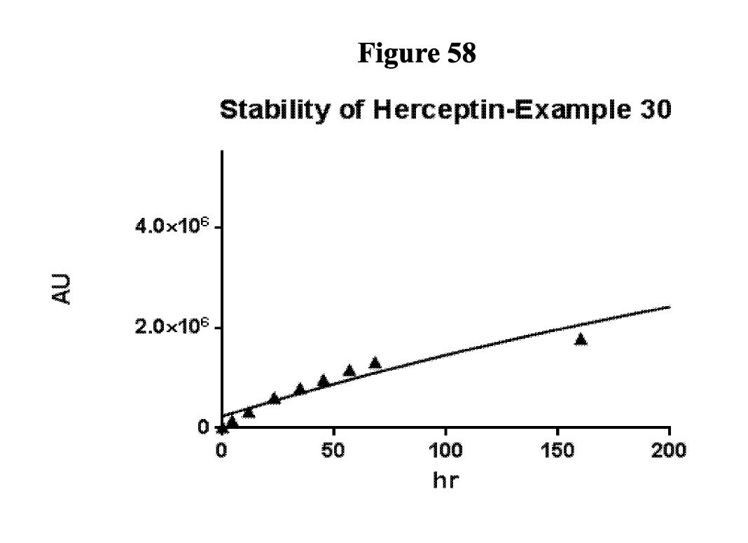

In another experiment, the release of free drug from the ADC of Herceptin-Example 30 in PBS at 37° C. The calculated t_{1/2} is 290 hours. (FIG. 58).

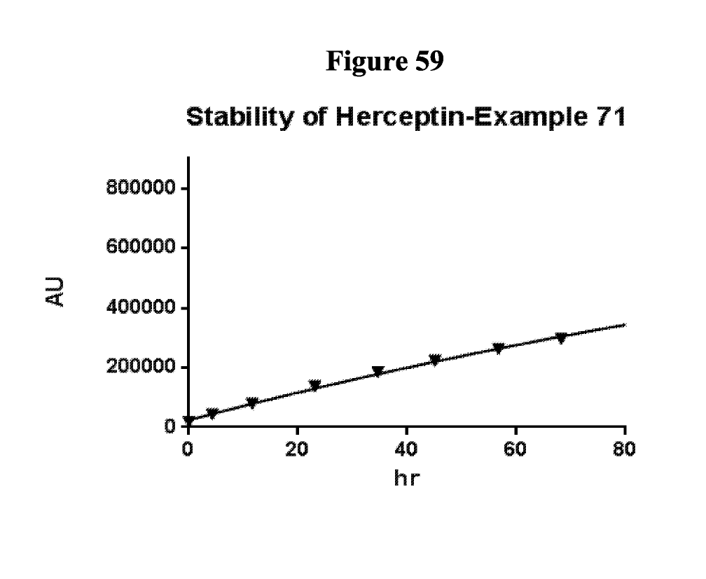

In another experiment, the release of free drug from the ADC of Herceptin-Example 71 in PBS at 37° C. The calculated t_{1/2} is 140 hours. (FIG. 59).

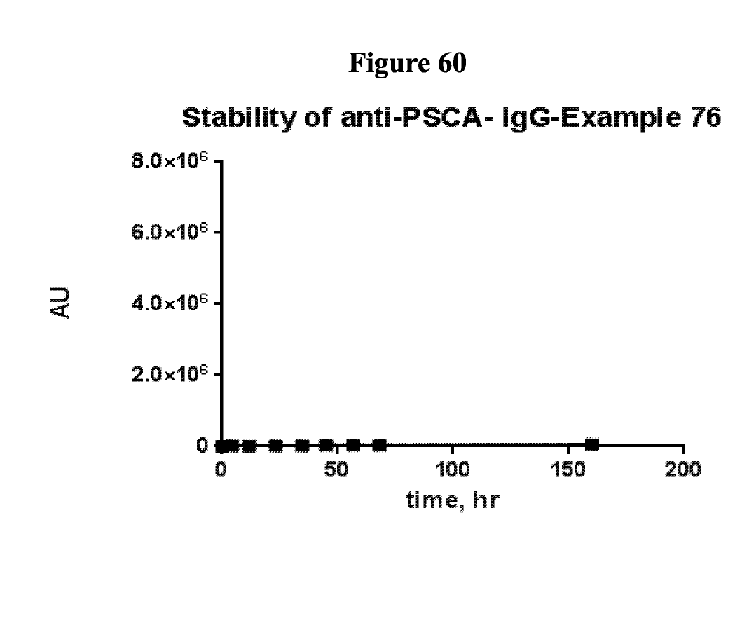

In another experiment, the release of free drug from the ADC of anti-PSCA-Example 76 in PBS at 37° C. The calculated t_{1/2} is 13000 hours. (FIG. 60).

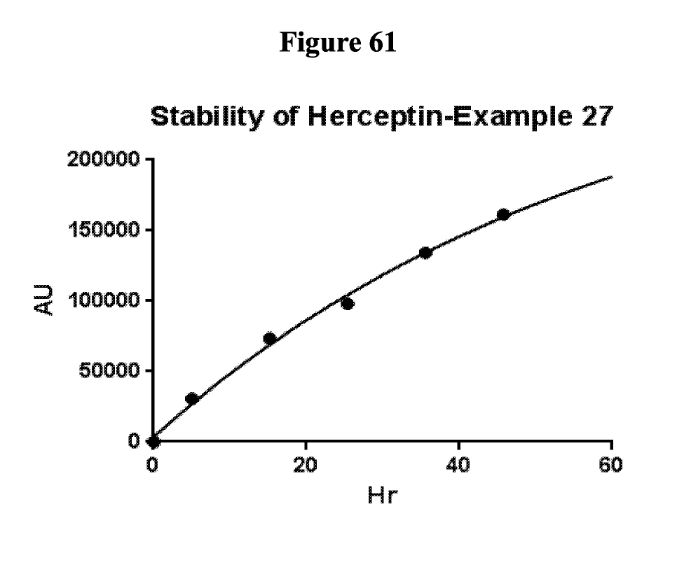

In another experiment, the release of free drug from the ADC of Herceptin-Example 27 in PBS at 37° C. The calculated t_{1/2} is 41 hours. (FIG. 61).

Conclusion. The results show that certain compounds of the present invention show substantially greater stability with regard to drug releasing from the ADC than the Prior Art ADC. Specifically, see FIG. 56 and FIG. 60. Accordingly, compounds of the present invention (e.g. Example 76, Example 1) are shown to be better tolerated after administration. Thus, compounds of the present invention can be used for therapeutic purposes to treat human cancers.

1. A compound of Formula (I): 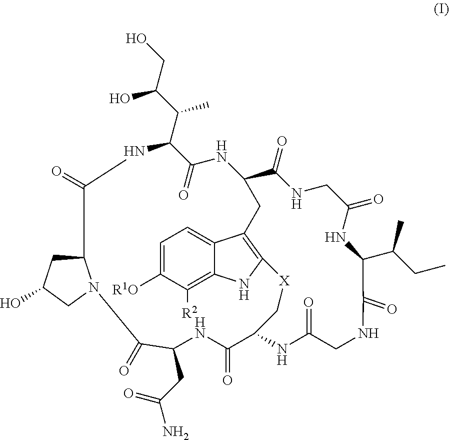
wherein:
X is S, SO, or SO₂;
(a) R¹ is H and R² is a chemical moiety of Formula (▴): 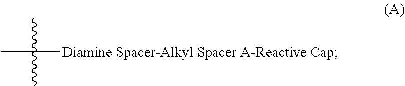
wherein
the diamine spacer is —NR^{x}—(C₂₋₂₀alkylene or C₂₋₂₀alkenylene)-NR^{y}-,
wherein the nitrogen of the —NR^{y}— group is attached to the alkyl spacer;
one carbon unit within the C₂₋₂₀alkylene or C₂₋₂₀alkenylene is optionally replaced with an NR^{z};
R^{x} is H or C₁₋₄alkyl, or
R^{x} taken together with a carbon or R^{z} within the alkylene or alkenylene forms a 3-8-membered heterocycloalkyl ring,
R^{y} is H or C₁₋₄alkyl,
or R^{x} and R^{y} taken together form a C₂₋₄alkylene; and
R^{z} is H or C₁₋₄alkyl;
the alkyl spacer A is absent, or is —C(O)C₁₋₂₀alkylene- or —C(O)C₂₋₂₀alkenylene-, wherein the carbonyl is attached to the nitrogen of the NR^{y} group in the diamine spacer and the alkylene or alkenylene is attached to the reactive cap, and wherein one or more carbon units within the alkylene or alkenylene is optionally replaced with C₃₋₇cycloalkylene, —C(O)NH—, —NHC(O)—, —C(O)O—, —OC(O)—, —C(O)—, —NH—, or —O—; and
the reactive cap is —N₃, —C≡CH, —CO₂H, —ONH₂, 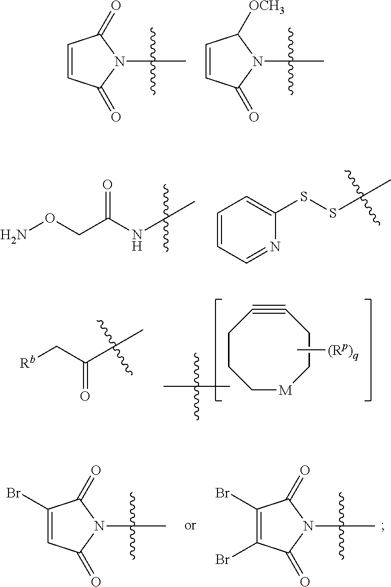
wherein R^{b} is a leaving group;
M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and
R^{m} is phenyl or a bond;
or
(b) R² is H and R¹ is a chemical moiety of Formula (B): 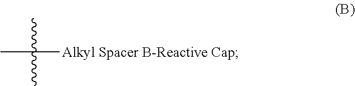
wherein the reactive cap is defined as above; and
alkyl spacer B is absent, or is —C₁₋₂₀alkylene- or —C₂₋₂₀alkenylene-, wherein one or more carbon units within the alkylene or alkenylene is replaced with C₃₋₇cycloalkylene, —C(O)NH—, —NHC(O)—, —C(O)O—, —OC(O)—, —C(O)—, —NH—, or —O—;
or a salt thereof.

2. A compound of Formula (IA): 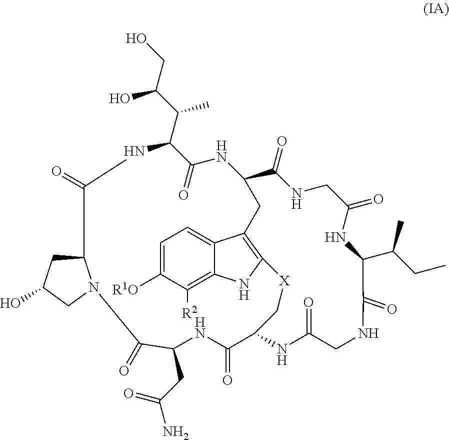
wherein:
X is S, SO, or SO₂;
(a) R¹ is H and R² is a chemical moiety of Formula (A-1): 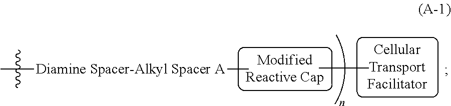
wherein
the diamine spacer and alkyl spacer A are defined as for Formula (I);
the modified reactive cap is —C(O)NH—, 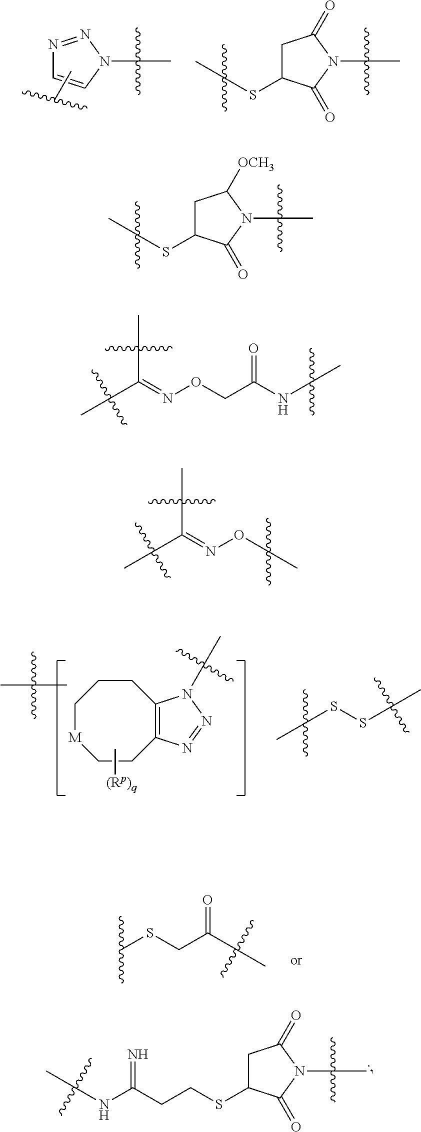
wherein M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond;
the cellular transport facilitator is an antibody, a peptide, a cationic polymer, or a liposome; and
n is an integer from 1 to 20;
or
(b) R₂ is H and R₁ is a chemical moiety of formula (B-1): 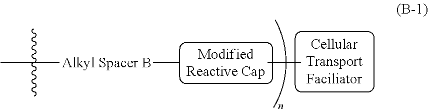
wherein alkyl spacer B is defined as for Formula I; and
the modified reactive cap, cellular transport facilitator, and n are as defined for Formula (A-1).

3. A compound of Formula (II): 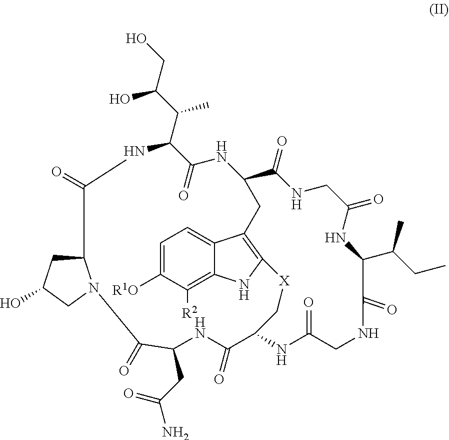
wherein:
X is S, SO, or SO₂;
(a) R¹ is H and R² is 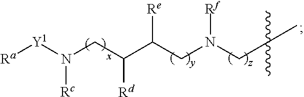
wherein x is 0, 1, or 2;
y is 0 or 1;
z is 0 or 1;
R^{c} is H or methyl;
R^{d} is H;
R^{e} is H;
R^{f} is H or methyl;
or R^{d} and R^{f} taken together form a bond, —CH₂—, or —CH₂CH₂—;
or R^{e} and R^{f} taken together form a bond;
or R^{c} and R^{f} taken together form —CH₂CH₂—;
Y¹ is absent, or is —C(O)C₁₋₁₆alkylene or —C(O)C₂₋₁₆alkenylene in which one or more carbon units are optionally replaced with C₃₋₇cycloalkylene, —C(O)NH—, —NHC(O)—, —C(O)O—, —OC(O)—, —C(O)—, NH, or O;
R^{a} is —N₃, —C≡CH, —CO₂H, —ONH₂, 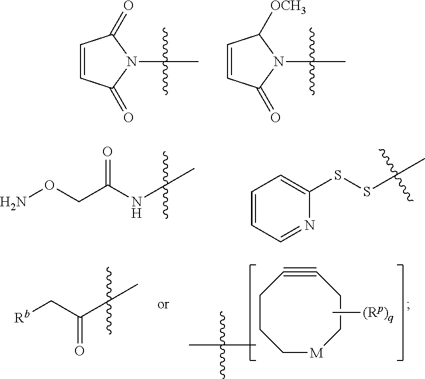
wherein R^{b} is a leaving group;
M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond; and
or
(b) R² is H and R¹ is 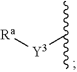
wherein Y³ is absent or is C₁₋₁₆alkylene or C₂₋₁₆alkenylene in which one or more carbon units are replaced with C₃₋₇cycloalkylene, —C(O)NH—, —NHC(O)—, —C(O)O—, —OC(O)—, —C(O)—, NH, or O; and
R^{a} is defined as above within the definition of R²;
or a pharmaceutically acceptable salt thereof.

4. A compound of claim 3, wherein
X is SO; and
(a) R¹ is H and R² is 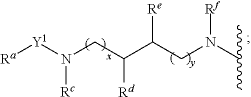
in which
(i) Y¹ is pentyl-(CO)—, R^{c} is H, R^{d} and R^{f} are taken together to form —CH₂CH₂—, R^{e} is H, x is 0, and y is 1, or
(ii) Y¹ is pentyl-(CO)—, R^{d} is H, R^{c} and R^{f} are taken together to form —CH₂CH₂—, R^{e} is H, x is 0, and y is 0; and
R^{a} is —N₃, —C≡CH, —CO₂H, —ONH₂, 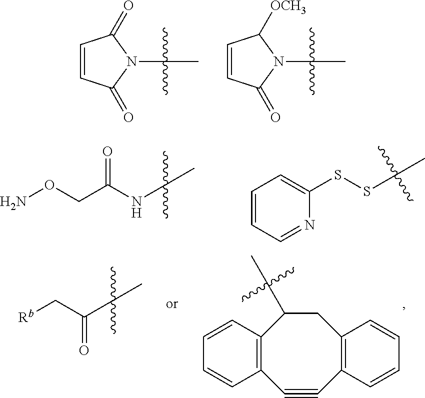
wherein R^{b} is a leaving group;
or
(b) R² is H and R¹ is 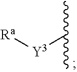
in which Y³ is -hexyl-NHC(O)-pentyl- or -pentyl-C(O)NH-hexyl-; and
R^{a} is defined above;
or a pharmaceutically acceptable salt thereof.

5. A compound of Formula (IIA): 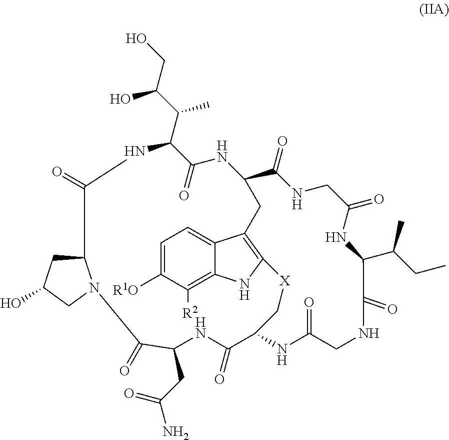
wherein:
X is S, SO, or SO₂;
(a) R¹ is H and R² is 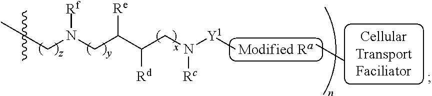
wherein x, y, z, R^{c}, R^{d}, R^{e}, R^{f}, and Y¹ are defined as for Formula (II); and
Modified R^{a} is —C(O)NH—, 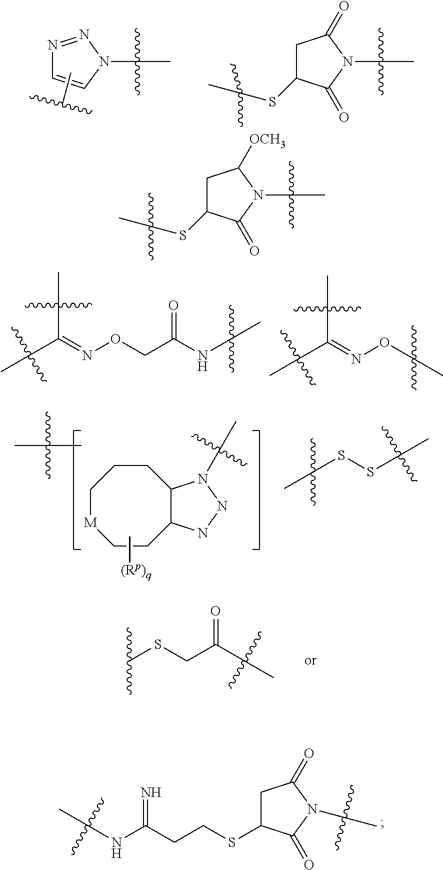
wherein M is CH₂ or NH;
q is 0, 1, 2, 3, or 4; and
each R^{p} is independently fluoro, hydroxy, methoxy, oxo, —O—CH₂—R^{m}—CO₂H, —CH₂—R^{m}—CO₂H, or —C(O)—(CH₂)₂—CO₂H; or two adjacent R^{p} groups taken together with the carbons to which they are attached form a phenyl or cyclopropyl ring, each optionally substituted with C₁₋₄alkyl, hydroxy, hydroxymethyl, or aminomethyl; and R^{m} is phenyl or a bond;
n is an integer from 1 to 20; and
the cellular transport facilitator is an antibody, a peptide, a cationic polymer, or a liposome;
or
(b) R² is H and R¹ is 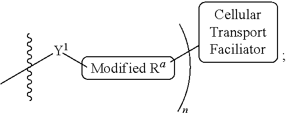
wherein Y³ is defined as for Formula (II); and
modified R^{a}, n, and cellular transport facilitator are defined as above for R².

6. A compound of claim 5, wherein
X is SO; and
(a) R¹ is H and R² is 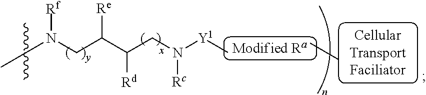
in which
(iii) Y¹ is pentyl-(CO)—, R^{c} is H, R^{d} and R^{f} are taken together to form —CH₂CH₂—, R^{e} is H, x is 0, and y is 1, or
(iv) Y¹ is pentyl-(CO)—, R^{d} is H, R^{c} and R^{f} are taken together to form —CH₂CH₂—, R^{e} is H, x is 0, and y is 0; and
the modified R^{a} is —C(O)NH—, or is: 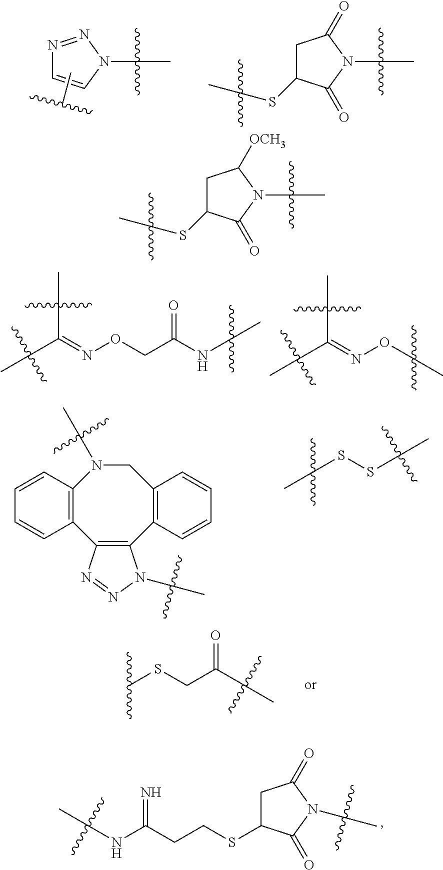
the cellular transport facilitator is an antibody, and
n is an integer from 1 to 20;
or
(b) R² is H and R¹ is 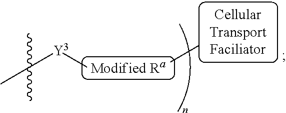
in which Y³ is -hexyl-NHC(O)-pentyl- or -pentyl-C(O)NH-hexyl-; and
the modified R^{a}, the cellular transport facilitator and n are each defined above;
or a pharmaceutically acceptable salt thereof.

7. A compound selected from the group consisting of:
7′C-(4-(6-(maleimido)hexanoyl)piperazin-1-yl)-α-amanitin;
7′C-(4-(6-(maleimido)hexanamido)piperidin-1-yl)-α-amanitin;
7′C-(4-(6-(6-(maleimido)hexanamido)hexanoyl)piperazin-1-yl)-α-amanitin;
7′C-(4-(4-((maleimido)methyl)cyclohexanecarbonyl)piperazin-1-yl)-α-amanitin;
7′C-(4-(6-(4-((maleimido)methyl)cyclohexanecarboxamido)hexanoyl)piperazin-1-yl)-α-amanitin;
7′C-(4-(2-(6-(maleimido)hexanamido)ethyl)piperidin-1-yl)-α-amanitin;
7′C-(4-(2-(6-(6-(maleimido)hexanamido)hexanamido)ethyl)piperidin-1-yl)-α-amanitin;
7′C-(4-(2-(4-((maleimido)methyl)cyclohexanecarboxamido)ethyl)piperidin-1-yl)-α-amanitin;
7′C-(4-(2-(6-(4-((maleimido)methyl)cyclohexanecarboxamido)hexanamido)ethyl)piperidin-1-yl)-α-amanitin;
7′C-(4-(2-(3-carboxypropanamido)ethyl)piperidin-1-yl)-α-amanitin;
7′C-(4-(2-(2-bromoacetamido)ethyl)piperidin-1-yl)-α-amanitin;
7′C-(4-(2-(3-(pyridin-2-yldisulfanyl)propanamido)ethyl)piperidin-1-yl)-α-amanitin;
7′C-(4-(2-(4-(maleimido)butanamido)ethyl)piperidin-1-yl)-α-amanitin;
7′C-(4-(2-(maleimido)acetyl)piperazin-1-yl)-α-amanitin;
7′C-(4-(3-(maleimido)propanoyl)piperazin-1-yl)-α-amanitin;
7′C-(4-(4-(maleimido)butanoyl)piperazin-1-yl)-α-amanitin;
7′C-(4-(2-(6-(4-((maleimido)methyl)cyclohexanecarboxamido)hexanamido)ethyl)piperidin-1-yl)-α-amanitin;
7′C-(3-((6-(maleimido)hexanamido)methyl)pyrrolidin-1-yl)-α-amanitin;
7′C-(3-((6-(6-(maleimido)hexanamido)hexanamido)methyl)pyrrolidin-1-yl)-α-amanitin;
7′C-(3-((4-((maleimido)methyl)cyclohexanecarboxamido)methyl)pyrrolidin-1-yl)-α-amanitin;
7′C-(3-((6-((4-(maleimido)methyl)cyclohexanecarboxamido)hexanamido) methyl)pyrrolidin-1-yl)-α-amanitin;
7′C-(4-(2-(6-(2-(aminooxy)acetamido)hexanamido)ethyl)piperidin-1-yl)-α-amanitin;
7′C-(4-(2-(4-(2-(aminooxy)acetamido)butanamido)ethyl)piperidin-1-yl)-α-amanitin;
7′C-(4-(4-(2-(aminooxy)acetamido)butanoyl)piperazin-1-yl)-α-amanitin;
7′C-(4-(6-(2-(aminooxy)acetamido)hexanoyl)piperazin-1-yl)-α-amanitin;
7′C-((4-(6-(maleimido)hexanamido)piperidin-1-yl)methyl)-α-amanitin;
7′C-((4-(2-(6-(maleimido)hexanamido)ethyl)piperidin-1-yl)methyl)-α-amanitin;
7′C-((4-(6-(maleimido)hexanoyl)piperazin-1-yl)methyl)-α-amanitin;
(R)-7′C-((3-((6-(maleimido)hexanamido)methyl)pyrrolidin-1-yl)methyl)-α-amanitin;
(S)-7′C-((3-((6-(maleimido)hexanamido)methyl)pyrrolidin-1-yl)methyl)-α-amanitin;
7′C-((4-(2-(6-(6-(maleimido)hexanamido)hexanamido)ethyl)piperidin-1-yl)methyl)-α-amanitin;
7′C-((4-(2-(4-((maleimido)methyl)cyclohexanecarboxamido)ethyl)piperidin-1-yl)methyl)-α-amanitin;
7′C-((4-(2-(6-(4-((maleimido)methyl)cyclohexanecarboxamido)hexanamido)ethyl)piperidin-1-yl)methyl)-α-amanitin;
7′C-((4-(2-(6-(maleimido)hexanamido)ethyl)piperazin-1-yl)methyl)-α-amanitin;
7′C-((4-(2-(6-(6-(maleimido)hexanamido)hexanamido)ethyl)piperazin-1-yl)methyl)-α-amanitin;
7′C-((4-(2-(4-((maleimido)methyl)cyclohexanecarboxamido)ethyl)piperazin-1-yl)methyl)-α-amanitin;
7′C-((4-(2-(6-(4-((maleimido)methyl)cyclohexanecarboxamido)hexanamido)ethyl)piperazin-1-yl)methyl)-α-amanitin;
7′C-((3-((6-(6-(maleimido)hexanamido)hexanamido)-S-methyl)pyrrolidin-1-yl)methyl)-α-amanitin;
7′C-((3-((6-(6-(maleimido)hexanamido)hexanamido)-R-methyl)pyrrolidin-1-yl)methyl)-α-amanitin;
7′C-((3-((4-((maleimido)methyl)cyclohexanecarboxamido)-S-methyl)pyrrolidin-1-yl)methyl)-α-amanitin;
7′C-((3-((4-((maleimido)methyl)cyclohexanecarboxamido)-R-methyl)pyrrolidin-1-yl)methyl)-α-amanitin;
7′C-((3-((6-(4-((maleimido)methyl)cyclohexanecarboxamido)hexanamido)methyl)pyrrolidin-1-yl)methyl)-α-amanitin;
7′C-((4-(2-(3-carboxypropanamido)ethyl)piperazin-1-yl)methyl)-α-amanitin;
7′C-((4-(6-(6-(maleimido)hexanamido)hexanoyl)piperazin-1-yl)methyl)-α-amanitin;
7′C-((4-(6-(4-((maleimido)methyl)cyclohexanecarboxamido)hexanoyl)piperazin-1-yl)methyl)-α-amanitin;
7′C-((4-(2-(maleimido)acetyl)piperazin-1-yl)methyl)-α-amanitin;
7′C-((4-(3-(maleimido)propanoyl)piperazin-1-yl)methyl)-α-amanitin;
7′C-((4-(4-(maleimido)butanoyl)piperazin-1-yl)methyl)-α-amanitin;
7′C-((4-(2-(2-(maleimido)acetamido)ethyl)piperidin-1-yl)methyl)-α-amanitin;
7′C-((4-(2-(4-(maleimido)butanamido)ethyl)piperidin-1-yl)methyl)-α-amanitin;
7′C-((4-(2-(6-(4-((maleimido)methyl)cyclohexanecarboxamido)hexanamido)ethyl)piperidin-1-yl)methyl)-α-amanitin;
7′C-((3-((6-(maleimido)hexanamido)methyl)azetidin-1-yl)methyl)-α-amanitin;
7′C-((3-(2-(6-(maleimido)hexanamido)ethyl)azetidin-1-yl)methyl)-α-amanitin;
7′C-((3-((4-((maleimido)methyl)cyclohexanecarboxamido)methyl)azetidin-1-yl)methyl)-α-amanitin;
7′C-((3-(2-(4-((maleimido)methyl)cyclohexanecarboxamido)ethyl)azetidin-1-yl)methyl)-α-amanitin;
7′C-((3-(2-(6-(4-((maleimido)methyl)cyclohexanecarboxamido)hexanamido)ethyl)azetidin-1-yl)methyl)-α-amanitin;
7′C-(((2-(6-(maleimido)-N-methylhexanamido)ethyl)(methyl)amino)methyl)-α-amanitin;
7′C-(((4-(6-(maleimido)-N-methylhexanamido)butyl(methyl)amino)methyl)-α-amanitin;
7′C-((2-(2-(6-(maleimido)hexanamido)ethyl)aziridin-1-yl)methyl)-α-amanitin;
7′C-((2-(2-(6-(4-((maleimido)methyl)cyclohexanecarboxamido)hexanamido)ethyl)aziridin-1-yl)methyl)-α-amanitin;
7′C-((4-(6-(6-(2-(aminooxy)acetamido)hexanamido)hexanoyl)piperazin-1-yl)methyl)-α-amanitin;
7′C-((4-(1-(aminooxy)-2-oxo-6,9,12,15-tetraoxa-3-azaheptadecan-17-oyl)piperazin-1-yl)methyl)-α-amanitin;
7′C-((4-(2-(2-(aminooxy)acetamido)acetyl)piperazin-1-yl)methyl)-α-amanitin;
7′C-((4-(3-(2-(aminooxy)acetamido)propanoyl)piperazin-1-yl)methyl)-α-amanitin;
7′C-((4-(4-(2-(aminooxy)acetamido)butanoyl)piperazin-1-yl)methyl)-α-amanitin;
7′C-((4-(2-(6-(2-(aminooxy)acetamido)hexanamido)ethyl)piperidin-1-yl)methyl)-α-amanitin;
7′C-((4-(2-(2-(2-(aminooxy)acetamido)acetamido)ethyl)piperidin-1-yl)methyl)-α-amanitin;
7′C-((4-(2-(4-(2-(aminooxy)acetamido)butanamido)ethyl)piperidin-1-yl)methyl)-α-amanitin;
7′C-((4-(20-(aminooxy)-4,19-dioxo-6,9,12,15-tetraoxa-3,18-diazaicosyl)piperidin-1-yl)methyl)-α-amanitin;
7′C-(((2-(6-(2-(aminooxy)acetamido)-N-methylhexanamido)ethyl)(methyl)amino)methyl)-α-amanitin;
7′C-(((4-(6-(2-(aminooxy)acetamido)-N-methylhexanamido)butyl)(methyl)amino)methyl)-α-amanitin;
7′C-((3-((6-(4-((maleimido)methyl)cyclohexanecarboxamido)hexanamido)methyl)pyrrolidin-1-yl)-S-methyl)-α-amanitin;
7′C-((3-((6-(4-((maleimido)methyl)cyclohexanecarboxamido)hexanamido)-R-methyl)pyrrolidin-1-yl)methyl)-α-amanitin;
7′C-((4-(2-(2-bromoacetamido)ethyl)piperazin-1-yl)methyl)-α-amanitin;
7′C-((4-(2-(2-bromoacetamido)ethyl)piperidin-1-yl)methyl)-α-amanitin;
7′C-((4-(2-(3-(pyridine-2-yldisulfanyl)propanamido)ethyl)piperidin-1-yl)methyl)-α-amanitin;
6′O-(6-(6-(maleimido)hexanamido)hexyl)-α-amanitin;
6′O-(5-(4-((maleimido)methyl)cyclohexanecarboxamido)pentyl)-α-amanitin;
6′O-(2-((6-(maleimido)hexyl)oxy)-2-oxoethyl)-α-amanitin;
6′O-((6-(maleimido)hexyl)carbamoyl)-α-amanitin;
6′O-((6-(4-((maleimido)methyl)cyclohexanecarboxamido)hexyl)carbamoyl)-α-amanitin;
6′O-(6-(2-bromoacetamido)hexyl)-α-amanitin;
7′C-(4-(6-(azido)hexanamido)piperidin-1-yl)-α-amanitin;
7′C-(4-(hex-5-ynoylamino)piperidin-1-yl)-α-amanitin;
7′C-(4-(2-(6-(maleimido)hexanamido)ethyl)piperazin-1-yl)-α-amanitin;
7′C-(4-(2-(6-(6-(maleimido)hexanamido)hexanamido)ethyl)piperazin-1-yl)-α-amanitin;
6′O-(6-(6-(11,12-didehydro-5,6-dihydro-dibenz[b,f]azocin-5-yl)-6-oxohexanamido)hexyl)-α-amanitin;
6′O-(6-(hex-5-ynoylamino)hexyl)-α-amanitin;
6′O-(6-(2-(aminooxy)acetylamido)hexyl)-α-amanitin;
6′O-((6-aminooxy)hexyl)-α-amanitin; and
6′O-(6-(2-iodoacetamido)hexyl)-α-amanitin;
and pharmaceutically acceptable salts thereof.

8. The compound of claim 2, or a pharmaceutically acceptable salt thereof, wherein the chemical entity bound to the cellular transport facilitator is selected from the group consisting of:
7′C-(4-(6-(maleimido)hexanoyl)piperazin-1-yl)-α-amanitin;
7′C-(4-(6-(maleimido)hexanamido)piperidin-1-yl)-α-amanitin;
7′C-(4-(6-(6-(maleimido)hexanamido)hexanoyl)piperazin-1-yl)-α-amanitin;
7′C-(4-(4-((maleimido)methyl)cyclohexanecarbonyl)piperazin-1-yl)-α-amanitin;
7′C-(4-(6-(4-((maleimido)methyl)cyclohexanecarboxamido)hexanoyl)piperazin-1-yl)-α-amanitin;
7′C-(4-(2-(6-(maleimido)hexanamido)ethyl)piperidin-1-yl)-α-amanitin;
7′C-(4-(2-(6-(6-(maleimido)hexanamido)hexanamido)ethyl)piperidin-1-yl)-α-amanitin;
7′C-(4-(2-(4-((maleimido)methyl)cyclohexanecarboxamido)ethyl)piperidin-1-yl)-α-amanitin;
7′C-(4-(2-(6-(4-((maleimido)methyl)cyclohexanecarboxamido)hexanamido)ethyl)piperidin-1-yl)-α-amanitin;
7′C-(4-(2-(3-carboxypropanamido)ethyl)piperidin-1-yl)-α-amanitin;
7′C-(4-(2-(2-bromoacetamido)ethyl)piperidin-1-yl)-α-amanitin;
7′C-(4-(2-(3-(pyridin-2-yldisulfanyl)propanamido)ethyl)piperidin-1-yl)-α-amanitin;
7′C-(4-(2-(4-(maleimido)butanamido)ethyl)piperidin-1-yl)-α-amanitin;
7′C-(4-(2-(maleimido)acetyl)piperazin-1-yl)-α-amanitin;
7′C-(4-(3-(maleimido)propanoyl)piperazin-1-yl)-α-amanitin;
7′C-(4-(4-(maleimido)butanoyl)piperazin-1-yl)-α-amanitin;
7′C-(4-(2-(6-(4-((maleimido)methyl)cyclohexanecarboxamido)hexanamido)ethyl)piperidin-1-yl)-α-amanitin;
7′C-(3-((6-(maleimido)hexanamido)methyl)pyrrolidin-1-yl)-α-amanitin;
7′C-(3-((6-(6-(maleimido)hexanamido)hexanamido)methyl)pyrrolidin-1-yl)-α-amanitin;
7′C-(3-((4-((maleimido)methyl)cyclohexanecarboxamido)methyl)pyrrolidin-1-yl)-α-amanitin;
7′C-(3-((6-(4-((maleimido)methyl)cyclohexanecarboxamido)hexanamido)methyl)pyrrolidin-1-yl)-α-amanitin;
7′C-(4-(2-(6-(2-(aminooxy)acetamido)hexanamido)ethyl)piperidin-1-yl)-α-amanitin;
7′C-(4-(2-(4-(2-(aminooxy)acetamido)butanamido)ethyl)piperidin-1-yl)-α-amanitin;
7′C-(4-(4-(2-(aminooxy)acetamido)butanoyl)piperazin-1-yl)-α-amanitin;
7′C-(4-(6-(2-(aminooxy)acetamido)hexanoyl)piperazin-1-yl)-α-amanitin;
7′C-((4-(6-(maleimido)hexanamido)piperidin-1-yl)methyl)-α-amanitin;
7′C-((4-(2-(6-(maleimido)hexanamido)ethyl)piperidin-1-yl)methyl)-α-amanitin;
7′C-((4-(6-(maleimido)hexanoyl)piperazin-1-yl)methyl)-α-amanitin;
(R)-7′C-((3-((6-(maleimido)hexanamido)methyl)pyrrolidin-1-yl)methyl)-α-amanitin;
(S)-7′C-((3-((6-(maleimido)hexanamido)methyl)pyrrolidin-1-yl)methyl)-α-amanitin;
7′C-((4-(2-(6-(6-(maleimido)hexanamido)hexanamido)ethyl)piperidin-1-yl)methyl)-α-amanitin;
7′C-((4-(2-(4-((maleimido)methyl)cyclohexanecarboxamido)ethyl)piperidin-1-yl)methyl)-α-amanitin;
7′C-((4-(2-(6-(4-((maleimido)methyl)cyclohexanecarboxamido)hexanamido)ethyl)piperidin-1-yl)methyl)-α-amanitin;
7′C-((4-(2-(6-(maleimido)hexanamido)ethyl)piperazin-1-yl)methyl)-α-amanitin;
7′C-((4-(2-(6-(6-(maleimido)hexanamido)hexanamido)ethyl)piperazin-1-yl)methyl)-α-amanitin;
7′C-((4-(2-(4-((maleimido)methyl)cyclohexanecarboxamido)ethyl)piperazin-1-yl)methyl)-α-amanitin;
7′C-((4-(2-(6-(4-((maleimido)methyl)cyclohexanecarboxamido)hexanamido)ethyl)piperazin-1-yl)methyl)-α-amanitin;
7′C-((3-((6-(6-(maleimido)hexanamido)hexanamido)-S-methyl)pyrrolidin-1-yl)methyl)-α-amanitin;
7′C-((3-((6-(6-(maleimido)hexanamido)hexanamido)-R-methyl)pyrrolidin-1-yl)methyl)-α-amanitin;
7′C-((3-((4-((maleimido)methyl)cyclohexanecarboxamido)-S-methyl)pyrrolidin-1-yl)methyl)-α-amanitin;
7′C-((3-((4-((maleimido)methyl)cyclohexanecarboxamido)-R-methyl)pyrrolidin-1-yl)methyl)-α-amanitin;
7′C-((3-((6-(4-((maleimido)methyl)cyclohexanecarboxamido)hexanamido)methyl)pyrrolidin-1-yl)methyl)-α-amanitin;
7′C-((4-(2-(3-carboxypropanamido)ethyl)piperazin-1-yl)methyl)-α-amanitin;
7′C-((4-(6-(6-(maleimido)hexanamido)hexanoyl)piperazin-1-yl)methyl)-α-amanitin;
7′C-((4-(6-(4-((maleimido)methyl)cyclohexanecarboxamido)hexanoyl)piperazin-1-yl)methyl)-α-amanitin;
7′C-((4-(2-(maleimido)acetyl)piperazin-1-yl)methyl)-α-amanitin;
7′C-((4-(3-(maleimido)propanoyl)piperazin-1-yl)methyl)-α-amanitin;
7′C-((4-(4-(maleimido)butanoyl)piperazin-1-yl)methyl)-α-amanitin;
7′C-((4-(2-(2-(maleimido)acetamido)ethyl)piperidin-1-yl)methyl)-α-amanitin;
7′C-((4-(2-(4-(maleimido)butanamido)ethyl)piperidin-1-yl)methyl)-α-amanitin;
7′C-((4-(2-(6-(4-((maleimido)methyl)cyclohexanecarboxamido)hexanamido)ethyl)piperidin-1-yl)methyl)-α-amanitin;
7′C-((3-((6-(maleimido)hexanamido)methyl)azetidin-1-yl)methyl)-α-amanitin;
7′C-((3-(2-(6-(maleimido)hexanamido)ethyl)azetidin-1-yl)methyl)-α-amanitin;
7′C-((3-((4-((maleimido)methyl)cyclohexanecarboxamido)methyl)azetidin-1-yl)methyl)-α-amanitin;
7′C-((3-(2-(4-((maleimido)methyl)cyclohexanecarboxamido)ethyl)azetidin-1-yl)methyl)-α-amanitin;
7′C-((3-(2-(6-(4-((maleimido)methyl)cyclohexanecarboxamido)hexanamido)ethyl)azetidin-1-yl)methyl)-α-amanitin;
7′C-(((2-(6-(maleimido)-N-methylhexanamido)ethyl)(methyl)amino)methyl)-α-amanitin;
7′C-(((4-(6-(maleimido)-N-methylhexanamido)butyl(methyl)amino)methyl)-α-amanitin;
7′C-((2-(2-(6-(maleimido)hexanamido)ethyl)aziridin-1-yl)methyl)-α-amanitin;
7′C-((2-(2-(6-(4-((maleimido)methyl)cyclohexanecarboxamido)hexanamido)ethyl)aziridin-1-yl)methyl)-α-amanitin;
7′C-((4-(6-(6-(2-(aminooxy)acetamido)hexanamido)hexanoyl)piperazin-1-yl)methyl)-α-amanitin;
7′C-((4-(1-(aminooxy)-2-oxo-6,9,12,15-tetraoxa-3-azaheptadecan-17-oyl)piperazin-1-yl)methyl)-α-amanitin;
7′C-((4-(2-(2-(aminooxy)acetamido)acetyl)piperazin-1-yl)methyl)-α-amanitin;
7′C-((4-(3-(2-(aminooxy)acetamido)propanoyl)piperazin-1-yl)methyl)-α-amanitin;
7′C-((4-(4-(2-(aminooxy)acetamido)butanoyl)piperazin-1-yl)methyl)-α-amanitin;
7′C-((4-(2-(6-(2-(aminooxy)acetamido)hexanamido)ethyl)piperidin-1-yl)methyl)-α-amanitin;
7′C-((4-(2-(2-(2-(aminooxy)acetamido)acetamido)ethyl)piperidin-1-yl)methyl)-α-amanitin;
7′C-((4-(2-(4-(2-(aminooxy)acetamido)butanamido)ethyl)piperidin-1-yl)methyl)-α-amanitin;
7′C-((4-(20-(aminooxy)-4,19-dioxo-6,9,12,15-tetraoxa-3,18-diazaicosyl)piperidin-1-yl)methyl)-α-amanitin;
7′C-(((2-(6-(2-(aminooxy)acetamido)-N-methylhexanamido)ethyl)(methyl)amino)methyl)-α-amanitin;
7′C-(((4-(6-(2-(aminooxy)acetamido)-N-methylhexanamido)butyl)(methyl)amino)methyl)-α-amanitin;
7′C-((3-((6-(4-((maleimido)methyl)cyclohexanecarboxamido)hexanamido)methyl)pyrrolidin-1-yl)-S-methyl)-α-amanitin;
7′C-((3-((6-(4-((maleimido)methyl)cyclohexanecarboxamido)hexanamido)-R-methyl)pyrrolidin-1-yl)methyl)-α-amanitin;
7′C-((4-(2-(2-bromoacetamido)ethyl)piperazin-1-yl)methyl)-α-amanitin;
7′C-((4-(2-(2-bromoacetamido)ethyl)piperidin-1-yl)methyl)-α-amanitin;
7′C-((4-(2-(3-(pyridine-2-yldisulfanyl)propanamido)ethyl)piperidin-1-yl)methyl)-α-amanitin;
6′O-(6-(6-(maleimido)hexanamido)hexyl)-α-amanitin;
6′O-(5-(4-((maleimido)methyl)cyclohexanecarboxamido)pentyl)-α-amanitin;
6′O-(2-((6-(maleimido)hexyl)oxy)-2-oxoethyl)-α-amanitin;
6′O-((6-(maleimido)hexyl)carbamoyl)-α-amanitin;
6′O-((6-(4-((maleimido)methyl)cyclohexanecarboxamido)hexyl)carbamoyl)-α-amanitin;
6′O-(6-(2-bromoacetamido)hexyl)-α-amanitin;
7′C-(4-(6-(azido)hexanamido)piperidin-1-yl)-α-amanitin;
7′C-(4-(hex-5-ynoylamino)piperidin-1-yl)-α-amanitin;
7′C-(4-(2-(6-(maleimido)hexanamido)ethyl)piperazin-1-yl)-α-amanitin;
7′C-(4-(2-(6-(6-(maleimido)hexanamido)hexanamido)ethyl)piperazin-1-yl)-α-amanitin;
6′O-(6-(6-(11,12-didehydro-5,6-dihydro-dibenz[b,f]azocin-5-yl)-6-oxohexanamido)hexyl)-α-amanitin;
6′O-(6-(hex-5-ynoylamino)hexyl)-α-amanitin;
6′O-(6-(2-(aminooxy)acetylamido)hexyl)-α-amanitin;
6′O-((6-aminooxy)hexyl)-α-amanitin; and
6′O-(6-(2-iodoacetamido)hexyl)-α-amanitin.

9. A pharmaceutical composition comprising a compound according to any one of claims 2, 5, or 8, or a pharmaceutically acceptable salt thereof, and a pharmaceutically acceptable carrier.

10. A method of treating a disorder in a subject, comprising administering to a subject in need of such treatment an effective amount of a compound of any one of claims 2, 5, or 8, or a pharmaceutically acceptable salt thereof, wherein the disorder is cancer, an autoimmune disease, or an infectious disease.

11. A method for making a conjugate of a drug and a cellular transport facilitator (CTF); the conjugate having the structure as described in any of claims 2, 5, or 6, the method comprising the steps of:
(a) reacting a reactive cap of an activated drug moiety D with a CTF, whereby the conjugate of the drug and CTF is formed;
or comprising the steps of:
(b) reacting the CTF with an activating reagent to form a CTF intermediate (CTF-I); and
(c) reacting CTF-I with a reactive cap of an activated drug moiety D, whereby the conjugate of the drug and CTF is formed;
wherein the activated drug moiety D is a compound as described in any of claims 1, 3, 4 or 7.

12. The method of claim 11, wherein the CTF is an antibody or an antigen-binding fragment thereof.
